(19) United States
(12) Patent Application Publication
(10) Pub. No.: US 2025/0115905 A1
(43) Pub. Date: Apr. 10, 2025

(54) Methods of Rescuing Stop Codons via Genetic Reassignment with ACE-tRNA

(71) Applicants: THE WISTAR INSTITUTE OF ANATOMY AND BIOLOGY, Philadelphia, PA (US); University of Iowa Research Foundation, Iowa City, IA (US)

(72) Inventors: Alfredo Perales PUCHALT, Philadelphia, PA (US); John LUECK, Rochester, NY (US); Christopher A. AHERN, Iowa City, IA (US); David B. WEINER, Merion, PA (US)

(21) Appl. No.: 18/430,760

(22) Filed: Feb. 2, 2024

Related U.S. Application Data
(63) Continuation of application No. 16/760,932, filed on May 1, 2020, now abandoned.
(60) Provisional application No. 62/580,887, filed on Nov. 2, 2017.
(60) Provisional application No. 62/687,015, filed on Jun. 19, 2018.

(51) Int. Cl. C12N 15/113; A61K 9/127; A61K 31/7105; A61P 43/00

(52) U.S. Cl. CPC C12N 15/113; A61K 9/127; A61K 31/7105; A61P 43/00

(57) ABSTRACT

The present invention provides compositions and methods for treating diseases and disorders associated with premature termination codons comprising administration of nucleic acid molecules comprising or encoding anticodon edited tRNAs.

### CROSS-REFERENCE TO RELATED APPLICATION

This application is a continuation of U.S. national stage application No. 16/760,932, filed Nov. 5, 2018, which is a U.S. national phase application filed under 35 U.S.C. § 371 claiming priority to International Patent Application No. PCT/US18/59085, filed Nov. 2, 2018, which is entitled to priority under 35 U.S.C. § 119 (e) to U.S. Provisional Application Ser. No. 62/580,887, filed Nov. 2, 2017, and to U.S. Provisional Application Ser. No. 62/687,015, filed Jun. 20, 2018, each of which is incorporated by reference herein in its entirety.

### STATEMENT REGARDING FEDERALLY SPONSORED RESEARCH OR DEVELOPMENT

This invention was made with government support under R01 GM106569 awarded by the National Institutes of Health. The Government has certain rights in the invention.

### REFERENCE TO A “SEQUENCE LISTING” SUBMITTED AS AN XML FILE

The Sequence Listing written in the XML file: “206193-0018-01US_SequenceListing.XML”; created on Jan. 29, 2024, and 885,810 bytes in size, is hereby incorporated by reference.

### BACKGROUND

DNA molecules carry genetic information in the form of the sequence of the nucleotide bases that make up the DNA polymer. Only four nucleotide bases are utilized in DNA: adenine, guanine, cytosine, and thymine. This information, in the form of codons of three contiguous bases is transcribed into messenger RNA (mRNA), and then translated by transfer RNA (tRNA) and ribosomes to form proteins. Four nucleotide bases are utilized in RNA: adenine, guanine, cytosine, and uracil. The genetic code is the relation between a triplet codon and a particular amino acid. Sixty-four possible codon triplets form the genetic code, where three stop (also called terminating) codons, which provide a signal to the translation machinery (cellular ribosomes) to stop protein production at the particular codon. The other sixty-one triplets in the code correspond to one of the 20 standard amino acid. See FIG. 1.

DNA is translated by ribosomes, causing each amino acid to be linked together one by one to form polypeptides, according to the genetic instructions specifically provided by the DNA. When the ribosome reaches a stop codon, the elongation of the protein terminates. The three stop codons are UAG (amber), UAA (ochre) and UGA (opal). Mutations that occur that change an amino acid-encoding codon to stop codon are called “nonsense mutations.” These nonsense mutations can result in a significant truncation/shortening of the polypeptide sequence, and can cause a profound change in genetic phenotype. Thus, even though a gene directing expression may be present, a crucial protein may not be produced because when the ribosome reaches the mutant stop signal, it terminates translation resulting in an unfinished protein.

Transfer RNAs translate mRNA into a protein on a ribosome. Each tRNA contains an “anti-codon” region that hybridizes with a complementary codon on the mRNA. A tRNA that carries its designated amino acid is called a “charged” tRNA. If the tRNA is one of the 61 amino-acid-associated (i.e., not a stop-signal-associated) tRNAs, it will normally attach its amino acid to the growing peptide. The structural gene of tRNA is about 72-90 nucleotides long and folds into a cloverleaf structure. tRNAs are transcribed by RNA polymerase III and contain their own intragenic split promoters that become a part of the mature tRNA coding sequence (Sharp S. J., Schaack J., Coolen L., Burke D. J. and Soll D., “Structure and transcription of eukaryotic (RNA genes”, Crit. Rev. Biochem, 19:107-144 (1985); Geiduschek E. O., and Tocchini-Valentini, “Transcription by RNA polymerase III, Annu. Rev. Biochem. 57:873-914 (1988)).

“Nonsense suppressors” are alleles of tRNA genes that contain an altered anticodon, such that instead of triggering a “stop” signal, they insert an amino acid in response to a termination codon. For example, an ochre mutation results in the creation of a UAA codon in an mRNA. An ochre suppressor gene produces tRNA with an AUU anticodon that inserts an amino acid at the UAA site, which permits the continued translation of the mRNA despite the presence of a codon that would normally trigger a stop in translation.

A number of nonsense suppressor tRNA alleles have been identified in prokaryotes and eukaryotes such as yeast and C. elegans. The different suppressor tRNAs vary in their suppression efficiency. In E. coli and other systems, the amber suppressors are relatively more efficient, ochre suppressors are less efficient while opal are the least, this suggests that the amber codons are used infrequently to terminate protein synthesis, while ochre and opal codons are more frequently used as natural termination signals.

Unwanted errors in the DNA blueprint can cause disease. For example, the occurrence of an unexpected “stop” signal in the middle of the protein, rather than at the end of the blueprint, results in the production of a truncated or shortened protein that has an altered function, or no function at all. Many human diseases, including cystic fibrosis (Cheng et al., 1990, Cell, 63, 827-834), Duchenne muscular dystrophy, spinal muscular atrophy (Lefebvre et al., 1995, Cell, 80, 155-165), infantile neuronal ceroid lipofuscinosis (Das et al., 1998, J Clin Invest, 102, 361-370), β o-thalessemia (Chang and Kan, 1979, Proc Natl Acad Sci USA, 76, 2886-2889), cystinosis (Kalatzis et al., 2002, Hum Mutat, 20, 439-446), X-linked nephrogenic diabetes insipidus (Pan et al., 1992, Nat Genet, 2, 103-106), Hurler syndrome (Ballabio et al., 2009, Biochim Biophys Acta, 1793, 684-696), Usher syndrome 11, polycystic kidney disease, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and rickets result from unwanted stop signals in DNA reading frames for proteins. Additionally, nonsense mutations occur within the tumor suppressor genes p53 and ATM, further implicating their role in disease. As such, PTCs represent a unique constellation of diseases which afflict over 30 million people worldwide, accounting for 10-15% of all genetic diseases.

There is thus a need in the art for compositions and methods that are generalizable to multiple tRNA gene families for treatment of diseases and disorders associated with PTCs. The present invention addresses this unmet need in the art

### SUMMARY

In certain embodiments, the present invention provides a modified transfer RNA (tRNA) comprising a T-arm, a D-arm, and anticodon arm and an acceptor arm, wherein the T-arm comprises a T-stem having nucleotides that interact with Elongation Factor 1-alpha 1 (EF1alpha).□ EF1alpha recruits aminoacyl-tRNA to the ribosome and protects the tRNA from being deacylated. Rational nucleotide replacement results in a tuned tRNA: EF1□ interaction that enhances tRNA delivery to the ribosome and protection from deacylation.

In certain embodiments, the present invention provides a modified transfer RNA (tRNA) of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54 or 55, wherein the thymidines are replaced with uracils.

In certain embodiments, the present invention provides a modified transfer RNA (tRNA) of any one of SEQ ID NO: 1-538, wherein the thymidines are replaced with uracils.

In certain embodiments, the modified tRNA is any one of SEQ ID NOs: 56-60, 62-66, 84-86, 90-111, 113, 128-143, 147-149, 153-156, 161-174, 176, 178, 181, 184-186, 192, 196-197, 199-201, 205, 213-240, 246, 255-256, 258-285, 299, 305-312, 314, 318-332, 335-344, 346, 350-354, 357-360, 362, 365-370, 372-383, 388-390, 392, 394-401, 403-407, 414-416, 418, 422, 425, 428-433, 437, 444-445, 452, 455, 459-463, 470, 472-474, 476, 487-492, 525, 530-539, 545-550, 553-555, 561-563, and 567-579, wherein the thymidines are replaced with uracils.

In certain embodiments, the present invention provides a modified transfer RNA (tRNA) comprising a T-stem, a D-stem, and anticodon-loop and an acceptor stem, wherein (a) wherein the anticodon-arm comprises a tri-nucleotide anticodon, wherein the anticodon is 5′-UCA-3′ and recognizes TGA stop codons, and wherein the acceptor arm is operably linked to a arginine, tryptophan or glycine; (b) wherein the anticodon-arm comprises a tri-nucleotide anticodon, wherein the anticodon is 5′-UUA-3′ and recognizes TAA stop codons, and wherein the acceptor arm is operably linked to a glutamine or, glutamate; or (c) wherein the anticodon-arm comprises a tri-nucleotide anticodon, wherein the anticodon is 5′-CUA-3′ and recognizes TAG stop codons, and wherein the acceptor arm is operably linked to a tryptophan, glutamate or glutamine. In certain embodiments, the T-arm comprises rationally altered nucleotide sequences that tune the interaction with the EFI, enhancing its suppression activity and thereby increasing its therapeutic potential. tRNAs with tuned interaction with the EF1alpha have enhanced nonsense suppression and provide enhanced therapeutic properties.

In certain embodiments, the present invention provides an oligonucleotide sequence that encodes the modified tRNA as described above, wherein the oligonucleotide has a total length of less than 150 nucleotides. In certain embodiments, the oligonucleotide is DNA.

In certain embodiments, the present invention provides an oligonucleotide comprising a first oligonucleotide sequence and a second oligonucleotide sequence, wherein the first and second oligonucleotide sequences independently encode a modified tRNA as described above, wherein the first and second oligonucleotides independently have a total length of less than 150 nucleotides, and wherein the two sequences are in tandem.

In certain embodiments, the present invention provides an expression cassette comprising a promoter and a nucleic acid encoding the modified tRNA or oligonucleotides as described above.

In certain embodiments, the present invention provides a vector comprising the oligonucleotide or the expression cassette described above.

In certain embodiments, the vector is a viral or plasmid vector. In certain embodiments, the present invention provides a composition comprising a modified tRNA, an oligonucleotide, or a vector described above, and a pharmaceutically acceptable carrier.

In certain embodiments, the carrier is a liposome.

In certain embodiments, the invention provides a cell comprising the vector described above.

The present invention provides a method of treating a stop-codon-associated genetic disease, comprising administering the modified tRNA composition described above to a patient in need thereof.

In certain embodiments, the genetic disease associated with a premature stop codon is cystic fibrosis, muscular dystrophy, β-thalassemia or Liddle's syndrome.

In certain embodiments, the present invention provides a method of restoring translation to a nucleotide sequence that includes a nonsense mutation in a cell, comprising introducing to the cell the composition described above.

In certain embodiments, the present invention provides a method of identifying anti-codon edited (ACE) tRNAs by high-throughput cloning and screening using suppression of a nonsense codon in luciferase enzymes including NanoLuc.

In certain embodiments, the present invention provides methods of treating a disease associated with a PTC in a subject in need thereof, comprising administering to the subject at least one composition comprising an ACE-tRNA or nucleic acid molecule encoding the same.

In certain embodiments, the disease is a disease or disorder associated with a UGA PTC, and the method comprises administering at least one ACE-tRNA specific for UGA.

In certain embodiments, the disease is a disease or disorder associated with a UAA PTC, and the method comprises administering at least one ACE-tRNA specific for UAA.

In certain embodiments, the disease is a disease or disorder associated with a UAG PTC, and the method comprises administering at least one ACE-tRNA specific for UAG.

In certain embodiments, the method comprises administering at least 2 ACE-tRNA, wherein each of the at least two ACE-tRNA are specific for incorporation of at least two different amino acid molecules onto a polypeptide chain.

In certain embodiments, the method comprises administering at least 2 ACE-tRNA, wherein each of the at least two ACE-tRNA are specific for incorporation of the same amino acid molecule onto a polypeptide chain.

In certain embodiments, at least 2 ACE-tRNA are encoded on the same nucleic acid molecule.

In certain embodiments, at least 2 ACE-tRNA are encoded on different nucleic acid molecules.

In certain embodiments, the method comprises administering at least one ACE-tRNA specific for UGA selected from the group consisting of ACE-tRNA^{Arg}, ACE-tRNA_{Gly} and ACE-tRNA_{Trp}.

In certain embodiments, the disease is selected from the group consisting of Duchenne and Becker muscular dystrophies, retinoblastoma, neurofibromatosis, ataxia-telangiectasia, Tay-Sachs disease, cystic fibrosis, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and rickets.

### BRIEF DESCRIPTION OF DRAWINGS

FIG. 1. Table of the Genetic Code.

FIG. 2. tRNAs have a general four-arm structure comprising a T-arm, a D-arm, and anticodon-arm, and an acceptor arm. These arms are also referred to as ‘loops’ throughout.

FIG. 3. ACE-tRNA for nonsense suppression (H. sapiens tRNA^{Trp}_{TGA}).

FIG. 4. Anti-codon edited (ACE)-tRNA encoded in a vector used to identify functional ACE tRNA sequences. This vector sequence includes a Nanoluciferase reporter system. The depicted vector was used to identify ACE tRNA with TGA suppression. TAA and TAG variants were used for the appropriate tRNA screens (see FIGS. 14 through 17).

FIG. 5. Schematic of the rescue of proteins and ion channels with stop codons via suppressor tRNA.

FIGS. 6A and 6B. Nonsense codon rescue with human ACE-tRNA. FIG. 6A. Schematic of the Anti-Codon Edited (ACE) Trp tRNA and cherry-TGA-cGFP-HA construct. FIG. 6B. Rescue of the cherry TGA eGFP-HA construct by ACE tryptophan tRNA #4.

FIG. 7. Nonsense codon rationale and prevalence observed in human disease. The twenty natural amino acids codons ranked as to their contribution to human disease, with dark cross-hatched codons being most prevalent (TGG, TAC, TAT, TCA, and TTA) and stippled codons being least prevalent. All cross-hatched codon sequences require a single nucleotide mutation to convert to a stop codon from the intended amino acid. Right panel, the most common disease causative nonsense codons within the cystic fibrosis transmembrane conductance regulator (CFTR). Herein, novel tRNA sequences have been discovered for the repair of the indicated mutation.

FIG. 8. Identification of tRNA sequences for the repair of tryptophan-TGA and glycine-TGA. Left axis indicates fold above background for luciferase activity. A majority RNA with mutant anti-codon loops lack rescue activity.

FIG. 9. CFTR 1282x rescue with Trpchr17.trna39 and Glychr19.trna2 ACE-tRNAs. Biochemical western blot data of CFTR W1282X channels co-expressed in HEK cells with the indicated tRNA. Expression vectors containing four copies of the indicated tRNA display higher rescue of the CFTR protein. “C” band indicates rescue of the fully mature, glycosylated CFTR protein. Antibody used was M3A7 from Cystic Fibrosis Therapeutics at a 1:1000 dilution.

FIGS. 10A and 10B. Expression of ACE-tRNA_{Trp} and ACE-tRNA_{Gly} results in specific incorporation of cognate amino acids into nonsense codons. FIG. 10A) Co-expression of model protein histidinol dehydrogenase (HDH)-His-Strep N94-TGA and ACE-tRNA_{Trp} (left) and ACE-tRNA_{Gly} (right) results in full-length HDH protein (asterisks) that is detectable by silver stain following affinity purification. FIG. 10B) Spectra of WT HDH (top), HDH-N94+ACE-tRNA_{Gly} (middle), and HDH-N94+ACE-tRNA_{Trp} (bottom). Spectra highlight amino acid mass differences at position N94 that match specifically with Glycine (−57 Da) and Tryptophan (+72 Da), indicating insertion of ACE-tRNA cognate amino acids.

FIG. 11. Cloning workflow for the construction of tRNA libraries.

FIGS. 12A-12B. Targeted mutations of nucleotides within the t-stem region further enhance ACE-tRNA rescue function. FIG. 12A. Trpchr17.tRNA 39 was systematically mutagenized within the t-stem region. These efforts identified ACE IRNA TS-10 52-62 G-C, (FIG. 12B) and cross-hatched bar in plot, which displays ˜250% increased biological activity.

FIGS. 13A-13F. ACE-tRNAs are selective for nonsense codons and more efficient than aminoglycoside nonsense suppression. FIG. 13A) ACE-tRNA_{Trp} #5 and FIG. 13B) ACE-tRNA_{Gly} #16 were cloned into NanoLuc reporter plasmids containing TGA, TAA or TAG nonsense codons. Nonsense suppression was only measured in NanoLuc-TGA contructs following transfection. FIG. 13C & FIG. 13D) Suppression of NanoLuc-TGA by addition of gentimicin (40 uM) and G418 (150 uM) and co-transfection with ACE-tRNA_{Trp} #5 and ACE-tRNA^{Gly} #16, was measured at FIG. 13C) 24 and FIG. 13D) 48hours in HEK293 cells. FIG. 13E & FIG. 13F) HEK293 cells stably expressing NanoLuc-TGA were treated with gentimicin (40 uM) and G418 (150 uM) and transfected with ACE-tRNA_{Trp} #5 and ACE-tRNA_{Gly} #16. Nonsense suppression was measured at FIG. 13E) 24 and FIG. 13F) 48 hours post treatment.

FIG. 14. ACE-tRNA-Arg-TGA. Identification of ACE-tRNA for repair of arginine-TGA nonsense codons.

FIG. 15. ACE-tRNA-Gln TAG. Identification of ACE-tRNA for repair of glutamine TAG nonsense codons.

FIG. 16. ACE-tRNA-Gln TAA Identification of ACE-tRNA for repair of glutamine TAA nonsense codons.

FIG. 17. ACE-tRNA-Glu TAG Identification of ACE-tRNA for repair of glutamate-TAG nonsense codons.

FIG. 18. ACE-tRNA-Gln TAA Identification of ACE-tRNA for repair of glutamate TAA nonsense codons.

FIG. 19. ACE-tRNA-Trp TAG Identification of ACE tRNA for the repair of tryptophan TAG nonsense codons.

FIGS. 20A-20D. Delivery of ACE-tRNA as small RNA supports robust suppression of G542X and W1282X nonsense mutations. FIG. 20A) CFTR cRNA with G542X or W1282X cystic fibrosis causing nonsense mutations was co-injected in Xenopus oocytes with serial dilutions of pre-folded ACE-tRNAGly and ACE-tRNATrp, respectively. Two-electrode voltage-clamp recordings of CFTR Cl− current were performed after 36 hours. Current-voltage relationships illustrate that increasing amounts of FIG. 20B) ACE-tRNA_{Trp} and FIG. 20C) ACE-tRNAGly pre-folded RNA results in increased CFTR function (measured CFTR Cl− currents) with WT CFTR achieved in ACE-tRNAGly experiments. FIG. 20D) Dose response of G542X ACE-tRNAGly (filled circles) and W1282X ACE-tRNATrp (open squares) rescue (CFTR Cl-currents elicited at +40 mV were normalized to WT CFTR Cl− currents at +40 mV). The dose dependence of ACE-tRNAGly (EC50=˜20 ng; Hill coefficient ˜1.4) shows clear saturation at WT CFTR levels, while ACE-tRNATrp is right shifted (EC50=˜94 ng; Hill coefficient 1.24).

FIGS. 21A-21B. A nonsense mutation suppression screen to identify candidate anticodon edited tRNAs (ACE-tRNAs). FIG. 21A, Schematic illustrates requisite interactions of ACE-tRNAs with translational machinery. Following delivery, ACE-tRNAs are recognized by an endogenous aminoacyl-tRNA synthetase and charged (aminoacylated) with their cognate amino acid. The aminoacylated ACE-tRNA is recognized by the endogenous clongation factor 1-alpha, which protects the ACE-IRNA from being de-acylated and delivers the aminoacyl ACE-tRNA to the ribosome for suppression of a premature termination codon, in this instance UGA. FIG. 21B, Individual ACE-tRNAs were cloned into the High Throughput Cloning Nonsense Reporter plasmid using Golden Gate paired with CcdB negative selection. The all-in-one plasmid contains the NLuc luciferase reporter with either a UGA, UAG or UAA PTC at p. 162 between the enzymatic large bit and requisite C-terminal small bit.

FIG. 22 Screens of ACE-tRNA gene families with the high throughput cloning nonsense mutation reporter platform. The indicated anticodon edited PTC sequences were tested for each ACE-tRNA family that is one nucleotide away from the endogenous anticodon sequence, FIG. 25. Multiple high performing suppressor tRNA were identified for each class. Data are shown in Log 10 scale in terms of normalized NLuc luminescence. Each tRNA dataset were obtained in triplicates and are displayed at SEM, with the corresponding ANOVA statistical analysis in Table 2. Coded identities and corresponding tRNA sequences are shown in FIG. 26 and Table 9, respectively.

FIGS. 23A-23C Cognate Encoding and High-Fidelity Suppression by Engineered IRNA. FIG. 23A, Tryptic fragment of histidinol dehydrogenase (HDH), where “X” indicates suppressed PTC codon. MS/MS spectra of the tryptic fragment with masses of indicated y and b ions for WT (top), N94G (middle) and N94W (bottom) HDH. b9 ion mass is shifted by the predicted mass of −57 Da and +72 Da from the WT asparagine, indicating the encoding of cognate amino acids glycine and tryptophan by ACE-tRNA^{Gly} and ACE-tRNA^{Trp}, respectively. FIG. 23B, ACE-TGA-tRNA^{Gly} (Glychr19.t2) selectively suppresses the UGA stop codon in transiently transfected HEK293 cells. FIG. 23C) ACE-tRNAGly transfection outperforms both gentamicin (40 uM) and G418 (140 uM) following a 48 hour incubation in Hek293 cells stably expressing NLuc-UGA.

FIGS. 24A-24B. Ribosome profiling of ACE-tRNA on transcriptome-wide 3′UTRs. FIG. 24A, Ribosome footprint densities on 3′UTRs are plotted as log 2-fold change for reads of treated cells versus control (puc57GG empty vector) as described in the materials and methods. Transcripts were grouped by their endogenous TAA, TAG, and TGA stop codons. Each point represents the mean of two replicates for a transcript. Error bars show Mean±SD of the log 2-fold changes. FIG. 24B, The average log 2-fold change of normalized ribosome footprint occupancy was plotted for each nucleotide from −50 to +50 nt surrounding stop codons of transcriptome (18,101 sequences). The cartoon illustrates the ˜15 nt offset from the 5′ end of ribosome footprint to the first base position of stop codon in the ribosome A-site.

FIG. 25. Codon usage for common PTC. Cross-hatching indicates the most common codons and corresponding amino acid type that can be converted to stop codons via nucleotide substitution. Engineered tRNA have been developed for each type.

FIG. 26. Number referenced ACE-tRNA activity plot.

FIG. 27. Alignment of Glycine tRNA sequences. 21 tRNAGly human sequences demonstrate high sequence homology amongst tRNA clades. Pattern in in tRNA image corresponds to patterned boxes in sequences.

FIG. 28. Side-chain identity at p. 162 in Nanoluciferase does not effect activity. Total luminescence activity is indicated for each mutation at site.

FIGS. 29A-29B. Analysis of ACE-tRNATIP sequences from multiple species and suppressor RNA mutations. FIGS. 29A. Sequence alignment. FIG. 29B. NLuc-UGA+ACE-tRNA^{Trp}/NLuc-UGA.

FIGS. 30A-30C. Histidinol dehydrogenase (HDH) His(8)-streptactin expression construct allows for efficient one-step isolation of protein from HEK293 cells. FIG. 30A) Protein sequence of HDH expression construct. Underlined sequence indicates coverage by mass spectrometry. The bold, underlined asparagine (a.a. position 94) is the residue mutated to a TGA PTC for determining ACE-tRNA fidelity. The dual affinity tag is indicated in bold italics. Silver stain of HDH protein following PTC suppression with FIG. 30B) Trpchr17.trna39 and FIG. 30C) Glychr19.trna2.

FIG. 31. Stop codon specificity is maintained for ACE-tRNA^{Trp}. Suppression activity 36 for tRNA TrpTGA Trpchr17.trna39, the top performing TrpTGA suppressor tRNA, FIG. 22. This tRNA was co-expressed with the indicated pNano-STOP plasmid.

FIGS. 32A-32D. ACE-tRNAs are more efficient than aminoglycoside PTC suppression. FIG. 32A) Raw and FIG. 32B) normalized luminescence measured 24 hours following addition of gentamicin (40 uM), G418 (150 uM) and transfection with Trpchr17.trna39 and Glychr19.trna2 in HEK293 cells stably expressing PTC reporter Nluc-UGA. FIG. 32C) Raw and FIG. 32D) normalized luminescence measured 24 hours following addition of gentamicin (40 uM), G418 (150 uM) and co-transfection with Trpchr17.trna39 and Glychr19.trna2 in HEK293 cells.

FIG. 33. Comparison of time courses of ACE-tRNA activitiy following delivery as RNA or cDNA. ACE-tRNAs were delivered to HEK293 cells that stably express pNanoLuc-UGA, however only 5 μl of the reaction mix was added to the cells to reduce the effect of transfection reagents on cell viability. ACE-tRNA delivered as RNA (open symbols), was more rapid in rescuing expression of the PTC reporter than cDNA constructs (close circles). However, ACE-tRNA activity continued to rise over the 48 hourrs when expressed from cDNA and decreased as an RNA deliverable.

FIGS. 34A-34F. In vivo delivery and suppression with ACE-tRNA as cDNA and RNA. FIG. 34A) Representative images of mice injected with NLuc-UGA with ACE-tRNAArg or pUC57 empty vector, NLuc-WT or water in the tibialis anterior muscle followed by electroporation at days 1, 2 and 7 after DNA administration. FIG. 34B) Quantification of luminescence emission by the tibialis anterior muscles of the abovementioned mouse groups at different timepoints after DNA injection and electroporation (n=3 mice per group). FIG. 34C) Rescued luminesce of stably expressed NLuc-UGA following transfection of Trpchr17.trna39cRNA and Glychr19.trna2 RNA transcripts (n=3). FIG. 34D) Representative western blot analysis of CFTR protein expressed in HEK293 cells 36 hours following transfection of WT, G542X, G542X+Glychr19.trna2, W1282X and W1282X+Trpchr17.trna39 CFTR cDNA. FIG. 34E) Exemplary families of CFTR Cl-current traces recorded using two-electrode voltage-clamp, 36 hours following injection with WT, G542X, G542X+ACE-tRNA-Glychr19.trna2, W1282X and W1282X+ACE-tRNA-Trpchr17.trna39 CFTR cRNA. Currents were elicited using 5 mV voltage steps from −60 to +35 mV. The vertical and horizontal scale bars indicate 10 μA and 50 ms, respectively. FIG. 34F) Dose response of G542X ACE-tRNAGly (filled circles) and W1282X ACE-tRNATrp (open squares) rescue (CFTR Cl− currents elicited at +35 mV were normalized to WT CFTR Cl− currents at +35 mV). ACE-tRNAGly rescue achieves WT-level of expressed CFTR current.

FIG. 35. ACE tRNA rescues PTC luciferase protein expression in muscle.

FIG. 36. ACE tRNA rescues PTC luciferase protein expression in skin.

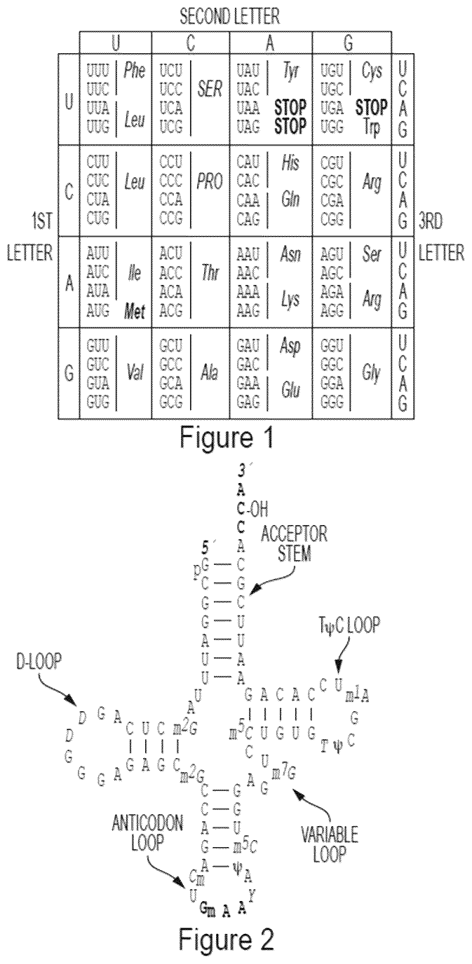

### DETAILED DESCRIPTION OF THE INVENTION

Over the years, researchers have identified hundreds of unique point mutations that resulted in nonsense codons being established in human genes. These types of mutations result, for example, in Duchenne and Becker muscular dystrophies, retinoblastoma, neurofibromatosis, ataxia-telangiectasia, Tay-Sachs disease, cystic fibrosis, Wilm's tumor, hemophilia A, hemophilia B, Menkes disease, Ullrich's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and rickets. The BRACA-1 and BRACA-2 genes associated with breast cancer also have similar mutations.

The nucleotide sequences encoding several hundred human tRNAs are known and generally available to those of skill in the art through sources such as Genbank. The structure of tRNAs is highly conserved and tRNAs are often functional across species. Thus, bacterial or other eukaryotic tRNA sequences are also potential sources for the oligonucleotides for the stabilized tRNAs of the invention. The determination of whether a particular tRNA sequence is functional in a desired mammalian cell can be ascertained through routine experimentation. Further additional potential tRNA sequences that are not yet known can be modified as described herein in order to be stabilized through routine experimentation.

tRNA genes have strong promoters that are active in all cell types. The promoters for eukaryotic tRNA genes are contained within the structural sequences encoding the tRNA molecule itself. Although there are elements that regulate transcriptional activity within the 5′ upstream region, the length of an active transcriptional unit may be considerably less than 500 base pairs and thus accommodation within a delivery vector is straightforward. Once they have been transcribed and processed, tRNAs have low rates of degradation. Finally, gene therapy with a nonsense suppressor maintains the endogenous physiological controls over the target gene that contains the nonsense codon.

### Nonsense Mutations

Transfer RNA (tRNA) is a type of RNA molecule that functions in the decoding of a messenger RNA (mRNA) sequence into a protein. RNAs function at specific sites in the ribosome during translation, which synthesizes a protein from an mRNA molecule. Nonsense mutations, also called Premature Termination Codons (PTCs), make up ˜10-15% of the single base pair mutations that cause human disease, and cystic fibrosis follows suit. (Peltz et al., Annu Rev Med., 64:407-25, 2013). In general, nonsense mutations have more serious ramifications than missense mutations because of the almost complete loss of gene expression and activity and with the possibility of dominant negative effects of truncated products. PTCs result in premature translation termination and accelerated mRNA transcript decay through the Nonsense Mediated Decay (NMD) pathway.

The current studies show that the specific site within an RNA transcript to which a tRNA delivers its amino acid can be changed through molecular editing of the anti-codon sequence within the tRNA. This approach allowed for a premature termination codon (PTC) to be effectively and therapeutically reverted back into the originally lost amino acid. Anticodon-edited tRNA (ACE-tRNA) form a new class of biological therapeutics.

Engineered tRNAs allow for “re-editing” of a disease-causing nonsense codon to a specific amino acid. These engineered tRNAs target only one type of stop codon, such as TGA over TAC or TAA. The small size of these tRNA molecules makes them amenable to ready expression, as the tRNA+the promoter is only ˜300 bp. Briefly, an oligonucleotide is synthesized that comprises the structural component of a tRNA gene functional in human cells. The sequence of this oligonucleotide is designed based upon the known sequence with substitutions made in the anticodon region of the tRNA causing the specific tRNA to recognize a nonsense or other specific mutation.

Several small molecule screens have been performed to suppress nonsense stop codons through interactions with the ribosome, the most outstanding molecules being G418, Gentamicin and PTC124. PTC124 or Ataluren has recently been relieved from Phase 3 clinical trials as use for a cystic fibrosis therapeutic. Ataluren and aminoglycosides promote read-through of each of the three nonsense codons by putting in a near cognate amino acid that turn a nonsense mutation into a missense mutation. (Roy et al., PNAS 2016 Nov. 1;113 (44): 12508-12513)

Anticodon-Edited tRNA (ACE-tRNA)

tRNAs have a general four-arm structure comprising a T-arm, a D-arm, and anticodon-arm, and an acceptor arm (FIG. 2).

The T-arm is made up of a “T-stem” and a “TYΨC loop.” In certain embodiments, the T-stem is modified to increase the stability of the tRNA. In certain embodiments, the ACE-tRNA has a modified T-stem that increases the biological activity to suppress stop sites relative to the endogenous T-stem sequence.

The present invention in one embodiment includes compositions comprising stabilized tRNAs, which can be used with higher effectiveness in order to treat a wide variety of nonsense mutation-associated diseases. The following sequences in Tables 1-8 are written as DNA, but as RNA (transcribed DNA) the “T:thymidine” is “U:uracil.” Therefore, tRNAs transcribed from the following sequences all contain uracils in place of the thymidines.

In certain embodiments, the tRNA has the following sequences (wherein the thymidines are replaced with uracils):

TS-36:
(SEQ ID NO: 1)
GGCCTCGTGGCGCAACGGTAGCGCGTCTGACTtCAGATCAGAAGGtTGCGg
GTTCAAATCCCGTCGGGGTCA
TS-37:
(SEQ ID NO: 2)
GGCCTCGTGGCGCAACGGTAGCGCGTCTGACTtCAGATCAGAAGGtTaCGg
GTTCAAATCcCGTCGGGGTCA
TS-38:
(SEQ ID NO: 3)
GGCCTCGTGGCGCAACGGTAGCGCGTCTGACTtCAGATCAGAAGGtTcCGg
GTTCAAATCcCGgCGGGGTCA

TABLE 1

| | | | SEQ |
|---|---|---|---|
| | | | ID |
| Ranking | Identifier | Sequence | NO. |
| #1 | ArgTGAchr9. | CGTCGGCTCTGTGGCGCAATGGATAGCGCATTGGACTTC | 4 |
| | trna6/ | AAATTCAAAGGTTGTGGGTTCGAGTCCCACCAGAGTCG | |
| | nointron | | |
| #2 | ArgTGAchr17. | CGTCGCCCCAGTGGCCTAATGGATAAGGCACTGGCCTTC | 5 |
| | trna19 | AAAGCCAGGGATTGTGGGTTCGAGTCCCACCTGGGGTG | |
| #3 | ArgTGAchr1. | CGTCGGCTCCGTGGCGCAATGGATAGCGCATTGGACTTC | 6 |
| | trna10/ | AAATTCAAAGGTTCCGGGTTCGAGTCCCGGCGGAGTCG | |
| | nointron | | |
| #4 | ArgTGAchr7. | CGTCGCCCCAGTGGCCTAATGGATAAGGCATTGGCCTTC | 7 |
| | trna5 | AAAGCCAGGGATTGTGGGTTCGAGTCCCATCTGGGGTG | |
| #4 | ArgTGAchr17. | CGTCGGCTCTGTGGCGCAATGGATAGCGCATTGGACTTC | 8 |
| | trna3/ | AAATTCAAAGGTTGTGGGTTCGAATCCCACCAGAGTCG | |
| | nointron | | |
| #5 | ArgTGAchr9. | CGTCGGCTCTGTGGCGCAATGGATAGCGCATTGGACTTC | 9 |
| | trna6/ | AAGCTGAGCCTAGTGTGGTCATTCAAAGGTTGTGGGTTC | |
| | withintron | GAGTCCCACCAGAGTCG | |
| #5 | ArgTGAchr16. | CGTCGCCCCGGTGGCCTAATGGATAAGGCATTGGCCTTC | 10 |
| | trna3 | AAAGCCAGGGATTGTGGGTTCGAGTCCCACCCGGGGTA | |
| #6 | ArgTGAchr1. | CGTCGGCTCCGTGGCGCAATGGATAGCGCATTGGACTTC | 11 |
| | trna10/ | AAGAGGCTGAAGGCATTCAAAGGTTCCGGGTTCGAGTCC | |
| | withintron | CGGCGGAGTCG | |
| #7 | ArgTGAchr17. | CGTCGGCTCTGTGGCGCAATGGATAGCGCATTGGACTTC | 12 |
| | trna3/ | AAGTGACGAATAGAGCAATTCAAAGGTTGTGGGTTCGAA | |
| | withinron | TCCCACCAGAGTCG | |
| | ArgTGAchr15. | CGTCGGCCGCGTGGCCTAATGGATAAGGCGTCTGACTTC | 13 |
| | trna4 | AGATCAGAAGATTGCAGGTTCGAGTCCTGCCGCGGTCG | |
| | ArgTGAchr17. | CGTCGACCGCGTGGCCTAATGGATAAGGCGTCTGACTTC | 14 |
| | trna17 | AGATCAGAAGATTGAGGGTTCGAGTCCCTTCGTGGTCG | |
| | ArgTGAchr11. | CGTCGGCTCTGTGGCGCAATGGATAGCGCATTGGACTTC | 15 |
| | trna3/ | AAGATAGTTAGAGAAATTCAAAGGTTGTGGGTTCGAGTC | |
| | withintron | CCACCAGAGTCG | |

TABLE 2

| | | | SEQ |
|---|---|---|---|
| | | | ID |
| Ranking | Identifier | Sequence | NO. |
| #1 | GlnTAGchr1. | CGTCGGTTCCATGGTGTAATGGTgAGCACTCTGGACTctaA | 16 |
| | trna17 | ATCCAGCGaTCCGAGTTCGAGTCTCGGTGGAACCT | |
| #2 | GlnTAGchr6. | CGTCGGCCCCATGGTGTAATGGTtAGCACTCTGGACTctaA | 17 |
| | trna175 | ATCCAGCGaTCCGAGTTCAAATCTCGGTGGGACCT | |
| #3 | GlnTAGchr6. | CGTCGGTCCCATGGTGTAATGGTtAGCACTCTGGACTctaA | 18 |
| | trna63 | ATCCAGCAaTCCGAGTTCGAATCTCGGTGGGACCT | |
| #4 | GlnTAGchr17. | CGTCGGTCCCATGGTGTAATGGTtAGCACTCTGGACTctaA | 19 |
| | trna14 | ATCCAGCGaTCCGAGTTCAAATCTCGGTGGGACCT | |
| #5 | GlnTAGchr6. | CGTCGGCCCCATGGTGTAATGGTcAGCACTCTGGACTctaA | 20 |
| | trna132 | ATCCAGCGaTCCGAGTTCAAATCTCGGTGGGACCC | |
| | GlnTAGchr1. | CGTCGGTTCCATGGTGTAATGGTaAGCACTCTGGACTctaA | 21 |
| | trna101 | ATCCAGCGaTCCGAGTTCGAGTCTCGGTGGAACCT | |
| | GlnTAGchr6. | CGTCGGTTCCATGGTGTAATGGTtAGCACTCTGGACTctaAA | 22 |
| | trna42 | TCCGGTAaTCCGAGTTCAAATCTCGGTGGAACCT | |
| | GlnTAGchr6. | CGTCGGTTCCATGGTGTAATGGTtAGCACTCTGGACTctaAA | 23 |
| | trna147 | TCCAGCGaTCCGAGTTCAAGTCTCGGTGGAACCT | |

TABLE 3

| | | | SEQ |
|---|---|---|---|
| | | | ID |
| Ranking | Identifier | Sequence | NO. |
| #1 | GlnTAAchr1. | CGTCGGTTCCATGGTGTAATGGTaAGCACTCTGGACTttaAA | 24 |
| | trna101 | TCCAGCGaTCCGAGTTCGAGTCTCGGTGGAACCT | |
| #2 | GlnTAAchr6. | CGTCGGCCCCATGGTGTAATGGTtAGCACTCTGGACTttaAA | 25 |
| | trna175 | TCCAGCGaTCCGAGTTCAAATCTCGGTGGGACCT | |
| #3 | GlnTAAchr1. | CGTCGGTTCCATGGTGTAATGGTgAGCACTCTGGACTttaAA | 26 |
| | trna17 | TCCAGCGaTCCGAGTTCGAGTCTCGGTGGAACCT | |
| #4 | GlnTAAchr6. | CGTCGGTTCCATGGTGTAATGGTtAGCACTCTGGACTttaAA | 27 |
| | trna1 | TCCAGCGaTCCGAGTTCAAATCTCGGTGGAACCT | |
| #5 | GlnTAAchr17. | CGTCGGTCCCATGGTGTAATGGTtAGCACTCTGGACTttaAA | 28 |
| | trna14 | TCCAGCGaTCCGAGTTCAAATCTCGGTGGGACCT | |
| #5.2 | GlnTAAchr6. | CGTCGGTCCCATGGTGTAATGGTtAGCACTCTGGACTttaAA | 29 |
| | trna63 | TCCAGCAaTCCGAGTTCGAATCTCGGTGGGACCT | |
| | GlnTAAchr6. | CGTCGGTTCCATGGTGTAATGGTtAGCACTCTGGACTttaAA | 30 |
| | trna42 | TCCGGTAaTCCGAGTTCAAATCTCGGTGGAACCT | |
| | GlnTAAchr6. | CGTCGGCCCCATGGTGTAATGGTcAGCACTCTGGACTttaAA | 31 |
| | trna132 | TCCAGCGaTCCGAGTTCAAATCTCGGTGGGACCC | |
| | GlnTAAchr6. | CGTCGGTTCCATGGTGTAATGGTtAGCACTCTGGACTttaAA | 32 |
| | trna147 | TCCAGCGaTCCGAGTTCAAGTCTCGGTGGAACCT | |

TABLE 4

| | | | SEQ |
|---|---|---|---|
| | | | ID |
| Ranking | Identifier | Sequence | NO. |
| #1 | TrpTAGchr17. | CGTCGACCTCGTGGCGCAATGGTAGCGCGTCTGACTctAGA | 33 |
| | trna10 | TCAGAAGGtTGCGTGTTCAAGTCACGTCGGGGTCA | |
| #2 | TrpTAGchr6. | CGTCGACCTCGTGGCGCAACGGTAGCGCGTCTGACTctAGA | 34 |
| | trna171 | TCAGAAGGtTGCGTGTTCAAATCACGTCGGGGTCA | |
| #3 | TrpTAGchr17. | CGTCGGCCTCGTGGCGCAACGGTAGCGCGTCTGACTctAGA | 35 |
| | trna39 | TCAGAAGGtTGCGTGTTCAAATCACGTCGGGGTCA | |
| #4 | TrpTAGchr12. | CGTCGACCTCGTGGCGCAACGGTAGCGCGTCTGACTctAGA | 36 |
| | trna6 | TCAGAAGGcTGCGTGTTCGAATCACGTCGGGGTCA | |
| | TrpTAGchr7. | CGTCGACCTCGTGGCGCAACGGCAGCGCGTCTGACTctAGA | 37 |
| | trna3 | TCAGAAGGtTGCGTGTTCAAATCACGTCGGGGTCA | |

TABLE 5

| | | | SEQ |
|---|---|---|---|
| | | | ID |
| Ranking | Identifier | Sequence | NO. |
| #1 | GluTAGchr13. | CGTCTCCCACATGGTCTAGCGGTtAGGATTCCTGGTTctaAC | 38 |
| | trna2 | CCAGGCGGCCCGGGTTCGACTCCCGGTGTGGGAA | |
| #2 | GluTAGchr2. | CGTCTCCCATATGGTCTAGCGGTtAGGATTCCTGGTTctaAC | 39 |
| | trna18 | CCAGGTGGCCCGGGTTCGACTCCCGGTATGGGAA | |
| #3 | GluTAGchr1. | CGTCTCCCTGGTGGTCTAGTGGCtAGGATTCGGCGCTctaAC | 40 |
| | trna123 | CGCCGCGGCCCGGGTTCGATTCCCGGTCAGGGAA | |
| #4 | GluTAGchr1. | CGTCTCCCTGGTGGTCTAGTGGTtAGGATTCGGCGCTctaAC | 41 |
| | trna106 | CGCCGCGGCCCGGGTTCGATTCCCGGTCAGGGAA | |
| | GluTAGchr1. | CGTCTCCCTGGTGGTCTAGTGGCtAGGATTCGGCGCTctaAC | 42 |
| | trna5 | CGCCGCGGCCCGGGTTCGATTCCCGGCCAGGGAA | |

TABLE 6

| | | | SEQ |
|---|---|---|---|
| | | | ID |
| Ranking | Identifier | Sequence | NO. |
| | GluTAAchr13. | CGTCTCCCACATGGTCTAGCGGTtAGGATTCCTGGTTctaAC | 43 |
| | trna2 | CCAGGCGGCCCGGGTTCGACTCCCGGTGTGGGAA | |
| | GluTAAchr2. | CGTCTCCCATATGGTCTAGCGGTtAGGATTCCTGGTTctaAC | 44 |
| | trna18 | CCAGGTGGCCCGGGTTCGACTCCCGGTATGGGAA | |
| | GluTAAchr1. | CGTCTCCCTGGTGGTCTAGTGGTtAGGATTCGGCGCTctaAC | 45 |
| | trna106 | CGCCGCGGCCCGGGTTCGATTCCCGGTCAGGGAA | |
| | GluTAAchr1. | CGTCTCCCTGGTGGTCTAGTGGTtAGGATTCGGCGCTctaAC | 46 |
| | trna55 | CGCCGCGGCCCGGGTTCGATTCCCGGTCAGGAAA | |
| | GluTAAchr1. | CGTCTCCCTGGTGGTCTAGTGGCtAGGATTCGGCGCTctaAC | 47 |
| | trna5 | CGCCGCGGCCCGGGTTCGATTCCCGGCCAGGGAA | |

TABLE 7

| | | | SEQ |
|---|---|---|---|
| | | | ID |
| Ranking | Identifier | Sequence | NO. |
| #1 | TrpTGAchr17. | GGCCTCGTGGCGCAACGGTAGCGCGTCTGACTtCAGATCA | 48 |
| | trna39 | GAAGGtTGCGTGTTCAAATCACGTCGGGGTCA | |
| #2 | TrpTGAchr17. | GACCTCGTGGCGCAATGGTAGCGCGTCTGACTtCAGATCA | 49 |
| | trna10 | GAAGGtTGCGTGTTCAAGTCACGTCGGGGTCA | |
| #3 | TrpTGAchr6. | GACCTCGTGGCGCAACGGTAGCGCGTCTGACTtCAGATCA | 50 |
| | trna171 | GAAGGtTGCGTGTTCAAATCACGTCGGGGTCA | |
| | TrpTGAchr12. | GACCTCGTGGCGCAACGGTAGCGCGTCTGACTtCAGATCA | 51 |
| | trna6 | GAAGGcTGCGTGTTCGAATCACGTCGGGGTCA | |
| | TrpTGAchr7. | GACCTCGTGGCGCAACGGCAGCGCGTCTGACTtCAGATCA | 52 |
| | trna3 | GAAGGtTGCGTGTTCAAATCACGTCGGGGTCA | |

TABLE 8

| | | | SEQ |
|---|---|---|---|
| | | | ID |
| Ranking | Identifier | Sequence | NO. |
| #1 | GlyTGAchr19. | GCGTTGGTGGTATAGTGGTtAGCATAGCTGCCTTCaAAG | 53 |
| | trna2 | CAGTTGaCCCGGGTTCGATTCCCGGCCAACGCA | |
| #2 | GlyTGAchr1. | GCGTTGGTGGTATAGTGGTgAGCATAGCTGCCTTCaAAG | 54 |
| | trna107 | CAGTTGaCCCGGGTTCGATTCCCGGCCAACGCA | |
| #3 | GlyTGAchr17. | GCGTTGGTGGTATAGTGGTaAGCATAGCTGCCTTCaAAG | 55 |
| | trna9 | CAGTTGaCCCGGGTTCGATTCCCGGCCAACGCA | |

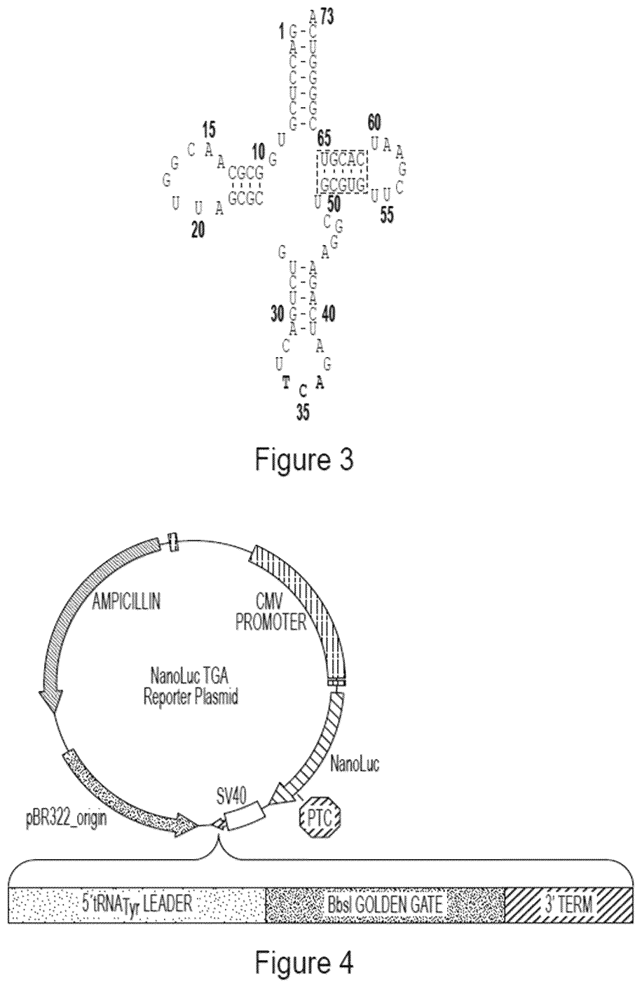

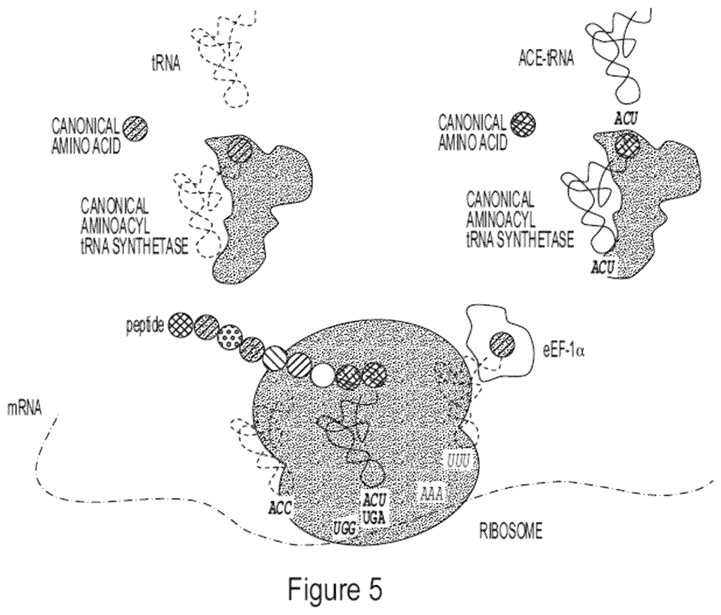

In one embodiment, the ACE-tRNA for nonsense suppression is as depicted in FIG. 3 (H. sapiens tRNA^{Trp}_{TGA}).

According to the invention, human UAA, UAG, and UGA suppressor tRNAs have been designed. The screen has identified codon edited tRNA for the repair of Trp-TGA, Trp-TAG, Arg-TGA, Gln-TAG, Gln-TA, Glu-TAG, Glu-TAA. The tRNAs are approximately 100 nucleotides in length and can be introduced to cells to suppress nonsense codons mutations where the wild-type amino acid should be present. The oligonucleotides can be introduced directly to recipient cells or can be ligated in tandem to increase efficacy of the oligonucleotide.

### Expression Cassettes and Vectors

In certain embodiments, the ACT-tRNA is encoded by an expression cassette. In yet another embodiment, the suppressor tRNA of the invention may be introduced to the cells using standard conventional genetic engineering techniques through use of vectors. Because of the internal promoter sequences of tRNA encoding sequences, the tRNA sequence need not be included in a separate transcription unit, although one may be provided.

In one embodiment of the present invention, the nucleotide expression system of the invention is included within an appropriate gene transfer vehicle which is then used to transduce cells to express the suppressor tRNA. The gene delivery vehicle can be any delivery vehicle known in the art, and can include naked DNA that is facilitated by a receptor and/or lipid mediated transfection, as well as any of a number of vectors. Such vectors include but are not limited to eukaryotic vectors, prokaryotic vectors (such as for example bacterial vectors) and viral vectors including, but not limited to, retroviral vectors, adenoviral vectors, adeno-associated viral vectors, lentivirus vectors (human and other including porcine), Herpes virus vectors, Epstein-Barr viral vectors, SV40 virus vectors, pox virus vectors, and pseudotyped viral vectors.

In certain embodiments, the ACT-tRNA (PTC) is encoded in a vector. FIG. 4. In certain embodiments, the viral vector is a retroviral or adenoviral vector. Examples of retroviral vectors that may be employed include, but are not limited to, Moloney Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus.

### Retroviruses; Retroviral Vectors

The term “retrovirus” is used in reference to RNA viruses that utilize reverse transcriptase during their replication cycle. The retroviral genomic RNA is converted into double-stranded DNA by reverse transcriptase. This double-stranded DNA form of the virus is capable of being integrated into the chromosome of the infected cell; once integrated, it is referred to as a “provirus.” The provirus serves as a template for RNA polymerase II and directs the expression of RNA molecules that encode the structural proteins and enzymes needed to produce new viral particles. At each end of the provirus are structures called “long terminal repeats” or “LTRs.” The LTR contains numerous regulatory signals including transcriptional control elements, polyadenylation signals and sequences needed for replication and integration of the viral genome. There are several genera included within the family Retroviridae, including Cisternavirus A, Oncovirus A, Oncovirus B, Oncovirus C, Oncovirus D, Lentivirus, and Spumavirus. Some of the retroviruses are oncogenic (i.e., tumorigenic), while others are not. The oncoviruses induce sarcomas, leukemias, lymphomas, and mammary carcinomas in susceptible species. Retroviruses infect a wide variety of species, and may be transmitted both horizontally and vertically. They are integrated into the host DNA, and are capable of transmitting sequences of host DNA from cell to cell. This has led to the development of retroviruses as vectors for various purposes including gene therapy.

Retroviruses, including human foamy virus (HFV) and human immunodeficiency virus (HIV) have gained much recent attention, as their target cells are not limited to dividing cells and their restricted host cell tropism can be readily expanded via pseudotyping with vesicular stomatitis virus G (VSV-G) envelope glycoproteins (See e.g., J. C. Burns et al., Proc. Natl. Acad. Sci. USA 90:8033-8037 [1993]; A. M. L. Lever, Gene Therapy. 3:470-471 [1996]; and D. Russell and A. D. Miller, J. Virol., 70:217-222 [1996]).

Vector systems generally have a DNA vector containing a small portion of the retroviral sequence (the viral long terminal repeat or “LTR” and the packaging or “psi” signal) and a packaging cell line. The gene to be transferred is inserted into the DNA vector. The viral sequences present on the DNA vector provide the signals necessary for the insertion or packaging of the vector RNA into the viral particle and for the expression of the inserted gene. The packaging cell line provides the viral proteins required for particle assembly (D. Markowitz et al., J. Virol., 62:1120 [1988]). In one embodiment of the present invention, an FIV system employing a three-plasmid transfection production method in 293T cells was used (Johnston et al., J Virol. 1999 73:4991-5000). Replication incompetent virus was successfully produced.

The vector DNA is introduced into the packaging cell by any of a variety of techniques (e.g., calcium phosphate coprecipitation, lipofection, electroporation). The viral proteins produced by the packaging cell mediate the insertion of the vector sequences in the form of RNA into viral particles, which are shed into the culture supernatant.

For cells that are naturally dividing, or are stimulated to divide by growth factors, simple retroviruses like murine leukemia virus (MLV) vectors are suitable delivery systems. A major limitation in the use of many commonly used retroviral vectors in gene transfer, however, is that many of the vectors are restricted to dividing cells. If a non-dividing cell is the target cell, then a lentivirus, which is capable of infecting non-dividing cells, may be used.

As used herein, the term “lentivirus” refers to a group (or genus) of retroviruses that give rise to slowly developing disease. Viruses included within this group include HIV (human immunodeficiency virus; including HIV type 1, and HIV type 2), the etiologic agent of the human acquired immunodeficiency syndrome (AIDS); visna-maedi, that causes encephalitis (visna) or pneumonia (maedi) in sheep, the caprine arthritis-encephalitis virus, which causes immune deficiency, arthritis, and encephalopathy in goats; equine infectious anemia virus, which causes autoimmune hemolytic anemia, and encephalopathy in horses; feline immunodeficiency virus (FIV), which causes immune deficiency in cats; bovine immune deficiency virus (BIV), which causes lymphadenopathy, lymphocytosis, and possibly central nervous system infection in cattle; and simian immunodeficiency virus (SIV), which cause immune deficiency and encephalopathy in sub-human primates. Diseases caused by these viruses are characterized by a long incubation period and protracted course. Usually, the viruses latently infect monocytes and macrophages, from which they spread to other cells. HIV, FIV, and SIV also readily infect T lymphocytes (i.e., T-cells).

Lentiviruses including HIV, SIV, FIV and equine infectious anemia virus (EIAV) depend on several viral regulatory genes in addition to the simple structural gag-pol-env genes for efficient intracellular replication. Thus, lentiviruses use more complex strategies than classical retroviruses for gene regulation and viral replication, with the packaging signals apparently spreading across the entire viral genome. These additional genes display a web of regulatory functions during the lentiviral life cycle. For example, upon HIV-1 infection, transcription is up-regulated by the expression of Tat through interaction with an RNA target (TAR) in the LTR. Expression of the full-length and spliced mRNAs is then regulated by the function of Rev, which interacts with RNA elements present in the gag region and in the env region (RRE) (S. Schwartz et al., J. Virol., 66:150-159 [1992]). Nuclear export of gag-pol and env mRNAs is dependent on the Rev function. In addition to these two essential regulatory genes, a list of accessory genes, including vif, vpr, vpx, vpu, and nef, are also present in the viral genome and their effects on efficient virus production and infectivity have been demonstrated, although they are not absolutely required for virus replication (K. and F. Wong-Staal, Microbiol. Rev., 55:193-205 (1991]; R. A. Subbramanian and E. A. Cohen, J. Virol. 68:6831-6835 [1994]; and D. Trono, Cell 82:189-192 [1995]). A detailed description of the structure of an exemplary lentivirus, HIV-1, is given in U.S. Pat. No. 6,531,123.

A “source” or “original” retrovirus is a wild-type retrovirus from which a pseudotyped retrovirus is derived, or is used as a starting point, during construction of the packaging or transgene vector, for the preparation of one or more of the genetic elements of the vector. The genetic element may be employed unchanged, or it may be mutated (but not beyond the point where it lacks a statistically significant sequence similarity to the original element). A vector may have more than one source retrovirus, and the different source retroviruses may be, e.g., MLV, FIV, HIV-1 and HIV-2, or HIV and SIV. The term “genetic element” includes but is not limited to a gene.

A cognate retrovirus is the wild-type retrovirus with which the vector in question has the greatest percentage sequence identity at the nucleic acid level. Normally, this will be the same as the source retrovirus. However, if a source retrovirus is extensively mutated, it is conceivable that the vector will then more closely resemble some other retrovirus. It is not necessary that the cognate retrovirus be the physical starting point for the construction; one may choose to synthesize a genetic element, especially a mutant element, directly, rather than to first obtain the original element and then modify it. The term “cognate” may similarly be applied to a protein, gene, or genetic element (e.g., splice donor site or packaging signal). When referring to a cognate protein, percentage sequence identities are determined at the amino acid level.

The term “cognate” retrovirus may be difficult to interpret in the extreme case, i.e., if all retroviral genetic elements have been replaced with surrogate non-lentiviral genetic elements. In this case, the source retrovirus strain mentioned previously is arbitrarily considered to be the cognate retrovirus.

The term “replication” as used herein in reference to a virus or vector, refers not to the normal replication of proviral DNA in a chromosome as a consequence of cell reproduction, or the autonomous replication of a plasmid DNA as a result of the presence of a functional origin of replication. Instead “replication” refers to the completion of a complete viral life cycle, wherein infectious viral particles containing viral RNA enter a cell, the RNA is reverse transcribed into DNA, the DNA integrates into the host chromosome as a provirus, the infected cell produces virion proteins and assembles them with full length viral genomic RNA into new, equally infectious particles.

The term “replication-competent” refers to a wild-type virus or mutant virus that is capable of replication, such that replication of the virus in an infected cell result in the production of infectious virions that, after infecting another, previously uninfected cell, causes the latter cell to likewise produce such infectious virions. The present invention contemplates the use of replication-defective virus.

As used herein, the term “attenuated virus” refers to any virus (e.g., an attenuated lentivirus) that has been modified so that its pathogenicity in the intended subject is substantially reduced. The virus may be attenuated to the point it is nonpathogenic from a clinical standpoint, i.e., that subjects exposed to the virus do not exhibit a statistically significant increased level of pathology relative to control subjects.

The present invention contemplates the preparation and use of a modified retrovirus. In some embodiments, the retrovirus is an mutant of murine leukemia virus, human immunodefciency virus type 1, human immunodeficiency virus type 2, feline immunodeficiency virus, simian immunodeficiency virus, visna-macdi, caprine arthritis-encephalitis virus, equine infectious anemia virus, and bovine immune deficiency virus, or a virus comprised of portions of more than one retroviral species (e.g., a hybrid, comprised of portions of MLV, FIV, HIV-1 and HIV-2, or HIV-1 and/or SIV).

A reference virus is a virus whose genome is used in describing the components of a mutant virus. For example, a particular genetic element of the mutant virus may be said to differ from the cognate element of the reference virus by various substitutions, deletions or insertions. It is not necessary that the mutant virus actually be derived from the reference virus.

The preferred reference FIV sequence is found in Talbott et al., Proc Natl Acad Sci USA. 1989 86:5743-7; Genbank access #NC_001482. In certain embodiments, a three-plasmid transient transfection method can be used to produce replication incompetent pseudotyped retrovirues (e.g., FIV). General methods are described in Wang et al., J Clin Invest. 1999 104:R55-62 and Johnston et al., J Virol. 1999 73:4991-5000.

### Retroviral Vector System

The present invention contemplates a retroviral gene amplification and transfer system comprising a transgene vector, one or more compatible packaging vectors, an envelope vector, and a suitable host cell. The vectors used may be derived from a retrovirus (e.g., a lentivirus). Retrovirus vectors allow (1) transfection of the packaging vectors and envelope vectors into the host cell to form a packaging cell line that produces essentially packaging-vector-RNA-free viral particles, (2) transfection of the transgene vector into the packaging cell line, (3) the packaging of the transgene vector RNA by the packaging cell line into infectious viral particles, and (4) the administration of the particles to target cells so that such cells are transduced and subsequently express a transgene.

Either the particles are administered directly to the subject, in vivo, or the subject's cells are removed, infected in vitro with the particles, and returned to the body of the subject.

The packaging vectors and transgene vectors of the present invention will generate replication-incompetent viruses. The vectors chosen for incorporation into a given vector system of the present invention are such that it is not possible, without further mutation of the packaging vector(s) or transgene vector, for the cotransfected cells to generate a replication-competent virus by homologous recombination of the packaging vector(s) and transgene vector alone. The envelope protein used in the present system can be a retroviral envelope, a synthetic or chimeric envelope, or the envelope from a non-retroviral enveloped virus (e.g., baculovirus).

### Packaging Signal

As used herein, the term “packaging signal” or “packaging sequence” refers to sequences located within the retroviral genome or a vector that are required for, or at least facilitate, insertion of the viral or vector RNA into the viral capsid or particle. The packaging signals in an RNA identify that RNA as one that is to be packaged into a virion. The term “packaging signal” is also used for convenience to refer to a vector DNA sequence that is transcribed into a functional packaging signal. Certain packaging signals may be part of a gene, but are recognized in the form of RNA, rather than as a peptide moiety of the encoded protein.

The key distinction between a packaging vector and a transgene vector is that in the packaging vector, the major packaging signal is inactivated, and, in the transgene vector, the major packaging sign al is functional. Ideally, in the packaging vector, all packaging signals would be inactivated, and, in the transgene vector, all packaging signals would be functional. However, countervailing considerations, such as maximizing viral titer, or inhibiting homologous recombination, may lend such constructs less desirable.

### Packaging System; Packaging Vectors; Packaging Cell Line

A packaging system is a vector, or a plurality of vectors, which collectively provide in expressible form all of the genetic information required to produce a virion that can encapsidate suitable RNA, transport it from the virion-producing cell, transmit it to a target cell, and, in the target cell, cause the RNA to be reverse transcribed and integrated into the host genome in a such a manner that a transgene incorporated into the aforementioned RNA can be expressed. However, the packaging system must be substantially incapable of packaging itself. Rather, it packages a separate transgene vector.

In the present invention, the packaging vector will provide functional equivalents of the gag and pol genes (a “GP” vector). The env gene(s) will be provided by the envelope vector. In theory, a three vector system (“G”, “P”, and “E” vectors) is possible if one is willing to construct distinct gag and pol genes on separate vectors, and operably link them to different regulatable promoters (or one to a regulatable and the other to a constitutive promoter) such that their relative levels of expression can be adjusted appropriately.

A packaging cell line is a suitable host cell transfected by a packaging system that, under achievable conditions, produces viral particles. As used herein, the term “packaging cell lines” is typically used in reference to cell lines that express viral structural proteins (e.g., gag, pol and env), but do not contain a packaging signal. For example, a cell line has been genetically engineered to carry at one chromosomal site within its genome, a 5′-LTR-gag-pol-3′-LTR fragment that lacks a functional psi⁺ sequence (designated as Δ-psi), and a 5′-LTR-env-3′-LTR fragment that is also Δ-psi located at another chromosomal site. While both of these segments are transcribed constitutively, because the psi⁺ region is missing and the viral RNA molecules produced are less than full-size, empty viral particles are formed.

If a host cell is transfected by the packaging vector(s) alone, it produces substantially only viral particles without the full-length packaging vector. In one example, less than 10% of the viral particles produced by the packaging cell contain full length packaging vector-derived RNA. However, since the packaging vector lacks a functional primer-binding site, even if these particles infect a new cell, the packaging vector RNA will not be reverse transcribed back into DNA and therefore the new cell will not produce virion. Thus, by itself, the packaging vector is a replication-incompetent virus.

In some embodiments, the packaging cell and/or cell line contains a transgene vector. The packaging cell line will package the transgene vector into infectious particles. Such a cell line is referred to herein as a “transgenic virion production cell line.”

It is contemplated that packaging may be inducible, as well as non-inducible. In inducible packaging cells and packaging cell lines, retroviral particles are produced in response to at least one inducer. In non-inducible packaging cell lines and packaging cells, no inducer is required in order for retroviral particle production to occur.

The packaging vectors necessarily differ from wild-type, replication-competent retroviral genomes by virtue of the inactivation of at least one packaging signal of the cognate wild-type genome. More than one packaging signal may be inactivated. In one example, only the retroviral genes provided by the packaging vector are those encoding structural, or essential regulatory, proteins.

### Transgene Vectors

A transgene vector is an expression vector that bears an expressible non-retroviral gene of interest and includes at least one functional retroviral packaging signal, so that, after the transgene vector is transfected into a packaging cell line, the transgene vector is transcribed into RNA, and this RNA is packaged into an infectious viral particle. These particles, in turn, infect target cells, their RNA is reverse transcribed into DNA, and the DNA is incorporated into the host cell genome as a proviral element, thereby transmitting the gene of interest to the target cells.

As used herein, the term “transduction” refers to the delivery of a gene(s) using a viral or retroviral vector by means of infection rather than by transfection. In certain embodiments, retroviral vectors are transduced. Thus, a “transduced gene” is a gene that has been introduced into the cell via retroviral or vector infection and provirus integration. In certain embodiments, viral vectors (e.g., “transgene vectors”) transduce genes into “target cells” or host cells. The, present invention encompasses transgene vectors that are suitable for use in the present invention that are linked to any gene of interest (or a “marker gene” or “reporter gene,” used to indicate infection or expression of a gene).

As used herein, the term “long-term transduction” refers to vectors that are capable of remaining transduced in host or target cells for time periods that are longer than those observed with other vectors. For example, the present invention provides retroviral vectors that are capable of remaining transduced for at least 120 days, at least one year, or for the life of the subject or the necessary time course of treatment. The duration of expression is a function of the choice of promoter and the target cell type, more so than the choice of vector.

The term “stable transduction” or “stably transduced” refers to the introduction and integration of foreign DNA into the genome of the transducted cell. The term “stable transductant” refers to a cell that has stably integrated foreign DNA into the genomic DNA.

The term “transient transduction” or “transiently transduced” refers to the introduction of foreign DNA into a cell where the foreign DNA fails to integrate into the genome of the transducted cell. The foreign DNA persists in the nucleus of the transducted cell for several days. During this time the foreign DNA is subject to the regulatory controls that govern the expression of endogenous genes in the chromosomes. The term “transient transductant” refers to cells that have taken up foreign DNA but have failed to integrate this DNA.

In some embodiments, the target and/or host cells of the present invention are “non-dividing” cells. These cells include cells such as neuronal cells that do not normally divide. However, it is not intended that the present invention be limited to non-dividing cells (including, but not limited to muscle cells, white blood cells, spleen cells, liver cells, eye cells, epithelial cells).

In some embodiments, the vector and the vector progeny are capable of transducing a plurality of target cells so as to achieve vector titers of at least 10⁵ cfu/ml. The multiplicity of infection (MOI) may be at least one (i.e., one hit on average per cell), or even at least two.

### Expression Cassettes and Vectors

The present invention also provides an expression cassette comprising a sequence encoding ACE-tRNA.

In certain embodiments, the expression cassette further contains a promoter. In certain embodiments, the promoter is a regulatable promoter. In certain embodiments, the promoter is a constitutive promoter. In certain embodiments, the promoter is a PGK, CMV, RSV, H1 or U6 promoter (Pol II and Pol III promoters).

The present invention provides a vector containing the expression cassette described above. In certain embodiments, the vector is a viral vector. In certain embodiments, the viral vector is an adenoviral, lentiviral, adeno-associated viral (AAV), poliovirus, HSV, or murine Maloney-based viral vector.

“Expression cassette” as used herein means a nucleic acid sequence capable of directing expression of a particular nucleotide sequence in an appropriate host cell, which may include a promoter operably linked to the nucleotide sequence of interest that may be operably linked to termination signals. It also may include sequences required for proper translation of the nucleotide sequence. The coding region usually codes for a protein of interest. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter that initiates transcription only when the host cell is exposed to some particular stimulus. In the case of a multicellular organism, the promoter can also be specific to a particular tissue or organ or stage of development.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

### Adeno Associated Virus (AAV)

Adeno associated virus (AAV) is a small nonpathogenic virus of the parvoviridae family. AAV is distinct from the other members of this family by its dependence upon a helper virus for replication. In the absence of a helper virus, AAV may integrate in a locus specific manner into the q arm of chromosome 19. The approximately 5 kb genome of AAV consists of one segment of single stranded DNA of either plus or minus polarity. The ends of the genome are short inverted terminal repeats that can fold into hairpin structures and serve as the origin of viral DNA replication. Physically, the parvovirus virion is non-enveloped and its icosohedral capsid is approximately 20 nm in diameter.

To date, numerous serologically distinct AAVs have been identified, and more than a dozen have been isolated from humans or primates. The genome of AAV2 is 4680 nucleotides in length and contains two open reading frames (ORFs). The left ORF encodes the non-structural Rep proteins, Rep 40, Rep 52, Rep 68 and Rep 78, which are involved in regulation of replication and transcription in addition to the production of single-stranded progeny genomes. Furthermore, two of the Rep proteins have been associated with the preferential integration of AAV genomes into a region of the q arm of human chromosome 19. Rep68/78 has also been shown to possess NTP binding activity as well as DNA and RNA helicase activities. The Rep proteins possess a nuclear localization signal as well as several potential phosphorylation sites. Mutation of one of these kinase sites resulted in a loss of replication activity.

The ends of the genome are short inverted terminal repeats (ITR) which have the potential to fold into T-shaped hairpin structures that serve as the origin of viral DNA replication. Within the ITR region two elements have been described which are central to the function of the ITR, a GAGC repeat motif and the terminal resolution site (trs). The repeat motif has been shown to bind Rep when the ITR is in either a linear or hairpin conformation. This binding serves to position Rep68/78 for cleavage at the trs, which occurs in a site-and strand-specific manner. In addition to their role in replication, these two elements appear to be central to viral integration. Contained within the chromosome 19 integration locus is a Rep binding site with an adjacent trs. These elements have been shown to be functional and necessary for locus specific integration.

The AAV virion is a non-enveloped, icosohedral particle approximately 25 nm in diameter, consisting of three related proteins referred to as VP1, VP2 and VP3. The right ORF encodes the capsid proteins VP1, VP2, and VP3. These proteins are found in a ratio of 1:1:10 respectively and are all derived from the right-hand ORF. The capsid proteins differ from each other by the use of alternative splicing and an unusual start codon. Deletion analysis has shown that removal or alteration of VP1 which is translated from an alternatively spliced message results in a reduced yield of infections particles. Mutations within the VP3 coding region result in the failure to produce any single-stranded progeny DNA or infectious particles. An AAV particle is a viral particle comprising an AAV capsid protein. An AAV capsid polypeptide can encode the entire VP1, VP2 and VP3 polypeptide. The particle can be a particle comprising AAV2 and other AAV capsid proteins (i.e., a chimeric protein, such as AAV1 and AAV2). Variations in the amino acid sequence of the AAV2 capsid protein are contemplated herein, as long as the resulting viral particle comprises the AAV2 capsid remains antigenically or immunologically distinct from AAV1, as can be routinely determined by standard methods. Specifically, for example, ELISA and Western blots can be used to determine whether a viral particle is antigenically or immunologically distinct from AAV1. Furthermore, the AAV2 viral particle preferably retains tissue tropism distinct from AAV1.

An AAV2 particle is a viral particle comprising an AAV2 capsid protein. An AAV2 capsid polypeptide encoding the entire VP1, VP2, and VP3 polypeptide can overall have at least about 63% homology (or identity) to the polypeptide having the amino acid sequence encoded by nucleotides set forth in NC_001401 (nucleotide sequence encoding AAV2 capsid protein). The capsid protein can have about 70% homology, about 75% homology, 80% homology, 85% homology, 90% homology, 95% homology, 98% homology, 99% homology, or even 100% homology to the protein encoded by the nucleotide sequence set forth in NC_001401. The capsid protein can have about 70% identity, about 75% identity, 80% identity, 85% identity, 90% identity, 95% identity, 98% identity, 99% identity, or even 100% identity to the protein encoded by the nucleotide sequence set forth in NC_001401. The particle can be a particle comprising another AAV and AAV2 capsid protein, i.e., a chimeric protein. Variations in the amino acid sequence of the AAV2 capsid protein are contemplated herein, as long as the resulting viral particle comprising the AAV2 capsid remains antigenically or immunologically distinct from AAV4, as can be routinely determined by standard methods. Specifically, for example, ELISA and Western blots can be used to determine whether a viral particle is antigenically or immunologically distinct from AAV1. Furthermore, the AAV2 viral particle preferably retains tissue tropism distinction from AAV1, such as that exemplified in the examples herein, though an AAV2 chimeric particle comprising at least one AAV2 coat protein may have a different tissue tropism from that of an AAV2 particle consisting only of AAV2 coat proteins.

In certain embodiments, the invention further provides an AAV2 particle containing, i.e., encapsidating, a vector comprising a pair of AAV2 inverted terminal repeats. The nucleotide sequence of AAV2 ITRs is known in the art. Furthermore, the particle can be a particle comprising both AAV1 and AAV2 capsid protein, i.e., a chimeric protein. Moreover, the particle can be a particle encapsidating a vector comprising a pair of AAV inverted terminal repeats from other AAVs (e.g., AAV1-AAV9 and AAVrh10). The vector encapsidated in the particle can further comprise an exogenous nucleic acid inserted between the inverted terminal repeats.

The following features of AAV have made it an attractive vector for gene transfer. AAV vectors have been shown in vitro to stably integrate into the cellular genome; possess a broad host range; transduce both dividing and non-dividing cells in vitro and in vivo and maintain high levels of expression of the transduced genes. Viral particles are heat stable, resistant to solvents, detergents, changes in pH, temperature, and can be concentrated on CsCl gradients or by other means. The present invention provides methods of administering AAV particles, recombinant AAV vectors, and recombinant AAV virions. For example, an AAV2 particle is a viral particle comprising an AAV2 capsid protein, or an AAV1 particle is a viral particle comprising an AAV1 capsid protein. A recombinant AAV2 vector is a nucleic acid construct that comprises at least one unique nucleic acid of AAV2. A recombinant AAV2 virion is a particle containing a recombinant AAV2 vector. To be considered within the term “AAV2 ITRs” the nucleotide sequence must retain one or both features described herein that distinguish the AAV2 ITR from the AAV1 ITR: (1) three (rather than four as in AAV1) “GAGC” repeats and (2) in the AAV2 ITR Rep binding site the fourth nucleotide in the first two “GAGC” repeats is a C rather than a T.

The promoter to drive expression of the sequence encoding the tRNA to be delivered can be any desired promoter, selected by known considerations, such as the level of expression of a nucleic acid functionally linked to the promoter and the cell type in which the vector is to be used. Promoters can be an exogenous or an endogenous promoter. Promoters can include, for example, known strong promoters such as SV40 or the inducible metallothionein promoter, or an AAV promoter, such as an AAV p5 promoter. Additional examples of promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc. Additional examples include regulated promoters.

The AAV vector can further comprise an exogenous (heterologous) nucleic acid functionally linked to the promoter. By “heterologous nucleic acid” is meant that any heterologous or exogenous nucleic acid can be inserted into the vector for transfer into a cell, tissue or organism. The nucleic acid can encode a tRNA, for example. By “functionally linked” is meant such that the promoter can promote expression of the heterologous nucleic acid, as is known in the art, such as appropriate orientation of the promoter relative to the heterologous nucleic acid. Furthermore, the heterologous nucleic acid preferably has all appropriate sequences for expression of the nucleic acid, as known in the art, to functionally encode, i.e., allow the nucleic acid to be expressed. The nucleic acid can include, for example, expression control sequences, such as an enhancer. The nucleic acid can encode more than one gene product, limited only by the size of nucleic acid that can be packaged.

An AAV1 particle is a viral particle comprising an AAV1 capsid protein. Variations in the amino acid sequence of the AAV1 capsid protein are contemplated herein, as long as the resulting viral particle comprising the AAV1 capsid remains antigenically or immunologically distinct from other AAV capsids, as can be routinely determined by standard methods. Specifically, for example, ELISA and Western blots can be used to determine whether a viral particle is antigenically or immunologically distinct from other AAV serotypes.

The term “polypeptide” as used herein refers to a polymer of amino acids and includes full-length proteins and fragments thereof. Thus, “protein” and “polypeptide” are often used interchangeably herein.

The present method provides a method of delivering a nucleic acid to a cell comprising administering to the cell an AAV particle containing a vector comprising the nucleic acid inserted between a pair of AAV inverted terminal repeats, thereby delivering the nucleic acid to the cell. Administration to the cell can be accomplished by any means, including simply contacting the particle, optionally contained in a desired liquid such as tissue culture medium, or a buffered saline solution, with the cells. The particle can be allowed to remain in contact with the cells for any desired length of time, and typically, the particle is administered and allowed to remain indefinitely. For such in vitro methods, the virus can be administered to the cell by standard viral transduction methods, as known in the art and as exemplified herein. Titers of virus to administer can vary, particularly depending upon the cell type, but will be typical of that used for AAV transduction in general. Additionally the titers used to transduce the particular cells in the present examples can be utilized. The cells can include any desired cell in humans as well as other large (non-rodent) mammals, such as primates, horse, sheep, goat, pig, and dog.

The present invention further provides a method of delivering a nucleic acid to a cell in a subject comprising administering to the subject an AAV particle comprising the nucleic acid inserted between a pair of AAV inverted terminal repeats, thereby delivering the nucleic acid to a cell in the subject.

Certain embodiments of the present disclosure provide a cell comprising a viral vector as described herein.

### AAV Vectors

In one embodiment, a viral vector of the disclosure is an AAV vector. An “AAV” vector refers to an adeno-associated virus, and may be used to refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV, which is identified by, and distinguished from other AAVs based on capsid protein reactivity with defined antisera, e.g., there are eight known serotypes of primate AAVs, AAV-1 to AAV-9 and AAVrh10. For example, serotype AAV2 is used to refer to an AAV, which contains capsid proteins encoded from the cap gene of AAV2 and a genome containing 5′ and 3′ ITR sequences from the same AAV2 serotype. As used herein, for example, rAAV1 may be used to refer an AAV having both capsid proteins and 5′-3′ ITRs from the same serotype or it may refer to an AAV having capsid proteins from one serotype and 5′-3′ ITRs from a different AAV serotype, e.g., capsid from AAV serotype 2 and ITRs from AAV serotype 5. For each example illustrated herein, the description of the vector design and production describes the serotype of the capsid and 5′-3′ ITR sequences. The abbreviation “rAAV” refers to recombinant adeno-associated virus, also referred to as a recombinant AAV vector (or “rAAV vector”).

An “AAV virus” or “AAV viral particle” refers to a viral particle composed of at least one AAV capsid protein (preferably by all of the capsid proteins of a wild-type AAV) and an encapsidated polynucleotide. If the particle comprises heterologous polynucleotide (i.e., a polynucleotide other than a wild-type AAV genome such as a transgene to be delivered to a mammalian cell), it is typically referred to as “rAAV”.

In one embodiment, the AAV expression vectors are constructed using known techniques to at least provide as operatively linked components in the direction of transcription, control elements including a transcriptional initiation region, the DNA of interest and a transcriptional termination region. The control elements are selected to be functional in a mammalian cell. The resulting construct which contains the operatively linked components is flanked (5′ and 3′) with functional AAV ITR sequences.

By “adeno-associated virus inverted terminal repeats” or “AAV ITRs” is meant the art-recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. As used herein, an “AAV ITR” need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV vector need not necessarily be identical or derived from the same AAV serotype or isolate, so long as they function as intended, i.e., to allow for excision and rescue of the sequence of interest from a host cell genome or vector, and to allow integration of the heterologous sequence into the recipient cell genome when AAV Rep gene products are present in the cell.

In one embodiment, AAV ITRs can be derived from any of several AAV serotypes, including without limitation, AAV1, AAV2, AAV3, AAV4, AAV5, AAV7, etc. Furthermore, 5′ and 3′ ITRs which flank a selected nucleotide sequence in an AAV expression vector need not necessarily be identical or derived from the same AAV serotype or isolate, so long as they function as intended, i.e., to allow for excision and rescue of the sequence of interest from a host cell genome or vector, and to allow integration of the DNA molecule into the recipient cell genome when AAV Rep gene products are present in the cell.

In one embodiment, AAV capsids can be derived from AAV2. Suitable DNA molecules for use in AAV vectors will be less than about 5 kilobases (kb), less than about 4.5 kb, less than about 4 kb, less than about 3.5 kb, less than about 3 kb, less than about 2.5 kb in size and are known in the art.

In one embodiment, the selected nucleotide sequence is operably linked to control elements that direct the transcription or expression thereof in the subject in vivo. Such control elements can comprise control sequences normally associated with the selected gene. Alternatively, heterologous control sequences can be employed. Useful heterologous control sequences generally include those derived from sequences encoding mammalian or viral genes. Examples include, but are not limited to, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Such promoter sequences are commercially available from, e.g., Stratagene (San Diego, Calif.).

In one embodiment, both heterologous promoters and other control elements, such as tissue-specific and inducible promoters, enhancers and the like, will be of particular use. Examples of heterologous promoters include the CMV promoter. Examples of inducible promoters include DNA responsive elements for ecdysone, tetracycline, hypoxia and aufin.

In one embodiment, the AAV expression vector that harbors the DNA molecule of interest bounded by AAV ITRs, can be constructed by directly inserting the selected sequence(s) into an AAV genome, which has had the major AAV open reading frames (“ORFs”), excised therefrom. Other portions of the AAV genome can also be deleted, so long as sufficient portions of the ITRs remain to allow for replication and packaging functions. Such constructs can be designed using techniques well known in the art.

Alternatively, AAV ITRs can be excised from the viral genome or from an AAV vector containing the same and fused 5′ and 3′ of a selected nucleic acid construct that is present in another vector using standard ligation techniques. For example, ligations can be accomplished in 20 mM Tris-Cl pH 7.5, 10 mM MgCl2, 10 mM DTT, 33 μg/ml BSA, 10 mM-50 mM NaCl, and either 40 uM ATP, 0.01-0.02 (Weiss) units T4 DNA ligase at 0° C. (for “sticky end” ligation) or 1 mM ATP, 0.3-0.6 (Weiss) units T4 DNA ligase at 14° C. (for “blunt end” ligation). Intermolecular “sticky end” ligations are usually performed at 30-100 μg/ml total DNA concentrations (5-100 nM total end concentration). AAV vectors which contain ITRs.

Additionally, chimeric genes can be produced synthetically to include AAV ITR sequences arranged 5′ and 3′ of one or more selected nucleic acid sequences. The complete chimeric sequence is assembled from overlapping oligonucleotides prepared by standard methods.

In order to produce rAAV virions, an AAV expression vector is introduced into a suitable host cell using known techniques, such as by transfection. A number of transfection techniques are generally known in the art. See, e.g., Sambrook et al. (1989) Molecular Cloning, a laboratory manual, Cold Spring Harbor Laboratories, New York. Particularly suitable transfection methods include calcium phosphate co-precipitation, direct micro-injection into cultured cells, electroporation, liposome mediated gene transfer, lipid-mediated transduction, and nucleic acid delivery using high-velocity microprojectiles.

In one embodiment, suitable host cells for producing rAAV virions include microorganisms, yeast cells, insect cells, and mammalian cells, that can be, or have been, used as recipients of a heterologous DNA molecule. The term includes the progeny of the original cell that has been transfected. Thus, a “host cell” as used herein generally refers to a cell that has been transfected with an exogenous DNA sequence. Cells from the stable human cell line, 293 (readily available through, e.g., the American Type Culture Collection under Accession Number ATCC CRL1573) can be used in the practice of the present disclosure. Particularly, the human cell line 293 is a human embryonic kidney cell line that has been transformed with adenovirus type-5 DNA fragments, and expresses the adenoviral Ela and Elb genes. The 293 cell line is readily transfected, and provides a particularly convenient platform in which to produce rAAV virions.

By “AAV rep coding region” is meant the art-recognized region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome. Suitable homologues of the AAV rep coding region include the human herpesvirus 6 (HHV-6) rep gene which is also known to mediate AAV-2 DNA replication.

By “AAV cap coding region” is meant the art-recognized region of the AAV genome that encodes the capsid proteins VP1, VP2, and VP3, or functional homologues thereof. These Cap expression products supply the packaging functions, which are collectively required for packaging the viral genome.

In one embodiment, AAV helper functions are introduced into the host cell by transfecting the host cell with an AAV helper construct either prior to, or concurrently with, the transfection of the AAV expression vector. AAV helper constructs are thus used to provide at least transient expression of AAV rep and/or cap genes to complement missing AAV functions that are necessary for productive AAV infection. AAV helper constructs lack AAV ITRs and can neither replicate nor package themselves. These constructs can be in the form of a plasmid, phage, transposon, cosmid, virus, or virion. A number of AAV helper constructs have been described, such as the commonly used plasmids pAAV/Ad and pIM29+45 that encode both Rep and Cap expression products. A number of other vectors have been described that encode Rep and/or Cap expression products.

Methods of delivery of viral vectors include injecting the AAV into the subject. Generally, rAAV virions may be introduced into cells using either in vivo or in vitro transduction techniques. If transduced in vitro, the desired recipient cell will be removed from the subject, transduced with rAAV virions and reintroduced into the subject. Alternatively, syngeneic or xenogeneic cells can be used where those cells will not generate an inappropriate immune response in the subject.

Suitable methods for the delivery and introduction of transduced cells into a subject have been described. For example, cells can be transduced in vitro by combining recombinant AAV virions with cells e.g., in appropriate media, and screening for those cells harboring the DNA of interest can be screened using conventional techniques such as Southern blots and/or PCR, or by using selectable markers. Transduced cells can then be formulated into pharmaceutical compositions, described more fully below, and the composition introduced into the subject by various techniques, such as by grafting, intramuscular, intravenous, subcutaneous and intraperitoneal injection.

In one embodiment, pharmaceutical compositions will comprise sufficient genetic material to produce a therapeutically effective amount of the nucleic acid of interest, i.e., an amount sufficient to reduce or ameliorate symptoms of the disease state in question or an amount sufficient to confer the desired benefit. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

It should be understood that more than one transgene could be expressed by the delivered viral vector. Alternatively, separate vectors, each expressing one or more different transgenes, can also be delivered to the subject as described herein. Furthermore, it is also intended that the viral vectors delivered by the methods of the present disclosure be combined with other suitable compositions and therapies.

As is apparent to those skilled in the art in view of the teachings of this specification, an effective amount of viral vector that must be added can be empirically determined. Administration can be effected in one dose, continuously or intermittently throughout the course of treatment. Methods of determining the most effective means and dosages of administration are well known to those of skill in the art and will vary with the viral vector, the composition of the therapy, the target cells, and the subject being treated. Single and multiple administrations can be carried out with the dose level and pattern being selected by the treating physician.

In certain embodiments, the rAAV is administered at a dose of about 0.3-2 ml of 1×105-1×1016 vg/ml. In certain embodiments, the rAAV is administered at a dose of about 1-3 ml of 1×107-1×1014 vg/ml. In certain embodiments, the rAAV is administered at a dose of about 1-2 ml of 1×108-1×1013 vg/ml.

Formulations containing the rAAV particles will contain an effective amount of the rAAV particles in a vehicle, the effective amount being readily determined by one skilled in the art. The rAAV particles may typically range from about 1% to about 95% (w/w) of the composition, or even higher or lower if appropriate. The quantity to be administered depends upon factors such as the age, weight and physical condition of the animal or the human subject considered for treatment. Effective dosages can be established by one of ordinary skill in the art through routine trials establishing dose response curves. The subject is treated by administration of the rAAV particles in one or more doses. Multiple doses may be administered as is required to maintain adequate enzyme activity.

Vehicles including water, aqueous saline, artificial CSF, or other known substances can be employed with the subject invention. To prepare a formulation, the purified composition can be isolated, lyophilized and stabilized. The composition may then be adjusted to an appropriate concentration, optionally combined with an anti-inflammatory agent, and packaged for use.

The present invention provides a method of increasing the level of a target protein in a cell by introducing a protein, or nucleic acid molecule encoding a protein described above into a cell in an amount sufficient to increase the level of the target protein in the cell. In certain embodiments, the accumulation of target protein is increased by at least 10%. The accumulation of target protein is increased by at least 10%, 20%, 30%, 40%, 50%, 60%, 70%, 80%, 90% 95%, or 99%. 39

### Nucleic Acids Encoding Therapeutic Agents

The term “nucleic acid” refers to deoxyribonucleotides or ribonucleotides and polymers thereof in either single-or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides.

A “nucleic acid fragment” is a portion of a given nucleic acid molecule. The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters.

In one embodiment, the nucleic acid molecule is an RNA molecule comprising an ACE-tRNA.

In one embodiment, the nucleic acid molecule is a DNA or cDNA molecule which encodes one or more ACE-tRNA. In one embodiment, the nucleic acid molecule is a nucleic acid molecule encoding 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more or 20 or more ACE-tRNA molecules under the control of a single promoter.

In one embodiment, the invention provides a nucleic acid molecule encoding two or more ACE-tRNA separated by cleavage sites. In one embodiment, a cleavage site is a P2A site. In one embodiment, a cleavage site is a protease cleavage site. In various embodiments, a protease cleavage site may include, but is not limited to, a site targeted by a Caspase (e.g. Caspase 1 through Caspase 10), enterokinase, factor Xa, granzyme B, HRV3C protease, hydroxylamine, pancreatic elastase, pepsin A, prolyl endopeptidase, proteinase K, TEV protease, thermolysin, or thrombin. In one embodiment, a protease for cleavage of the protease cleavage sites is encoded downstream of two or more genes encoding reporter molecules as part of the same polypeptide. In one embodiment, the nucleotide sequence encoding the protease is linked to the nucleotide sequence of two or more genes encoding ACE-tRNA by a protease cleavage site.

In one embodiment, the nucleic acid molecule comprises one or more reporter molecule. A reporter is a molecule whose expression a cell confers a detectable trait to the cell. In various embodiments, reporters include, but are not limited to, chloramphenicol-acetyl transferase (CAT), β-galactosyltransferase, horseradish peroxidase, luciferase, NanoLuc®, alkaline phosphatase, and fluorescent proteins including, but not limited to, green fluorescent proteins (e.g. GFP, TagGFP, T-Sapphire, Azami Green, Emerald, mWasabi, mClover3), red fluorescent proteins (e.g. tdTomato, mRFP1, JRed, HcRed1, AsRed2, AQ143, mCherry, mRuby3, mPlum), yellow fluorescent proteins (e.g. EYFP, mBanana, mCitrine, PhiYFP, TagYFP, Topaz, Venus), orange fluorescent proteins (e.g. DsRed, Tomato, Kusabria Orange, mOrange, mTangerine, TagRFP), cyan fluorescent proteins (e.g. CFP, mTFP1, Cerulean, CyPet, AmCyan1), blue fluorescent proteins (e.g. Azurite, mtagBFP2, EBFP, EBFP2, Y66H), near-infrared fluorescent proteins (e.g. iRFP670, iRFP682, iRFP702, iRFP713 and iRFP720), infrared fluorescent proteins (e.g. IFP1.4) and photoactivatable fluorescent proteins (e.g. Kaede, Eos, IrisFP, PS-CFP).

### Methods for Introducing Genetic Material Into Cells

The exogenous genetic material (e.g., a DNA encoding one or more therapeutic ACE-tRNAs) is introduced into the cell in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation; DEAE-dextran; electroporation; cationic liposome-mediated transfection; and tungsten particle-facilitated microparticle bombardment. Strontium phosphate DNA co-precipitation is another possible transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion, an “enhancer” is simply any non-translated DNA sequence that works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes that encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eucaryotic cells. These include the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter, is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

An ACE-tRNA construct of the present invention can be inserted into any type of target or host cell. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20 □C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The nucleic acid molecule of the invention can be administered via electroporation, such as by a method described in U.S. Pat. No. 7,664,545, the contents of which are incorporated herein by reference. The electroporation can be by a method and/or apparatus described in U.S. Pat. Nos. 6,302,874; 5,676,646; 6,241,701; 6,233,482; 6,216,034; 6,208,893; 6,192,270; 6,181,964; 6,150,148; 6,120,493; 6,096,020; 6,068,650; and 5,702,359, the contents of which are incorporated herein by reference in their entirety. The electroporation may be carried out via a minimally invasive device.

The minimally invasive electroporation device (“MID”) may be an apparatus for injecting the composition described above and associated fluid into body tissue. The device may comprise a hollow needle, DNA cassette, and fluid delivery means, wherein the device is adapted to actuate the fluid delivery means in use so as to concurrently (for example, automatically) inject DNA into body tissue during insertion of the needle into the said body tissue. This has the advantage that the ability to inject the DNA and associated fluid gradually while the needle is being inserted leads to a more even distribution of the fluid through the body tissue. The pain experienced during injection may be reduced due to the distribution of the DNA being injected over a larger area.

The MID may inject the composition into tissue without the use of a needle. The MID may inject the composition as a small stream or jet with such force that the composition pierces the surface of the tissue and enters the underlying tissue and/or muscle. The force behind the small stream or jet may be provided by expansion of a compressed gas, such as carbon dioxide through a micro-orifice within a fraction of a second. Examples of minimally invasive electroporation devices, and methods of using them, are described in published U.S. patent application Ser. No. 20080234655; U.S. Pat. Nos. 6,520,950; 7,171,264; 6,208,893; 6,009,347; 6,120,493; 7,245,963; 7,328,064; and 6,763,264, the contents of each of which are herein incorporated by reference.

The MID may comprise an injector that creates a high-speed jet of liquid that painlessly pierces the tissue. Such needle-free injectors are commercially available. Examples of needle-free injectors that can be utilized herein include those described in U.S. Pat. Nos. 3,805,783; 4,447,223; 5,505,697; and 4,342,310, the contents of each of which are herein incorporated by reference.

A desired composition in a form suitable for direct or indirect electrotransport may be introduced (e.g., injected) using a needle-free injector into the tissue to be treated, usually by contacting the tissue surface with the injector so as to actuate delivery of a jet of the agent, with sufficient force to cause penetration of the composition into the tissue. For example, if the tissue to be treated is mucosa, skin or muscle, the agent is projected towards the mucosal or skin surface with sufficient force to cause the agent to penetrate through the stratum corneum and into dermal layers, or into underlying tissue and muscle, respectively.

Needle-free injectors are well suited to deliver compositions to all types of tissues, particularly to skin and mucosa. In some embodiments, a needle-free injector may be used to propel a liquid that contains the composition to the surface and into the subject's skin or mucosa. Representative examples of the various types of tissues that can be treated using the invention methods include pancreas, larynx, nasopharynx, hypopharynx, oropharynx, lip, throat, lung, heart, kidney, muscle, breast, colon, prostate, thymus, testis, skin, mucosal tissue, ovary, blood vessels, or any combination thereof.

The MID may have needle electrodes that electroporate the tissue. By pulsing between multiple pairs of electrodes in a multiple electrode array, for example set up in rectangular or square patterns, provides improved results over that of pulsing between a pair of electrodes. Disclosed, for example, in U.S. Pat. No. 5,702,359 entitled “Needle Electrodes for Mediated Delivery of Drugs and Genes” is an array of needles wherein a plurality of pairs of needles may be pulsed during the therapeutic treatment. In that application, which is incorporated herein by reference as though fully set forth, needles were disposed in a circular array, but have connectors and switching apparatus enabling a pulsing between opposing pairs of needle electrodes. A pair of needle electrodes for delivering recombinant expression vectors to cells may be used. Such a device and system is described in U.S. Pat. No. 6,763,264, the contents of which are herein incorporated by reference. Alternatively, a single needle device may be used that allows injection of the DNA and electroporation with a single needle resembling a normal injection needle and applies pulses of lower voltage than those delivered by presently used devices, thus reducing the electrical sensation experienced by the patient.

The MID may comprise one or more electrode arrays. The arrays may comprise two or more needles of the same diameter or different diameters. The needles may be evenly or unevenly spaced apart. The needles may be between 0.005 inches and 0.03 inches, between 0.01 inches and 0.025 inches; or between 0.015 inches and 0.020 inches. The needle may be 0.0175 inches in diameter. The needles may be 0.5 mm, 1.0 mm, 1.5 mm, 2.0 mm, 2.5 mm, 3.0 mm, 3.5 mm, 4.0 mm, or more spaced apart.

The MID may consist of a pulse generator and a two or more-needle composition injectors that deliver the composition and electroporation pulses in a single step. The pulse generator may allow for flexible programming of pulse and injection parameters via a flash card operated personal computer, as well as comprehensive recording and storage of electroporation and patient data. The pulse generator may deliver a variety of volt pulses during short periods of time. For example, the pulse generator may deliver three 15 volt pulses of 100 ms in duration. An example of such a MID is the Elgen 1000 system by Inovio Biomedical Corporation, which is described in U.S. Pat. No. 7,328,064, the contents of which are herein incorporated by reference.

The MID may be a CELLECTRA (Inovio Pharmaceuticals, Blue Bell PA) device and system, which is a modular electrode system, that facilitates the introduction of a macromolecule, such as a DNA, into cells of a selected tissue in a body or plant. The modular electrode system may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The macromolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the macromolecule into the cell between the plurality of electrodes. Cell death due to overheating of cells is minimized by limiting the power dissipation in the tissue by virtue of constant-current pulses. The Cellectra device and system is described in U.S. Pat. No. 7,245,963, the contents of which are herein incorporated by reference.

The MID may be an Elgen 1000 system (Inovio Pharmaceuticals). The Elgen 1000 system may comprise device that provides a hollow needle; and fluid delivery means, wherein the apparatus is adapted to actuate the fluid delivery means in use so as to concurrently (for example automatically) inject fluid, the described composition herein, into body tissue during insertion of the needle into the said body tissue. The advantage is the ability to inject the fluid gradually while the needle is being inserted leads to a more even distribution of the fluid through the body tissue. It is also believed that the pain experienced during injection is reduced due to the distribution of the volume of fluid being injected over a larger area.

In addition, the automatic injection of fluid facilitates automatic monitoring and registration of an actual dose of fluid injected. This data can be stored by a control unit for documentation purposes if desired.

It will be appreciated that the rate of injection could be either linear or non-linear and that the injection may be carried out after the needles have been inserted through the skin of the subject to be treated and while they are inserted further into the body tissue.

Suitable tissues into which fluid may be injected by the apparatus of the present invention include tumor tissue, skin or liver tissue but may be muscle tissue.

The apparatus further comprises needle insertion means for guiding insertion of the needle into the body tissue. The rate of fluid injection is controlled by the rate of needle insertion. This has the advantage that both the needle insertion and injection of fluid can be controlled such that the rate of insertion can be matched to the rate of injection as desired. It also makes the apparatus easier for a user to operate. If desired means for automatically inserting the needle into body tissue could be provided.

A user could choose when to commence injection of fluid. Ideally however, injection is commenced when the tip of the needle has reached muscle tissue and the apparatus may include means for sensing when the needle has been inserted to a sufficient depth for injection of the fluid to commence. This means that injection of fluid can be prompted to commence automatically when the needle has reached a desired depth (which will normally be the depth at which muscle tissue begins). The depth at which muscle tissue begins could for example be taken to be a preset needle insertion depth such as a value of 4 mm which would be deemed sufficient for the needle to get through the skin layer.

The sensing means may comprise an ultrasound probe. The sensing means may comprise a means for sensing a change in impedance or resistance. In this case, the means may not as such record the depth of the needle in the body tissue but will rather be adapted to sense a change in impedance or resistance as the needle moves from a different type of body tissue into muscle. Either of these alternatives provides a relatively accurate and simple to operate means of sensing that injection may commence. The depth of insertion of the needle can further be recorded if desired and could be used to control injection of fluid such that the volume of fluid to be injected is determined as the depth of needle insertion is being recorded.

The apparatus may further comprise: a base for supporting the needle; and a housing for receiving the base therein, wherein the base is moveable relative to the housing such that the needle is retracted within the housing when the base is in a first rearward position relative to the housing and the needle extends out of the housing when the base is in a second forward position within the housing. This is advantageous for a user as the housing can be lined up on the skin of a patient, and the needles can then be inserted into the patient's skin by moving the housing relative to the base.

As stated above, it is desirable to achieve a controlled rate of fluid injection such that the fluid is evenly distributed over the length of the needle as it is inserted into the skin. The fluid delivery means may comprise piston driving means adapted to inject fluid at a controlled rate. The piston driving means could for example be activated by a servo motor. However, the piston driving means may be actuated by the base being moved in the axial direction relative to the housing. It will be appreciated that alternative means for fluid delivery could be provided. Thus, for example, a closed container which can be squeezed for fluid delivery at a controlled or non-controlled rate could be provided in the place of a syringe and piston system.

The apparatus described above could be used for any type of injection. It is however envisaged to be particularly useful in the field of electroporation and so it may further comprises means for applying a voltage to the needle. This allows the needle to be used not only for injection but also as an electrode during, electroporation. This is particularly advantageous as it means that the electric field is applied to the same area as the injected fluid. There has traditionally been a problem with electroporation in that it is very difficult to accurately align an electrode with previously injected fluid and so users have tended to inject a larger volume of fluid than is required over a larger area and to apply an electric field over a higher area to attempt to guarantee an overlap between the injected substance and the electric field. Using the present invention, both the volume of fluid injected and the size of electric field applied may be reduced while achieving a good fit between the electric field and the fluid.

Regardless of the method used to introduce exogenous nucleic acids into a host cell, in order to confirm the presence of the recombinant nucleic acic sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or other assays known to those of skill in the art.

### Disease Conditions and Methods of Treatment

The present invention in one embodiment includes compositions and methods for treating cystic fibrosis by reversing the effects of mutations present that are associated with nonsense mutations through introduction of the synthetic oligonucleotide suppressor tRNAs of the invention.

Certain embodiments of the present disclosure provide a method of treating a disease in a mammal comprising administering a protein or vector encoding a therapeutic agent (e.g., a modified and/or stabilized ACE-tRNA) as described herein to the mammal. In certain embodiments, the mammal is human.

Certain embodiments of the present disclosure provide a use of a therapeutic agent or vector encoding a therapeutic agent as described herein to prepare a medicament useful for treating disease in a mammal. Diseases or disorders associated with PTCs include, but are not limited to, variants of Duchenne and Becker muscular dystrophies due to a PTC in dystrophin, retinoblastoma due to a PTC in RB1, neurofibromatosis due to a PTC in NF1 or NF2, ataxia-telangiectasia due to a PTC in ATM, Tay-Sachs disease due to a PTC in HEXA, cystic fibrosis due to a PTC in CFTR, Wilm's tumor due to a PTC in WT1, hemophilia A due to a PTC in factor VIII, hemophilia B due to a PTC in factor IX, p53-associated cancers due to a PTC in p53, Menkes disease, Ullrich's disease, β-Thalassemia due to a PTC in betaglobin, type 2A and type 3 von Willebrand disease due to a PTC in Willebrand factor, Robinow syndrome, brachydactyly type B (shortening of digits and metacarpals), inherited susceptibility to mycobacterial infection due to a PTC in IFNGR1, inherited retinal disease due to a PTC in CRX, inherited bleeding tendency due to a PTC in Coagulation factor X, inherited blindness due to a PTC in Rhodopsin, congenital neurosensory deafness and colonic agangliosis due to a PTC in SOX10 and inherited neural develop-mental defect including neurosensory deafness, colonic agangliosis, peripheral neuropathy and central dysmyelinating leukodystrophy due to a PTC in SOX10, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and ricketsand many others.

The present disclosure also provides a mammalian cell containing a vector described herein. The cell may be human.

Certain aspects of the disclosure relate to polynucleotides, polypeptides, vectors, and genetically engineered cells (modified in vivo), and the use of them. In particular, the disclosure relates to a method for gene therapy that is capable of both systemic delivery of a therapeutically effective dose of the therapeutic agent.

According to one aspect, a cell expression system for expressing a therapeutic agent in a mammalian recipient is provided. The expression system (also referred to herein as a “genetically modified cell”) comprises a cell and an expression vector for expressing the therapeutic agent. Expression vectors include, but are not limited to, viruses, plasmids, and other vehicles for delivering heterologous genetic material to cells. Accordingly, the term “expression vector” as used herein refers to a vehicle for delivering heterologous genetic material to a cell. In particular, the expression vector is a recombinant adenoviral, adeno-associated virus, or lentivirus or retrovirus vector.

The expression vector further includes a promoter for controlling transcription of the heterologous gene. The promoter may be an inducible promoter (described herein). The expression system is suitable for administration to the mammalian recipient. The expression system may comprise a plurality of non-immortalized genetically modified cells, each cell containing at least one recombinant gene encoding at least one therapeutic agent.

The cell expression system is formed in vivo. According to yet another aspect, a method for treating a mammalian recipient in vivo is provided. The method includes introducing an expression vector for expressing a heterologous gene product into a cell of the patient in situ, such as via intravenous administration. To form the expression system in vivo, an expression vector for expressing the therapeutic agent is introduced in vivo into the mammalian recipient i.v.

According to yet another aspect, a method for treating a mammalian recipient in vivo is provided. The method includes introducing the target therapeutic agent into the patient in vivo.

The expression vector for expressing the heterologous gene may include an inducible promoter for controlling transcription of the heterologous gene product. Accordingly, delivery of the therapeutic agent in situ is controlled by exposing the cell in situ to conditions, which induce transcription of the heterologous gene.

The present disclosure provides methods of treating a disease in a mammal by administering an expression vector to a cell or patient. For the gene therapy methods, a person having ordinary skill in the art of molecular biology and gene therapy would be able to determine, without undue experimentation, the appropriate dosages and routes of administration of the expression vector used in the novel methods of the present disclosure.

The present disclosure provides methods of treating a disease in a mammal by administering at least one ACE-tRNA to a cell or patient. For the gene therapy methods, a person having ordinary skill in the art of molecular biology and gene therapy would be able to determine, without undue experimentation, the appropriate dosages and routes of administration of the ACE-tRNA used in the novel methods of the present disclosure.

In one embodiment, the disclosure provides methods of treating a disease in a mammal by administering at least two, at least 3, at least 4, or more than 4 ACE-tRNA or nucleic acid molecules encoding ACE-tRNA to a cell or patient. In one embodiment, the methods include administration of multiple ACE-tRNA or nucleic acid molecules encoding multiple ACE-tRNA, wherein each of the multiple ACE-tRNA are specific for incorporation of a distict amino acid from other ACE-tRNA in the composition. For example, in one embodiment, the method includes administration of a combination of a first ACE-tRNA for incorporation of Arg into a polypeptide and a second ACE-tRNA for incorporation of Gly into a polypeptide. In one embodiment, multiple ACE-tRNA are specific for the PTC (e.g., UGA). In one embodiment, multiple ACE-tRNA are specific for different PTC.

According to one embodiment, the cells are transformed or otherwise genetically modified in vivo. The cells from the mammalian recipient are transformed (i.e., transduced or transfected) in vivo with a vector containing exogenous genetic material for expressing a heterologous (e.g., recombinant) gene encoding a therapeutic agent and the therapeutic agent is delivered in situ.

As used herein, “exogenous genetic material” refers to a nucleic acid or an oligonucleotide, either natural or synthetic, that is not naturally found in the cells; or if it is naturally found in the cells, it is not transcribed or expressed at biologically significant levels by the cells. Thus, “exogenous genetic material” includes, for example, a non-naturally occurring nucleic acid that can be transcribed into a tRNA.

The above-disclosed therapeutic agents and conditions amenable to gene therapy are merely illustrative and are not intended to limit the scope of the instant disclosure. The selection of a suitable therapeutic agent for treating a known condition is deemed to be within the scope of one of ordinary skill of the art without undue experimentation.

In certain embodimen ts, the therapy has potential use for the treatment/management of diseases that are caused by Premature Termination Codons (PTCs), including, but not limited to, Duchenne and Becker muscular dystrophies, retinoblastoma, neurofibromatosis, ataxia-telangiectasia, Tay-Sachs disease, cystic fibrosis, Wilm's tumor, hemophilia A, hemophilia B, Menkes disease, Ullric's disease, β-Thalassemia, type 2A and type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness and colonic agangliosis and inherited neural develop-mental defect including neurosensory deafness, colonic agangliosis, peripheral neuropathy and central dysmyelinating leukodystrophy, Liddle's syndrome, xeroderma pigmentosum, Fanconi's anemia, anemia, hypothyroidism, p53-associated cancers (e.g., p53 squamal cell carcinoma, p53 hepatocellular carcinoma, p53 ovarian carcinoma), esophageal carcinoma, osteocarcinoma, ovarian carcinoma, hepatocellular carcinoma, breast cancer, hepatocellular carcinoma, fibrous histiocytoma, ovarian carcinoma, SRY sex reversal, triosephosphate isomerase-anemia, diabetes and rickets. This therapy is advantageous in that it provides improved stop codon suppression specificity. The therapeutic ACE-tRNAs of the present invention target a specific stop-codon, TGA for instance, thus reducing off-target effects at stop-codons unrelated to disease. The present therapy is also advantageous in that it provides amino-acid specificity. The expressed tRNA is engineered to specifically replace the amino acid that was lost via insertion of the disease stop codon, thus negating any spurious effects on protein stability, folding and trafficking.

In certain embodiments, the present system is modular, and thus can be “personalized” to every possible disease PTC. For instnce, there are nine individual tryptophan tRNAs in the human genome that are recognized by the Trp synthetase, all of which suppress the mRNA UGG codon. Thus, each of these nine Trp tRNA provides an opportunity for codon re-editing tolerance (UGG␣UGA). Additionally, given their proximity to stop codons in the genetic code, the mutation of arginine codons to PTC nonsense codons are common in disease. There are over thirty Arg IRNA that could be tested for codon editing tolerance and suppression efficacy.

A further advantage of the present invention is that it provides facile expression and cell specific delivery, because the entire system (IRNA +promoter sequence) is compact.

### Dosages, Formulations and Routes of Administration of the Agents of the Invention

The agents of the invention are administered so as to result in a reduction in at least one symptom associated with a genetic disease (e.g., cystic fibrosis). The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems that are well known to the art.

The present invention envisions treating a disease or disorder associated with a PTC by the administration of an agent, e.g., ACE-tRNA, an expression vector, or a viral particle of the invention. Administration of the therapeutic agents in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated.

One or more suitable unit dosage forms having the therapeutic agent(s) of the invention, which, as discussed below, may optionally be formulated for sustained release (for example using microencapsulation), can be administered by a variety of routes including parenteral, including by intravenous and intramuscular routes, as well as by direct injection into the diseased tissue. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well-known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water.

### Methods of Administration

Provided herein are methods of treating, protecting against, and/or preventing a PTC associated disease in a subject in need thereof by administering one or more composition described herein to the subject.

The composition dose can be between 1 μg to 10 mg active component/kg body weight/time, and can be 20 μg to 10 mg component/kg body weight/time. The composition can be administered every 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days. The number of composition doses for effective treatment can be 1, 2, 3, 4, 5, 6, 7, 8, 9, or 10.

The composition can be formulated in accordance with standard techniques well known to those skilled in the pharmaceutical art. Such compositions can be administered in dosages and by techniques well known to those skilled in the medical arts taking into consideration such factors as the age, sex, weight, and condition of the particular subject, and the route of administration.

The composition can be administered prophylactically or therapeutically. In therapeutic applications, the compositions are administered to a subject in need thereof in an amount sufficient to elicit a therapeutic effect. An amount adequate to accomplish this is defined as “therapeutically effective dose.” Amounts effective for this use will depend on, e.g., the particular composition of the composition regimen administered, the manner of administration, the stage and severity of the disease, the general state of health of the subject, and the judgment of the prescribing physician.

The composition can be administered by methods well known in the art as described in Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997), the contents of all of which are incorporated herein by reference in their entirety. The DNA of the composition can be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The composition can be delivered via a variety of routes. Typical delivery routes include parenteral administration, e.g., intradermal, intramuscular or subcutaneous delivery. Other routes include oral administration, intranasal, and intravaginal routes. For the DNA of the composition in particular, the composition can be delivered to the interstitial spaces of tissues of an individual (Felgner et al., U.S. Pat. Nos. 5,580,859 and 5,703,055, the contents of all of which are incorporated herein by reference in their entirety). The composition can also be administered to muscle, or can be administered via intradermal or subcutaneous injections, or transdermally, such as by iontophoresis. Epidermal administration of the composition can also be employed. Epidermal administration can involve mechanically or chemically irritating the outermost layer of epidermis to stimulate an immune response to the irritant (Carson et al., U.S. Pat. No. 5,679,647, the contents of which are incorporated herein by reference in its entirety).

The composition can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the composition.

The composition can be a liquid preparation such as a suspension, syrup or elixir. The composition can also be a preparation for parenteral, subcutaneous, intradermal, intramuscular or intravenous administration (e.g., injectable administration), such as a sterile suspension or emulsion.

The composition can be incorporated into liposomes, microspheres or other polymer matrices (Felgner et al., U.S. Pat. No. 5,703,055; Gregoriadis, Liposome Technology, Vols. Ito III (2nd ed. 1993), the contents of which are incorporated herein by reference in their entirety). Liposomes can consist of phospholipids or other lipids, and can be nontoxic, physiologically acceptable and metabolizable carriers that are relatively simple to make and administer.

The ACE-tRNA or nucleic acid molecule encoding the ACE-tRNA may be administered by different routes including orally, parenterally, sublingually, transdermally, rectally, transmucosally, topically, via inhalation, via buccal administration, intrapleurally, intravenous, intraarterial, intraperitoneal, subcutaneous, intramuscular, intranasal, intrathecal, and intraarticular or combinations thereof. For veterinary use, the composition may be administered as a suitably acceptable formulation in accordance with normal veterinary practice. The veterinarian can readily determine the dosing regimen and route of administration that is most appropriate for a particular animal. The composition may be administered by traditional syringes, needleless injection devices, “microprojectile bombardment gone guns”, or other physical methods such as electroporation (“EP”), “hydrodynamic method”, or ultrasound.

The ACE-tRNA or nucleic acid molecule encoding the ACE-tRNA may be delivered to the mammal by several well-known technologies including DNA injection (also referred to as DNA vaccination) with and without in vivo electroporation, liposome mediated, nanoparticle facilitated, recombinant vectors such as recombinant adenovirus, recombinant adenovirus associated virus and recombinant vaccinia. The ACE-tRNA or nucleic acid molecule encoding the ACE-tRNA may be delivered via DNA injection and along with in vivo electroporation.

### Electroporation

Administration of the composition via electroporation may be accomplished using electroporation devices that can be configured to deliver to a desired tissue of a mammal a pulse of energy effective to cause reversible pores to form in cell membranes, and preferable the pulse of energy is a constant current similar to a preset current input by a user. The electroporation device may comprise an electroporation component and an electrode assembly or handle assembly. The electroporation component may include and incorporate one or more of the various elements of the electroporation devices, including: controller, current waveform generator, impedance tester, waveform logger, input element, status reporting element, communication port, memory component, power source, and power switch. The electroporation may be accomplished using an in vivo electroporation device, for example CELLECTRA EP system (Inovio Pharmaceuticals, Plymouth Meeting, PA) or Elgen electroporator (Inovio Pharmaceuticals, Plymouth Meeting, PA) to facilitate transfection of cells by the plasmid.

The electroporation component may function as one element of the electroporation devices, and the other elements are separate elements (or components) in communication with the electroporation component. The electroporation component may function as more than one element of the electroporation devices, which may be in communication with still other elements of the electroporation devices separate from the electroporation component. The elements of the electroporation devices existing as parts of one electromechanical or mechanical device may not limited as the elements can function as one device or as separate elements in communication with one another. The electroporation component may be capable of delivering the pulse of energy that produces the constant current in the desired tissue, and includes a feedback mechanism. The electrode assembly may include an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism may receive the measured impedance and can adjust the pulse of energy delivered by the electroporation component to maintain the constant current.

A plurality of electrodes may deliver the pulse of energy in a decentralized pattern. The plurality of electrodes may deliver the pulse of energy in the decentralized pattern through the control of the electrodes under a programmed sequence, and the programmed sequence is input by a user to the electroporation component. The programmed sequence may comprise a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

The feedback mechanism may be performed by either hardware or software. The feedback mechanism may be performed by an analog closed-loop circuit. The feedback occurs every 50 μs, 20 μs, 10 μs or 1 μs, but is preferably a real-time feedback or instantaneous (i.e., substantially instantaneous as determined by available techniques for determining response time). The neutral electrode may measure the impedance in the desired tissue and communicates the impedance to the feedback mechanism, and the feedback mechanism responds to the impedance and adjusts the pulse of energy to maintain the constant current at a value similar to the preset current. The feedback mechanism may maintain the constant current continuously and instantaneously during the delivery of the pulse of energy.

Examples of electroporation devices and electroporation methods that may facilitate delivery of the compositions of the present invention, include those described in U.S. Pat. No. 7,245,963 by Draghia-Akli, et al., U.S. Patent Pub. 2005/0052630 submitted by Smith, et al., the contents of which are hereby incorporated by reference in their entirety. Other electroporation devices and electroporation methods that may be used for facilitating delivery of the compositions include those provided in U.S. patent application Ser. No. 11/874,072, filed Oct. 17, 2007, which claims the benefit under 35 USC 119 (c) to U.S. Provisional Applications Ser. Nos. 60/852,149, filed Oct. 17, 2006, and 60/978,982, filed Oct. 10, 2007, all of which are hereby incorporated in their entirety.

U.S. Pat. No. 7,245,963 by Draghia-Akli, et al. describes modular electrode systems and their use for facilitating the introduction of a biomolecule into cells of a selected tissue in a body or plant. The modular electrode systems may comprise a plurality of needle electrodes; a hypodermic needle; an electrical connector that provides a conductive link from a programmable constant-current pulse controller to the plurality of needle electrodes; and a power source. An operator can grasp the plurality of needle electrodes that are mounted on a support structure and firmly insert them into the selected tissue in a body or plant. The biomolecules are then delivered via the hypodermic needle into the selected tissue. The programmable constant-current pulse controller is activated and constant-current electrical pulse is applied to the plurality of needle electrodes. The applied constant-current electrical pulse facilitates the introduction of the biomolecule into the cell between the plurality of electrodes. The entire content of U.S. Pat. No. 7,245,963 is hereby incorporated by reference.

U.S. Patent Pub. 2005/0052630 submitted by Smith, et al. describes an electroporation device which may be used to effectively facilitate the introduction of a biomolecule into cells of a selected tissue in a body or plant. The electroporation device comprises an electro-kinetic device (“EKD device”) whose operation is specified by software or firmware. The EKD device produces a series of programmable constant-current pulse patterns between electrodes in an array based on user control and input of the pulse parameters, and allows the storage and acquisition of current waveform data. The electroporation device also comprises a replaceable electrode disk having an array of needle electrodes, a central injection channel for an injection needle, and a removable guide disk. The entire content of U.S. Patent Pub. 2005/0052630 is hereby incorporated by reference.

The electrode arrays and methods described in U.S. Pat. No. 7,245,963 and U.S. Patent Pub. 2005/0052630 may be adapted for deep penetration into not only tissues such as muscle, but also other tissues or organs. Because of the configuration of the electrode array, the injection needle (to deliver the biomolecule of choice) is also inserted completely into the target organ, and the injection is administered perpendicular to the target issue, in the area that is pre-delineated by the electrodes The electrodes described in U.S. Pat. No. 7,245,963 and U.S. Patent Pub. 2005/005263 are preferably 20 mm long and 21 gauge.

Additionally, contemplated in some embodiments that incorporate electroporation devices and uses thereof, there are electroporation devices that are those described in the following patents: U.S. Pat. No. 5,273,525 issued Dec. 28, 1993, U.S. Pat. No. 6,110,161 issued Aug. 29, 2000, U.S. Pat. No. 6,261,281 issued Jul. 17, 2001, and U.S. Pat. No. 6,958,060 issued Oct. 25, 2005, and U.S. Pat. No. 6,939,862 issued Sep. 6, 2005. Furthermore, patents covering subject matter provided in U.S. Pat. No. 6,697,669 issued Feb. 24, 2004, which concerns delivery of DNA using any of a variety of devices, and U.S. Pat. No. 7,328,064 issued Feb. 5, 2008, drawn to method of injecting DNA are contemplated herein. The above-patents are incorporated by reference in their entirety.

### Method of Preparing the ACE-tRNA

Provided herein are methods for preparing the DNA plasmids that comprise the ACE-tRNA discussed herein. The DNA plasmids, after the final subcloning step into the mammalian expression plasmid, can be used to inoculate a cell culture in a large-scale fermentation tank, using known methods in the art.

The DNA plasmids for use with the EP devices of the present invention can be formulated or manufactured using a combination of known devices and techniques, but preferably they are manufactured using an optimized plasmid manufacturing technique that is described in a US published application Ser. No. 20090004716, which was filed on May 23, 2007. In some examples, the DNA plasmids used in these studies can be formulated at concentrations greater than or equal to 10 mg/mL. The manufacturing techniques also include or incorporate various devices and protocols that are commonly known to those of ordinary skill in the art, in addition to those described in U.S. Ser. No. 60/939,792, including those described in a licensed patent, U.S. Pat. No. 7,238,522, which issued on Jul. 3, 2007. The above-referenced application and patent, U.S. Ser. No. 60/939,792 and U.S. Pat. No. 7,238,522, respectively, are hereby incorporated in their entirety.

### DEFINITIONS

Disease state: For the purposes of the present invention, a “disease state” or “disease phenotype” is a characteristic of a mammalian cell that results from a stop codon within the coding region of a gene inside the cell (e.g., that results from a nonsense mutation). For example, an increasing number of human genetic diseases are thought to be caused by nonsense mutations (see, for example, Atkinson et al., Nuc. Acids Res. 22:1327, 1994). To give but a few examples, β-thalessemia, Duchenne muscular dystrophy, xeroderma pigmentosum, Fanconi's anemia, and cystic fibrosis can all be caused by nonsense mutations in identified genes.

Endogenous tRNA synthetase: A tRNA synthetase is considered to be “endogenous” to a cell if it is present in the cell into which a tRNA is introduced according to the present invention. As will be the apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

Suppressor tRNA: A “suppressor tRNA” is one whose anti-codon is complementary with a codon that would otherwise terminate translation, so that detectable read-through occurs under the conditions of the experiment. Standard termination codons are amber (UAG), ochre (UAA), and opal (UGA) codons. However, non-standard termination codons (e.g., 4-nucleotide codons) have also been employed in the literature (see, for example, Moore et al., J. Mol. Biol. 298:195, 2000; Hohsaka et al., J. Am. Chem. Soc. 121:12194, 1999).

The invention is now illustrated by the following non-limiting Examples.

### EXAMPLE 1

The genetic code uses four nucleotides that in turn form triplet codons, which form the basis for DNA to protein translation. There are 64 codons in total, 61 of which are used to encode amino acids, and three (TAG, TGA and TAA) of which encode protein termination “stop” or “nonsense” codons.

Five to ten percent of cystic fibrosis cases are caused by “nonsense” mutations that lead to premature truncation of the cystic fibrosis transmembrane conductance regulator (CFTR) protein. An example of this “class 1” mutation is p.Trp1282X, a premature termination codon (PTC) which causes a loss of CFTR function and severe cystic fibrosis phenotypes. Some compounds, such as ataluren, promote stop read-through of disease producing nonsense mutations but have been only modestly successful as therapeutics due to a number of caveats, including poor stop-codon specificity and unexpectedly low efficiency of codon skipping in vivo. However, the widespread use of these compounds and the discovery that endogenous stop-codon read-through is common in metazoans, suggests that assisted suppression could be viable if delivered to a subset of cell types, i.e., airway epithelium. Yet, when therapeutically assisted stop-codon read-through is successful, the nonselective incorporation of an amino acid at the location of the nonsense codon has the potential to affect protein folding, trafficking and function (as is the case with CFTR 1282X); and thus, requires additional therapeutic intervention. Thus, there is an acute unmet need to understand the nature of disease PTCs and potentially therapeutic suppressors and generally, more effective treatments of PTC diseases.

This Example characterizes anticodon edited (ACE) Trp-tRNA for the rescue of CFTR p.Trp1282X channels. Such tRNAs are engineered to ‘suppress’ the disease-causing TGA stop codon and incorporate the original amino acid, Trp at p.Trp1282X CFTR, in effect, genetically reconstructing the wild-type CFTR protein. Data demonstrate that this general approach (nonsense suppression) produces robust rescue of transcripts that carry in-frame stop codons, through either transient transfection of a tRNA and its cognate synthetase in adherent cells, or their virus-based delivery to more native airway cell-types, such as A549 airway cells. This approach offers a number of significant benefits over existing strategies: 1) Improved codon specificity—the expressed tRNA may be directed towards a specific stop-codon, thus reducing off-target effects at stop-codons unrelated to disease. 2) Amino-acid specificity—the expressed tRNA and/or synthetase can be engineered to replace the amino acid that was lost via insertion of the disease stop codon, thus negating any spurious effects on CFTR stability, folding and trafficking. 3) Tunability—the system can be theoretically personalized for each type of tRNA and PTC mutation. 4) Facile expression—the entire system is compact (<1 kb) and can be easily packaged and expressed transiently or via nanoparticle delivery of tRNA. 5) Proof of principle for a general strategy—in-frame stop codons are a major cause of human disease and few treatment options exist; the experiments performed here on p.Trp1282X are expected to lead to insights into the mechanisms of other CFTR nonsense codons.

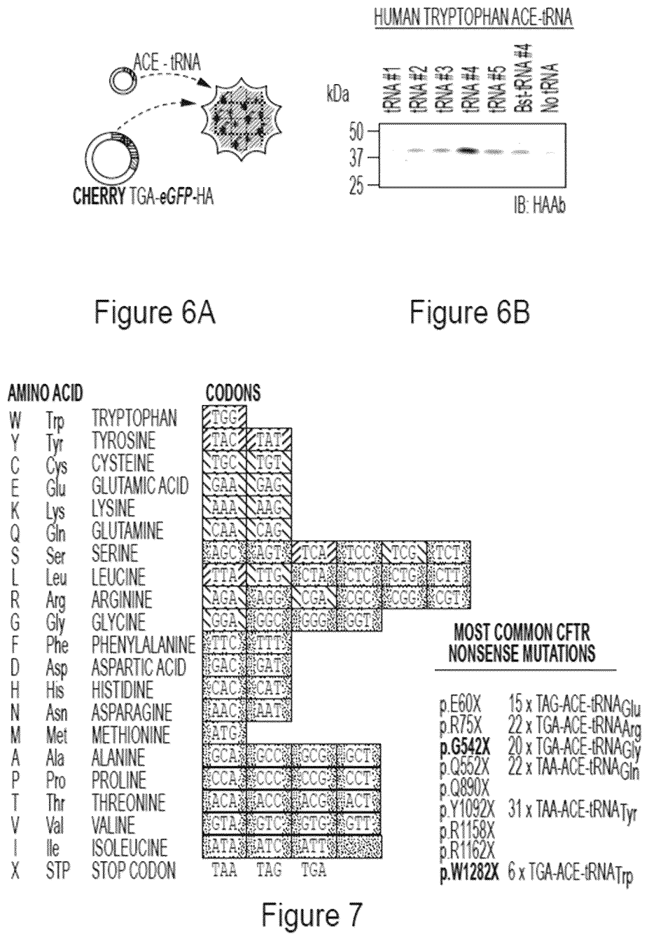

Data shows that ACE-tRNA stop-codon suppressor tRNA are efficient at “rescuing” transcripts which contain introduced stop-sites (FIGS. 6A and 6B) suggesting that such tRNA have the potential to interfere with nonsense mediated decay (NMD) as the major biological hurdle in the therapeutic rescue of disease stop sites. Thus opening the possibility for the use of suppressor tRNA to gain more molecular insights into NMD in disease.

### RESULTS

Eukaryotic tRNA that had been anticodon edited to suppress stop sites, TGA for instance, and not its designated codon were developed. They were tested in five human tryptophan tRNA on a test construct consisting of a fluorescent protein (cherry) in frame with eGFP sequence that are separated by a linker containing a TGA site. To indicate the production of the full-length protein an HA epitope was added to the C-terminus of the eGFP reading frame. This test system is useful because visual appearance of the cherry signal indicates plasmid delivery and expression and in combination with the eGFP rescue shows TGA suppression. Data in FIGS. 6A and 6B show western blot data using this test construct to assay the ability of five anticodon edited Trp tRNA human to suppress the TGA stop site in the short linker between cherry and cGFP reading frames. Of these constructs, the candidates 1, 2, 3 & 5 show modest activity in this regard. This may be due to structural intolerance to the mutation or the possibility that altering the anticodon, even just by a single base, disrupted the ability of the Trp synthetase to recognize and/or acylate the tRNA with tryptophan. However, number 4 of these test tRNA (tRNA #4) shows significant suppression activity of the TGA site, producing a full-length cherry-eGFP-HA protein (FIG. 6B). Further, no read-through was seen in the absence of co-expressed tRNA, last lane, FIG. 6B.

### METHODS

The Trp tRNA were examined for codon editing tolerance (TGG→TGA) and their ability to suppress a targeted TGA test site in a transiently transfected tandem-fluorophore (mCherry-TGA-GFP) and CFTR Trp1282X. Initial screening of 5/9 Trp tRNA discovered an anticodon edited Trp-tRNA that was transiently transfected in HEK cells and has ‘stand alone’ functionality to rescue a cherry-TGA-eGFP-HA test construct, FIG. 6B. The selective presence of the HA epitope indicates successful rescue, as well as confocal examination of both cherry and eGFP fluorescence at the single cell level (not shown). This result provides proof of principle data that a) some ACE-tRNA can tolerate anticodon editing b) that these tRNA retain the ability to be acylated with Trp by endogenous tryptophan synthetases and c) these tRNA can suppress TGA sites embedded within open protein reading frames.

The remaining four Trp-tRNA are functionally examined for tolerance of anticodon editing from TAA to TGA suppressors. These anticodon edited tRNA are tested for their ability to rescue the cherry-TGA-eGFPHA clone. Biochemical (western blot) data are obtained for cherry and eGFP signals as well as HA epitopes. Here, cherry expression serves as the positive transfection control. Confocal imaging verifies cherry and eGFP fluorescence at the single cell level.

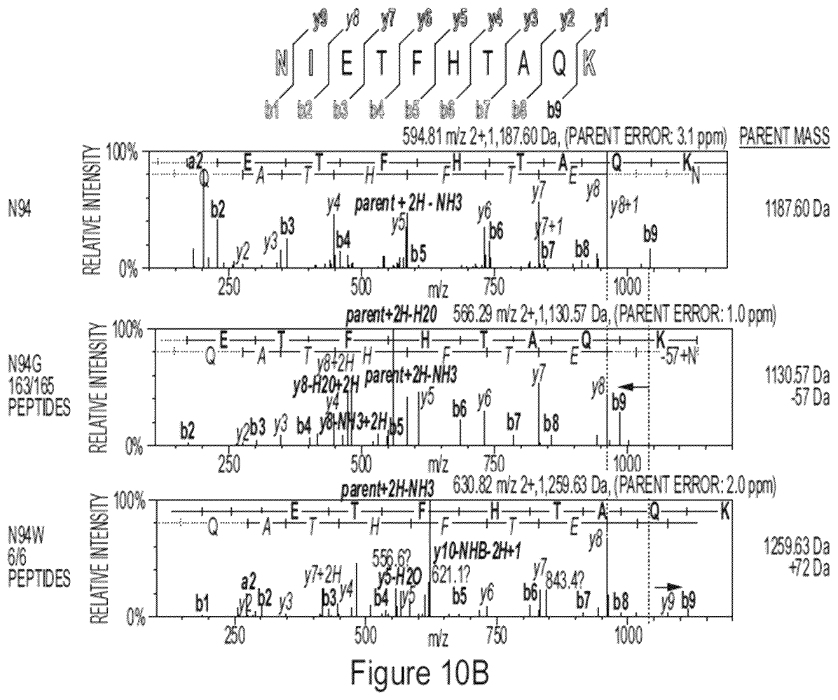

The fidelity of endogenous Trp synthetases to charge ACE-Trp tRNA with the tryptophan amino acid is determined by mass spectroscopic analysis of tryptic fragments of purified rescued cherry-Trp-cGFPHAprotein. Predicted mass for the tryptic fragment generated from the linker between the cherry and eGFP reading frames is: KPINQWPANTHER with a predicted mass of 1590.8135; bold W indicates incorporation site, FIG. 10. Thus, this represents the first example of a nonsense codon repair and replacement with the wild-type amino acid and therefore is a significant advance over existing approaches, such as the therapeutic Ataluran. The later example, the compound promotes read-through of the nonsense codon with the incorrect amino acid, thus the discovery and identification of new tRNA sequences that provide stringent repair is significant.

Rescue of transiently transfected CFTR 1282X channels by ACE-tRNA identified above are assessed by standard biochemical methods for full maturation of the B and C glycosylated CFTR bands 20. Thus, the channel has been repaired with the wildtype amino acid, is fully functional and successfully trafficked to the plasma membrane.

The next step is to functionally characterize CFTR Trp1282X channels rescued with ACE-tRNA systems identified above using electrophysiological (single cell patch clamp and Ussing chamber) and biochemical approaches. The efficacy of expressed tRNA to diminish nonsense-mediated decay (NMD) of 1282X mRNA would be assessed with quantitative rtPCR. Reprogramed human airway cells are used to test expressed codon edited Trp-tRNA rescue of native 1282X CFTR channels.

It is demonstrated that anticodon editing is tolerated in an identified human Trp tRNA and this 75-base pair transfer RNA is capable of suppressing an in-frame TGA codon within a test construct. These experiments extrapolate this discovery to characterize the ability of this ACE-tRNA to interact with CFTR 1282TGA mRNA and produce functional CFTR channels in model cells (FRT and A549) as well as p. 1282X human reprogrammed airway cells.

Biochemical determination of rescue levels in transiently expressed CFTR 1282X channels as well as those in reprogrammed airway cells. Antibody M3A7 is used to recognize the rescued (epitope is aa 1370-1380) and to detect all CFTR, rescued and non-rescued, antibody binding to the N-terminus like MM13-4 (epitope aa 25-36), available through EMD Millipore. Alternatively, L12B4 (epitope aa 386-412, EMD Millipore) or 660 (epitope aa 576-585,) are available through Cystic Fibrosis Foundation Therapeutics.

Surface functionality is examined through electrophysiological approaches, patch-clamp and Ussing chamber recording. 1282X mRNA stability and abundance is assayed by quantitative rtPCR of RNA extracts from transiently expressing cells and reprogrammed airway cells.

Bioinformatic analysis of RNA transcriptome data from human airway cells identifies abundance, context and identity of TGA codon containing transcripts. The top 10 expressing transcripts using TGA for their normal stop sites are followed up at the level of individual transcript with protein biochemistry before and after ACE-tRNA expression. Biochemical and immunohistological probes of cellular apoptosis are also used to examine the impact of ACE-tRNA in cell death.

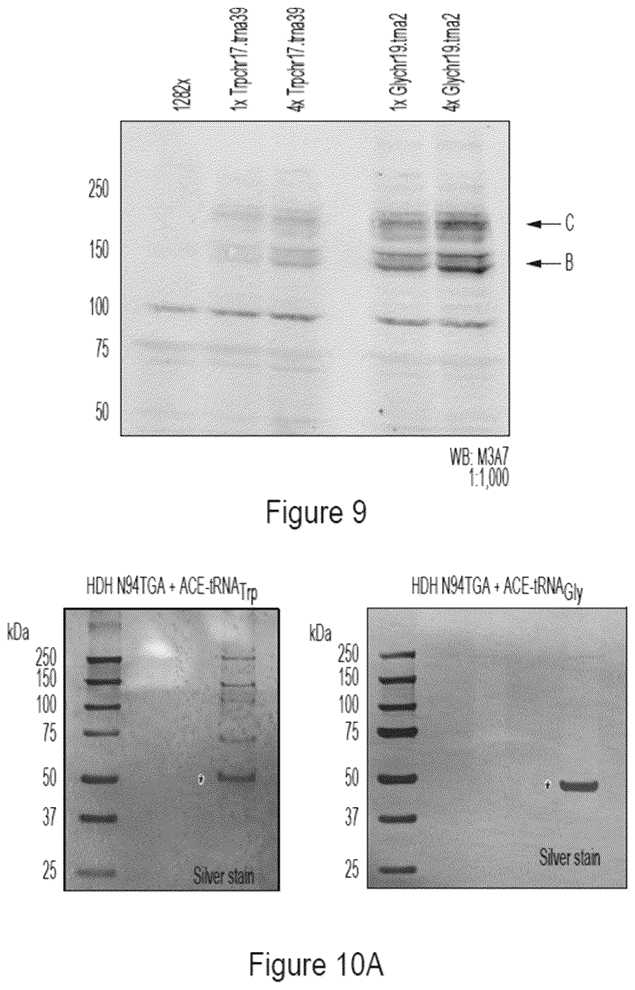

In conclusion, the data show that ion channel genes with in-frame stop sites are amenable to this type of “rescue” (FIG. 9) and components of the system can be expressed virally in airway cells. Further, a highly simplified form of this idea, an ACE-tRNA of human origin, demonstrates the “stand alone” ability to rescue in-frame CFTR TGA codons in mammalian cell lines (FIG. 9). This approach has many advantages over existing stop-codon strategies and merits closer examination in terms of the ability of ACE-tRNA to 1) abrogate nonsense mediated decay 2) function in lung cell preparations and 3) to specifically rescue CFTR 1282X.

### EXAMPLE 2

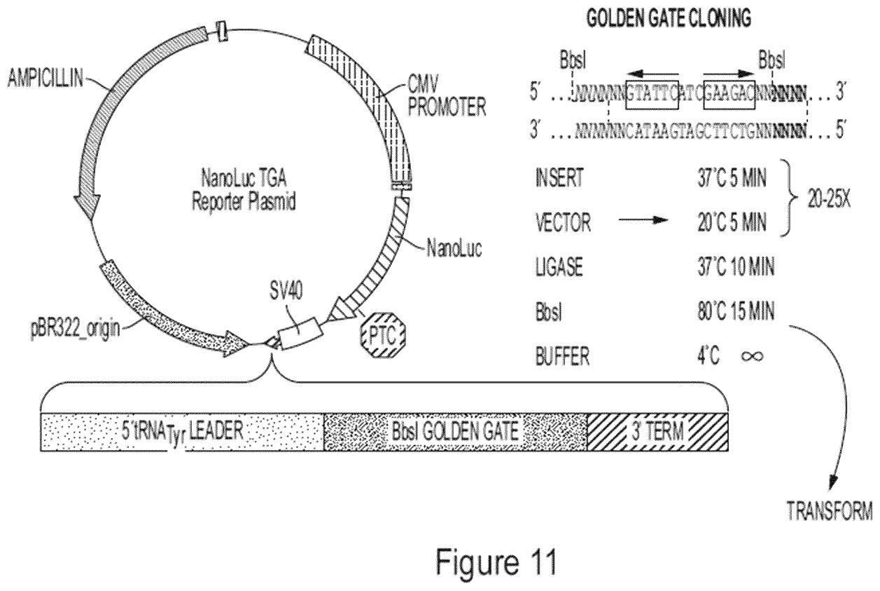

Several different nonsense mutations cause CF, thus underlying roughly 10% of all CF disease. FIG. 7. These cases are concentrated into ten specific genetic lesions: E60X, R75X, G542X, R553X, Q890X, Y1092X, R1158X, R1162X and W1282X. A screen was developed to screen existing human tRNA sequences for modification and tolerance to anti-codon editing. To this end, roughly 144 ACE-tRNAs were candidates to test for those that could be used to promote the repair of the disease causative nonsense codon and the expression of the full-length protein. Specifically, using the scheme described in FIG. 11, tRNA libraries were generated to identify novel tRNA sequences that encode for ACE tRNA with the ability to repair the top CF causative nonsense mutations. Specifically, 10 ng of annealed oligos encoding the ACE-tRNAs were combined with 50 ng of NanoLuc reporter plasmid, 1 μl 10× CutSmart Buffer (NEB), 1 μl T4 ligase (NEB), 10 mM ATP and 1 μl BbsI (NEB) and cycled in a thermocycler as described in FIG. 11. 1 μl of the reaction was transformed into competent E. coli and the transformants were plated on ampicillin agar plates. One transformant was picked per plate was picked, grown in 1 ml of LB under ampicillin selection, miniprepped and sequence verified.

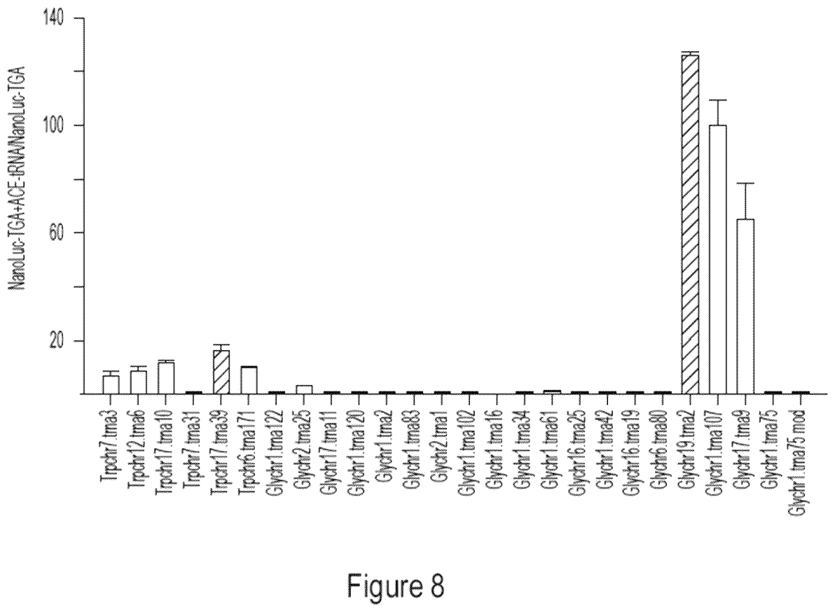

Screening studies were first performed to identify the best ACE-tRNA Candidates from tryptophan and glycine. 125 ng of sequence verified miniprep cDNA of NanoLuc reporter plasmid with ACE-tRNA was transfected into HEK cells using calcium phosphate. HEK cells were plated in 96 well plates at 4×10⁴ the day prior. 24 hours after transfection the media was replaced with 20 μl of PBS and 15 μl of NanoGlo reagent (Promega) was added. Plates were read on a SpectraMax i3 (Molecular Devices). Data are of replicates of 3 or greater. FIG. 8. The data show that most tRNA demonstrate poor codon editing tolerance. However, clear high-performing tRNA emerge from the screen, with identification of ACE-Trp and ACE-Gly tRNA which demonstrate rescue of nonsense codon containing protein of 20-fold to 130-fold over background.

To assess is these novel tRNA could rescue CFTR channels harboring nonsense codons, they were co-expressed in mammalian HEK cells with a CFTR W1282X cDNA plasmid. The cellular preparations were analyzed by standard biochemical approaches via Western blot assessment of CFTR protein. This method is highly advantageous for this purpose because the CFTR protein displays a multi-banded pattern that is well-established. Specifically, the “B” and “C” bands represent the full-length and fully mature, post-translationally proceeded CFTR protein at the cell surface, respectively. In this case, both rescue with Trpchr17.trna39 and Glychr19.trna2 ACT-tRNAs produce robust populations of ‘B’ and ‘C’ CFTR immunopositive (antibody MA37) bands, indicating the promotion by said tRNA of the full-length, successfully trafficked ion channel protein. FIG. 9.

### EXAMPLE 3

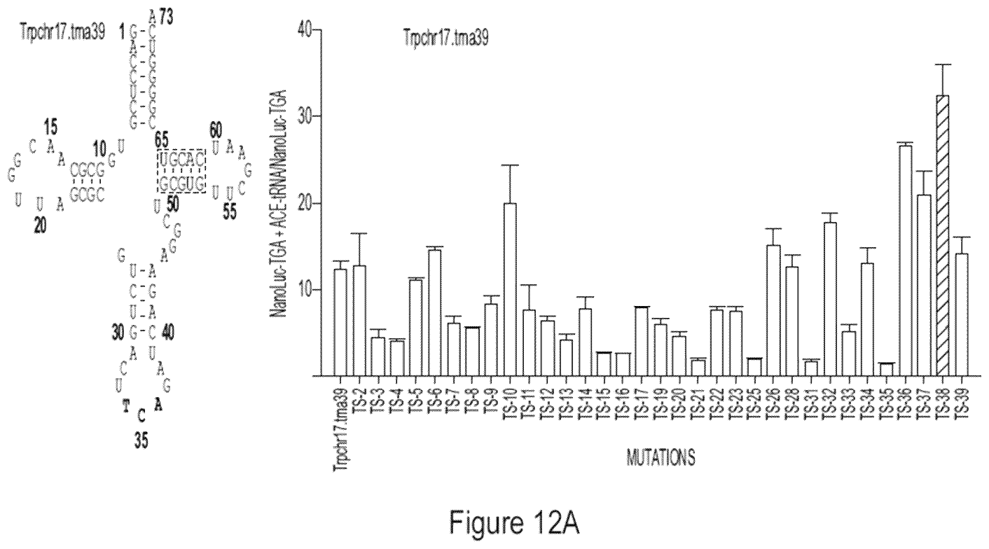

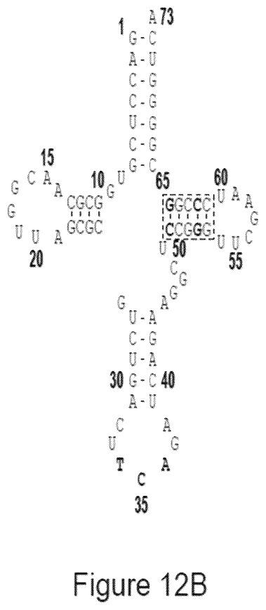

T-stem modification significantly improves nonsense suppression. FIG. 10. An additional modification of the tRNA was made to further enable their function for the purpose of suppression of nonsense codons and the promotion of protein expression. Without being bound by theory, it was hypothesized that rationally introduced mutations within the tRNA ‘t-stem’ loop, shown in FIG. 10, will yield a tRNA molecule that is more stable and functionally more potent for nonsense codon suppression. To this end, single and double mutations were directly engineered into the t-stem loop of tRNA Trpchr17.trna39—an ACE-tRNA identified with activity for the rescue of tryptophan TGA nonsense codons. Thirty-eight tRNA t-stem variants were thus generated and screened in HEK cells transiently transfected with the nonsense rescue reporter construct shown in FIG. 4. 24 hours post-transfection, cells were assayed for luciferase activity, shown in FIG. 10. The data show strong variation and identify novel tRNA sequences with varied t-stem loop sequences with enhanced suppression activity. Notably, one such mutant, TS-38 52-62 G-C enhances the suppression ability of Trpchr17.trna39 by roughly 250% (FIG. 12). This is a generalizable modification, that is, new tRNA sequences identified, by example 1 and 2, can be made better (for their ability to rescue nonsense codons) through further rationale modifications. Such approaches aid in the therapeutic utility of ACE-tRNA directed to tissue types with low abundance target RNA or where tRNA delivery may be limiting.

### EXAMPLE 4

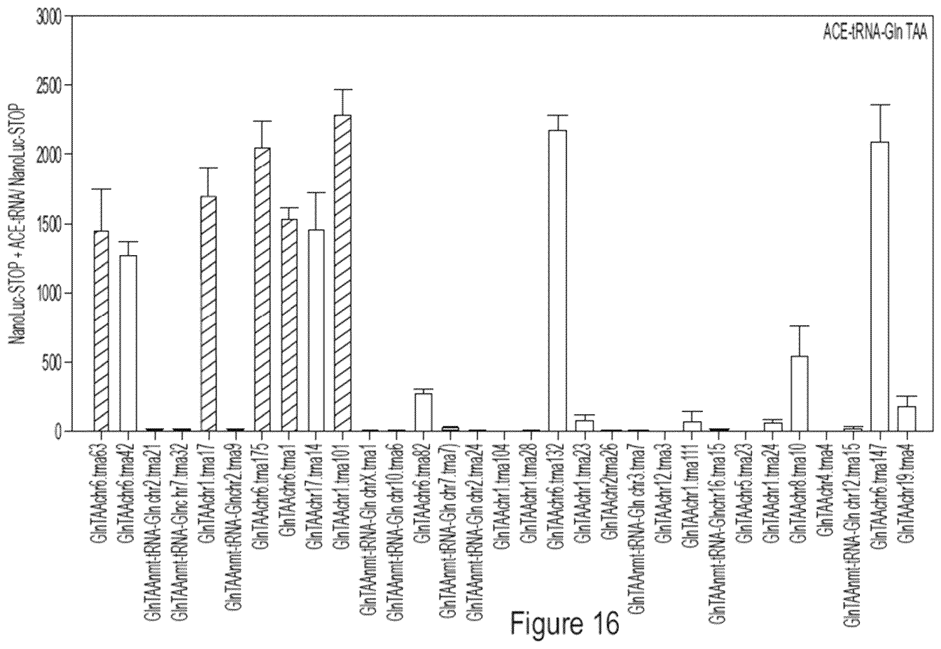

In order to enable the identification of the nucleotide composition and functional ability to suppress nonsense codons by new types of tRNA, an All-In-One Plasmid With A One Pot Cloning Reaction was invented for High Throughput Cloning FIG. 11. This approach enables the facile investigation of ACE-tRNA activity via luciferase activity in a standard 96 well format. Briefly, synthetic nucleotide sequences encoding for tRNA are ligated into the NanoLuc Reporter plasmid, with an example of the TGA nonsense reporter plasmid variant shown in FIG. 11. TAA (Opal) and TAG (amber) stop codon rescue vectors have been successfully designed and implemented in FIGS. 16-19. The benefits are the approach is that DNA oligos encoding for tRNA libraries can be ligated in the NanoLuc reporter plasmid with the presence of the restriction enzyme and ligase with the reaction pushed to nearly 100% incorporation of tRNA insert (FIG. 11)—thus the ‘one-pot’ designation. The reaction is transformed into E. coli, with the resultant cDNA purified by standard methods. Another benefit of the invented method is that the tRNA and reporter gen are within the single expression cassette, therefore lowering biological variability and improving data quality obtaining in resulting screens of tRNA suppression activity. The purified cDNA plasmids are then screened in high-throughput 96 well format for their ability to repair nonsense codons by inferred luciferase activity. The approach is suitable for the high-throughput screening of hundreds to thousands of tRNA for novel therapeutic activity.

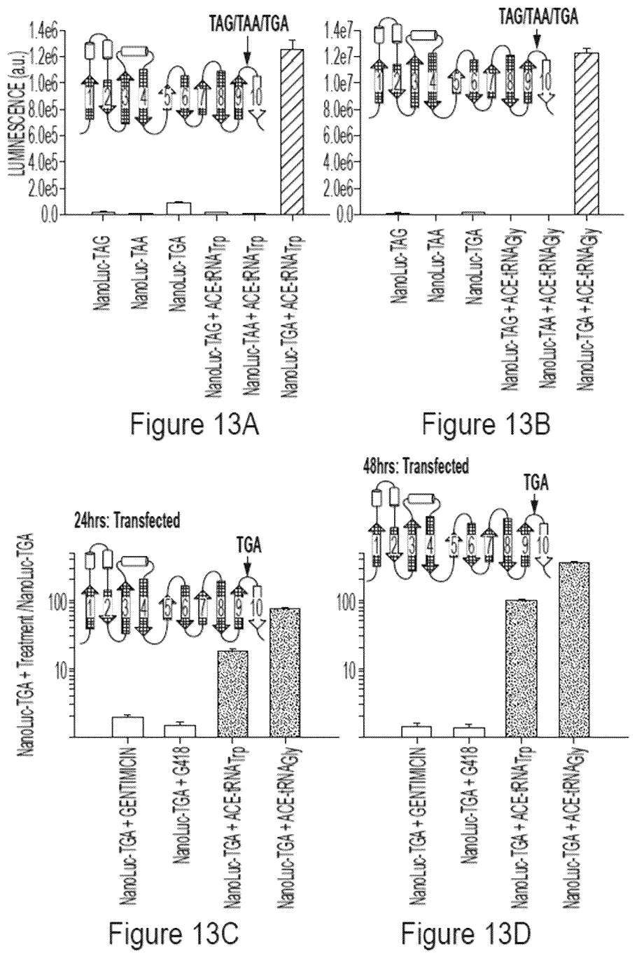

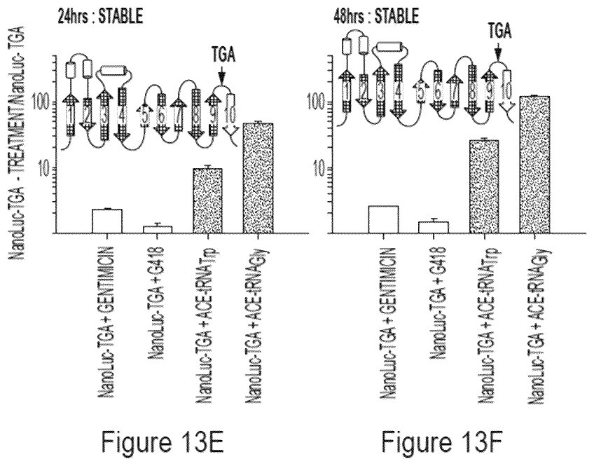

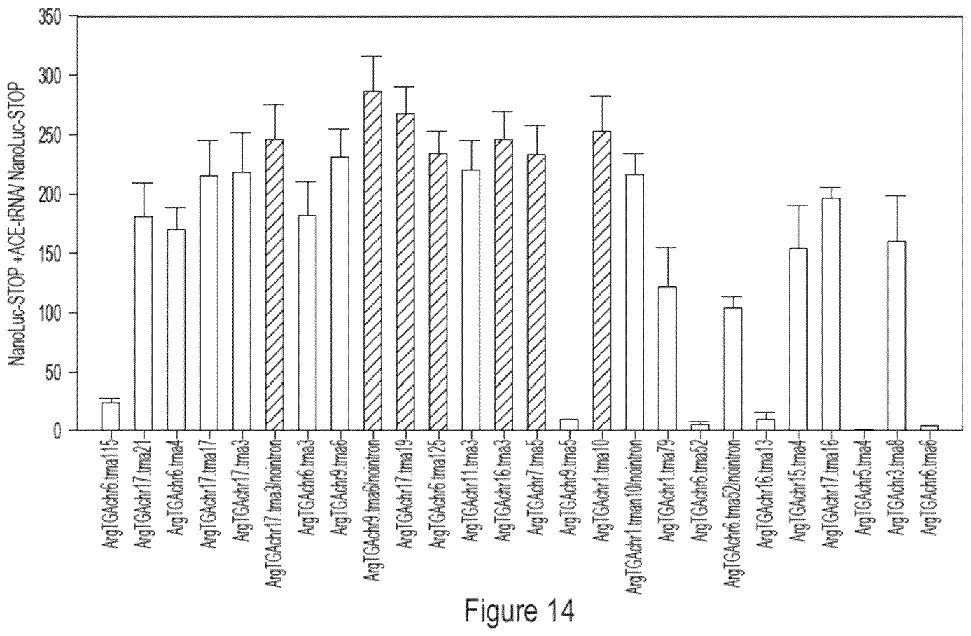

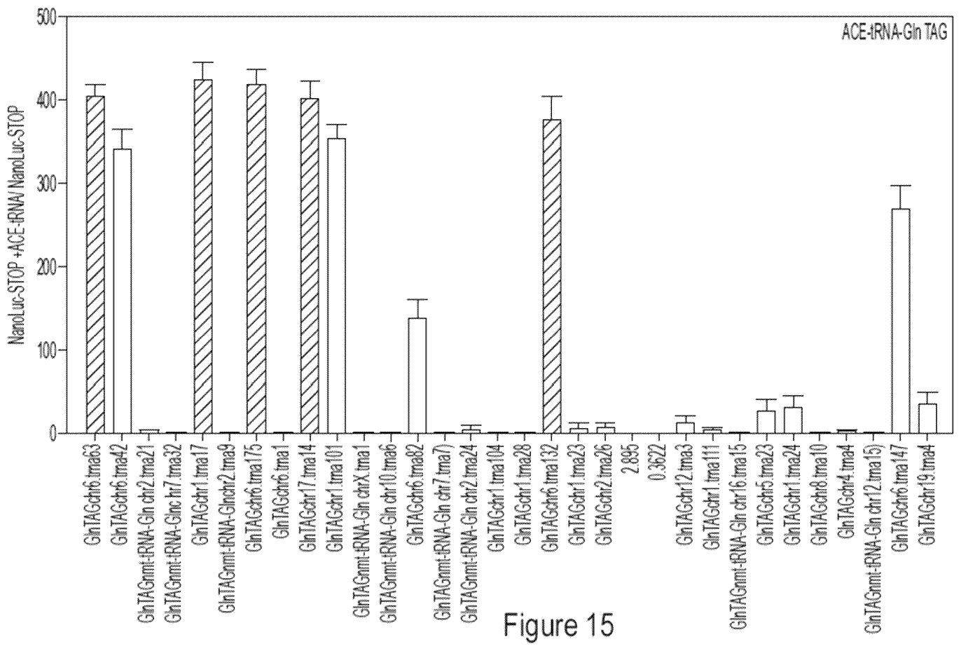

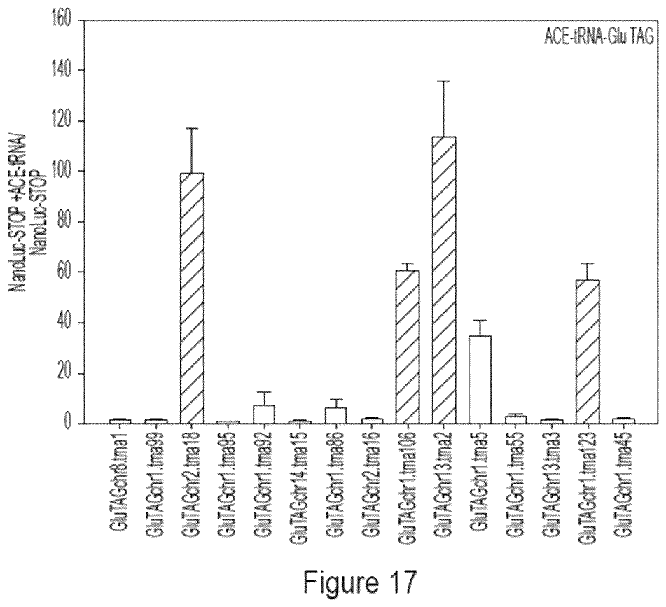

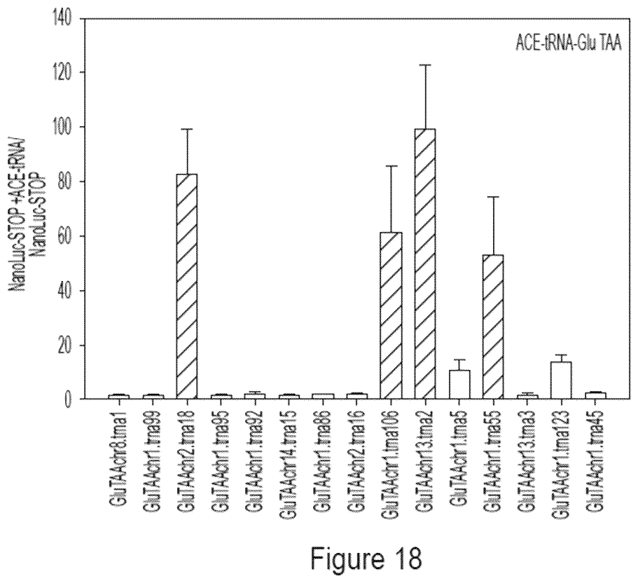

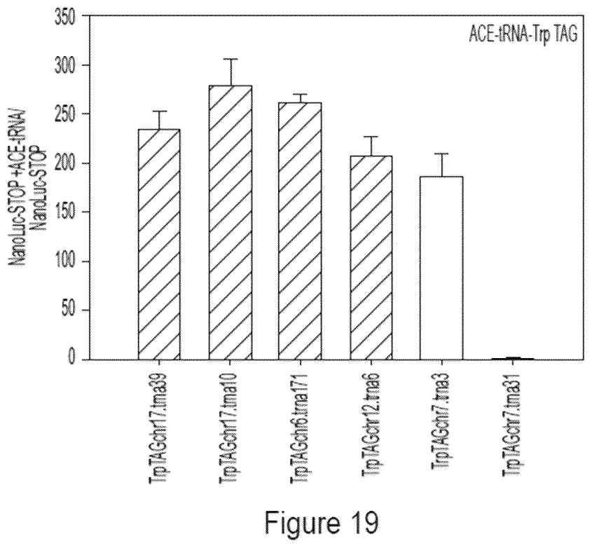

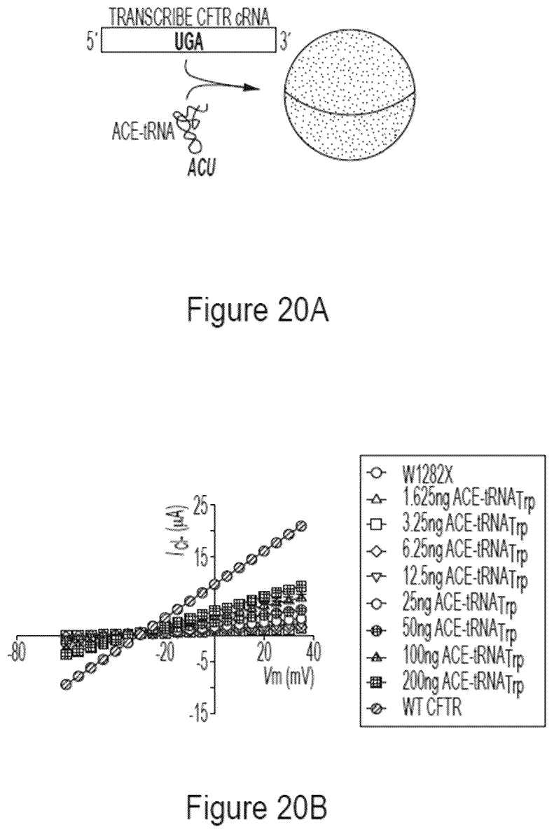

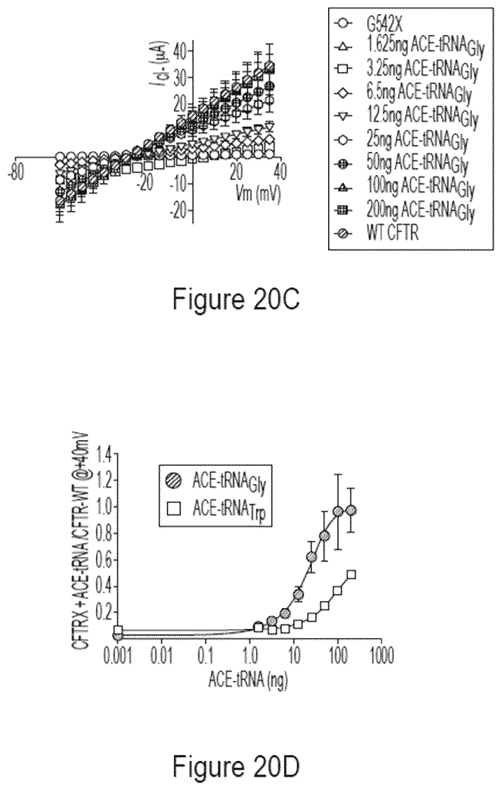

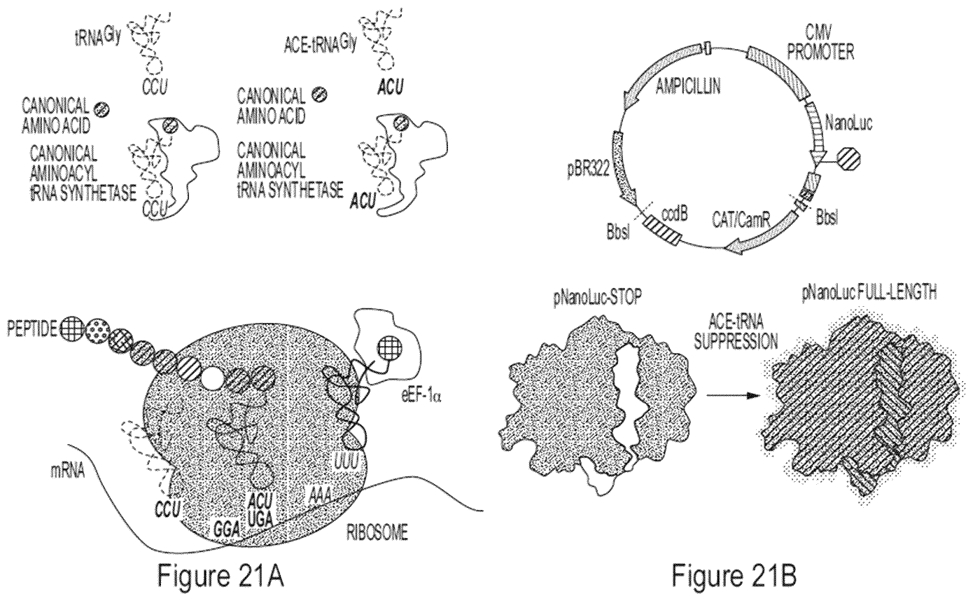

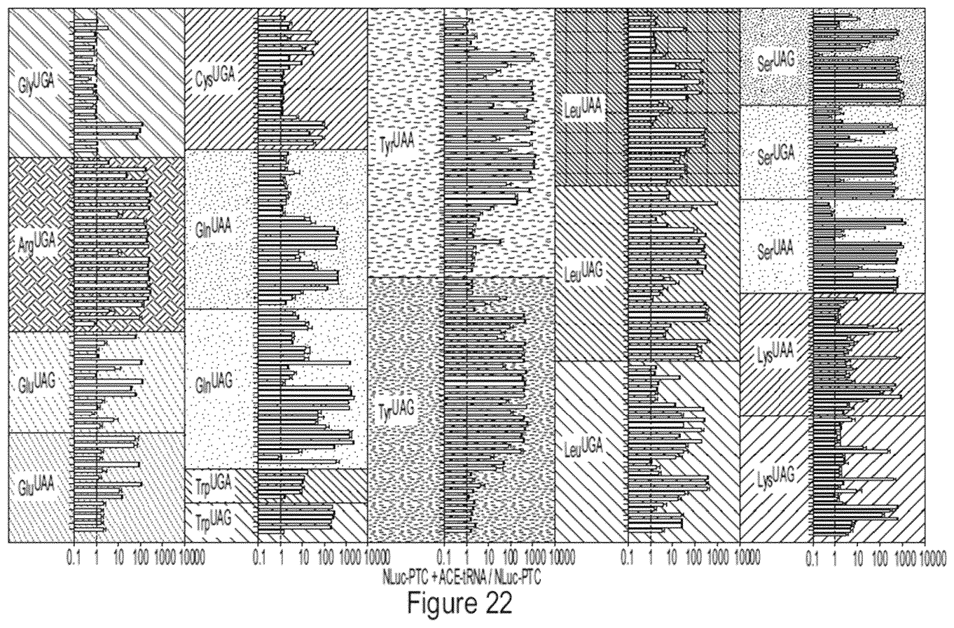

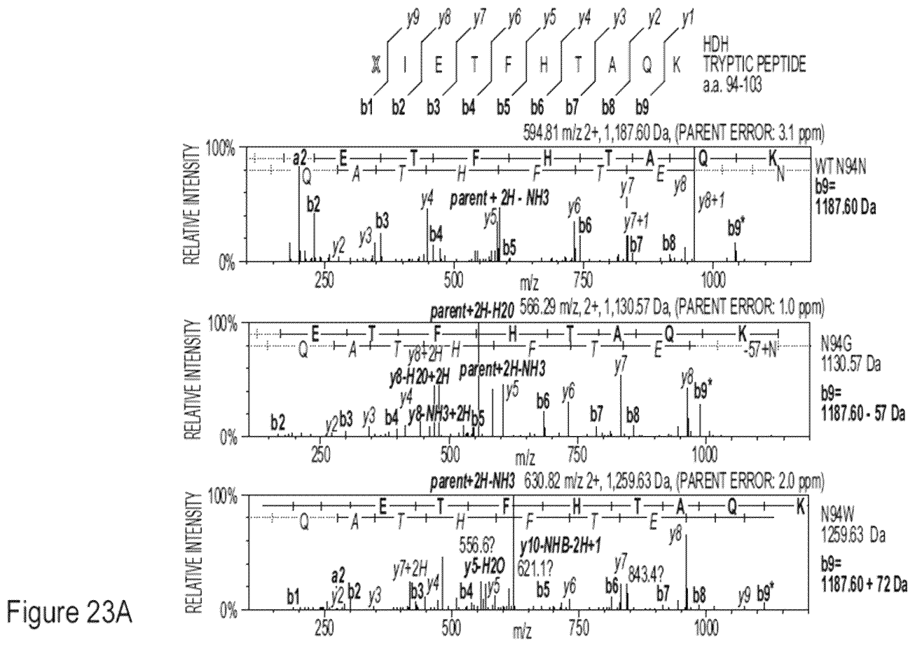

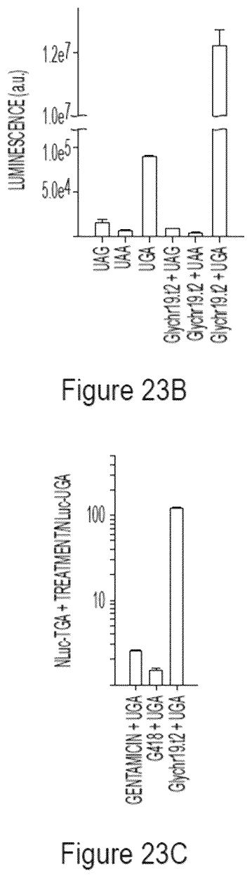

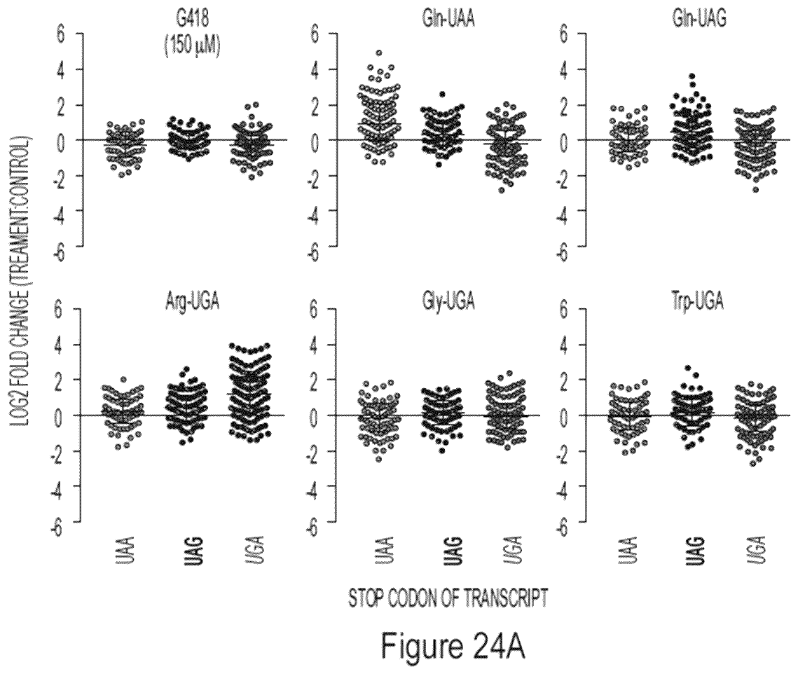

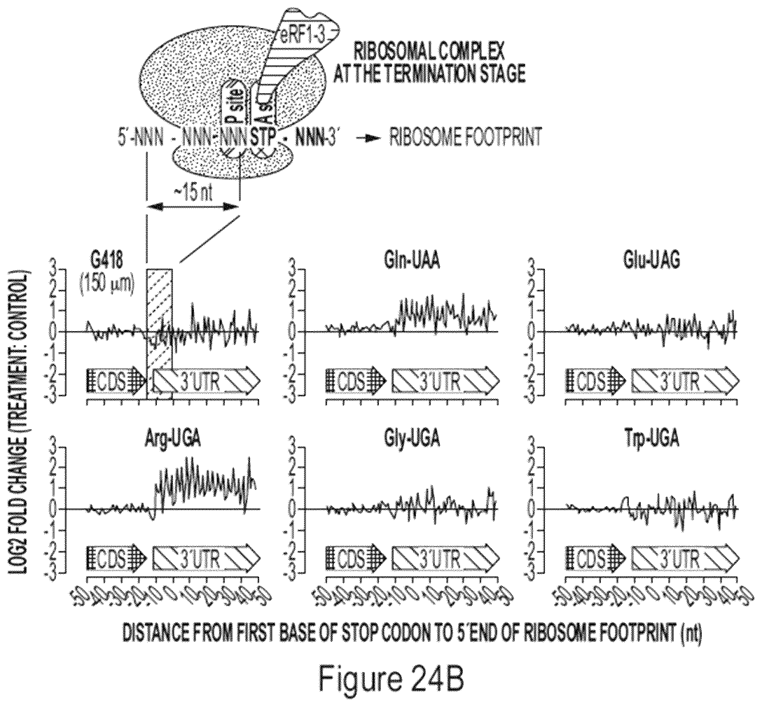

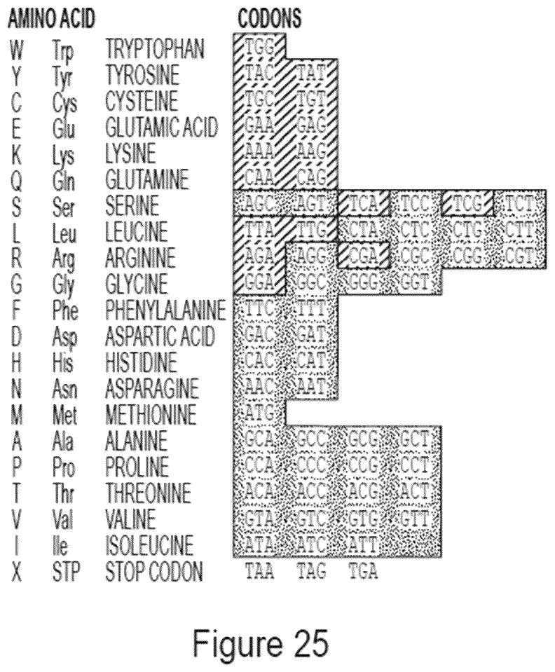

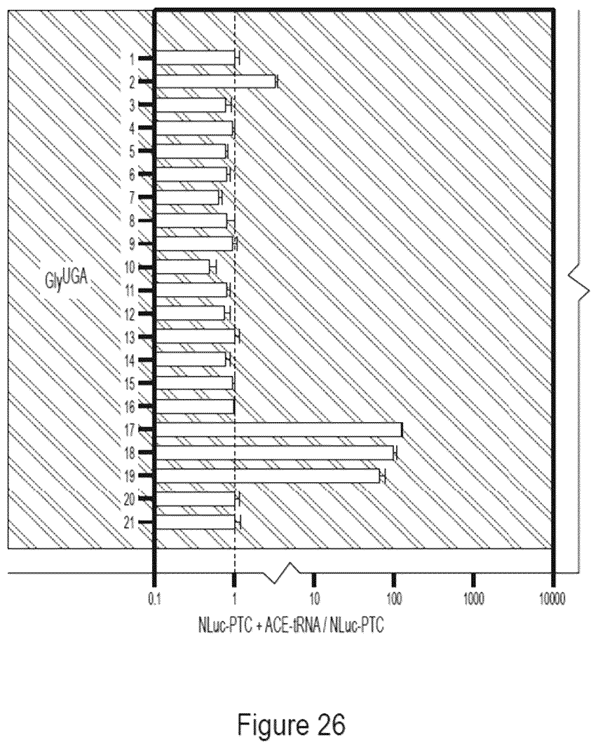

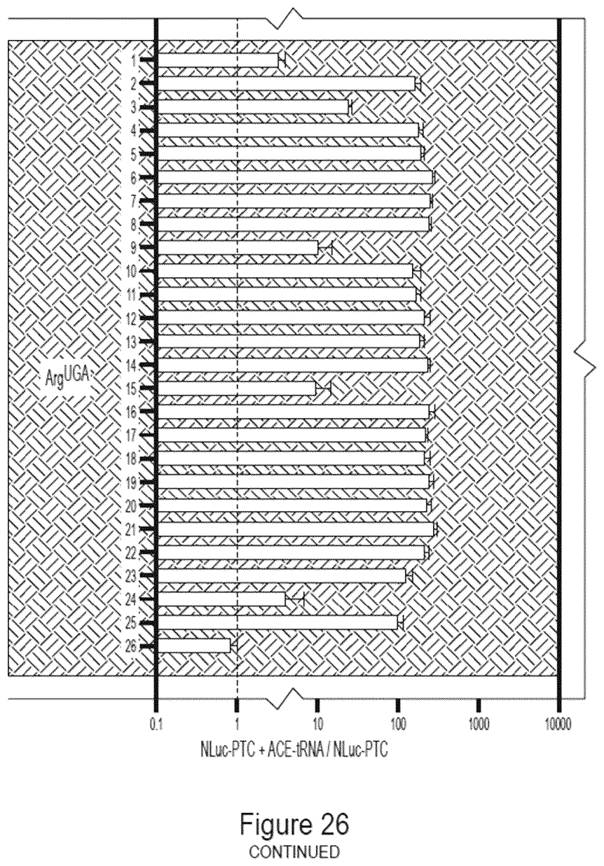

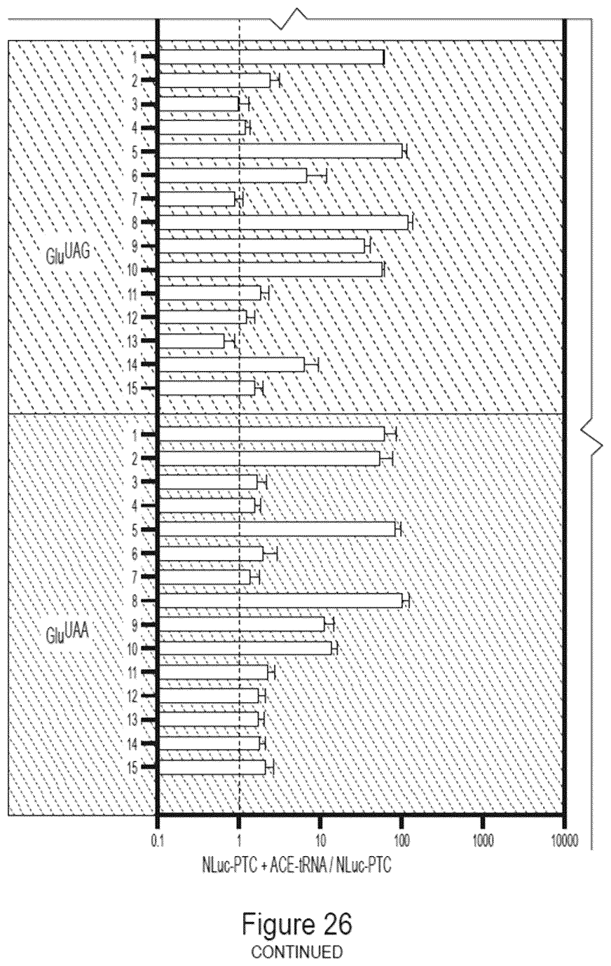

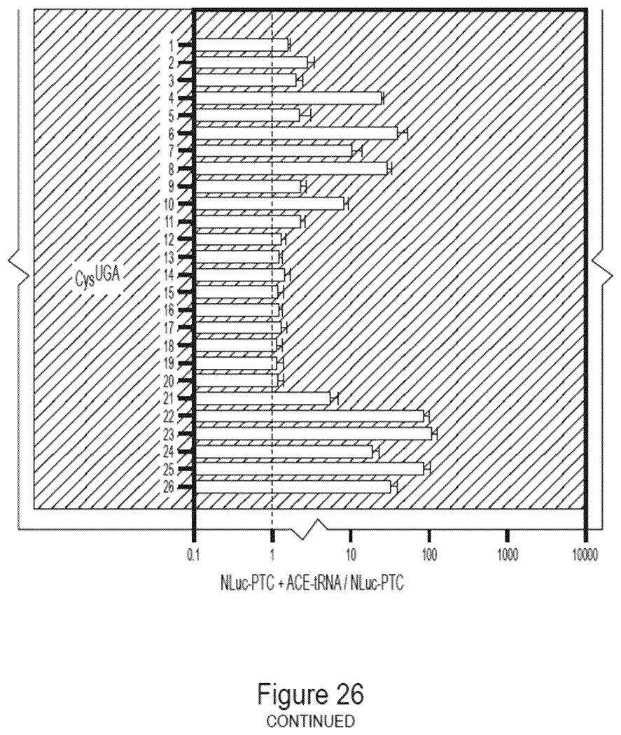

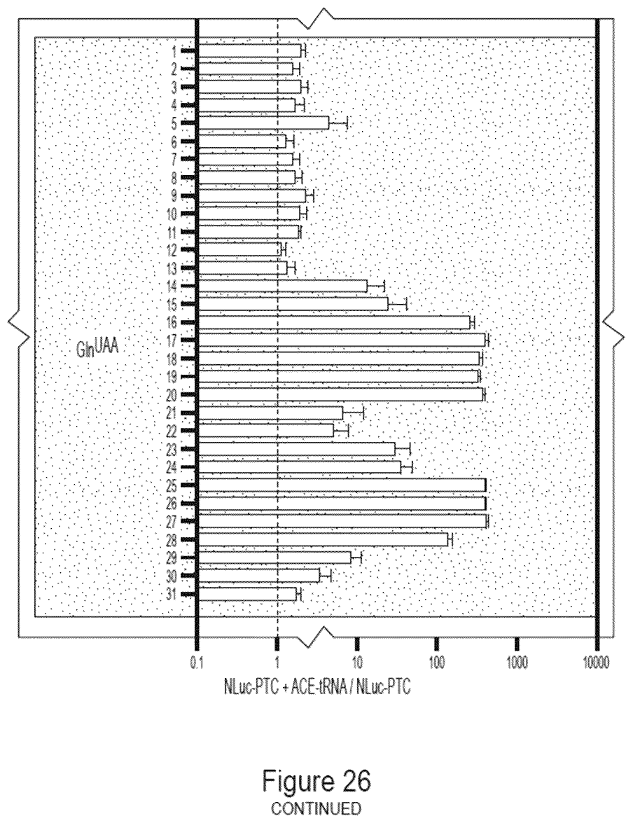

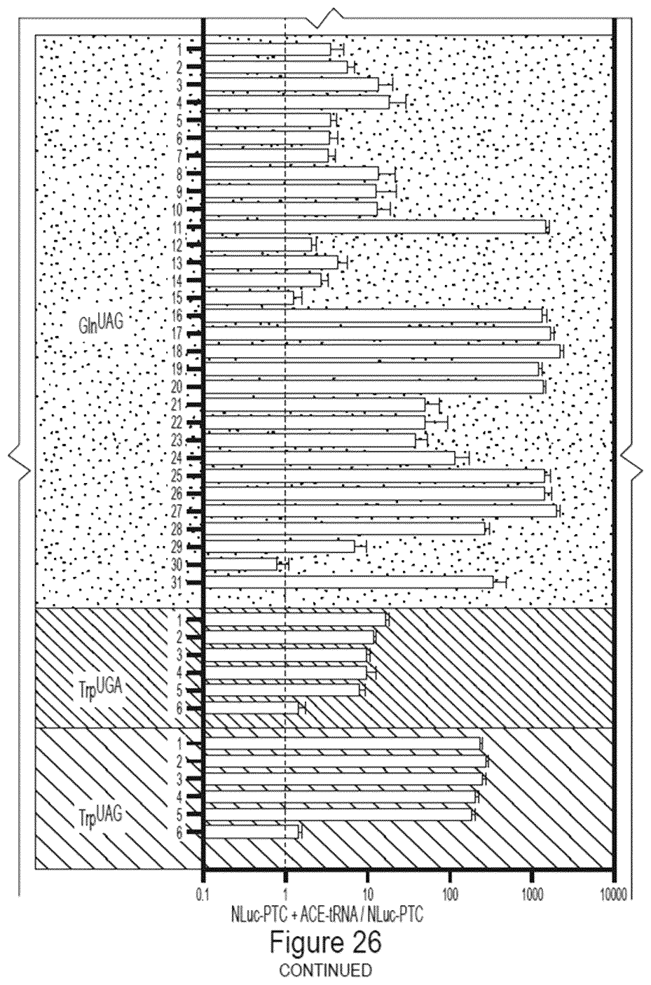

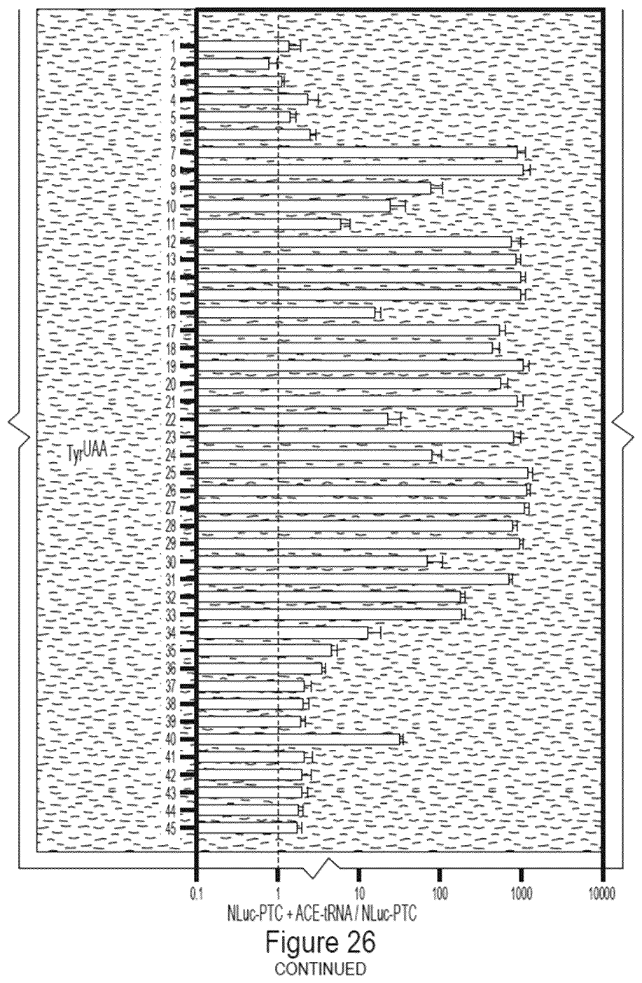

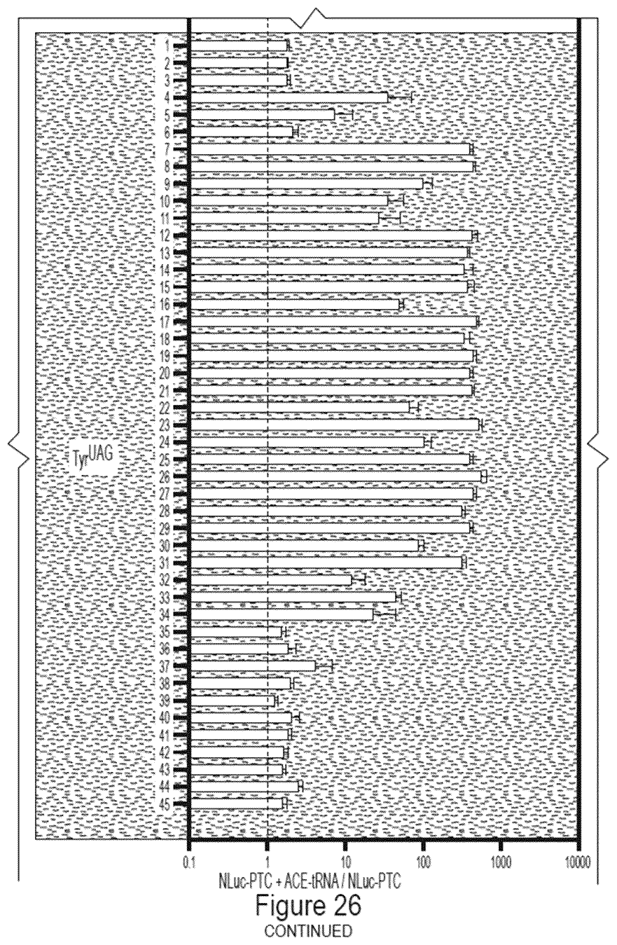

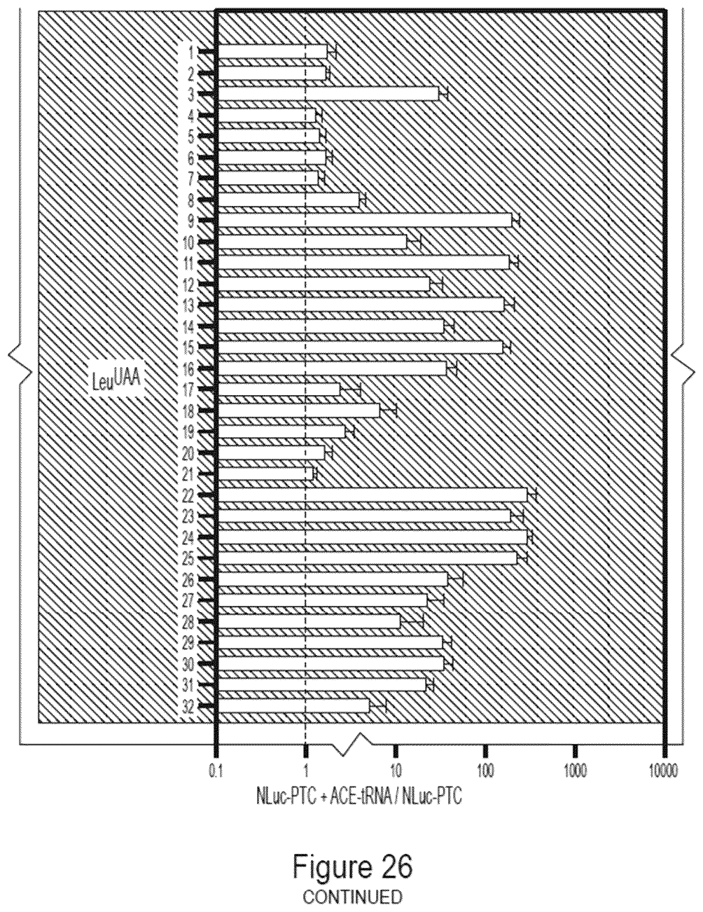

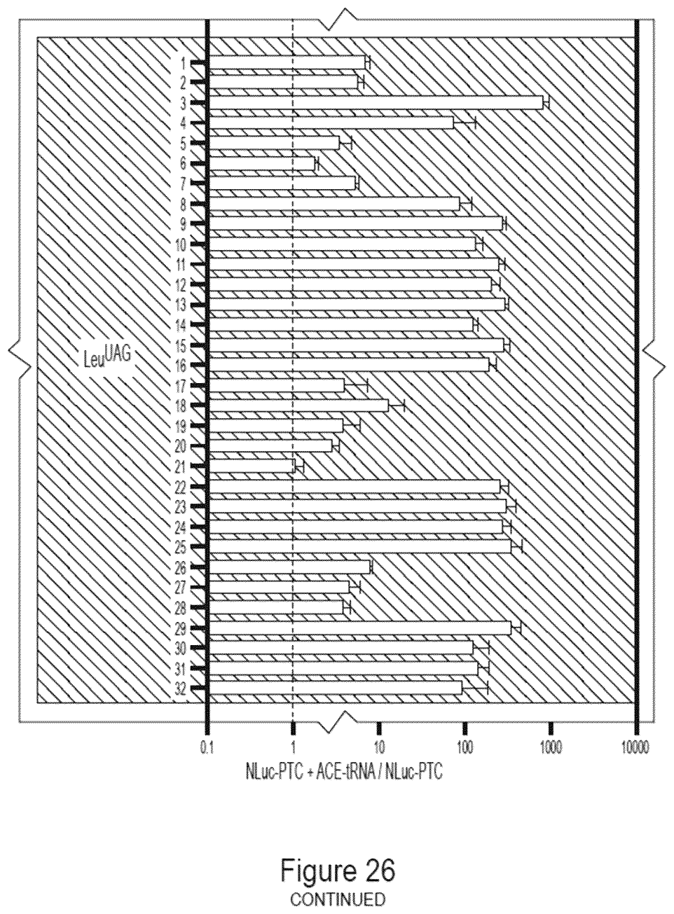

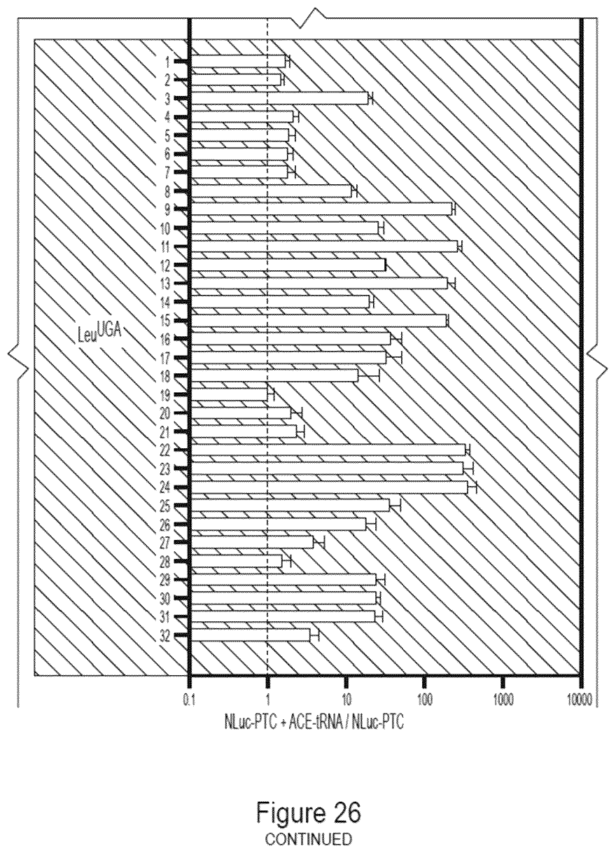

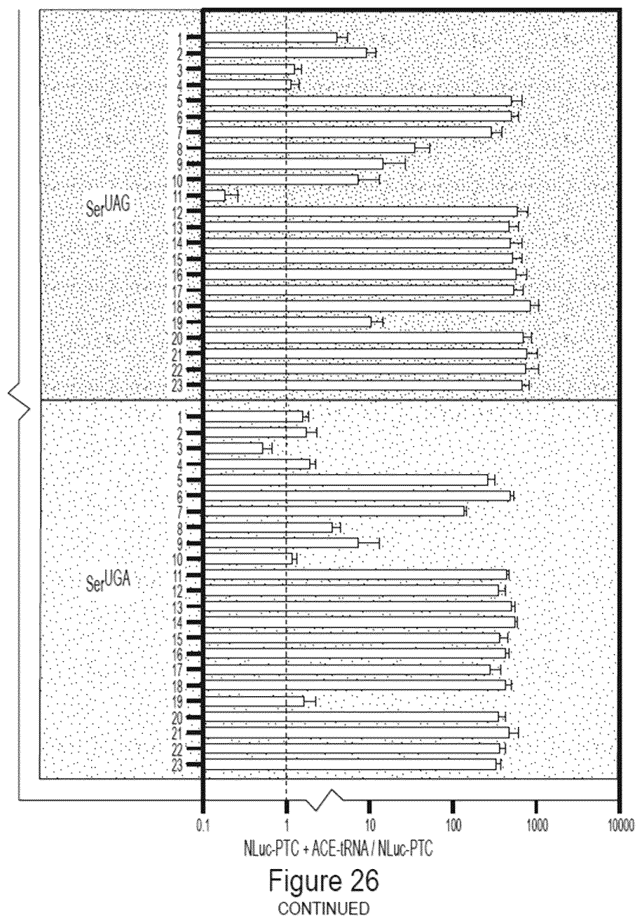

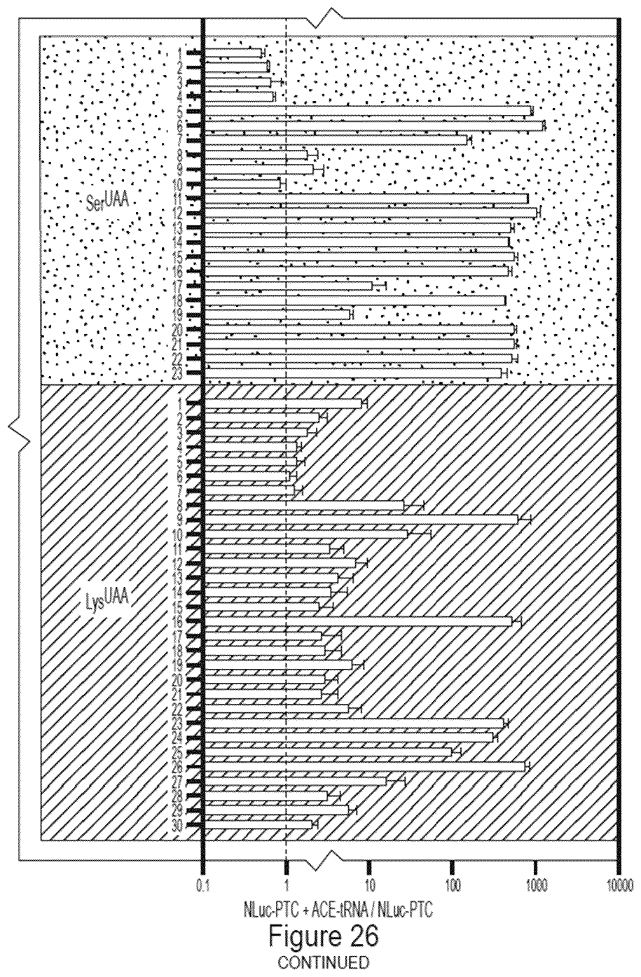

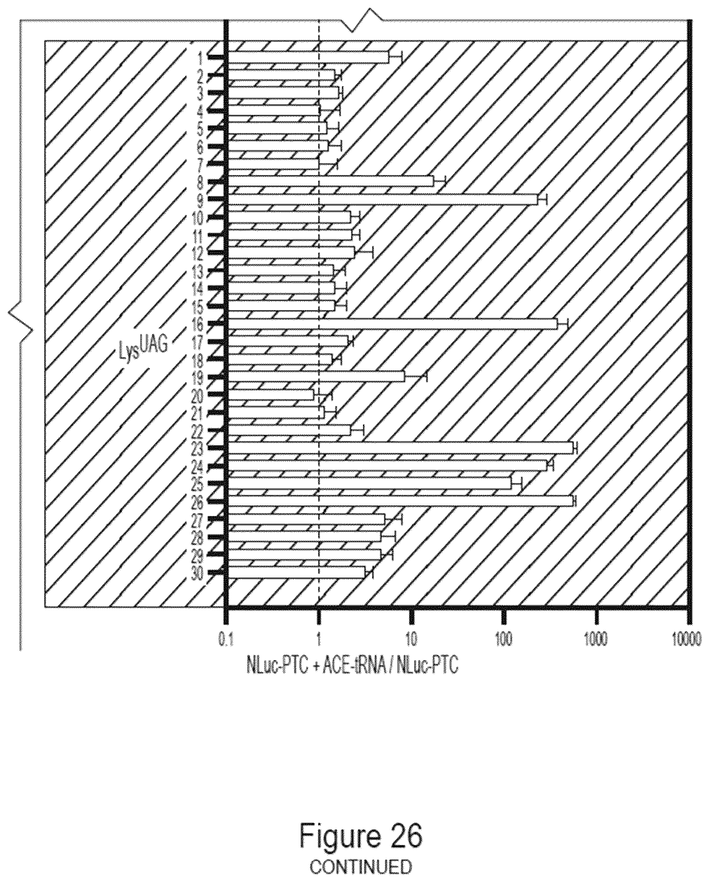

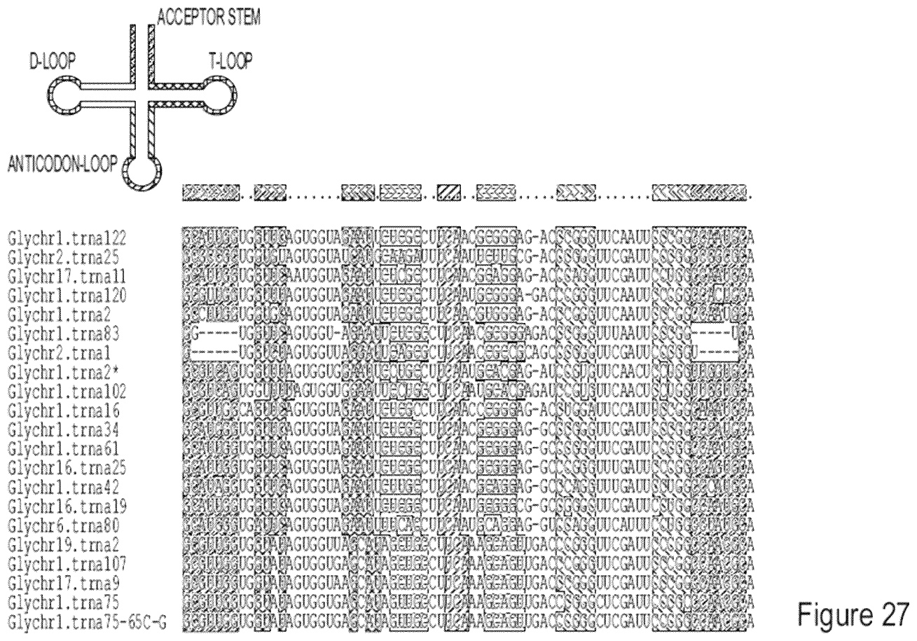

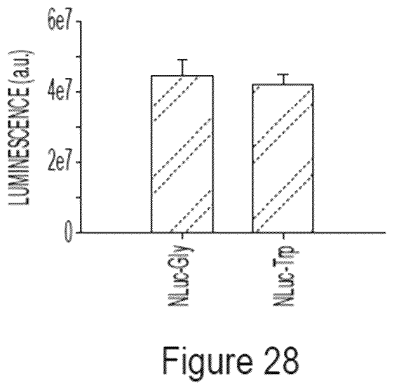

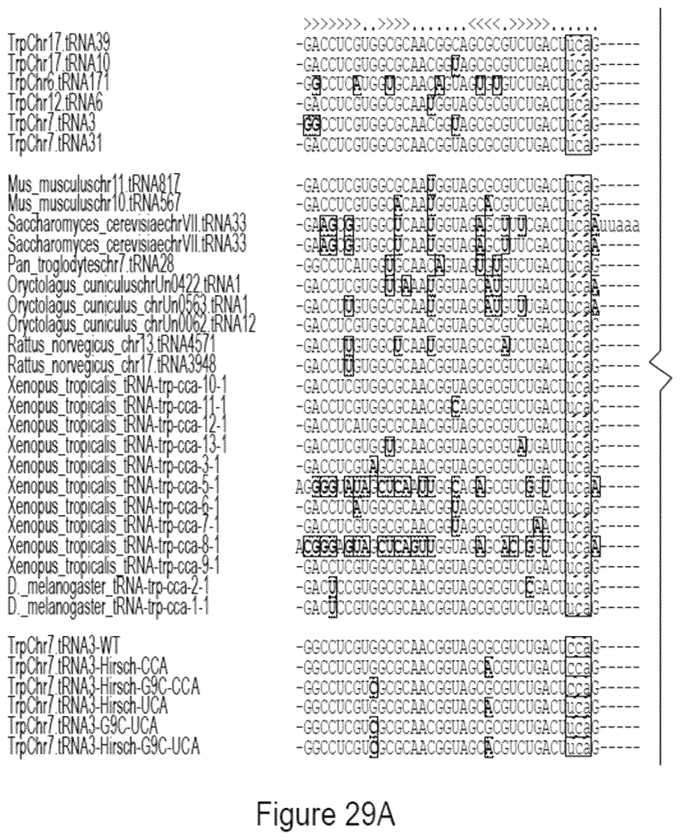

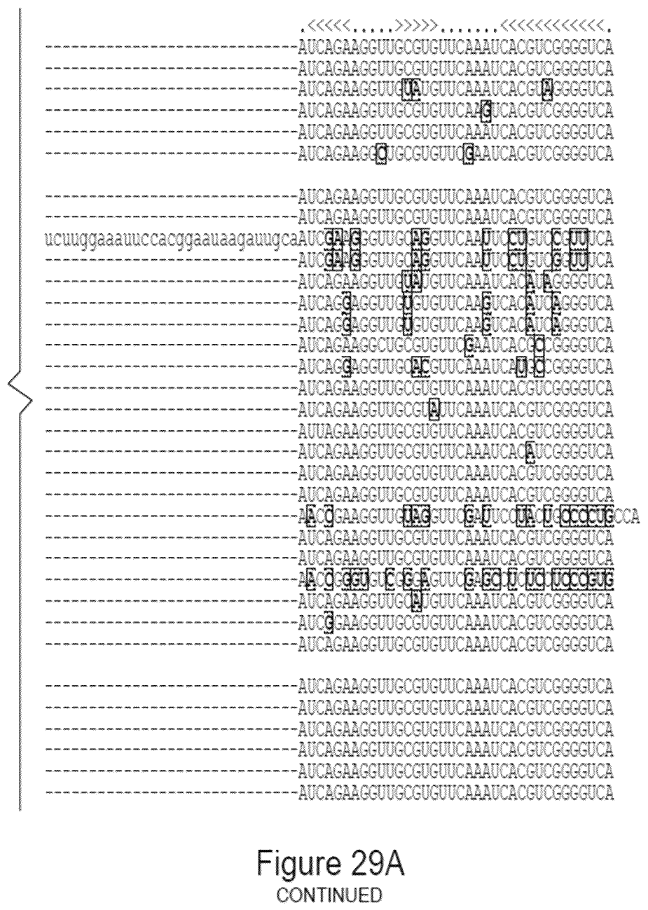

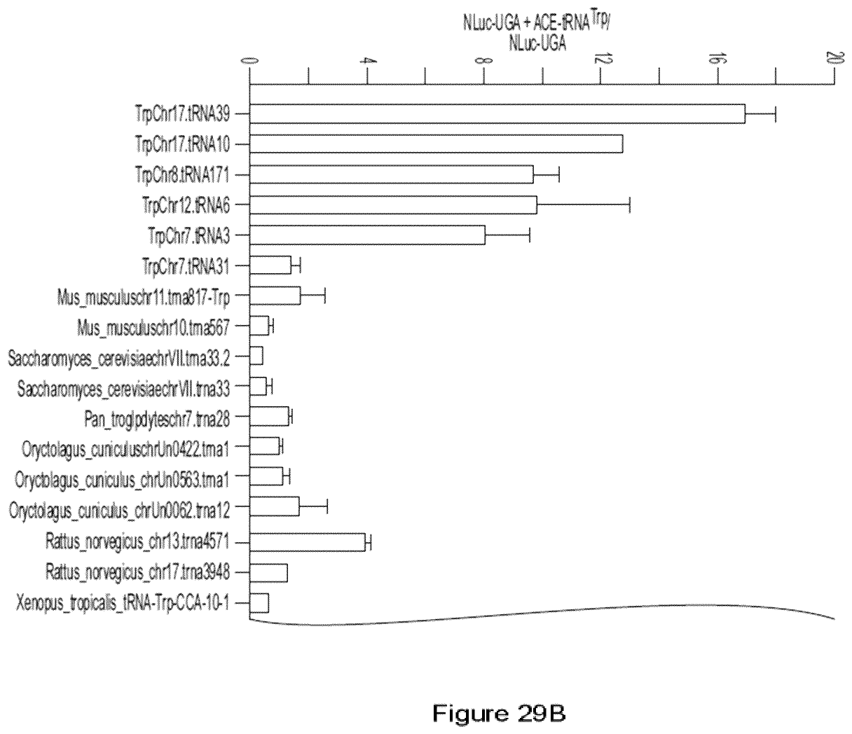

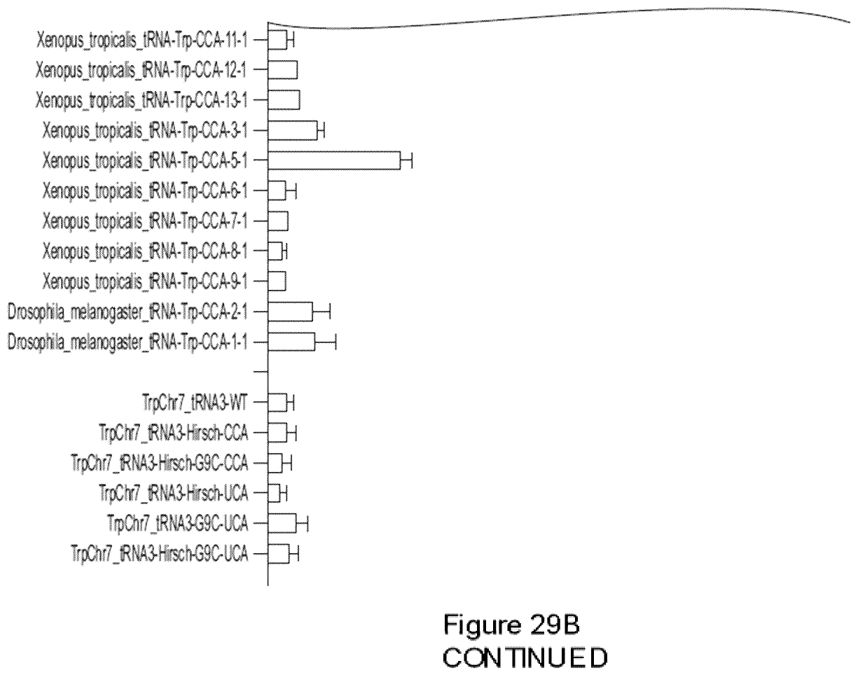

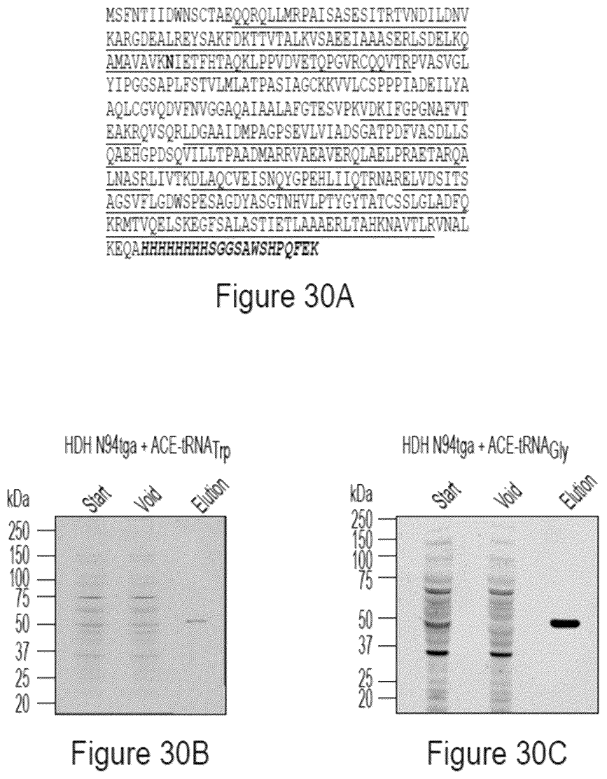

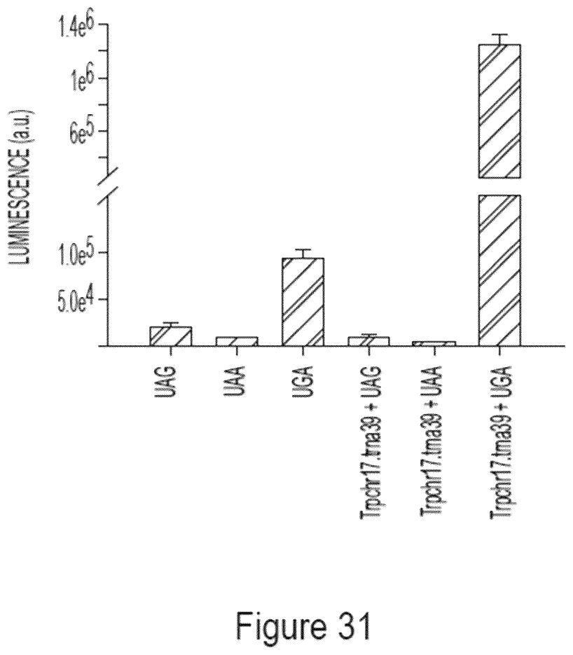

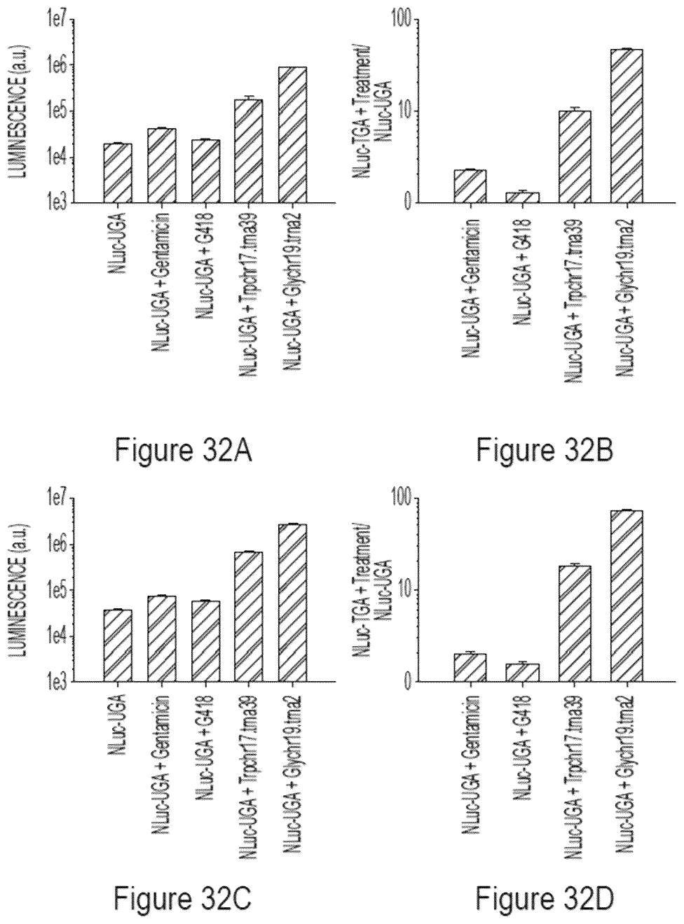

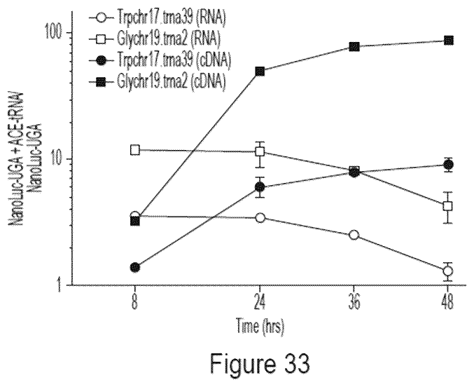

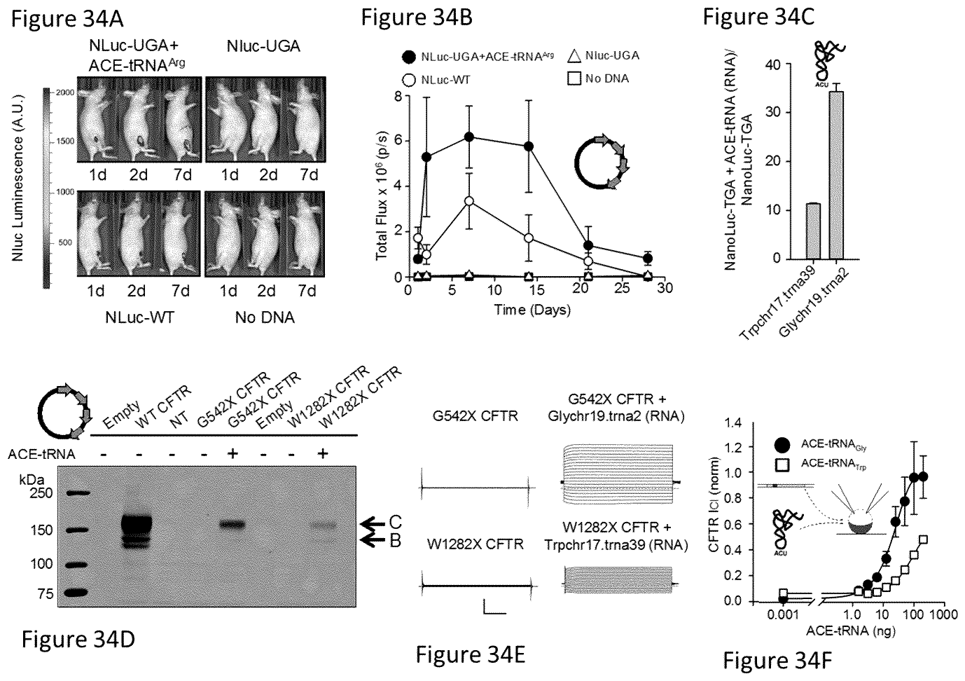

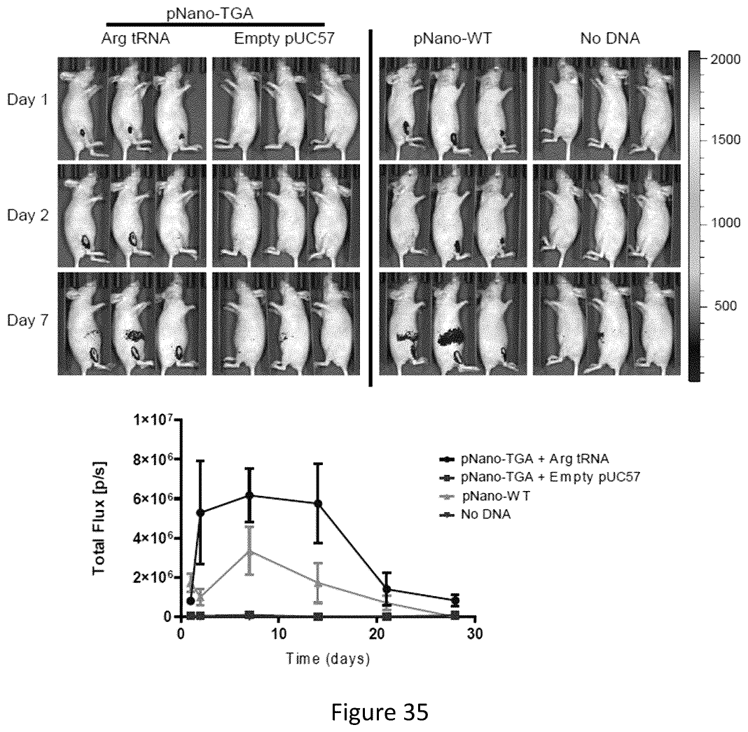

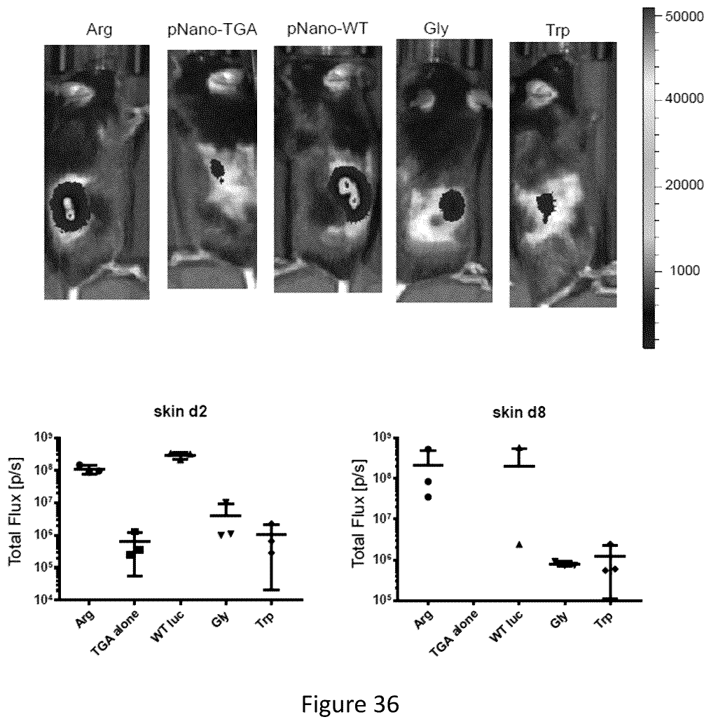

The ‘one-pot’ cloning and expression system described in FIG. 11 has been used successfully to identify unique tRNA sequences for the repair of Tryptophan and Glycine ACE-tRNA (FIG. 13), ACE-tRNA-Arg (FIG. 14), ACE-tRNA-Gln TAG (FIG. 15), ACE-tRNA-Gln TAA (FIG. 16), ACE-tRNA-Glu TAG (FIG. 17), ACE-tRNA-Gln TAA (FIG. 18) and ACE-tRNA-Trp TAG (FIG. 19). FIGS. 20A-20D show that delivery of ACE-tRNA as small RNA supports robust suppression of G542X and W1282X nonsense mutations.

1. A method of treating a disease associated with a premature termination codon (PTC) in a subject in need thereof, the method comprising administering to the subject a composition for generating one or more anticodon edited tRNA (ACE-tRNA) or fragments thereof in a subject, comprising one or more nucleic acid molecules or fragments thereof, wherein the composition is formulated for administration by electroporation.

2. The method of claim 1, wherein the composition comprises a cDNA molecule encoding an ACE-tRNA.

3. The method of claim 1, wherein the composition comprises a RNA molecule comprising an ACE-tRNA.

4. The method of claim 1 wherein the one or more nucleic acid molecules are engineered to be in an expression vector.

5. The method of claim 1, wherein the composition further comprises a pharmaceutically acceptable excipient.

6. (canceled)

7. The method of claim 1, wherein the disease is a disease or disorder associated with a UGA PTC, and further wherein the method comprises administering at least one ACE-tRNA specific for UGA.

8. The method of claim 1, wherein the disease is a disease or disorder associated with a UAA PTC, and further wherein the method comprises administering at least one ACE-tRNA specific for UAA.

9. The method of claim 1, wherein the disease is a disease or disorder associated with a UAG PTC, and further wherein the method comprises administering at least one ACE-tRNA specific for UAG.

10. The method of claim 1, wherein the method comprises administering at least two ACE-tRNA, wherein each of the at least two ACE-RNA are specific for at least two different amino acid molecules onto a polypeptide chain.

11. The method of claim 1, wherein the method comprises administering at least two ACE-tRNA, wherein each of the at least two ACE-tRNA are specific for incorporating the same amino acid molecule onto a polypeptide chain.

12. The method of claim 1, wherein the the one or more ACE-tRNA are encoded on the same nucleic acid molecule.

13. The method of claim 1, wherein the the one or more ACE-tRNA are encoded on different nucleic acid molecules.

14. The method of claim 7, wherein the at least one ACE-tRNA specific for UGA selected from the group consisting of ACE-tRNAArg, ACE-tRNAGly and ACE-tRNATrp.

15. The method of claim 1, wherein the disease is selected from the group consisting of Duchenne or Becker muscular dystrophy, retinoblastoma, neurofibromatosis, ataxia telangiectasia, Tay-Sachs disease, cystic fibrosis, Wilm's tumor, hemophilia A, hemophilia B, Menkes disease, Ullrich's disease, β-Thalassemia, type 2A or type 3 von Willebrand disease, Robinow syndrome, brachydactyly type B (shortening of digits and metacarpals), inherited susceptibility to mycobacterial infection, inherited retinal disease, inherited bleeding tendency, inherited blindness, congenital neurosensory deafness, colonic agangliosis, inherited neural develop mental defect including neurosensory deafness, peripheral neuropathy, central demyelinating leukodystrophy, Liddle's syndrome, xeroderma pigmentosum, Fanconi's anemia, anemia, hypothyroidism, p53-associated cancers (e.g., p53-associated squamous cell carcinoma, p53-associated hepatocellular carcinoma, p53-associated ovarian carcinoma), esophageal carcinoma, osteocarcinoma, ovarian carcinoma, hepatocellular carcinoma, breast cancer, fibrous histiocytoma, SRY sex reversal, triosephosphate isomerase-anemia, diabetes, and rickets.

16. The method of claim 1, wherein the subject is a mammal.


SEQUENCE LISTING (655 sequences)

SEQ ID NO: 1 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcgcgtctgacttcagatcagaaggttgcgggttcaaatcccgtcggggtca

SEQ ID NO: 2 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcgcgtctgacttcagatcagaaggttacgggttcaaatcccgtcggggtca

SEQ ID NO: 3 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcgcgtctgacttcagatcagaaggttccgggttcaaatcccggcggggtca

SEQ ID NO: 4 - DNA, 77 bp, synthetic construct
  misc_feature 1..77: note = Description of Artificial Sequence: Synthetic oligonucleotide
  source 1..77: mol_type = other DNA
cgtcggctctgtggcgcaatggatagcgcattggacttcaaattcaaaggttgtgggttcgagtcccaccagagtcg

SEQ ID NO: 5 - DNA, 77 bp, synthetic construct
  misc_feature 1..77: note = Description of Artificial Sequence: Synthetic oligonucleotide
  source 1..77: mol_type = other DNA
cgtcgccccagtggcctaatggataaggcactggccttcaaagccagggattgtgggttcgagtcccacctggggtg

SEQ ID NO: 6 - DNA, 77 bp, synthetic construct
  misc_feature 1..77: note = Description of Artificial Sequence: Synthetic oligonucleotide
  source 1..77: mol_type = other DNA
cgtcggctccgtggcgcaatggatagcgcattggacttcaaattcaaaggttccgggttcgagtcccggcggagtcg

SEQ ID NO: 7 - DNA, 77 bp, synthetic construct
  misc_feature 1..77: note = Description of Artificial Sequence: Synthetic oligonucleotide
  source 1..77: mol_type = other DNA
cgtcgccccagtggcctaatggataaggcattggccttcaaagccagggattgtgggttcgagtcccatctggggtg

SEQ ID NO: 8 - DNA, 77 bp, synthetic construct
  misc_feature 1..77: note = Description of Artificial Sequence: Synthetic oligonucleotide
  source 1..77: mol_type = other DNA
cgtcggctctgtggcgcaatggatagcgcattggacttcaaattcaaaggttgtgggttcgaatcccaccagagtcg

SEQ ID NO: 9 - DNA, 95 bp, synthetic construct
  misc_feature 1..95: note = Description of Artificial Sequence: Synthetic oligonucleotide
  source 1..95: mol_type = other DNA
cgtcggctctgtggcgcaatggatagcgcattggacttcaagctgagcctagtgtggtcattcaaaggttgtgggttcgagtcccaccagagtcg

SEQ ID NO: 10 - DNA, 77 bp, synthetic construct
  misc_feature 1..77: note = Description of Artificial Sequence: Synthetic oligonucleotide
  source 1..77: mol_type = other DNA
cgtcgccccggtggcctaatggataaggcattggccttcaaagccagggattgtgggttcgagtcccacccggggta

SEQ ID NO: 11 - DNA, 89 bp, synthetic construct
  misc_feature 1..89: note = Description of Artificial Sequence: Synthetic oligonucleotide
  source 1..89: mol_type = other DNA
cgtcggctccgtggcgcaatggatagcgcattggacttcaagaggctgaaggcattcaaaggttccgggttcgagtcccggcggagtcg

SEQ ID NO: 12 - DNA, 92 bp, synthetic construct
  misc_feature 1..92: note = Description of Artificial Sequence: Synthetic oligonucleotide
  source 1..92: mol_type = other DNA
cgtcggctctgtggcgcaatggatagcgcattggacttcaagtgacgaatagagcaattcaaaggttgtgggttcgaatcccaccagagtcg

SEQ ID NO: 13 - DNA, 77 bp, synthetic construct
  misc_feature 1..77: note = Description of Artificial Sequence: Synthetic oligonucleotide
  source 1..77: mol_type = other DNA
cgtcggccgcgtggcctaatggataaggcgtctgacttcagatcagaagattgcaggttcgagtcctgccgcggtcg

SEQ ID NO: 14 - DNA, 77 bp, synthetic construct
  misc_feature 1..77: note = Description of Artificial Sequence: Synthetic oligonucleotide
  source 1..77: mol_type = other DNA
cgtcgaccgcgtggcctaatggataaggcgtctgacttcagatcagaagattgagggttcgagtcccttcgtggtcg

SEQ ID NO: 15 - DNA, 90 bp, synthetic construct
  misc_feature 1..90: note = Description of Artificial Sequence: Synthetic oligonucleotide
  source 1..90: mol_type = other DNA
cgtcggctctgtggcgcaatggatagcgcattggacttcaagatagttagagaaattcaaaggttgtgggttcgagtcccaccagagtcg

SEQ ID NO: 16 - DNA, 76 bp, synthetic construct
  misc_feature 1..76: note = Description of Artificial Sequence: Synthetic oligonucleotide
  source 1..76: mol_type = other DNA
cgtcggttccatggtgtaatggtgagcactctggactctaaatccagcgatccgagttcgagtctcggtggaacct

SEQ ID NO: 17 - DNA, 76 bp, synthetic construct
  misc_feature 1..76: note = Description of Artificial Sequence: Synthetic oligonucleotide
  source 1..76: mol_type = other DNA
cgtcggccccatggtgtaatggttagcactctggactctaaatccagcgatccgagttcaaatctcggtgggacct

SEQ ID NO: 18 - DNA, 76 bp, synthetic construct
  misc_feature 1..76: note = Description of Artificial Sequence: Synthetic oligonucleotide
  source 1..76: mol_type = other DNA
cgtcggtcccatggtgtaatggttagcactctggactctaaatccagcaatccgagttcgaatctcggtgggacct

SEQ ID NO: 19 - DNA, 76 bp, synthetic construct
  misc_feature 1..76: note = Description of Artificial Sequence: Synthetic oligonucleotide
  source 1..76: mol_type = other DNA
cgtcggtcccatggtgtaatggttagcactctggactctaaatccagcgatccgagttcaaatctcggtgggacct

SEQ ID NO: 20 - DNA, 76 bp, synthetic construct
  misc_feature 1..76: note = Description of Artificial Sequence: Synthetic oligonucleotide
  source 1..76: mol_type = other DNA
cgtcggccccatggtgtaatggtcagcactctggactctaaatccagcgatccgagttcaaatctcggtgggaccc

SEQ ID NO: 21 - DNA, 76 bp, synthetic construct
  misc_feature 1..76: note = Description of Artificial Sequence: Synthetic oligonucleotide
  source 1..76: mol_type = other DNA
cgtcggttccatggtgtaatggtaagcactctggactctaaatccagcgatccgagttcgagtctcggtggaacct

SEQ ID NO: 22 - DNA, 76 bp, synthetic construct
  misc_feature 1..76: note = Description of Artificial Sequence: Synthetic oligonucleotide
  source 1..76: mol_type = other DNA
cgtcggttccatggtgtaatggttagcactctggactctaaatccggtaatccgagttcaaatctcggtggaacct

SEQ ID NO: 23 - DNA, 76 bp, synthetic construct
  misc_feature 1..76: note = Description of Artificial Sequence: Synthetic oligonucleotide
  source 1..76: mol_type = other DNA
cgtcggttccatggtgtaatggttagcactctggactctaaatccagcgatccgagttcaagtctcggtggaacct

SEQ ID NO: 24 - DNA, 76 bp, synthetic construct
  misc_feature 1..76: note = Description of Artificial Sequence: Synthetic oligonucleotide
  source 1..76: mol_type = other DNA
cgtcggttccatggtgtaatggtaagcactctggactttaaatccagcgatccgagttcgagtctcggtggaacct

SEQ ID NO: 25 - DNA, 76 bp, synthetic construct
  misc_feature 1..76: note = Description of Artificial Sequence: Synthetic oligonucleotide
  source 1..76: mol_type = other DNA
cgtcggccccatggtgtaatggttagcactctggactttaaatccagcgatccgagttcaaatctcggtgggacct

SEQ ID NO: 26 - DNA, 76 bp, synthetic construct
  misc_feature 1..76: note = Description of Artificial Sequence: Synthetic oligonucleotide
  source 1..76: mol_type = other DNA
cgtcggttccatggtgtaatggtgagcactctggactttaaatccagcgatccgagttcgagtctcggtggaacct

SEQ ID NO: 27 - DNA, 76 bp, synthetic construct
  misc_feature 1..76: note = Description of Artificial Sequence: Synthetic oligonucleotide
  source 1..76: mol_type = other DNA
cgtcggttccatggtgtaatggttagcactctggactttaaatccagcgatccgagttcaaatctcggtggaacct

SEQ ID NO: 28 - DNA, 76 bp, synthetic construct
  misc_feature 1..76: note = Description of Artificial Sequence: Synthetic oligonucleotide
  source 1..76: mol_type = other DNA
cgtcggtcccatggtgtaatggttagcactctggactttaaatccagcgatccgagttcaaatctcggtgggacct

SEQ ID NO: 29 - DNA, 76 bp, synthetic construct
  misc_feature 1..76: note = Description of Artificial Sequence: Synthetic oligonucleotide
  source 1..76: mol_type = other DNA
cgtcggtcccatggtgtaatggttagcactctggactttaaatccagcaatccgagttcgaatctcggtgggacct

SEQ ID NO: 30 - DNA, 76 bp, synthetic construct
  misc_feature 1..76: note = Description of Artificial Sequence: Synthetic oligonucleotide
  source 1..76: mol_type = other DNA
cgtcggttccatggtgtaatggttagcactctggactttaaatccggtaatccgagttcaaatctcggtggaacct

SEQ ID NO: 31 - DNA, 76 bp, synthetic construct
  misc_feature 1..76: note = Description of Artificial Sequence: Synthetic oligonucleotide
  source 1..76: mol_type = other DNA
cgtcggccccatggtgtaatggtcagcactctggactttaaatccagcgatccgagttcaaatctcggtgggaccc

SEQ ID NO: 32 - DNA, 76 bp, synthetic construct
  misc_feature 1..76: note = Description of Artificial Sequence: Synthetic oligonucleotide
  source 1..76: mol_type = other DNA
cgtcggttccatggtgtaatggttagcactctggactttaaatccagcgatccgagttcaagtctcggtggaacct

SEQ ID NO: 33 - DNA, 76 bp, synthetic construct
  misc_feature 1..76: note = Description of Artificial Sequence: Synthetic oligonucleotide
  source 1..76: mol_type = other DNA
cgtcgacctcgtggcgcaatggtagcgcgtctgactctagatcagaaggttgcgtgttcaagtcacgtcggggtca

SEQ ID NO: 34 - DNA, 76 bp, synthetic construct
  misc_feature 1..76: note = Description of Artificial Sequence: Synthetic oligonucleotide
  source 1..76: mol_type = other DNA
cgtcgacctcgtggcgcaacggtagcgcgtctgactctagatcagaaggttgcgtgttcaaatcacgtcggggtca

SEQ ID NO: 35 - DNA, 76 bp, synthetic construct
  misc_feature 1..76: note = Description of Artificial Sequence: Synthetic oligonucleotide
  source 1..76: mol_type = other DNA
cgtcggcctcgtggcgcaacggtagcgcgtctgactctagatcagaaggttgcgtgttcaaatcacgtcggggtca

SEQ ID NO: 36 - DNA, 76 bp, synthetic construct
  misc_feature 1..76: note = Description of Artificial Sequence: Synthetic oligonucleotide
  source 1..76: mol_type = other DNA
cgtcgacctcgtggcgcaacggtagcgcgtctgactctagatcagaaggctgcgtgttcgaatcacgtcggggtca

SEQ ID NO: 37 - DNA, 76 bp, synthetic construct
  misc_feature 1..76: note = Description of Artificial Sequence: Synthetic oligonucleotide
  source 1..76: mol_type = other DNA
cgtcgacctcgtggcgcaacggcagcgcgtctgactctagatcagaaggttgcgtgttcaaatcacgtcggggtca

SEQ ID NO: 38 - DNA, 76 bp, synthetic construct
  misc_feature 1..76: note = Description of Artificial Sequence: Synthetic oligonucleotide
  source 1..76: mol_type = other DNA
cgtctcccacatggtctagcggttaggattcctggttctaacccaggcggcccgggttcgactcccggtgtgggaa

SEQ ID NO: 39 - DNA, 76 bp, synthetic construct
  misc_feature 1..76: note = Description of Artificial Sequence: Synthetic oligonucleotide
  source 1..76: mol_type = other DNA
cgtctcccatatggtctagcggttaggattcctggttctaacccaggtggcccgggttcgactcccggtatgggaa

SEQ ID NO: 40 - DNA, 76 bp, synthetic construct
  misc_feature 1..76: note = Description of Artificial Sequence: Synthetic oligonucleotide
  source 1..76: mol_type = other DNA
cgtctccctggtggtctagtggctaggattcggcgctctaaccgccgcggcccgggttcgattcccggtcagggaa

SEQ ID NO: 41 - DNA, 76 bp, synthetic construct
  misc_feature 1..76: note = Description of Artificial Sequence: Synthetic oligonucleotide
  source 1..76: mol_type = other DNA
cgtctccctggtggtctagtggttaggattcggcgctctaaccgccgcggcccgggttcgattcccggtcagggaa

SEQ ID NO: 42 - DNA, 76 bp, synthetic construct
  misc_feature 1..76: note = Description of Artificial Sequence: Synthetic oligonucleotide
  source 1..76: mol_type = other DNA
cgtctccctggtggtctagtggctaggattcggcgctctaaccgccgcggcccgggttcgattcccggccagggaa

SEQ ID NO: 43 - DNA, 76 bp, synthetic construct; identical to SEQ ID NO: 38
  misc_feature 1..76: note = Description of Artificial Sequence: Synthetic oligonucleotide
  source 1..76: mol_type = other DNA

SEQ ID NO: 44 - DNA, 76 bp, synthetic construct; identical to SEQ ID NO: 39
  misc_feature 1..76: note = Description of Artificial Sequence: Synthetic oligonucleotide
  source 1..76: mol_type = other DNA

SEQ ID NO: 45 - DNA, 76 bp, synthetic construct; identical to SEQ ID NO: 41
  misc_feature 1..76: note = Description of Artificial Sequence: Synthetic oligonucleotide
  source 1..76: mol_type = other DNA

SEQ ID NO: 46 - DNA, 76 bp, synthetic construct
  misc_feature 1..76: note = Description of Artificial Sequence: Synthetic oligonucleotide
  source 1..76: mol_type = other DNA
cgtctccctggtggtctagtggttaggattcggcgctctaaccgccgcggcccgggttcgattcccggtcaggaaa

SEQ ID NO: 47 - DNA, 76 bp, synthetic construct; identical to SEQ ID NO: 42
  misc_feature 1..76: note = Description of Artificial Sequence: Synthetic oligonucleotide
  source 1..76: mol_type = other DNA

SEQ ID NO: 48 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcgcgtctgacttcagatcagaaggttgcgtgttcaaatcacgtcggggtca

SEQ ID NO: 49 - DNA, 72 bp, synthetic construct
  misc_feature 1..72: note = Description of Artificial Sequence: Synthetic oligonucleotide
  source 1..72: mol_type = other DNA
gacctcgtggcgcaatggtagcgcgtctgacttcagatcagaaggttgcgtgttcaagtcacgtcggggtca

SEQ ID NO: 50 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgacttcagatcagaaggttgcgtgttcaaatcacgtcggggtca

SEQ ID NO: 51 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgacttcagatcagaaggctgcgtgttcgaatcacgtcggggtca

SEQ ID NO: 52 - DNA, 72 bp, synthetic construct
  misc_feature 1..72: note = Description of Artificial Sequence: Synthetic oligonucleotide
  source 1..72: mol_type = other DNA
gacctcgtggcgcaacggcagcgcgtctgacttcagatcagaaggttgcgtgttcaaatcacgtcggggtca

SEQ ID NO: 53 - DNA, 72 bp, synthetic construct
  misc_feature 1..72: note = Description of Artificial Sequence: Synthetic oligonucleotide
  source 1..72: mol_type = other DNA
gcgttggtggtatagtggttagcatagctgccttcaaagcagttgacccgggttcgattcccggccaacgca

SEQ ID NO: 54 - DNA, 72 bp, synthetic construct
  misc_feature 1..72: note = Description of Artificial Sequence: Synthetic oligonucleotide
  source 1..72: mol_type = other DNA
gcgttggtggtatagtggtgagcatagctgccttcaaagcagttgacccgggttcgattcccggccaacgca

SEQ ID NO: 55 - DNA, 72 bp, synthetic construct
  misc_feature 1..72: note = Description of Artificial Sequence: Synthetic oligonucleotide
  source 1..72: mol_type = other DNA
gcgttggtggtatagtggtaagcatagctgccttcaaagcagttgacccgggttcgattcccggccaacgca

SEQ ID NO: 56 - DNA, 72 bp, synthetic construct; identical to SEQ ID NO: 48
  misc_feature 1..72: note = Description of Artificial Sequence: Synthetic oligonucleotide
  source 1..72: mol_type = other DNA

SEQ ID NO: 57 - DNA, 72 bp, synthetic construct; identical to SEQ ID NO: 49
  misc_feature 1..72: note = Description of Artificial Sequence: Synthetic oligonucleotide
  source 1..72: mol_type = other DNA

SEQ ID NO: 58 - DNA, 72 bp, synthetic construct; identical to SEQ ID NO: 50
  misc_feature 1..72: note = Description of Artificial Sequence: Synthetic oligonucleotide
  source 1..72: mol_type = other DNA

SEQ ID NO: 59 - DNA, 72 bp, synthetic construct; identical to SEQ ID NO: 51
  misc_feature 1..72: note = Description of Artificial Sequence: Synthetic oligonucleotide
  source 1..72: mol_type = other DNA

SEQ ID NO: 60 - DNA, 72 bp, synthetic construct; identical to SEQ ID NO: 52
  misc_feature 1..72: note = Description of Artificial Sequence: Synthetic oligonucleotide
  source 1..72: mol_type = other DNA

SEQ ID NO: 61 - DNA, 72 bp, synthetic construct
  misc_feature 1..72: note = Description of Artificial Sequence: Synthetic oligonucleotide
  source 1..72: mol_type = other DNA
ggcctcatggtgcaacagtagtgtgtctgacttcagatcagaaggttgtatgttcaaatcacgtaggggtca

SEQ ID NO: 62 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcgcgtctgactctagatcagaaggttgcgtgttcaaatcacgtcggggtca

SEQ ID NO: 63 - DNA, 72 bp, synthetic construct
  misc_feature 1..72: note = Description of Artificial Sequence: Synthetic oligonucleotide
  source 1..72: mol_type = other DNA
gacctcgtggcgcaatggtagcgcgtctgactctagatcagaaggttgcgtgttcaagtcacgtcggggtca

SEQ ID NO: 64 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgactctagatcagaaggttgcgtgttcaaatcacgtcggggtca

SEQ ID NO: 65 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgactctagatcagaaggctgcgtgttcgaatcacgtcggggtca

SEQ ID NO: 66 - DNA, 72 bp, synthetic construct
  misc_feature 1..72: note = Description of Artificial Sequence: Synthetic oligonucleotide
  source 1..72: mol_type = other DNA
gacctcgtggcgcaacggcagcgcgtctgactctagatcagaaggttgcgtgttcaaatcacgtcggggtca

SEQ ID NO: 67 - DNA, 72 bp, synthetic construct
  misc_feature 1..72: note = Description of Artificial Sequence: Synthetic oligonucleotide
  source 1..72: mol_type = other DNA
ggcctcatggtgcaacagtagtgtgtctgactctagatcagaaggttgtatgttcaaatcacgtaggggtca

SEQ ID NO: 68 - DNA, 71 bp, synthetic construct
  misc_feature 1..71: note = Description of Artificial Sequence: Synthetic oligonucleotide
  source 1..71: mol_type = other DNA
gcattggtggttcagtggtagaattctcgccttcaacgcgggagacccgggttcaattcccggccaatgca

SEQ ID NO: 69 - DNA, 72 bp, synthetic construct
  misc_feature 1..72: note = Description of Artificial Sequence: Synthetic oligonucleotide
  source 1..72: mol_type = other DNA
gcgccgctggtgtagtggtatcatgcaagatttcaaattcttgcgacccgggttcgattcccgggcggcgca

SEQ ID NO: 70 - DNA, 71 bp, synthetic construct
  misc_feature 1..71: note = Description of Artificial Sequence: Synthetic oligonucleotide
  source 1..71: mol_type = other DNA
gcattggtggttcaatggtagaattctcgccttcaacgcaggagacccaggttcgattcctggccaatgca

SEQ ID NO: 71 - DNA, 71 bp, synthetic construct
  misc_feature 1..71: note = Description of Artificial Sequence: Synthetic oligonucleotide
  source 1..71: mol_type = other DNA
gcgttggtggtttagtggtagaattctcgccttcaatgcgggagacccgggttcaattcccggccactgca

SEQ ID NO: 72 - DNA, 71 bp, synthetic construct
  misc_feature 1..71: note = Description of Artificial Sequence: Synthetic oligonucleotide
  source 1..71: mol_type = other DNA
gccttggtggtgcagtggtagaattctcgccttcaacgtgggagacccgggttcaattcccggccaatgca

SEQ ID NO: 73 - DNA, 61 bp, synthetic construct
  misc_feature 1..61: note = Description of Artificial Sequence: Synthetic oligonucleotide
  source 1..61: mol_type = other DNA
ggtggttcagtggtagaattctcgccttcaacgcgggagacccgggtttaattcccggtca

SEQ ID NO: 74 - DNA, 61 bp, synthetic construct
  misc_feature 1..61: note = Description of Artificial Sequence: Synthetic oligonucleotide
  source 1..61: mol_type = other DNA
gtggtctagtggttaggattcagcgcttcaaccgccgcagcccgggttcgattcccggtca

SEQ ID NO: 75 - DNA, 71 bp, synthetic construct
  misc_feature 1..71: note = Description of Artificial Sequence: Synthetic oligonucleotide
  source 1..71: mol_type = other DNA
gcgtcagtggtttagtggtggaattcctgccttcaatgcacgagatccgtgttcaactcctggttggtgca

SEQ ID NO: 76 - DNA, 72 bp, synthetic construct
  misc_feature 1..72: note = Description of Artificial Sequence: Synthetic oligonucleotide
  source 1..72: mol_type = other DNA
gcgtcagtggttttagtggtggaattcctgccttcaatgcacgagatccgtgttcaactcctggttggtgca

SEQ ID NO: 77 - DNA, 71 bp, synthetic construct
  misc_feature 1..71: note = Description of Artificial Sequence: Synthetic oligonucleotide
  source 1..71: mol_type = other DNA
gcgttggcagttcagtggtagaattctcgccttcaacccgggagacctggattccatttccggcaaatgca

SEQ ID NO: 78 - DNA, 71 bp, synthetic construct
  misc_feature 1..71: note = Description of Artificial Sequence: Synthetic oligonucleotide
  source 1..71: mol_type = other DNA
gcatgggtggttcagtggtagaattctcgccttcaacgcgggaggcccgggttcgattcccggcccatgca

SEQ ID NO: 79 - DNA, 71 bp, synthetic construct
  misc_feature 1..71: note = Description of Artificial Sequence: Synthetic oligonucleotide
  source 1..71: mol_type = other DNA
gcattggtggttcagtggtagaattctcgccttcaacgcgggaggcccgggttcgattcccggccaatgca

SEQ ID NO: 80 - DNA, 71 bp, synthetic construct
  misc_feature 1..71: note = Description of Artificial Sequence: Synthetic oligonucleotide
  source 1..71: mol_type = other DNA
gcattggtggttcagtggtagaattctcgccttcaacgcgggaggcccgggtttgattcccggccagtgca

SEQ ID NO: 81 - DNA, 71 bp, synthetic construct
  misc_feature 1..71: note = Description of Artificial Sequence: Synthetic oligonucleotide
  source 1..71: mol_type = other DNA
gcataggtggttcagtggtagaattcttgccttcaacgcaggaggcccaggtttgattcctggcccatgca

SEQ ID NO: 82 - DNA, 71 bp, synthetic construct
  misc_feature 1..71: note = Description of Artificial Sequence: Synthetic oligonucleotide
  source 1..71: mol_type = other DNA
gcattggtggttcagtggtagaattctcgccttcaatgcgggcggccgggcttcgattcctggccaatgca

SEQ ID NO: 83 - DNA, 71 bp, synthetic construct
  misc_feature 1..71: note = Description of Artificial Sequence: Synthetic oligonucleotide
  source 1..71: mol_type = other DNA
gcatgggtgattcagtggtagaattttcaccttcaatgcaggaggtccaggttcatttcctggcctatgca

SEQ ID NO: 84 - DNA, 72 bp, synthetic construct; identical to SEQ ID NO: 53
  misc_feature 1..72: note = Description of Artificial Sequence: Synthetic oligonucleotide
  source 1..72: mol_type = other DNA

SEQ ID NO: 85 - DNA, 72 bp, synthetic construct; identical to SEQ ID NO: 54
  misc_feature 1..72: note = Description of Artificial Sequence: Synthetic oligonucleotide
  source 1..72: mol_type = other DNA

SEQ ID NO: 86 - DNA, 72 bp, synthetic construct; identical to SEQ ID NO: 55
  misc_feature 1..72: note = Description of Artificial Sequence: Synthetic oligonucleotide
  source 1..72: mol_type = other DNA

SEQ ID NO: 87 - DNA, 72 bp, synthetic construct
  misc_feature 1..72: note = Description of Artificial Sequence: Synthetic oligonucleotide
  source 1..72: mol_type = other DNA
gcgttggtggtatagtggtgagcatagttgccttcaaagcagttgacccgggctcgattcccgcccaacgca

SEQ ID NO: 88 - DNA, 72 bp, synthetic construct
  misc_feature 1..72: note = Description of Artificial Sequence: Synthetic oligonucleotide
  source 1..72: mol_type = other DNA
gcgttggtggtatagtggtgagcatagttgccttcaaagcagttgacccgggctcgattcccggccaacgca

SEQ ID NO: 89 - DNA, 73 bp, synthetic construct
  misc_feature 1..73: note = Description of Artificial Sequence: Synthetic oligonucleotide
  source 1..73: mol_type = other DNA
gggccagtggcgcaatggataacgcgtctgacttcagatcagaagattccaggttcgactcctggctggctcg

SEQ ID NO: 90 - DNA, 73 bp, synthetic construct
  misc_feature 1..73: note = Description of Artificial Sequence: Synthetic oligonucleotide
  source 1..73: mol_type = other DNA
gggccagtggcgcaatggataacgcgtctgacttcagatcagaagattctaggttcgactcctggctggctcg

SEQ ID NO: 91 - DNA, 73 bp, synthetic construct
  misc_feature 1..73: note = Description of Artificial Sequence: Synthetic oligonucleotide
  source 1..73: mol_type = other DNA
ggccgcgtggcctaatggataaggcgtctgatttcagatcagaagattgagggttcgagtcccttcgtggtcg

SEQ ID NO: 92 - DNA, 73 bp, synthetic construct
  misc_feature 1..73: note = Description of Artificial Sequence: Synthetic oligonucleotide
  source 1..73: mol_type = other DNA
gacccagtggcctaatggataaggcatcagccttcagagctggggattgtgggttcgagtcccatctgggtcg

SEQ ID NO: 93 - DNA, 73 bp, synthetic construct
  misc_feature 1..73: note = Description of Artificial Sequence: Synthetic oligonucleotide
  source 1..73: mol_type = other DNA
gccccagtggcctaatggataaggcactggccttcaaagccagggattgtgggttcgagtcccacctggggta

SEQ ID NO: 94 - DNA, 73 bp, synthetic construct
  misc_feature 1..73: note = Description of Artificial Sequence: Synthetic oligonucleotide
  source 1..73: mol_type = other DNA
gccccagtggcctaatggataaggcactggccttcaaagccagggattgtgggttcgagtcccacctggggtg

SEQ ID NO: 95 - DNA, 73 bp, synthetic construct
  misc_feature 1..73: note = Description of Artificial Sequence: Synthetic oligonucleotide
  source 1..73: mol_type = other DNA
gccccggtggcctaatggataaggcattggccttcaaagccagggattgtgggttcgagtcccacccggggta

SEQ ID NO: 96 - DNA, 73 bp, synthetic construct
  misc_feature 1..73: note = Description of Artificial Sequence: Synthetic oligonucleotide
  source 1..73: mol_type = other DNA
gccccagtggcctaatggataaggcattggccttcaaagccagggattgtgggttcgagtcccatctggggtg

SEQ ID NO: 97 - DNA, 73 bp, synthetic construct
  misc_feature 1..73: note = Description of Artificial Sequence: Synthetic oligonucleotide
  source 1..73: mol_type = other DNA
gccccagtggcctgatggataaggtactggccttcaaagccagggattgtgggttcgagttccacctggggta

SEQ ID NO: 98 - DNA, 73 bp, synthetic construct
  misc_feature 1..73: note = Description of Artificial Sequence: Synthetic oligonucleotide
  source 1..73: mol_type = other DNA
ggccgcgtggcctaatggataaggcgtctgacttcagatcagaagattgcaggttcgagtcctgccgcggtcg

SEQ ID NO: 99 - DNA, 73 bp, synthetic construct
  misc_feature 1..73: note = Description of Artificial Sequence: Synthetic oligonucleotide
  source 1..73: mol_type = other DNA
gaccacgtggcctaatggataaggcgtctgacttcagatcagaagattgagggttcgaatccctccgtggtta

SEQ ID NO: 100 - DNA, 73 bp, synthetic construct
  misc_feature 1..73: note = Description of Artificial Sequence: Synthetic oligonucleotide
  source 1..73: mol_type = other DNA
gaccgcgtggcctaatggataaggcgtctgacttcagatcagaagattgagggttcgagtcccttcgtggtcg

SEQ ID NO: 101 - DNA, 73 bp, synthetic construct
  misc_feature 1..73: note = Description of Artificial Sequence: Synthetic oligonucleotide
  source 1..73: mol_type = other DNA
gaccacgtggcctaatggataaggcgtctgacttcagatcagaagattgagggttcgaatcccttcgtggtta

SEQ ID NO: 102 - DNA, 73 bp, synthetic construct
  misc_feature 1..73: note = Description of Artificial Sequence: Synthetic oligonucleotide
  source 1..73: mol_type = other DNA
gaccacgtggcctaatggataaggcgtctgacttcagatcagaagattgagggttcgaatcccttcgtggttg

SEQ ID NO: 103 - DNA, 73 bp, synthetic construct
  misc_feature 1..73: note = Description of Artificial Sequence: Synthetic oligonucleotide
  source 1..73: mol_type = other DNA
ggccgtgtggcctaatggataaggcgtctgacttcagatcaaaagattgcaggtttgagttctgccacggtcg

SEQ ID NO: 104 - DNA, 85 bp, synthetic construct
  misc_feature 1..85: note = Description of Artificial Sequence: Synthetic oligonucleotide
  source 1..85: mol_type = other DNA
ggctccgtggcgcaatggatagcgcattggacttcaagaggctgaaggcattcaaaggttccgggttcgagtcccggcggagtcg

SEQ ID NO: 105 - DNA, 73 bp, synthetic construct
  misc_feature 1..73: note = Description of Artificial Sequence: Synthetic oligonucleotide
  source 1..73: mol_type = other DNA
ggctccgtggcgcaatggatagcgcattggacttcaaattcaaaggttccgggttcgagtcccggcggagtcg

SEQ ID NO: 106 - DNA, 88 bp, synthetic construct
  misc_feature 1..88: note = Description of Artificial Sequence: Synthetic oligonucleotide
  source 1..88: mol_type = other DNA
ggctctgtggcgcaatggatagcgcattggacttcaagtgacgaatagagcaattcaaaggttgtgggttcgaatcccaccagagtcg

SEQ ID NO: 107 - DNA, 73 bp, synthetic construct
  misc_feature 1..73: note = Description of Artificial Sequence: Synthetic oligonucleotide
  source 1..73: mol_type = other DNA
ggctctgtggcgcaatggatagcgcattggacttcaaattcaaaggttgtgggttcgaatcccaccagagtcg

SEQ ID NO: 108 - DNA, 91 bp, synthetic construct
  misc_feature 1..91: note = Description of Artificial Sequence: Synthetic oligonucleotide
  source 1..91: mol_type = other DNA
ggctctgtggcgcaatggatagcgcattggacttcaagctgagcctagtgtggtcattcaaaggttgtgggttcgagtcccaccagagtcg

SEQ ID NO: 109 - DNA, 73 bp, synthetic construct
  misc_feature 1..73: note = Description of Artificial Sequence: Synthetic oligonucleotide
  source 1..73: mol_type = other DNA
ggctctgtggcgcaatggatagcgcattggacttcaaattcaaaggttgtgggttcgagtcccaccagagtcg

SEQ ID NO: 110 - DNA, 86 bp, synthetic construct
  misc_feature 1..86: note = Description of Artificial Sequence: Synthetic oligonucleotide
  source 1..86: mol_type = other DNA
ggctctgtggcgcaatggatagcgcattggacttcaagatagttagagaaattcaaaggttgtgggttcgagtcccaccagagtcg

SEQ ID NO: 111 - DNA, 74 bp, synthetic construct
  misc_feature 1..74: note = Description of Artificial Sequence: Synthetic oligonucleotide
  source 1..74: mol_type = other DNA
gtctctgtggcgcaatggacgagcgcgctggacttcaaatccagaggttccgggttcgagtcccggcagagatg

SEQ ID NO: 112 - DNA, 87 bp, synthetic construct
  misc_feature 1..87: note = Description of Artificial Sequence: Synthetic oligonucleotide
  source 1..87: mol_type = other DNA
ggctctgtggcgcaatggatagcgcattggacttcaagcctaaatcaagagattcaaaggttgcgggttcgagtccctccagagtcg

SEQ ID NO: 113 - DNA, 73 bp, synthetic construct
  misc_feature 1..73: note = Description of Artificial Sequence: Synthetic oligonucleotide
  source 1..73: mol_type = other DNA
ggctctgtggcgcaatggatagcgcattggacttcaaattcaaaggttgcgggttcgagtccctccagagtcg

SEQ ID NO: 114 - DNA, 69 bp, synthetic construct
  misc_feature 1..69: note = Description of Artificial Sequence: Synthetic oligonucleotide
  source 1..69: mol_type = other DNA
ggcagcatagcagagtggttcaggttacaggttcaagatgtaaactgagttcaaatcccagttctgcca

SEQ ID NO: 115 - DNA, 57 bp, synthetic construct
  misc_feature 1..57: note = Description of Artificial Sequence: Synthetic oligonucleotide
  source 1..57: mol_type = other DNA
tggtgtaataggtagcacagagaattctagattctcaggggtaggttcaattcctat

SEQ ID NO: 116 - DNA, 72 bp, synthetic construct
  misc_feature 1..72: note = Description of Artificial Sequence: Synthetic oligonucleotide
  source 1..72: mol_type = other DNA
taggacatggtgtgataggtagcatggagaattctagattctcaggggtaggttcaattcctacagttctag

SEQ ID NO: 117 - DNA, 72 bp, synthetic construct
  misc_feature 1..72: note = Description of Artificial Sequence: Synthetic oligonucleotide
  source 1..72: mol_type = other DNA
taggacgtggtgtgataggtagcatggggaattctagattctcaggggtgggttcaattcctatagttctag

SEQ ID NO: 118 - DNA, 72 bp, synthetic construct
  misc_feature 1..72: note = Description of Artificial Sequence: Synthetic oligonucleotide
  source 1..72: mol_type = other DNA
taggacgtggtgtagtaggtagcatggagaatgctaaattctcaggggtaggttcaattcctatagttctag

SEQ ID NO: 119 - DNA, 72 bp, synthetic construct
  misc_feature 1..72: note = Description of Artificial Sequence: Synthetic oligonucleotide
  source 1..72: mol_type = other DNA
taggacatggtgtaataggtagaatggagaattctaaattctcaggggtaggttcaattcctatagttctag

SEQ ID NO: 120 - DNA, 72 bp, synthetic construct
  misc_feature 1..72: note = Description of Artificial Sequence: Synthetic oligonucleotide
  source 1..72: mol_type = other DNA
taggatgtggtgtattaggtagcacagagaattctagattctcaggggtaggttcgattcctataattctac

SEQ ID NO: 121 - DNA, 72 bp, synthetic construct
  misc_feature 1..72: note = Description of Artificial Sequence: Synthetic oligonucleotide
  source 1..72: mol_type = other DNA
taggacttggtgtaatgggtagcacagagaattctagattctcaggggtgggttcaattcctttcgtcctag

SEQ ID NO: 122 - DNA, 75 bp, synthetic construct
  misc_feature 1..75: note = Description of Artificial Sequence: Synthetic oligonucleotide
  source 1..75: mol_type = other DNA
tctaggatgtggtgtgataggtagcatggagaattctagattctcaggggtaggttcaattcctatattctagaa

SEQ ID NO: 123 - DNA, 72 bp, synthetic construct
  misc_feature 1..72: note = Description of Artificial Sequence: Synthetic oligonucleotide
  source 1..72: mol_type = other DNA
taggacgtggtgtgataggtagcatggagaattctagattctcagggatgggttcaattcctatagtcctag

SEQ ID NO: 124 - DNA, 72 bp, synthetic construct
  misc_feature 1..72: note = Description of Artificial Sequence: Synthetic oligonucleotide
  source 1..72: mol_type = other DNA
taggacgtggtgtgataggtagcacggagaattctagattctcagggatgggttcaattcctgtagttctag

SEQ ID NO: 125 - DNA, 72 bp, synthetic construct
  misc_feature 1..72: note = Description of Artificial Sequence: Synthetic oligonucleotide
  source 1..72: mol_type = other DNA
ggttccatggtgtaatggttagcactctggactctaaatccagcgatccgagttcaaatctcggtggaacct

SEQ ID NO: 126 - DNA, 72 bp, synthetic construct
  misc_feature 1..72: note = Description of Artificial Sequence: Synthetic oligonucleotide
  source 1..72: mol_type = other DNA
ggttccatggtgtaatggtgaccactttggactctaaatacagtgatcagagttcaagtctcactggaacct

SEQ ID NO: 127 - DNA, 72 bp, synthetic construct
  misc_feature 1..72: note = Description of Artificial Sequence: Synthetic oligonucleotide
  source 1..72: mol_type = other DNA
ggttccatggtgtaatggtgagggctttggactctaactacagtgatcagagttcaagtctcagtgggacct

SEQ ID NO: 128 - DNA, 73 bp, synthetic construct
  misc_feature 1..73: note = Description of Artificial Sequence: Synthetic oligonucleotide
  source 1..73: mol_type = other DNA
ggttccatggtgtaatggtaagcaccctggactctaaatccagcaaccagagttccagtctcagcgtggacct

SEQ ID NO: 129 - DNA, 72 bp, synthetic construct
  misc_feature 1..72: note = Description of Artificial Sequence: Synthetic oligonucleotide
  source 1..72: mol_type = other DNA
ggtagtgtagtctactggttaaacgcttgggctctaacattaacgtcctgggttcaaatcccagctttgtca

SEQ ID NO: 130 - DNA, 72 bp, synthetic construct
  misc_feature 1..72: note = Description of Artificial Sequence: Synthetic oligonucleotide
  source 1..72: mol_type = other DNA
ggttccatggtgtaatggttagcactctggactctaaatccagcgatccgagttcaagtctcggtggaacct

SEQ ID NO: 131 - DNA, 72 bp, synthetic construct
  misc_feature 1..72: note = Description of Artificial Sequence: Synthetic oligonucleotide
  source 1..72: mol_type = other DNA
ggttccatggtgtaatggtgagcactctggactctaaatccagcgatccgagttcgagtctcggtggaacct

SEQ ID NO: 132 - DNA, 72 bp, synthetic construct
  misc_feature 1..72: note = Description of Artificial Sequence: Synthetic oligonucleotide
  source 1..72: mol_type = other DNA
ggttccatggtgtaatggtaagcactctggactctaaatccagcgatccgagttcgagtctcggtggaacct

SEQ ID NO: 133 - DNA, 72 bp, synthetic construct
  misc_feature 1..72: note = Description of Artificial Sequence: Synthetic oligonucleotide
  source 1..72: mol_type = other DNA
ggttccatggtgtaatggttagcactctggactctaaatccggtaatccgagttcaaatctcggtggaacct

SEQ ID NO: 134 - DNA, 72 bp, synthetic construct
  misc_feature 1..72: note = Description of Artificial Sequence: Synthetic oligonucleotide
  source 1..72: mol_type = other DNA
ggccccatggtgtaatggtcagcactctggactctaaatccagcgatccgagttcaaatctcggtgggaccc

SEQ ID NO: 135 - DNA, 72 bp, synthetic construct
  misc_feature 1..72: note = Description of Artificial Sequence: Synthetic oligonucleotide
  source 1..72: mol_type = other DNA
ggttccatggtgtaatggtaagcactctggactctaaatccagccatctgagttcgagtctctgtggaacct

SEQ ID NO: 136 - DNA, 72 bp, synthetic construct
  misc_feature 1..72: note = Description of Artificial Sequence: Synthetic oligonucleotide
  source 1..72: mol_type = other DNA
ggttccatggtgtaatggtgagcactttggactctaaatacagtgatcagagttcaagtctcactgggacct

SEQ ID NO: 137 - DNA, 72 bp, synthetic construct
  misc_feature 1..72: note = Description of Artificial Sequence: Synthetic oligonucleotide
  source 1..72: mol_type = other DNA
ggttccatgggttaatggtgagcaccctggactctaaatcaagcgatccgagttcaaatctcggtggtacct

SEQ ID NO: 138 - DNA, 68 bp, synthetic construct
  misc_feature 1..68: note = Description of Artificial Sequence: Synthetic oligonucleotide
  source 1..68: mol_type = other DNA
gtttccatggtgtaatggtgagcactctggactctaaatccagaaatacattcaaagaattaagaaca

SEQ ID NO: 139 - DNA, 72 bp, synthetic construct
  misc_feature 1..72: note = Description of Artificial Sequence: Synthetic oligonucleotide
  source 1..72: mol_type = other DNA
ggtcccatggtgtaatggttagcactctggactctaaatccagcgatccgagttcaaatctcggtgggacct

SEQ ID NO: 140 - DNA, 72 bp, synthetic construct
  misc_feature 1..72: note = Description of Artificial Sequence: Synthetic oligonucleotide
  source 1..72: mol_type = other DNA
ggtcccatggtgtaatggttagcactctggactctaaatccagcaatccgagttcgaatctcggtgggacct

SEQ ID NO: 141 - DNA, 72 bp, synthetic construct
  misc_feature 1..72: note = Description of Artificial Sequence: Synthetic oligonucleotide
  source 1..72: mol_type = other DNA
ggccccatggtgtaatggttagcactctggactctaaatccagcgatccgagttcaaatctcggtgggacct

SEQ ID NO: 142 - DNA, 72 bp, synthetic construct
  misc_feature 1..72: note = Description of Artificial Sequence: Synthetic oligonucleotide
  source 1..72: mol_type = other DNA
ggtcccatggtgtaatggttagcactctgggctctaaatccagcaatccgagttcgaatcttggtgggacct

SEQ ID NO: 143 - DNA, 73 bp, synthetic construct
  misc_feature 1..73: note = Description of Artificial Sequence: Synthetic oligonucleotide
  source 1..73: mol_type = other DNA
ggctgtgtacctcagtgggcaagggtatggactctaaagccagactatttgggttcaaatcccagcttggcct

SEQ ID NO: 144 - DNA, 73 bp, synthetic construct
  misc_feature 1..73: note = Description of Artificial Sequence: Synthetic oligonucleotide
  source 1..73: mol_type = other DNA
gaccatgtggcctaagggaaaagacatctcactctaggtcagaagattgagggttcaagtcctttcatggtca

SEQ ID NO: 145 - DNA, 74 bp, synthetic construct
  misc_feature 1..74: note = Description of Artificial Sequence: Synthetic oligonucleotide
  source 1..74: mol_type = other DNA
ggtacagtgttaaaggggagaaaaattgctgactctaaatacagtagacctaggtttgaatcctggctttacca

SEQ ID NO: 146 - DNA, 57 bp, synthetic construct
  misc_feature 1..57: note = Description of Artificial Sequence: Synthetic oligonucleotide
  source 1..57: mol_type = other DNA
tggtgtaataggtagcacagagaattttagattctcaggggtaggttcaattcctat

SEQ ID NO: 147 - DNA, 72 bp, synthetic construct
  misc_feature 1..72: note = Description of Artificial Sequence: Synthetic oligonucleotide
  source 1..72: mol_type = other DNA
taggacatggtgtgataggtagcatggagaattttagattctcaggggtaggttcaattcctacagttctag

SEQ ID NO: 148 - DNA, 72 bp, synthetic construct
  misc_feature 1..72: note = Description of Artificial Sequence: Synthetic oligonucleotide
  source 1..72: mol_type = other DNA
taggacgtggtgtgataggtagcatggggaattttagattctcaggggtgggttcaattcctatagttctag

SEQ ID NO: 149 - DNA, 72 bp, synthetic construct
  misc_feature 1..72: note = Description of Artificial Sequence: Synthetic oligonucleotide
  source 1..72: mol_type = other DNA
taggacgtggtgtagtaggtagcatggagaatgttaaattctcaggggtaggttcaattcctatagttctag

SEQ ID NO: 150 - DNA, 72 bp, synthetic construct
  misc_feature 1..72: note = Description of Artificial Sequence: Synthetic oligonucleotide
  source 1..72: mol_type = other DNA
taggacatggtgtaataggtagaatggagaattttaaattctcaggggtaggttcaattcctatagttctag

SEQ ID NO: 151 - DNA, 72 bp, synthetic construct
  misc_feature 1..72: note = Description of Artificial Sequence: Synthetic oligonucleotide
  source 1..72: mol_type = other DNA
taggatgtggtgtattaggtagcacagagaattttagattctcaggggtaggttcgattcctataattctac

SEQ ID NO: 152 - DNA, 72 bp, synthetic construct
  misc_feature 1..72: note = Description of Artificial Sequence: Synthetic oligonucleotide
  source 1..72: mol_type = other DNA
taggacttggtgtaatgggtagcacagagaattttagattctcaggggtgggttcaattcctttcgtcctag

SEQ ID NO: 153 - DNA, 75 bp, synthetic construct
  misc_feature 1..75: note = Description of Artificial Sequence: Synthetic oligonucleotide
  source 1..75: mol_type = other DNA
tctaggatgtggtgtgataggtagcatggagaattttagattctcaggggtaggttcaattcctatattctagaa

SEQ ID NO: 154 - DNA, 72 bp, synthetic construct
  misc_feature 1..72: note = Description of Artificial Sequence: Synthetic oligonucleotide
  source 1..72: mol_type = other DNA
taggacgtggtgtgataggtagcatggagaattttagattctcagggatgggttcaattcctatagtcctag

SEQ ID NO: 155 - DNA, 72 bp, synthetic construct
  misc_feature 1..72: note = Description of Artificial Sequence: Synthetic oligonucleotide
  source 1..72: mol_type = other DNA
taggacgtggtgtgataggtagcacggagaattttagattctcagggatgggttcaattcctgtagttctag

SEQ ID NO: 156 - DNA, 72 bp, synthetic construct
  misc_feature 1..72: note = Description of Artificial Sequence: Synthetic oligonucleotide
  source 1..72: mol_type = other DNA
ggttccatggtgtaatggttagcactctggactttaaatccagcgatccgagttcaaatctcggtggaacct

SEQ ID NO: 157 - DNA, 72 bp, synthetic construct
  misc_feature 1..72: note = Description of Artificial Sequence: Synthetic oligonucleotide
  source 1..72: mol_type = other DNA
ggttccatggtgtaatggtgaccactttggactttaaatacagtgatcagagttcaagtctcactggaacct

SEQ ID NO: 158 - DNA, 72 bp, synthetic construct
  misc_feature 1..72: note = Description of Artificial Sequence: Synthetic oligonucleotide
  source 1..72: mol_type = other DNA
ggttccatggtgtaatggtgagggctttggactttaactacagtgatcagagttcaagtctcagtgggacct

SEQ ID NO: 159 - DNA, 73 bp, synthetic construct
  misc_feature 1..73: note = Description of Artificial Sequence: Synthetic oligonucleotide
  source 1..73: mol_type = other DNA
ggttccatggtgtaatggtaagcaccctggactttaaatccagcaaccagagttccagtctcagcgtggacct

SEQ ID NO: 160 - DNA, 72 bp, synthetic construct
  misc_feature 1..72: note = Description of Artificial Sequence: Synthetic oligonucleotide
  source 1..72: mol_type = other DNA
ggtagtgtagtctactggttaaacgcttgggctttaacattaacgtcctgggttcaaatcccagctttgtca

SEQ ID NO: 161 - DNA, 72 bp, synthetic construct
  misc_feature 1..72: note = Description of Artificial Sequence: Synthetic oligonucleotide
  source 1..72: mol_type = other DNA
ggttccatggtgtaatggttagcactctggactttaaatccagcgatccgagttcaagtctcggtggaacct

SEQ ID NO: 162 - DNA, 72 bp, synthetic construct
  misc_feature 1..72: note = Description of Artificial Sequence: Synthetic oligonucleotide
  source 1..72: mol_type = other DNA
ggttccatggtgtaatggtgagcactctggactttaaatccagcgatccgagttcgagtctcggtggaacct

SEQ ID NO: 163 - DNA, 72 bp, synthetic construct
  misc_feature 1..72: note = Description of Artificial Sequence: Synthetic oligonucleotide
  source 1..72: mol_type = other DNA
ggttccatggtgtaatggtaagcactctggactttaaatccagcgatccgagttcgagtctcggtggaacct

SEQ ID NO: 164 - DNA, 72 bp, synthetic construct
  misc_feature 1..72: note = Description of Artificial Sequence: Synthetic oligonucleotide
  source 1..72: mol_type = other DNA
ggttccatggtgtaatggttagcactctggactttaaatccggtaatccgagttcaaatctcggtggaacct

SEQ ID NO: 165 - DNA, 72 bp, synthetic construct
  misc_feature 1..72: note = Description of Artificial Sequence: Synthetic oligonucleotide
  source 1..72: mol_type = other DNA
ggccccatggtgtaatggtcagcactctggactttaaatccagcgatccgagttcaaatctcggtgggaccc

SEQ ID NO: 166 - DNA, 72 bp, synthetic construct
  misc_feature 1..72: note = Description of Artificial Sequence: Synthetic oligonucleotide
  source 1..72: mol_type = other DNA
ggttccatggtgtaatggtaagcactctggactttaaatccagccatctgagttcgagtctctgtggaacct

SEQ ID NO: 167 - DNA, 72 bp, synthetic construct
  misc_feature 1..72: note = Description of Artificial Sequence: Synthetic oligonucleotide
  source 1..72: mol_type = other DNA
ggttccatggtgtaatggtgagcactttggactttaaatacagtgatcagagttcaagtctcactgggacct

SEQ ID NO: 168 - DNA, 72 bp, synthetic construct
  misc_feature 1..72: note = Description of Artificial Sequence: Synthetic oligonucleotide
  source 1..72: mol_type = other DNA
ggttccatgggttaatggtgagcaccctggactttaaatcaagcgatccgagttcaaatctcggtggtacct

SEQ ID NO: 169 - DNA, 68 bp, synthetic construct
  misc_feature 1..68: note = Description of Artificial Sequence: Synthetic oligonucleotide
  source 1..68: mol_type = other DNA
gtttccatggtgtaatggtgagcactctggactttaaatccagaaatacattcaaagaattaagaaca

SEQ ID NO: 170 - DNA, 72 bp, synthetic construct
  misc_feature 1..72: note = Description of Artificial Sequence: Synthetic oligonucleotide
  source 1..72: mol_type = other DNA
ggtcccatggtgtaatggttagcactctggactttaaatccagcgatccgagttcaaatctcggtgggacct

SEQ ID NO: 171 - DNA, 72 bp, synthetic construct
  misc_feature 1..72: note = Description of Artificial Sequence: Synthetic oligonucleotide
  source 1..72: mol_type = other DNA
ggtcccatggtgtaatggttagcactctggactttaaatccagcaatccgagttcgaatctcggtgggacct

SEQ ID NO: 172 - DNA, 72 bp, synthetic construct
  misc_feature 1..72: note = Description of Artificial Sequence: Synthetic oligonucleotide
  source 1..72: mol_type = other DNA
ggccccatggtgtaatggttagcactctggactttaaatccagcgatccgagttcaaatctcggtgggacct

SEQ ID NO: 173 - DNA, 72 bp, synthetic construct
  misc_feature 1..72: note = Description of Artificial Sequence: Synthetic oligonucleotide
  source 1..72: mol_type = other DNA
ggtcccatggtgtaatggttagcactctgggctttaaatccagcaatccgagttcgaatcttggtgggacct

SEQ ID NO: 174 - DNA, 73 bp, synthetic construct
  misc_feature 1..73: note = Description of Artificial Sequence: Synthetic oligonucleotide
  source 1..73: mol_type = other DNA
ggctgtgtacctcagtgggcaagggtatggactttaaagccagactatttgggttcaaatcccagcttggcct

SEQ ID NO: 175 - DNA, 73 bp, synthetic construct
  misc_feature 1..73: note = Description of Artificial Sequence: Synthetic oligonucleotide
  source 1..73: mol_type = other DNA
gaccatgtggcctaagggaaaagacatctcactttaggtcagaagattgagggttcaagtcctttcatggtca

SEQ ID NO: 176 - DNA, 74 bp, synthetic construct
  misc_feature 1..74: note = Description of Artificial Sequence: Synthetic oligonucleotide
  source 1..74: mol_type = other DNA
ggtacagtgttaaaggggagaaaaattgctgactttaaatacagtagacctaggtttgaatcctggctttacca

SEQ ID NO: 177 - DNA, 72 bp, synthetic construct
  misc_feature 1..72: note = Description of Artificial Sequence: Synthetic oligonucleotide
  source 1..72: mol_type = other DNA
tccctggtggtctagtggttaggattcggcgctttaaccgccgcggcccgggttcgattcccggtcagggaa

SEQ ID NO: 178 - DNA, 72 bp, synthetic construct
  misc_feature 1..72: note = Description of Artificial Sequence: Synthetic oligonucleotide
  source 1..72: mol_type = other DNA
tccctggtggtctagtggttaggattcggcgctttaaccgccgcggcccgggttcgattcccggtcaggaaa

SEQ ID NO: 179 - DNA, 72 bp, synthetic construct
  misc_feature 1..72: note = Description of Artificial Sequence: Synthetic oligonucleotide
  source 1..72: mol_type = other DNA
cccctggtggtctagtgcttaggattcggtgctttaaccgctgctgcctgcgttcgattcccggtcagggaa

SEQ ID NO: 180 - DNA, 71 bp, synthetic construct
  misc_feature 1..71: note = Description of Artificial Sequence: Synthetic oligonucleotide
  source 1..71: mol_type = other DNA
tccttgatgtctagtggttaggatttggtgctttaactgcagcagcctgggttcatttctcagtcagggaa

SEQ ID NO: 181 - DNA, 72 bp, synthetic construct
  misc_feature 1..72: note = Description of Artificial Sequence: Synthetic oligonucleotide
  source 1..72: mol_type = other DNA
tcccatatggtctagcggttaggattcctggttttaacccaggtggcccgggttcgactcccggtatgggaa

SEQ ID NO: 182 - DNA, 73 bp, synthetic construct
  misc_feature 1..73: note = Description of Artificial Sequence: Synthetic oligonucleotide
  source 1..73: mol_type = other DNA
tccgtggtggtctagtggctaggattcggcgctttaaccgcctgcagctcgagttcgattcctggtcagggaa

SEQ ID NO: 183 - DNA, 69 bp, synthetic construct
  misc_feature 1..69: note = Description of Artificial Sequence: Synthetic oligonucleotide
  source 1..69: mol_type = other DNA
ccctgtggtctagtggctaagactttgtgctttaattgctgcatcctaggttcaattcccagtcaggga

SEQ ID NO: 184 - DNA, 72 bp, synthetic construct
  misc_feature 1..72: note = Description of Artificial Sequence: Synthetic oligonucleotide
  source 1..72: mol_type = other DNA
tcccacatggtctagcggttaggattcctggttttaacccaggcggcccgggttcgactcccggtgtgggaa

SEQ ID NO: 185 - DNA, 72 bp, synthetic construct
  misc_feature 1..72: note = Description of Artificial Sequence: Synthetic oligonucleotide
  source 1..72: mol_type = other DNA
tccctggtggtctagtggctaggattcggcgctttaaccgccgcggcccgggttcgattcccggccagggaa

SEQ ID NO: 186 - DNA, 72 bp, synthetic construct
  misc_feature 1..72: note = Description of Artificial Sequence: Synthetic oligonucleotide
  source 1..72: mol_type = other DNA
tccctggtggtctagtggctaggattcggcgctttaaccgccgcggcccgggttcgattcccggtcagggaa

SEQ ID NO: 187 - DNA, 72 bp, synthetic construct
  misc_feature 1..72: note = Description of Artificial Sequence: Synthetic oligonucleotide
  source 1..72: mol_type = other DNA
gcgttggtggtgtagtggtgagcacagctgcctttaaagcagttaacgcgggttcgattcccgggtaacgaa

SEQ ID NO: 188 - DNA, 72 bp, synthetic construct
  misc_feature 1..72: note = Description of Artificial Sequence: Synthetic oligonucleotide
  source 1..72: mol_type = other DNA
tccttggtggtctagtggctaggattcggtgctttaacctgtgcggcccgggttcaattcccgatgaaggaa

SEQ ID NO: 189 - DNA, 72 bp, synthetic construct
  misc_feature 1..72: note = Description of Artificial Sequence: Synthetic oligonucleotide
  source 1..72: mol_type = other DNA
tgtctggtggtcaagtggctaggatttggcgctttaactgccgcggcccgcgttcgattcccggtcagggaa

SEQ ID NO: 190 - DNA, 73 bp, synthetic construct
  misc_feature 1..73: note = Description of Artificial Sequence: Synthetic oligonucleotide
  source 1..73: mol_type = other DNA
tccctggtggtctagtggctaggattcggcgctttaaccgcctgcagctcgagttcgattcctggtcagggaa

SEQ ID NO: 191 - DNA, 71 bp, synthetic construct
  misc_feature 1..71: note = Description of Artificial Sequence: Synthetic oligonucleotide
  source 1..71: mol_type = other DNA
gcaatggtggttcagtggtagaattctcgcctttaacacaggagacccgggttcaattcctgacccatgta

SEQ ID NO: 192 - DNA, 72 bp, synthetic construct
  misc_feature 1..72: note = Description of Artificial Sequence: Synthetic oligonucleotide
  source 1..72: mol_type = other DNA
tccctggtggtctagtggttaggattcggcgctctaaccgccgcggcccgggttcgattcccggtcagggaa

SEQ ID NO: 193 - DNA, 72 bp, synthetic construct
  misc_feature 1..72: note = Description of Artificial Sequence: Synthetic oligonucleotide
  source 1..72: mol_type = other DNA
tccctggtggtctagtggttaggattcggcgctctaaccgccgcggcccgggttcgattcccggtcaggaaa

SEQ ID NO: 194 - DNA, 72 bp, synthetic construct
  misc_feature 1..72: note = Description of Artificial Sequence: Synthetic oligonucleotide
  source 1..72: mol_type = other DNA
cccctggtggtctagtgcttaggattcggtgctctaaccgctgctgcctgcgttcgattcccggtcagggaa

SEQ ID NO: 195 - DNA, 71 bp, synthetic construct
  misc_feature 1..71: note = Description of Artificial Sequence: Synthetic oligonucleotide
  source 1..71: mol_type = other DNA
tccttgatgtctagtggttaggatttggtgctctaactgcagcagcctgggttcatttctcagtcagggaa

SEQ ID NO: 196 - DNA, 72 bp, synthetic construct
  misc_feature 1..72: note = Description of Artificial Sequence: Synthetic oligonucleotide
  source 1..72: mol_type = other DNA
tcccatatggtctagcggttaggattcctggttctaacccaggtggcccgggttcgactcccggtatgggaa

SEQ ID NO: 197 - DNA, 73 bp, synthetic construct
  misc_feature 1..73: note = Description of Artificial Sequence: Synthetic oligonucleotide
  source 1..73: mol_type = other DNA
tccgtggtggtctagtggctaggattcggcgctctaaccgcctgcagctcgagttcgattcctggtcagggaa

SEQ ID NO: 198 - DNA, 69 bp, synthetic construct
  misc_feature 1..69: note = Description of Artificial Sequence: Synthetic oligonucleotide
  source 1..69: mol_type = other DNA
ccctgtggtctagtggctaagactttgtgctctaattgctgcatcctaggttcaattcccagtcaggga

SEQ ID NO: 199 - DNA, 72 bp, synthetic construct
  misc_feature 1..72: note = Description of Artificial Sequence: Synthetic oligonucleotide
  source 1..72: mol_type = other DNA
tcccacatggtctagcggttaggattcctggttctaacccaggcggcccgggttcgactcccggtgtgggaa

SEQ ID NO: 200 - DNA, 72 bp, synthetic construct
  misc_feature 1..72: note = Description of Artificial Sequence: Synthetic oligonucleotide
  source 1..72: mol_type = other DNA
tccctggtggtctagtggctaggattcggcgctctaaccgccgcggcccgggttcgattcccggccagggaa

SEQ ID NO: 201 - DNA, 72 bp, synthetic construct
  misc_feature 1..72: note = Description of Artificial Sequence: Synthetic oligonucleotide
  source 1..72: mol_type = other DNA
tccctggtggtctagtggctaggattcggcgctctaaccgccgcggcccgggttcgattcccggtcagggaa

SEQ ID NO: 202 - DNA, 72 bp, synthetic construct
  misc_feature 1..72: note = Description of Artificial Sequence: Synthetic oligonucleotide
  source 1..72: mol_type = other DNA
gcgttggtggtgtagtggtgagcacagctgcctctaaagcagttaacgcgggttcgattcccgggtaacgaa

SEQ ID NO: 203 - DNA, 72 bp, synthetic construct
  misc_feature 1..72: note = Description of Artificial Sequence: Synthetic oligonucleotide
  source 1..72: mol_type = other DNA
tccttggtggtctagtggctaggattcggtgctctaacctgtgcggcccgggttcaattcccgatgaaggaa

SEQ ID NO: 204 - DNA, 72 bp, synthetic construct
  misc_feature 1..72: note = Description of Artificial Sequence: Synthetic oligonucleotide
  source 1..72: mol_type = other DNA
tgtctggtggtcaagtggctaggatttggcgctctaactgccgcggcccgcgttcgattcccggtcagggaa

SEQ ID NO: 205 - DNA, 73 bp, synthetic construct
  misc_feature 1..73: note = Description of Artificial Sequence: Synthetic oligonucleotide
  source 1..73: mol_type = other DNA
tccctggtggtctagtggctaggattcggcgctctaaccgcctgcagctcgagttcgattcctggtcagggaa

SEQ ID NO: 206 - DNA, 74 bp, synthetic construct
  misc_feature 1..74: note = Description of Artificial Sequence: Synthetic oligonucleotide
  source 1..74: mol_type = other DNA
gcaatggtggttcagtggtagaattctcgcctctactaacacaggagacccgggttcaattcctgacccatgta

SEQ ID NO: 207 - DNA, 93 bp, synthetic construct
  misc_feature 1..93: note = Description of Artificial Sequence: Synthetic oligonucleotide
  source 1..93: mol_type = other DNA
ccttcaatagttcagctggtagagcagaggactttagctacttcctcagtaggagacgtccttaggttgctggttcgattccagcttgaagga

SEQ ID NO: 208 - DNA, 73 bp, synthetic construct
  misc_feature 1..73: note = Description of Artificial Sequence: Synthetic oligonucleotide
  source 1..73: mol_type = other DNA
ccttcaatagttcagctggtagagcagaggactttaggtccttaggttgctggttcgattccagcttgaagga

SEQ ID NO: 209 - DNA, 65 bp, synthetic construct
  misc_feature 1..65: note = Description of Artificial Sequence: Synthetic oligonucleotide
  source 1..65: mol_type = other DNA
ggtaaaatggctgagtaagctttagactttaaaatctaaagagagattgagctctctttttacca

SEQ ID NO: 210 - DNA, 66 bp, synthetic construct
  misc_feature 1..66: note = Description of Artificial Sequence: Synthetic oligonucleotide
  source 1..66: mol_type = other DNA
ggtaaaatgactgagtaagcattagactttaaatctaaagacagaggtcaagacctctttttacca

SEQ ID NO: 211 - DNA, 66 bp, synthetic construct
  misc_feature 1..66: note = Description of Artificial Sequence: Synthetic oligonucleotide
  source 1..66: mol_type = other DNA
ggtaaaatggctgagtaagcattagactttaaatctaaagacagaggtcaaggcctctttttacca

SEQ ID NO: 212 - DNA, 64 bp, synthetic construct
  misc_feature 1..64: note = Description of Artificial Sequence: Synthetic oligonucleotide
  source 1..64: mol_type = other DNA
ggtaaaatggctgagtaagcattagactttaaatctaaagacagaggtcaaggcctttttacca

SEQ ID NO: 213 - DNA, 91 bp, synthetic construct
  misc_feature 1..91: note = Description of Artificial Sequence: Synthetic oligonucleotide
  source 1..91: mol_type = other DNA
ccttcgatagctcagttggtagagcggaggactttagttggctgtgtccttagacatccttaggtcgctggttcgaatccggctcgaagga

SEQ ID NO: 214 - DNA, 73 bp, synthetic construct
  misc_feature 1..73: note = Description of Artificial Sequence: Synthetic oligonucleotide
  source 1..73: mol_type = other DNA
ccttcgatagctcagttggtagagcggaggactttagatccttaggtcgctggttcgaatccggctcgaagga

SEQ ID NO: 215 - DNA, 73 bp, synthetic construct
  misc_feature 1..73: note = Description of Artificial Sequence: Synthetic oligonucleotide
  source 1..73: mol_type = other DNA
gggggtatagctcagggctagagctttttgactttagagcaagaggtccctggttcaaatccaggttctccct

SEQ ID NO: 216 - DNA, 61 bp, synthetic construct
  misc_feature 1..61: note = Description of Artificial Sequence: Synthetic oligonucleotide
  source 1..61: mol_type = other DNA
tatagctcagtggtagagcatttaactttagatcaagaggtccctggatcaactctgggtg

SEQ ID NO: 217 - DNA, 73 bp, synthetic construct
  misc_feature 1..73: note = Description of Artificial Sequence: Synthetic oligonucleotide
  source 1..73: mol_type = other DNA
gtcagtgttgcacaacggttaagtgaagaggctttaaacccagactggatgggttcaattcccatctctgccg

SEQ ID NO: 218 - DNA, 89 bp, synthetic construct
  misc_feature 1..89: note = Description of Artificial Sequence: Synthetic oligonucleotide
  source 1..89: mol_type = other DNA
ccttcgatagctcagttggtagagcggaggactttagtggatagggcgtggcaatccttaggtcgctggttcgattccggctcgaagga

SEQ ID NO: 219 - DNA, 73 bp, synthetic construct
  misc_feature 1..73: note = Description of Artificial Sequence: Synthetic oligonucleotide
  source 1..73: mol_type = other DNA
ccttcgatagctcagttggtagagcggaggactttagatccttaggtcgctggttcgattccggctcgaagga

SEQ ID NO: 220 - DNA, 89 bp, synthetic construct
  misc_feature 1..89: note = Description of Artificial Sequence: Synthetic oligonucleotide
  source 1..89: mol_type = other DNA
ccttcgatagctcagttggtagagcggaggactttaggctcattaagcaaggtatccttaggtcgctggttcgaatccggctcggagga

SEQ ID NO: 221 - DNA, 73 bp, synthetic construct
  misc_feature 1..73: note = Description of Artificial Sequence: Synthetic oligonucleotide
  source 1..73: mol_type = other DNA
ccttcgatagctcagttggtagagcggaggactttagatccttaggtcgctggttcgaatccggctcggagga

SEQ ID NO: 222 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactttagattgtatagacatttgcggacatccttaggtcgctggttcgattccagctcgaagga

SEQ ID NO: 223 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactttagatccttaggtcgctggttcgattccagctcgaagga

SEQ ID NO: 224 - DNA, 93 bp, synthetic construct
  misc_feature 1..93: note = Description of Artificial Sequence: Synthetic oligonucleotide
  source 1..93: mol_type = other DNA
ccttcgatagctcagctggtagagcggaggactttagctacttcctcagcaggagacatccttaggtcgctggttcgattccggctcgaagga

SEQ ID NO: 225 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactttagatccttaggtcgctggttcgattccggctcgaagga

SEQ ID NO: 226 - DNA, 89 bp, synthetic construct
  misc_feature 1..89: note = Description of Artificial Sequence: Synthetic oligonucleotide
  source 1..89: mol_type = other DNA
ccttcgatagctcagctggtagagcggaggactttaggcgcgcgcccgtggccatccttaggtcgctggttcgattccggctcgaagga

SEQ ID NO: 227 - DNA, 73 bp, synthetic construct; identical to SEQ ID NO: 225
  misc_feature 1..73: note = Description of Artificial Sequence: Synthetic oligonucleotide
  source 1..73: mol_type = other DNA

SEQ ID NO: 228 - DNA, 95 bp, synthetic construct
  misc_feature 1..95: note = Description of Artificial Sequence: Synthetic oligonucleotide
  source 1..95: mol_type = other DNA
ccttcgatagctcagctggtagagcggaggactttaagcctgtagaaacatttgtggacatccttaggtcgctggttcgattccggctcgaagga

SEQ ID NO: 229 - DNA, 73 bp, synthetic construct; identical to SEQ ID NO: 225
  misc_feature 1..73: note = Description of Artificial Sequence: Synthetic oligonucleotide
  source 1..73: mol_type = other DNA

SEQ ID NO: 230 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactttagattgtacagacatttgcggacatccttaggtcgctggttcgattccggctcgaagga

SEQ ID NO: 231 - DNA, 73 bp, synthetic construct; identical to SEQ ID NO: 225
  misc_feature 1..73: note = Description of Artificial Sequence: Synthetic oligonucleotide
  source 1..73: mol_type = other DNA

SEQ ID NO: 232 - DNA, 89 bp, synthetic construct
  misc_feature 1..89: note = Description of Artificial Sequence: Synthetic oligonucleotide
  source 1..89: mol_type = other DNA
ccttcgatagctcagctggtagagcggaggactttagtacttaatgtgtggtcatccttaggtcgctggttcgattccggctcgaagga

SEQ ID NO: 233 - DNA, 73 bp, synthetic construct; identical to SEQ ID NO: 225
  misc_feature 1..73: note = Description of Artificial Sequence: Synthetic oligonucleotide
  source 1..73: mol_type = other DNA

SEQ ID NO: 234 - DNA, 89 bp, synthetic construct
  misc_feature 1..89: note = Description of Artificial Sequence: Synthetic oligonucleotide
  source 1..89: mol_type = other DNA
ccttcgatagctcagctggtagagcggaggactttaggggtttgaatgtggtcatccttaggtcgctggttcgaatccggctcggagga

SEQ ID NO: 235 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactttagatccttaggtcgctggttcgaatccggctcggagga

SEQ ID NO: 236 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactttagactgcggaaacgtttgtggacatccttaggtcgctggttcaattccggctcgaagga

SEQ ID NO: 237 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactttagatccttaggtcgctggttcaattccggctcgaagga

SEQ ID NO: 238 - DNA, 90 bp, synthetic construct
  misc_feature 1..90: note = Description of Artificial Sequence: Synthetic oligonucleotide
  source 1..90: mol_type = other DNA
ctttcgatagctcagttggtagagcggaggactttaggttcattaaactaaggcatccttaggtcgctggttcgaatccggctcgaagga

SEQ ID NO: 239 - DNA, 73 bp, synthetic construct
  misc_feature 1..73: note = Description of Artificial Sequence: Synthetic oligonucleotide
  source 1..73: mol_type = other DNA
ctttcgatagctcagttggtagagcggaggactttagatccttaggtcgctggttcgaatccggctcgaagga

SEQ ID NO: 240 - DNA, 89 bp, synthetic construct
  misc_feature 1..89: note = Description of Artificial Sequence: Synthetic oligonucleotide
  source 1..89: mol_type = other DNA
tcttcaatagctcagctggtagagcggaggactttaaggtgcacgcccgtggccattcttaggtgctggtttgattccgacttggagag

SEQ ID NO: 241 - DNA, 72 bp, synthetic construct
  misc_feature 1..72: note = Description of Artificial Sequence: Synthetic oligonucleotide
  source 1..72: mol_type = other DNA
tcttcaatagctcagctggtagagcggaggactttagattcttaggtgctggtttgattccgacttggagag

SEQ ID NO: 242 - DNA, 66 bp, synthetic construct
  misc_feature 1..66: note = Description of Artificial Sequence: Synthetic oligonucleotide
  source 1..66: mol_type = other DNA
ggtaaaatggctgagtgaagcattggactttaaatctaaagacaggggttaagcctctttttacca

SEQ ID NO: 243 - DNA, 66 bp, synthetic construct
  misc_feature 1..66: note = Description of Artificial Sequence: Synthetic oligonucleotide
  source 1..66: mol_type = other DNA
ggtaaaatggctgagcaagcattggactttaaatctaaagacagatgttgagccatctttttagca

SEQ ID NO: 244 - DNA, 66 bp, synthetic construct
  misc_feature 1..66: note = Description of Artificial Sequence: Synthetic oligonucleotide
  source 1..66: mol_type = other DNA
ggtaaaatggctgagtgaagcattggactttaaatctaaagacaggggctaagcctctttttacca

SEQ ID NO: 245 - DNA, 66 bp, synthetic construct
  misc_feature 1..66: note = Description of Artificial Sequence: Synthetic oligonucleotide
  source 1..66: mol_type = other DNA
ggtaaaatggctgagcaagcattagactttaaatctaaagacagaggttaaggcctctttttacca

SEQ ID NO: 246 - DNA, 66 bp, synthetic construct
  misc_feature 1..66: note = Description of Artificial Sequence: Synthetic oligonucleotide
  source 1..66: mol_type = other DNA
ggtaaaatggctgagtaagcattagactttaaatctaaagacagaggtcaaggcctcttttttcct

SEQ ID NO: 247 - DNA, 67 bp, synthetic construct
  misc_feature 1..67: note = Description of Artificial Sequence: Synthetic oligonucleotide
  source 1..67: mol_type = other DNA
ggtaaaatggctgagcaagcattagactttaaatctgaaaacagaggtcaaaggtctctttttacca

SEQ ID NO: 248 - DNA, 66 bp, synthetic construct; identical to SEQ ID NO: 211
  misc_feature 1..66: note = Description of Artificial Sequence: Synthetic oligonucleotide
  source 1..66: mol_type = other DNA

SEQ ID NO: 249 - DNA, 66 bp, synthetic construct
  misc_feature 1..66: note = Description of Artificial Sequence: Synthetic oligonucleotide
  source 1..66: mol_type = other DNA
ggtaaaatgactgaataagccttagactttaaatctgaagacagaggtcaaggcctctttttacca

SEQ ID NO: 250 - DNA, 66 bp, synthetic construct
  misc_feature 1..66: note = Description of Artificial Sequence: Synthetic oligonucleotide
  source 1..66: mol_type = other DNA
ggtaaaatggctgagtaagcattggactttaaatctaaagacagaggtcaagacctctttttacca

SEQ ID NO: 251 - DNA, 66 bp, synthetic construct
  misc_feature 1..66: note = Description of Artificial Sequence: Synthetic oligonucleotide
  source 1..66: mol_type = other DNA
ggtaaaatggctgagtaaagcattagactttaaatctaaggacagaggctaaacctctttttacca

SEQ ID NO: 252 - DNA, 93 bp, synthetic construct
  misc_feature 1..93: note = Description of Artificial Sequence: Synthetic oligonucleotide
  source 1..93: mol_type = other DNA
ccttcaatagttcagctggtagagcagaggactctagctacttcctcagtaggagacgtccttaggttgctggttcgattccagcttgaagga

SEQ ID NO: 253 - DNA, 73 bp, synthetic construct
  misc_feature 1..73: note = Description of Artificial Sequence: Synthetic oligonucleotide
  source 1..73: mol_type = other DNA
ccttcaatagttcagctggtagagcagaggactctaggtccttaggttgctggttcgattccagcttgaagga

SEQ ID NO: 254 - DNA, 65 bp, synthetic construct
  misc_feature 1..65: note = Description of Artificial Sequence: Synthetic oligonucleotide
  source 1..65: mol_type = other DNA
ggtaaaatggctgagtaagctttagactctaaaatctaaagagagattgagctctctttttacca

SEQ ID NO: 255 - DNA, 66 bp, synthetic construct
  misc_feature 1..66: note = Description of Artificial Sequence: Synthetic oligonucleotide
  source 1..66: mol_type = other DNA
ggtaaaatgactgagtaagcattagactctaaatctaaagacagaggtcaagacctctttttacca

SEQ ID NO: 256 - DNA, 66 bp, synthetic construct
  misc_feature 1..66: note = Description of Artificial Sequence: Synthetic oligonucleotide
  source 1..66: mol_type = other DNA
ggtaaaatggctgagtaagcattagactctaaatctaaagacagaggtcaaggcctctttttacca

SEQ ID NO: 257 - DNA, 64 bp, synthetic construct
  misc_feature 1..64: note = Description of Artificial Sequence: Synthetic oligonucleotide
  source 1..64: mol_type = other DNA
ggtaaaatggctgagtaagcattagactctaaatctaaagacagaggtcaaggcctttttacca

SEQ ID NO: 258 - DNA, 91 bp, synthetic construct
  misc_feature 1..91: note = Description of Artificial Sequence: Synthetic oligonucleotide
  source 1..91: mol_type = other DNA
ccttcgatagctcagttggtagagcggaggactctagttggctgtgtccttagacatccttaggtcgctggttcgaatccggctcgaagga

SEQ ID NO: 259 - DNA, 73 bp, synthetic construct
  misc_feature 1..73: note = Description of Artificial Sequence: Synthetic oligonucleotide
  source 1..73: mol_type = other DNA
ccttcgatagctcagttggtagagcggaggactctagatccttaggtcgctggttcgaatccggctcgaagga

SEQ ID NO: 260 - DNA, 74 bp, synthetic construct
  misc_feature 1..74: note = Description of Artificial Sequence: Synthetic oligonucleotide
  source 1..74: mol_type = other DNA
gggggtatagctcagggctagagctttttgactctaagagcaagaggtccctggttcaaatccaggttctccct

SEQ ID NO: 261 - DNA, 61 bp, synthetic construct
  misc_feature 1..61: note = Description of Artificial Sequence: Synthetic oligonucleotide
  source 1..61: mol_type = other DNA
tatagctcagtggtagagcatttaactctagatcaagaggtccctggatcaactctgggtg

SEQ ID NO: 262 - DNA, 73 bp, synthetic construct
  misc_feature 1..73: note = Description of Artificial Sequence: Synthetic oligonucleotide
  source 1..73: mol_type = other DNA
gtcagtgttgcacaacggttaagtgaagaggctctaaacccagactggatgggttcaattcccatctctgccg

SEQ ID NO: 263 - DNA, 89 bp, synthetic construct
  misc_feature 1..89: note = Description of Artificial Sequence: Synthetic oligonucleotide
  source 1..89: mol_type = other DNA
ccttcgatagctcagttggtagagcggaggactctagtggatagggcgtggcaatccttaggtcgctggttcgattccggctcgaagga

SEQ ID NO: 264 - DNA, 73 bp, synthetic construct
  misc_feature 1..73: note = Description of Artificial Sequence: Synthetic oligonucleotide
  source 1..73: mol_type = other DNA
ccttcgatagctcagttggtagagcggaggactctagatccttaggtcgctggttcgattccggctcgaagga

SEQ ID NO: 265 - DNA, 89 bp, synthetic construct
  misc_feature 1..89: note = Description of Artificial Sequence: Synthetic oligonucleotide
  source 1..89: mol_type = other DNA
ccttcgatagctcagttggtagagcggaggactctaggctcattaagcaaggtatccttaggtcgctggttcgaatccggctcggagga

SEQ ID NO: 266 - DNA, 73 bp, synthetic construct
  misc_feature 1..73: note = Description of Artificial Sequence: Synthetic oligonucleotide
  source 1..73: mol_type = other DNA
ccttcgatagctcagttggtagagcggaggactctagatccttaggtcgctggttcgaatccggctcggagga

SEQ ID NO: 267 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactctagattgtatagacatttgcggacatccttaggtcgctggttcgattccagctcgaagga

SEQ ID NO: 268 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactctagatccttaggtcgctggttcgattccagctcgaagga

SEQ ID NO: 269 - DNA, 93 bp, synthetic construct
  misc_feature 1..93: note = Description of Artificial Sequence: Synthetic oligonucleotide
  source 1..93: mol_type = other DNA
ccttcgatagctcagctggtagagcggaggactctagctacttcctcagcaggagacatccttaggtcgctggttcgattccggctcgaagga

SEQ ID NO: 270 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactctagatccttaggtcgctggttcgattccggctcgaagga

SEQ ID NO: 271 - DNA, 89 bp, synthetic construct
  misc_feature 1..89: note = Description of Artificial Sequence: Synthetic oligonucleotide
  source 1..89: mol_type = other DNA
ccttcgatagctcagctggtagagcggaggactctaggcgcgcgcccgtggccatccttaggtcgctggttcgattccggctcgaagga

SEQ ID NO: 272 - DNA, 73 bp, synthetic construct; identical to SEQ ID NO: 270
  misc_feature 1..73: note = Description of Artificial Sequence: Synthetic oligonucleotide
  source 1..73: mol_type = other DNA

SEQ ID NO: 273 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactctagcctgtagaaacatttgtggacatccttaggtcgctggttcgattccggctcgaagga

SEQ ID NO: 274 - DNA, 73 bp, synthetic construct; identical to SEQ ID NO: 270
  misc_feature 1..73: note = Description of Artificial Sequence: Synthetic oligonucleotide
  source 1..73: mol_type = other DNA

SEQ ID NO: 275 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactctagattgtacagacatttgcggacatccttaggtcgctggttcgattccggctcgaagga

SEQ ID NO: 276 - DNA, 73 bp, synthetic construct; identical to SEQ ID NO: 270
  misc_feature 1..73: note = Description of Artificial Sequence: Synthetic oligonucleotide
  source 1..73: mol_type = other DNA

SEQ ID NO: 277 - DNA, 89 bp, synthetic construct
  misc_feature 1..89: note = Description of Artificial Sequence: Synthetic oligonucleotide
  source 1..89: mol_type = other DNA
ccttcgatagctcagctggtagagcggaggactctagtacttaatgtgtggtcatccttaggtcgctggttcgattccggctcgaagga

SEQ ID NO: 278 - DNA, 73 bp, synthetic construct; identical to SEQ ID NO: 270
  misc_feature 1..73: note = Description of Artificial Sequence: Synthetic oligonucleotide
  source 1..73: mol_type = other DNA

SEQ ID NO: 279 - DNA, 89 bp, synthetic construct
  misc_feature 1..89: note = Description of Artificial Sequence: Synthetic oligonucleotide
  source 1..89: mol_type = other DNA
ccttcgatagctcagctggtagagcggaggactctaggggtttgaatgtggtcatccttaggtcgctggttcgaatccggctcggagga

SEQ ID NO: 280 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactctagatccttaggtcgctggttcgaatccggctcggagga

SEQ ID NO: 281 - DNA, 94 bp, synthetic construct
  misc_feature 1..94: note = Description of Artificial Sequence: Synthetic oligonucleotide
  source 1..94: mol_type = other DNA
ccttcgatagctcagctggtagagcggaggactctagactgcggaaacgtttgtggacatccttaggtcgctggttcaattccggctcgaagga

SEQ ID NO: 282 - DNA, 73 bp, synthetic construct
  misc_feature 1..73: note = Description of Artificial Sequence: Synthetic oligonucleotide
  source 1..73: mol_type = other DNA
ccttcgatagctcagctggtagagcggaggactctagatccttaggtcgctggttcaattccggctcgaagga

SEQ ID NO: 283 - DNA, 90 bp, synthetic construct
  misc_feature 1..90: note = Description of Artificial Sequence: Synthetic oligonucleotide
  source 1..90: mol_type = other DNA
ctttcgatagctcagttggtagagcggaggactctaggttcattaaactaaggcatccttaggtcgctggttcgaatccggctcgaagga

SEQ ID NO: 284 - DNA, 73 bp, synthetic construct
  misc_feature 1..73: note = Description of Artificial Sequence: Synthetic oligonucleotide
  source 1..73: mol_type = other DNA
ctttcgatagctcagttggtagagcggaggactctagatccttaggtcgctggttcgaatccggctcgaagga

SEQ ID NO: 285 - DNA, 88 bp, synthetic construct
  misc_feature 1..88: note = Description of Artificial Sequence: Synthetic oligonucleotide
  source 1..88: mol_type = other DNA
tcttcaatagctcagctggtagagcggaggactctaggtgcacgcccgtggccattcttaggtgctggtttgattccgacttggagag

SEQ ID NO: 286 - DNA, 72 bp, synthetic construct
  misc_feature 1..72: note = Description of Artificial Sequence: Synthetic oligonucleotide
  source 1..72: mol_type = other DNA
tcttcaatagctcagctggtagagcggaggactctagattcttaggtgctggtttgattccgacttggagag

SEQ ID NO: 287 - DNA, 66 bp, synthetic construct
  misc_feature 1..66: note = Description of Artificial Sequence: Synthetic oligonucleotide
  source 1..66: mol_type = other DNA
ggtaaaatggctgagtgaagcattggactctaaatctaaagacaggggttaagcctctttttacca

SEQ ID NO: 288 - DNA, 66 bp, synthetic construct
  misc_feature 1..66: note = Description of Artificial Sequence: Synthetic oligonucleotide
  source 1..66: mol_type = other DNA
ggtaaaatggctgagcaagcattggactctaaatctaaagacagatgttgagccatctttttagca

SEQ ID NO: 289 - DNA, 66 bp, synthetic construct
  misc_feature 1..66: note = Description of Artificial Sequence: Synthetic oligonucleotide
  source 1..66: mol_type = other DNA
ggtaaaatggctgagtgaagcattggactctaaatctaaagacaggggctaagcctctttttacca

SEQ ID NO: 290 - DNA, 66 bp, synthetic construct
  misc_feature 1..66: note = Description of Artificial Sequence: Synthetic oligonucleotide
  source 1..66: mol_type = other DNA
ggtaaaatggctgagcaagcattagactctaaatctaaagacagaggttaaggcctctttttacca

SEQ ID NO: 291 - DNA, 66 bp, synthetic construct
  misc_feature 1..66: note = Description of Artificial Sequence: Synthetic oligonucleotide
  source 1..66: mol_type = other DNA
ggtaaaatggctgagtaagcattagactctaaatctaaagacagaggtcaaggcctcttttttcct

SEQ ID NO: 292 - DNA, 67 bp, synthetic construct
  misc_feature 1..67: note = Description of Artificial Sequence: Synthetic oligonucleotide
  source 1..67: mol_type = other DNA
ggtaaaatggctgagcaagcattagactctaaatctgaaaacagaggtcaaaggtctctttttacca

SEQ ID NO: 293 - DNA, 66 bp, synthetic construct; identical to SEQ ID NO: 256
  misc_feature 1..66: note = Description of Artificial Sequence: Synthetic oligonucleotide
  source 1..66: mol_type = other DNA

SEQ ID NO: 294 - DNA, 66 bp, synthetic construct
  misc_feature 1..66: note = Description of Artificial Sequence: Synthetic oligonucleotide
  source 1..66: mol_type = other DNA
ggtaaaatgactgaataagccttagactctaaatctgaagacagaggtcaaggcctctttttacca

SEQ ID NO: 295 - DNA, 66 bp, synthetic construct
  misc_feature 1..66: note = Description of Artificial Sequence: Synthetic oligonucleotide
  source 1..66: mol_type = other DNA
ggtaaaatggctgagtaagcattggactctaaatctaaagacagaggtcaagacctctttttacca

SEQ ID NO: 296 - DNA, 66 bp, synthetic construct
  misc_feature 1..66: note = Description of Artificial Sequence: Synthetic oligonucleotide
  source 1..66: mol_type = other DNA
ggtaaaatggctgagtaaagcattagactctaaatctaaggacagaggctaaacctctttttacca

SEQ ID NO: 297 - DNA, 75 bp, synthetic construct
  misc_feature 1..75: note = Description of Artificial Sequence: Synthetic oligonucleotide
  source 1..75: mol_type = other DNA
gttaagatggcagagcctggtaattgcattaaacttaaaattttataatcagaggttcaactcctcttcttaaca

SEQ ID NO: 298 - DNA, 75 bp, synthetic construct
  misc_feature 1..75: note = Description of Artificial Sequence: Synthetic oligonucleotide
  source 1..75: mol_type = other DNA
gttaagatggcagagcccggcaattgcattagacttaaaactttataatcagaggttcaactcctctcattaaca

SEQ ID NO: 299 - DNA, 82 bp, synthetic construct
  misc_feature 1..82: note = Description of Artificial Sequence: Synthetic oligonucleotide
  source 1..82: mol_type = other DNA
ggtagcgtggccgagcggtctaaggcgctggattttagctccagtctcttcgggggcgtgggttcaaatcccaccgctgcca

SEQ ID NO: 300 - DNA, 82 bp, synthetic construct
  misc_feature 1..82: note = Description of Artificial Sequence: Synthetic oligonucleotide
  source 1..82: mol_type = other DNA
ggtagcgtggccgagtggtctaagacgctggattttagctccagtctcttcgggggcgtgggtttgaatcccaccgctgcca

SEQ ID NO: 301 - DNA, 73 bp, synthetic construct
  misc_feature 1..73: note = Description of Artificial Sequence: Synthetic oligonucleotide
  source 1..73: mol_type = other DNA
gggccagtggctcaatggataatgcgtctgactttaaatcagaagattccagccttgactcctggctggctca

SEQ ID NO: 302 - DNA, 82 bp, synthetic construct
  misc_feature 1..82: note = Description of Artificial Sequence: Synthetic oligonucleotide
  source 1..82: mol_type = other DNA
ggtagggtggccgagcggtctaaggcactgtattttaactccagtctcttcagaggcatgggtttgaatcccactgctgcca

SEQ ID NO: 303 - DNA, 65 bp, synthetic construct
  misc_feature 1..65: note = Description of Artificial Sequence: Synthetic oligonucleotide
  source 1..65: mol_type = other DNA
gccgagcggtctaaggctccggattttagcgccggtgtcttcggaggcatgggttcgaattccac

SEQ ID NO: 304 - DNA, 106 bp, synthetic construct
  misc_feature 1..106: note = Description of Artificial Sequence: Synthetic oligonucleotide
  source 1..106: mol_type = other DNA
gtcaggatggccgagtggtctaaggcgccagactttagctaagcttcctccgcggtggggattctggtctccaatggaggcgtgggttcgaatcccacttctgaca

SEQ ID NO: 305 - DNA, 83 bp, synthetic construct
  misc_feature 1..83: note = Description of Artificial Sequence: Synthetic oligonucleotide
  source 1..83: mol_type = other DNA
gtcaggatggccgagtggtctaaggcgccagactttagttctggtctccaatggaggcgtgggttcgaatcccacttctgaca

SEQ ID NO: 306 - DNA, 105 bp, synthetic construct
  misc_feature 1..105: note = Description of Artificial Sequence: Synthetic oligonucleotide
  source 1..105: mol_type = other DNA
gtcaggatggccgagtggtctaaggcgccagactttagcttggcttcctcgtgttgaggattctggtctccaatggaggcgtgggttcgaatcccacttctgaca

SEQ ID NO: 307 - DNA, 83 bp, synthetic construct; identical to SEQ ID NO: 305
  misc_feature 1..83: note = Description of Artificial Sequence: Synthetic oligonucleotide
  source 1..83: mol_type = other DNA

SEQ ID NO: 308 - DNA, 108 bp, synthetic construct
  misc_feature 1..108: note = Description of Artificial Sequence: Synthetic oligonucleotide
  source 1..108: mol_type = other DNA
gtcaggatggccgagtggtctaaggcgccagactttagcttactgcttcctgtgttcgggtcttctggtctccgtatggaggcgtgggttcgaatcccacttctgaca

SEQ ID NO: 309 - DNA, 84 bp, synthetic construct
  misc_feature 1..84: note = Description of Artificial Sequence: Synthetic oligonucleotide
  source 1..84: mol_type = other DNA
gtcaggatggccgagtggtctaaggcgccagactttagttctggtctccgtatggaggcgtgggttcgaatcccacttctgaca

SEQ ID NO: 310 - DNA, 107 bp, synthetic construct
  misc_feature 1..107: note = Description of Artificial Sequence: Synthetic oligonucleotide
  source 1..107: mol_type = other DNA
gtcaggatggccgagtggtctaaggcgccagactttagttgctacttcccaggtttggggcttctggtctccgcatggaggcgtgggttcgaatcccacttctgaca

SEQ ID NO: 311 - DNA, 84 bp, synthetic construct
  misc_feature 1..84: note = Description of Artificial Sequence: Synthetic oligonucleotide
  source 1..84: mol_type = other DNA
gtcaggatggccgagtggtctaaggcgccagactttagttctggtctccgcatggaggcgtgggttcgaatcccacttctgaca

SEQ ID NO: 312 - DNA, 106 bp, synthetic construct
  misc_feature 1..106: note = Description of Artificial Sequence: Synthetic oligonucleotide
  source 1..106: mol_type = other DNA
gtcaggatggccgagtggtctaaggcgccagactttaggtaagcaccttgcctgcgggctttctggtctccggatggaggcgtgggttcgaatcccacttctgaca

SEQ ID NO: 313 - DNA, 85 bp, synthetic construct
  misc_feature 1..85: note = Description of Artificial Sequence: Synthetic oligonucleotide
  source 1..85: mol_type = other DNA
gtcaggatggccgagtggtctaaggcgccagactttagtttctggtctccggatggaggcgtgggttcgaatcccacttctgaca

SEQ ID NO: 314 - DNA, 74 bp, synthetic construct
  misc_feature 1..74: note = Description of Artificial Sequence: Synthetic oligonucleotide
  source 1..74: mol_type = other DNA
gcctccttagtgcagtaggtagcgcatcagtctttaaatctgaatggtcctgagttcaagcctcagagggggca

SEQ ID NO: 315 - DNA, 84 bp, synthetic construct
  misc_feature 1..84: note = Description of Artificial Sequence: Synthetic oligonucleotide
  source 1..84: mol_type = other DNA
gtcaggatggccgagcagtcttaaggcgctgcgttttaatcgcaccctccgctggaggcgtgggttcgaatcccacttttgaca

SEQ ID NO: 316 - DNA, 60 bp, synthetic construct
  misc_feature 1..60: note = Description of Artificial Sequence: Synthetic oligonucleotide
  source 1..60: mol_type = other DNA
ggttccatggtgtaatggtgagcactctggactttaaatccagaagtagtgctggaacaa

SEQ ID NO: 317 - DNA, 83 bp, synthetic construct
  misc_feature 1..83: note = Description of Artificial Sequence: Synthetic oligonucleotide
  source 1..83: mol_type = other DNA
gtcagggtggctgagcagtctgaggggctgcgttttagtcgcagtctgccctggaggcgtgggttcgaatcccactcctgaaa

SEQ ID NO: 318 - DNA, 83 bp, synthetic construct
  misc_feature 1..83: note = Description of Artificial Sequence: Synthetic oligonucleotide
  source 1..83: mol_type = other DNA
accaggatggccgagtggttaaggcgttggactttagatccaatggacatatgtccgcgtgggttcgaaccccactcctggta

SEQ ID NO: 319 - DNA, 83 bp, synthetic construct
  misc_feature 1..83: note = Description of Artificial Sequence: Synthetic oligonucleotide
  source 1..83: mol_type = other DNA
accgggatggccgagtggttaaggcgttggactttagatccaatgggctggtgcccgcgtgggttcgaaccccactctcggta

SEQ ID NO: 320 - DNA, 83 bp, synthetic construct
  misc_feature 1..83: note = Description of Artificial Sequence: Synthetic oligonucleotide
  source 1..83: mol_type = other DNA
accagaatggccgagtggttaaggcgttggactttagatccaatggattcatatccgcgtgggttcgaaccccacttctggta

SEQ ID NO: 321 - DNA, 83 bp, synthetic construct
  misc_feature 1..83: note = Description of Artificial Sequence: Synthetic oligonucleotide
  source 1..83: mol_type = other DNA
accgggatggctgagtggttaaggcgttggactttagatccaatggacaggtgtccgcgtgggttcgagccccactcccggta

SEQ ID NO: 322 - DNA, 83 bp, synthetic construct
  misc_feature 1..83: note = Description of Artificial Sequence: Synthetic oligonucleotide
  source 1..83: mol_type = other DNA
actcatttggctgagtggttaaggcattggactttagatccaatggagtagtggctgtgtgggtttaaaccccactactggta

SEQ ID NO: 323 - DNA, 69 bp, synthetic construct
  misc_feature 1..69: note = Description of Artificial Sequence: Synthetic oligonucleotide
  source 1..69: mol_type = other DNA
gagaaagtcatcgtagttacgaagttggctttaacccagttttgggaggttcaattccttcctttctct

SEQ ID NO: 324 - DNA, 84 bp, synthetic construct
  misc_feature 1..84: note = Description of Artificial Sequence: Synthetic oligonucleotide
  source 1..84: mol_type = other DNA
accaggatggccaagtagttaaaggcactggactttagagccaatggacatatgtctgtgtgggtttgaaccccactcctggtg

SEQ ID NO: 325 - DNA, 82 bp, synthetic construct
  misc_feature 1..82: note = Description of Artificial Sequence: Synthetic oligonucleotide
  source 1..82: mol_type = other DNA
ggtagcgtggccgagcggtctaaggcgctggattttagctccagtctcttcggaggcgtgggttcgaatcccaccgctgcca

SEQ ID NO: 326 - DNA, 82 bp, synthetic construct
  misc_feature 1..82: note = Description of Artificial Sequence: Synthetic oligonucleotide
  source 1..82: mol_type = other DNA
ggtagtgtggccgagcggtctaaggcgctggattttagctccagtctcttcgggggcgtgggttcgaatcccaccactgcca

SEQ ID NO: 327 - DNA, 82 bp, synthetic construct
  misc_feature 1..82: note = Description of Artificial Sequence: Synthetic oligonucleotide
  source 1..82: mol_type = other DNA
ggtagcgtggccgagtggtctaaggcgctggattttagctccagtcatttcgatggcgtgggttcgaatcccaccgctgcca

SEQ ID NO: 328 - DNA, 82 bp, synthetic construct
  misc_feature 1..82: note = Description of Artificial Sequence: Synthetic oligonucleotide
  source 1..82: mol_type = other DNA
ggtagtgtggttgaatggtctaaggcactgaattttagctccagtctctttggggacgtgggtttaaatcccactgctgcaa

SEQ ID NO: 329 - DNA, 75 bp, synthetic construct
  misc_feature 1..75: note = Description of Artificial Sequence: Synthetic oligonucleotide
  source 1..75: mol_type = other DNA
gttaagatggcagagcctggtaattgcactaaacttaaaattttataatcagaggttcaactcctcttcttaaca

SEQ ID NO: 330 - DNA, 75 bp, synthetic construct
  misc_feature 1..75: note = Description of Artificial Sequence: Synthetic oligonucleotide
  source 1..75: mol_type = other DNA
gttaagatggcagagcccggcaattgcactagacttaaaactttataatcagaggttcaactcctctcattaaca

SEQ ID NO: 331 - DNA, 82 bp, synthetic construct
  misc_feature 1..82: note = Description of Artificial Sequence: Synthetic oligonucleotide
  source 1..82: mol_type = other DNA
ggtagcgtggccgagcggtctaaggcgctggattctagctccagtctcttcgggggcgtgggttcaaatcccaccgctgcca

SEQ ID NO: 332 - DNA, 82 bp, synthetic construct
  misc_feature 1..82: note = Description of Artificial Sequence: Synthetic oligonucleotide
  source 1..82: mol_type = other DNA
ggtagcgtggccgagtggtctaagacgctggattctagctccagtctcttcgggggcgtgggtttgaatcccaccgctgcca

SEQ ID NO: 333 - DNA, 73 bp, synthetic construct
  misc_feature 1..73: note = Description of Artificial Sequence: Synthetic oligonucleotide
  source 1..73: mol_type = other DNA
gggccagtggctcaatggataatgcgtctgactctaaatcagaagattccagccttgactcctggctggctca

SEQ ID NO: 334 - DNA, 82 bp, synthetic construct
  misc_feature 1..82: note = Description of Artificial Sequence: Synthetic oligonucleotide
  source 1..82: mol_type = other DNA
ggtagggtggccgagcggtctaaggcactgtattctaactccagtctcttcagaggcatgggtttgaatcccactgctgcca

SEQ ID NO: 335 - DNA, 65 bp, synthetic construct
  misc_feature 1..65: note = Description of Artificial Sequence: Synthetic oligonucleotide
  source 1..65: mol_type = other DNA
gccgagcggtctaaggctccggattctagcgccggtgtcttcggaggcatgggttcgaattccac

SEQ ID NO: 336 - DNA, 106 bp, synthetic construct
  misc_feature 1..106: note = Description of Artificial Sequence: Synthetic oligonucleotide
  source 1..106: mol_type = other DNA
gtcaggatggccgagtggtctaaggcgccagactctagctaagcttcctccgcggtggggattctggtctccaatggaggcgtgggttcgaatcccacttctgaca

SEQ ID NO: 337 - DNA, 83 bp, synthetic construct
  misc_feature 1..83: note = Description of Artificial Sequence: Synthetic oligonucleotide
  source 1..83: mol_type = other DNA
gtcaggatggccgagtggtctaaggcgccagactctagttctggtctccaatggaggcgtgggttcgaatcccacttctgaca

SEQ ID NO: 338 - DNA, 105 bp, synthetic construct
  misc_feature 1..105: note = Description of Artificial Sequence: Synthetic oligonucleotide
  source 1..105: mol_type = other DNA
gtcaggatggccgagtggtctaaggcgccagactctagcttggcttcctcgtgttgaggattctggtctccaatggaggcgtgggttcgaatcccacttctgaca

SEQ ID NO: 339 - DNA, 83 bp, synthetic construct; identical to SEQ ID NO: 337
  misc_feature 1..83: note = Description of Artificial Sequence: Synthetic oligonucleotide
  source 1..83: mol_type = other DNA

SEQ ID NO: 340 - DNA, 108 bp, synthetic construct
  misc_feature 1..108: note = Description of Artificial Sequence: Synthetic oligonucleotide
  source 1..108: mol_type = other DNA
gtcaggatggccgagtggtctaaggcgccagactctagcttactgcttcctgtgttcgggtcttctggtctccgtatggaggcgtgggttcgaatcccacttctgaca

SEQ ID NO: 341 - DNA, 84 bp, synthetic construct
  misc_feature 1..84: note = Description of Artificial Sequence: Synthetic oligonucleotide
  source 1..84: mol_type = other DNA
gtcaggatggccgagtggtctaaggcgccagactctagttctggtctccgtatggaggcgtgggttcgaatcccacttctgaca

SEQ ID NO: 342 - DNA, 107 bp, synthetic construct
  misc_feature 1..107: note = Description of Artificial Sequence: Synthetic oligonucleotide
  source 1..107: mol_type = other DNA
gtcaggatggccgagtggtctaaggcgccagactctagttgctacttcccaggtttggggcttctggtctccgcatggaggcgtgggttcgaatcccacttctgaca

SEQ ID NO: 343 - DNA, 84 bp, synthetic construct
  misc_feature 1..84: note = Description of Artificial Sequence: Synthetic oligonucleotide
  source 1..84: mol_type = other DNA
gtcaggatggccgagtggtctaaggcgccagactctagttctggtctccgcatggaggcgtgggttcgaatcccacttctgaca

SEQ ID NO: 344 - DNA, 106 bp, synthetic construct
  misc_feature 1..106: note = Description of Artificial Sequence: Synthetic oligonucleotide
  source 1..106: mol_type = other DNA
gtcaggatggccgagtggtctaaggcgccagactctaggtaagcaccttgcctgcgggctttctggtctccggatggaggcgtgggttcgaatcccacttctgaca

SEQ ID NO: 345 - DNA, 85 bp, synthetic construct
  misc_feature 1..85: note = Description of Artificial Sequence: Synthetic oligonucleotide
  source 1..85: mol_type = other DNA
gtcaggatggccgagtggtctaaggcgccagactctagtttctggtctccggatggaggcgtgggttcgaatcccacttctgaca

SEQ ID NO: 346 - DNA, 74 bp, synthetic construct
  misc_feature 1..74: note = Description of Artificial Sequence: Synthetic oligonucleotide
  source 1..74: mol_type = other DNA
gcctccttagtgcagtaggtagcgcatcagtctctaaatctgaatggtcctgagttcaagcctcagagggggca

SEQ ID NO: 347 - DNA, 84 bp, synthetic construct
  misc_feature 1..84: note = Description of Artificial Sequence: Synthetic oligonucleotide
  source 1..84: mol_type = other DNA
gtcaggatggccgagcagtcttaaggcgctgcgttctaatcgcaccctccgctggaggcgtgggttcgaatcccacttttgaca

SEQ ID NO: 348 - DNA, 60 bp, synthetic construct
  misc_feature 1..60: note = Description of Artificial Sequence: Synthetic oligonucleotide
  source 1..60: mol_type = other DNA
ggttccatggtgtaatggtgagcactctggactctaaatccagaagtagtgctggaacaa

SEQ ID NO: 349 - DNA, 83 bp, synthetic construct
  misc_feature 1..83: note = Description of Artificial Sequence: Synthetic oligonucleotide
  source 1..83: mol_type = other DNA
gtcagggtggctgagcagtctgaggggctgcgttctagtcgcagtctgccctggaggcgtgggttcgaatcccactcctgaaa

SEQ ID NO: 350 - DNA, 83 bp, synthetic construct
  misc_feature 1..83: note = Description of Artificial Sequence: Synthetic oligonucleotide
  source 1..83: mol_type = other DNA
accaggatggccgagtggttaaggcgttggactctagatccaatggacatatgtccgcgtgggttcgaaccccactcctggta

SEQ ID NO: 351 - DNA, 83 bp, synthetic construct
  misc_feature 1..83: note = Description of Artificial Sequence: Synthetic oligonucleotide
  source 1..83: mol_type = other DNA
accgggatggccgagtggttaaggcgttggactctagatccaatgggctggtgcccgcgtgggttcgaaccccactctcggta

SEQ ID NO: 352 - DNA, 83 bp, synthetic construct
  misc_feature 1..83: note = Description of Artificial Sequence: Synthetic oligonucleotide
  source 1..83: mol_type = other DNA
accagaatggccgagtggttaaggcgttggactctagatccaatggattcatatccgcgtgggttcgaaccccacttctggta

SEQ ID NO: 353 - DNA, 83 bp, synthetic construct
  misc_feature 1..83: note = Description of Artificial Sequence: Synthetic oligonucleotide
  source 1..83: mol_type = other DNA
accgggatggctgagtggttaaggcgttggactctagatccaatggacaggtgtccgcgtgggttcgagccccactcccggta

SEQ ID NO: 354 - DNA, 84 bp, synthetic construct
  misc_feature 1..84: note = Description of Artificial Sequence: Synthetic oligonucleotide
  source 1..84: mol_type = other DNA
actcatttggctgagtggttaaggcattggactctaagatccaatggagtagtggctgtgtgggtttaaaccccactactggta

SEQ ID NO: 355 - DNA, 69 bp, synthetic construct
  misc_feature 1..69: note = Description of Artificial Sequence: Synthetic oligonucleotide
  source 1..69: mol_type = other DNA
gagaaagtcatcgtagttacgaagttggctctaacccagttttgggaggttcaattccttcctttctct

SEQ ID NO: 356 - DNA, 84 bp, synthetic construct
  misc_feature 1..84: note = Description of Artificial Sequence: Synthetic oligonucleotide
  source 1..84: mol_type = other DNA
accaggatggccaagtagttaaaggcactggactctagagccaatggacatatgtctgtgtgggtttgaaccccactcctggtg

SEQ ID NO: 357 - DNA, 82 bp, synthetic construct
  misc_feature 1..82: note = Description of Artificial Sequence: Synthetic oligonucleotide
  source 1..82: mol_type = other DNA
ggtagcgtggccgagcggtctaaggcgctggattctagctccagtctcttcggaggcgtgggttcgaatcccaccgctgcca

SEQ ID NO: 358 - DNA, 82 bp, synthetic construct
  misc_feature 1..82: note = Description of Artificial Sequence: Synthetic oligonucleotide
  source 1..82: mol_type = other DNA
ggtagtgtggccgagcggtctaaggcgctggattctagctccagtctcttcgggggcgtgggttcgaatcccaccactgcca

SEQ ID NO: 359 - DNA, 82 bp, synthetic construct
  misc_feature 1..82: note = Description of Artificial Sequence: Synthetic oligonucleotide
  source 1..82: mol_type = other DNA
ggtagcgtggccgagtggtctaaggcgctggattctagctccagtcatttcgatggcgtgggttcgaatcccaccgctgcca

SEQ ID NO: 360 - DNA, 82 bp, synthetic construct
  misc_feature 1..82: note = Description of Artificial Sequence: Synthetic oligonucleotide
  source 1..82: mol_type = other DNA
ggtagtgtggttgaatggtctaaggcactgaattctagctccagtctctttggggacgtgggtttaaatcccactgctgcaa

SEQ ID NO: 361 - DNA, 70 bp, synthetic construct
  misc_feature 1..70: note = Description of Artificial Sequence: Synthetic oligonucleotide
  source 1..70: mol_type = other DNA
gagaaggtcacagaggttatgggattggctctaaaccagtctgtggggggttcgattccctcctttttca

SEQ ID NO: 362 - DNA, 70 bp, synthetic construct
  misc_feature 1..70: note = Description of Artificial Sequence: Synthetic oligonucleotide
  source 1..70: mol_type = other DNA
gagaaggtcatagaggttatgggattggctctaaaccagtctctggggggttcgattccctcctttttca

SEQ ID NO: 363 - DNA, 69 bp, synthetic construct
  misc_feature 1..69: note = Description of Artificial Sequence: Synthetic oligonucleotide
  source 1..69: mol_type = other DNA
gaaaaagtcataggggttatgaggctggctctaaaccagccttaggaggttcaattccttccttttttg

SEQ ID NO: 364 - DNA, 74 bp, synthetic construct
  misc_feature 1..74: note = Description of Artificial Sequence: Synthetic oligonucleotide
  source 1..74: mol_type = other DNA
ggccggttagctcagttggttagagcgtgctgctctaaatgccagggtcgaggtttcgatccccgtacgggcct

SEQ ID NO: 365 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactctaaatccattggggtttccccgcgcaggttcgaatcctgccgactacg

SEQ ID NO: 366 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactctaaatccattggggtttccccacgcaggttcgaatcctgccgactacg

SEQ ID NO: 367 - DNA, 83 bp, synthetic construct
  misc_feature 1..83: note = Description of Artificial Sequence: Synthetic oligonucleotide
  source 1..83: mol_type = other DNA
gtagtcgtggccgagtggttaaggtgatggactctaaaacccattggggtctccccgcgcaggttcgaatcctgccgactacg

SEQ ID NO: 368 - DNA, 72 bp, synthetic construct
  misc_feature 1..72: note = Description of Artificial Sequence: Synthetic oligonucleotide
  source 1..72: mol_type = other DNA
gggtgtatggctcaggggtagagaatttgactctagatcaagaggtccctggttcaaatccaggtgccccct

SEQ ID NO: 369 - DNA, 78 bp, synthetic construct
  misc_feature 1..78: note = Description of Artificial Sequence: Synthetic oligonucleotide
  source 1..78: mol_type = other DNA
agttgtagctgagtggttaaggcaacgagctctaaattcgttggtttctctctgtgcaggtttgaatcctgctaatta

SEQ ID NO: 370 - DNA, 70 bp, synthetic construct
  misc_feature 1..70: note = Description of Artificial Sequence: Synthetic oligonucleotide
  source 1..70: mol_type = other DNA
caagaaattcatagaggttatgggattggctctaaaccagtttcaggaggttcgattccttcctttttgg

SEQ ID NO: 371 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggcgttggactctaaatccaatggggtctccccgcgcaggttcgaatcctgctcacagcg

SEQ ID NO: 372 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggcgttggactctaaatccaatggggtctccccgcgcaggttcaaatcctgctcacagcg

SEQ ID NO: 373 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggtgttggactctaaatccaatgggggttccccgcgcaggttcaaatcctgctcacagcg

SEQ ID NO: 374 - DNA, 82 bp, synthetic construct
  misc_feature 1..82: note = Description of Artificial Sequence: Synthetic oligonucleotide
  source 1..82: mol_type = other DNA
gtcacggtggccgagtggttaaggcgttggactctaaatccaatggggtttccccgcacaggttcgaatcctgttcgtgacg

SEQ ID NO: 375 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggactctaaatccattgtgctctgcacgcgtgggttcgaatcccaccctcgtcg

SEQ ID NO: 376 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggactctaaatccattgtgctctgcacgcgtgggttcgaatcccaccttcgtcg

SEQ ID NO: 377 - DNA, 74 bp, synthetic construct
  misc_feature 1..74: note = Description of Artificial Sequence: Synthetic oligonucleotide
  source 1..74: mol_type = other DNA
ggccggttagctcagttggttagagcgtgctctaactaatgccagggtcgaggtttcgatccccgtacgggcct

SEQ ID NO: 378 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggactctaaatccattgtgctctgcacacgtgggttcgaatcccatcctcgtcg

SEQ ID NO: 379 - DNA, 78 bp, synthetic construct
  misc_feature 1..78: note = Description of Artificial Sequence: Synthetic oligonucleotide
  source 1..78: mol_type = other DNA
gaggcctggccgagtggttaaggcgatggactctaaatccattgtgctctgcacgcgtgggttcgaatcccatcctcg

SEQ ID NO: 380 - DNA, 82 bp, synthetic construct
  misc_feature 1..82: note = Description of Artificial Sequence: Synthetic oligonucleotide
  source 1..82: mol_type = other DNA
gcagcgatggccgagtggttaaggcgttggactctaaatccaatggggtctccccgcgcaggttcgaaccctgctcgctgcg

SEQ ID NO: 381 - DNA, 82 bp, synthetic construct; identical to SEQ ID NO: 365
  misc_feature 1..82: note = Description of Artificial Sequence: Synthetic oligonucleotide
  source 1..82: mol_type = other DNA

SEQ ID NO: 382 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactctaaatccattggggtctccccgcgcaggttcgaatcctgccgactacg

SEQ ID NO: 383 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactctaaatccattggggtttccccgcgcaggttcgaatcctgtcggctacg

SEQ ID NO: 384 - DNA, 70 bp, synthetic construct
  misc_feature 1..70: note = Description of Artificial Sequence: Synthetic oligonucleotide
  source 1..70: mol_type = other DNA
gagaaggtcacagaggttatgggattggcttcaaaccagtctgtggggggttcgattccctcctttttca

SEQ ID NO: 385 - DNA, 70 bp, synthetic construct
  misc_feature 1..70: note = Description of Artificial Sequence: Synthetic oligonucleotide
  source 1..70: mol_type = other DNA
gagaaggtcatagaggttatgggattggcttcaaaccagtctctggggggttcgattccctcctttttca

SEQ ID NO: 386 - DNA, 69 bp, synthetic construct
  misc_feature 1..69: note = Description of Artificial Sequence: Synthetic oligonucleotide
  source 1..69: mol_type = other DNA
gaaaaagtcataggggttatgaggctggcttcaaaccagccttaggaggttcaattccttccttttttg

SEQ ID NO: 387 - DNA, 74 bp, synthetic construct
  misc_feature 1..74: note = Description of Artificial Sequence: Synthetic oligonucleotide
  source 1..74: mol_type = other DNA
ggccggttagctcagttggttagagcgtgctgcttcaaatgccagggtcgaggtttcgatccccgtacgggcct

SEQ ID NO: 388 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggacttcaaatccattggggtttccccgcgcaggttcgaatcctgccgactacg

SEQ ID NO: 389 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggacttcaaatccattggggtttccccacgcaggttcgaatcctgccgactacg

SEQ ID NO: 390 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggtgatggacttcaaacccattggggtctccccgcgcaggttcgaatcctgccgactacg

SEQ ID NO: 391 - DNA, 72 bp, synthetic construct
  misc_feature 1..72: note = Description of Artificial Sequence: Synthetic oligonucleotide
  source 1..72: mol_type = other DNA
gggtgtatggctcaggggtagagaatttgacttcagatcaagaggtccctggttcaaatccaggtgccccct

SEQ ID NO: 392 - DNA, 78 bp, synthetic construct
  misc_feature 1..78: note = Description of Artificial Sequence: Synthetic oligonucleotide
  source 1..78: mol_type = other DNA
agttgtagctgagtggttaaggcaacgagcttcaaattcgttggtttctctctgtgcaggtttgaatcctgctaatta

SEQ ID NO: 393 - DNA, 70 bp, synthetic construct
  misc_feature 1..70: note = Description of Artificial Sequence: Synthetic oligonucleotide
  source 1..70: mol_type = other DNA
caagaaattcatagaggttatgggattggcttcaaaccagtttcaggaggttcgattccttcctttttgg

SEQ ID NO: 394 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggcgttggacttcaaatccaatggggtctccccgcgcaggttcgaatcctgctcacagcg

SEQ ID NO: 395 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggcgttggacttcaaatccaatggggtctccccgcgcaggttcaaatcctgctcacagcg

SEQ ID NO: 396 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggtgttggacttcaaatccaatgggggttccccgcgcaggttcaaatcctgctcacagcg

SEQ ID NO: 397 - DNA, 82 bp, synthetic construct
  misc_feature 1..82: note = Description of Artificial Sequence: Synthetic oligonucleotide
  source 1..82: mol_type = other DNA
gtcacggtggccgagtggttaaggcgttggacttcaaatccaatggggtttccccgcacaggttcgaatcctgttcgtgacg

SEQ ID NO: 398 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggacttcaaatccattgtgctctgcacgcgtgggttcgaatcccaccctcgtcg

SEQ ID NO: 399 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggacttcaaatccattgtgctctgcacgcgtgggttcgaatcccaccttcgtcg

SEQ ID NO: 400 - DNA, 74 bp, synthetic construct
  misc_feature 1..74: note = Description of Artificial Sequence: Synthetic oligonucleotide
  source 1..74: mol_type = other DNA
ggccggttagctcagttggttagagcgtgcttcaactaatgccagggtcgaggtttcgatccccgtacgggcct

SEQ ID NO: 401 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggacttcaaatccattgtgctctgcacacgtgggttcgaatcccatcctcgtcg

SEQ ID NO: 402 - DNA, 78 bp, synthetic construct
  misc_feature 1..78: note = Description of Artificial Sequence: Synthetic oligonucleotide
  source 1..78: mol_type = other DNA
gaggcctggccgagtggttaaggcgatggacttcaaatccattgtgctctgcacgcgtgggttcgaatcccatcctcg

SEQ ID NO: 403 - DNA, 82 bp, synthetic construct
  misc_feature 1..82: note = Description of Artificial Sequence: Synthetic oligonucleotide
  source 1..82: mol_type = other DNA
gcagcgatggccgagtggttaaggcgttggacttcaaatccaatggggtctccccgcgcaggttcgaaccctgctcgctgcg

SEQ ID NO: 404 - DNA, 82 bp, synthetic construct; identical to SEQ ID NO: 388
  misc_feature 1..82: note = Description of Artificial Sequence: Synthetic oligonucleotide
  source 1..82: mol_type = other DNA

SEQ ID NO: 405 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggacttcaaatccattggggtctccccgcgcaggttcgaatcctgccgactacg

SEQ ID NO: 406 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggacttcaaatccattggggtttccccgcgcaggttcgaatcctgtcggctacg

SEQ ID NO: 407 - DNA, 73 bp, synthetic construct
  misc_feature 1..73: note = Description of Artificial Sequence: Synthetic oligonucleotide
  source 1..73: mol_type = other DNA
gcccagctagctcagtcggtagagcataagactttaaatctcagggttgtggattcgtgccccatgctgggtg

SEQ ID NO: 408 - DNA, 71 bp, synthetic construct
  misc_feature 1..71: note = Description of Artificial Sequence: Synthetic oligonucleotide
  source 1..71: mol_type = other DNA
ctgcagctagctcagtcggtagagcatgagactttaaatctcagggtcatgggttcgtgccccatgttggg

SEQ ID NO: 409 - DNA, 70 bp, synthetic construct
  misc_feature 1..70: note = Description of Artificial Sequence: Synthetic oligonucleotide
  source 1..70: mol_type = other DNA
ccagcatgtctcagtcggtatagtgtgagactttaaatctcagggtcgtgggttcaagccccacattggg

SEQ ID NO: 410 - DNA, 73 bp, synthetic construct
  misc_feature 1..73: note = Description of Artificial Sequence: Synthetic oligonucleotide
  source 1..73: mol_type = other DNA
gtctagctagatcagttggtagagcataagactttaaatctcagggtcatgggtttgagccctacgttgggcg

SEQ ID NO: 411 - DNA, 73 bp, synthetic construct
  misc_feature 1..73: note = Description of Artificial Sequence: Synthetic oligonucleotide
  source 1..73: mol_type = other DNA
gcccagctagctcagccggtagagcacaagactttaaatctcagggtcgtgggtttgagccctgtgttgagca

SEQ ID NO: 412 - DNA, 69 bp, synthetic construct
  misc_feature 1..69: note = Description of Artificial Sequence: Synthetic oligonucleotide
  source 1..69: mol_type = other DNA
ccgaatagcttagttgatgaagcgtgagactttaaatctcagggtagtgggttcaagccccacattgga

SEQ ID NO: 413 - DNA, 74 bp, synthetic construct
  misc_feature 1..74: note = Description of Artificial Sequence: Synthetic oligonucleotide
  source 1..74: mol_type = other DNA
gcctggctacctcagttggtagagcatgggactttaaatcccagagtcagtgggttcaagcctcacattgagtg

SEQ ID NO: 414 - DNA, 73 bp, synthetic construct
  misc_feature 1..73: note = Description of Artificial Sequence: Synthetic oligonucleotide
  source 1..73: mol_type = other DNA
gcccggctagctcagtcggtagagcatgagaccttaaatctcagggtcgtgggttcgagccccacgttgggcg

SEQ ID NO: 415 - DNA, 73 bp, synthetic construct
  misc_feature 1..73: note = Description of Artificial Sequence: Synthetic oligonucleotide
  source 1..73: mol_type = other DNA
gcccggctagctcagtcggtagagcatgggactttaaatctcagggtcgtgggttcgagccccacgttgggcg

SEQ ID NO: 416 - DNA, 73 bp, synthetic construct
  misc_feature 1..73: note = Description of Artificial Sequence: Synthetic oligonucleotide
  source 1..73: mol_type = other DNA
gcccggctagctcagtcgatagagcatgagactttaaatctcagggtcgtgggttcgagccgcacgttgggcg

SEQ ID NO: 417 - DNA, 73 bp, synthetic construct
  misc_feature 1..73: note = Description of Artificial Sequence: Synthetic oligonucleotide
  source 1..73: mol_type = other DNA
gcccagctagctcagtcggtagagcatgagactttaaatctcagggtcatgggtttgagccccacgtttggtg

SEQ ID NO: 418 - DNA, 73 bp, synthetic construct
  misc_feature 1..73: note = Description of Artificial Sequence: Synthetic oligonucleotide
  source 1..73: mol_type = other DNA
gcctggctagctcagtcggcaaagcatgagactttaaatctcagggtcgtgggctcgagctccatgttgggcg

SEQ ID NO: 419 - DNA, 73 bp, synthetic construct
  misc_feature 1..73: note = Description of Artificial Sequence: Synthetic oligonucleotide
  source 1..73: mol_type = other DNA
gcccgactacctcagtcggtggagcatgggactttacatcccagggttgtgggttcgagccccacattgggca

SEQ ID NO: 420 - DNA, 73 bp, synthetic construct
  misc_feature 1..73: note = Description of Artificial Sequence: Synthetic oligonucleotide
  source 1..73: mol_type = other DNA
ccccggctggctcagtcagtagatcatgagactttaaatctcagggtcgtgggttcacgccccacactgggcg

SEQ ID NO: 421 - DNA, 65 bp, synthetic construct
  misc_feature 1..65: note = Description of Artificial Sequence: Synthetic oligonucleotide
  source 1..65: mol_type = other DNA
gcgctagtcagtagagcatgagactttaaatctcagggtcgtgggttcgagccccacatcgggcg

SEQ ID NO: 422 - DNA, 73 bp, synthetic construct
  misc_feature 1..73: note = Description of Artificial Sequence: Synthetic oligonucleotide
  source 1..73: mol_type = other DNA
gcctggatagctcagttggtagagcatcagactttaaatctgagggtccagggttcaagtccctgttcaggca

SEQ ID NO: 423 - DNA, 74 bp, synthetic construct
  misc_feature 1..74: note = Description of Artificial Sequence: Synthetic oligonucleotide
  source 1..74: mol_type = other DNA
gccaggatagttcaggtggtagagcatcagactttaaaacctgagggttcagggttcaagtctctgtttgggcg

SEQ ID NO: 424 - DNA, 73 bp, synthetic construct
  misc_feature 1..73: note = Description of Artificial Sequence: Synthetic oligonucleotide
  source 1..73: mol_type = other DNA
acccagatagctcagtcagtagagcatcagactttaaatctgagggtccaaggttcatgtccctttttgggtg

SEQ ID NO: 425 - DNA, 73 bp, synthetic construct
  misc_feature 1..73: note = Description of Artificial Sequence: Synthetic oligonucleotide
  source 1..73: mol_type = other DNA
acctgggtagcttagttggtagagcattggactttaaatttgagggcccaggtttcaagtccctgtttgggtg

SEQ ID NO: 426 - DNA, 74 bp, synthetic construct
  misc_feature 1..74: note = Description of Artificial Sequence: Synthetic oligonucleotide
  source 1..74: mol_type = other DNA
gcctgggtagctcagtcggtagagctatcagactttaagcctgaggattcagggttcaatcccttgctggggcg

SEQ ID NO: 427 - DNA, 62 bp, synthetic construct
  misc_feature 1..62: note = Description of Artificial Sequence: Synthetic oligonucleotide
  source 1..62: mol_type = other DNA
gatagctcagttgatagagcatcagactttaaatctgagggtccagggttcatgtccctgtt

SEQ ID NO: 428 - DNA, 73 bp, synthetic construct
  misc_feature 1..73: note = Description of Artificial Sequence: Synthetic oligonucleotide
  source 1..73: mol_type = other DNA
gttggggtaactcagttggtagagtagcagactttacatctgagggtccagggtttaagtccatgtccaggca

SEQ ID NO: 429 - DNA, 73 bp, synthetic construct
  misc_feature 1..73: note = Description of Artificial Sequence: Synthetic oligonucleotide
  source 1..73: mol_type = other DNA
gcctggatagctcagttggtagagcatcagactttaaatctgagggtccagggttcaagtccctgttcaggcg

SEQ ID NO: 430 - DNA, 73 bp, synthetic construct
  misc_feature 1..73: note = Description of Artificial Sequence: Synthetic oligonucleotide
  source 1..73: mol_type = other DNA
gcctggatagctcagtcggtagagcatcagactttaaatctgagggtccagggttcaagtccctgttcaggcg

SEQ ID NO: 431 - DNA, 73 bp, synthetic construct
  misc_feature 1..73: note = Description of Artificial Sequence: Synthetic oligonucleotide
  source 1..73: mol_type = other DNA
gcccggatagctcagtcggtagagcatcagactttaaatctgagggtccggggttcaagtccctgttcgggcg

SEQ ID NO: 432 - DNA, 73 bp, synthetic construct
  misc_feature 1..73: note = Description of Artificial Sequence: Synthetic oligonucleotide
  source 1..73: mol_type = other DNA
gcctgggtagctcagtcggtagagcatcagactttaaatctgagggtccagggttcaagtccctgtccaggcg

SEQ ID NO: 433 - DNA, 73 bp, synthetic construct
  misc_feature 1..73: note = Description of Artificial Sequence: Synthetic oligonucleotide
  source 1..73: mol_type = other DNA
gcctggatagctcagttggtagaacatcagactttaaatctgacggtgcagggttcaagtccctgttcaggcg

SEQ ID NO: 434 - DNA, 73 bp, synthetic construct
  misc_feature 1..73: note = Description of Artificial Sequence: Synthetic oligonucleotide
  source 1..73: mol_type = other DNA
gcccggagagctcagtgggtagagcatcagactttaaatctgagggtccagggttcaagtcctcgttcgggca

SEQ ID NO: 435 - DNA, 73 bp, synthetic construct
  misc_feature 1..73: note = Description of Artificial Sequence: Synthetic oligonucleotide
  source 1..73: mol_type = other DNA
acctgggtagctcagtaggtagaacatcagactttaaatctgagggtctagggttcaagtccctgtccaggcg

SEQ ID NO: 436 - DNA, 76 bp, synthetic construct
  misc_feature 1..76: note = Description of Artificial Sequence: Synthetic oligonucleotide
  source 1..76: mol_type = other DNA
gcctggatagctccttcggtagagcatcatcagactttaaatgtgagggtccagggttcaagttcctgtttgggcg

SEQ ID NO: 437 - DNA, 73 bp, synthetic construct
  misc_feature 1..73: note = Description of Artificial Sequence: Synthetic oligonucleotide
  source 1..73: mol_type = other DNA
gcccagctagctcagtcggtagagcataagactctaaatctcagggttgtggattcgtgccccatgctgggtg

SEQ ID NO: 438 - DNA, 71 bp, synthetic construct
  misc_feature 1..71: note = Description of Artificial Sequence: Synthetic oligonucleotide
  source 1..71: mol_type = other DNA
ctgcagctagctcagtcggtagagcatgagactctaaatctcagggtcatgggttcgtgccccatgttggg

SEQ ID NO: 439 - DNA, 70 bp, synthetic construct
  misc_feature 1..70: note = Description of Artificial Sequence: Synthetic oligonucleotide
  source 1..70: mol_type = other DNA
ccagcatgtctcagtcggtatagtgtgagactctaaatctcagggtcgtgggttcaagccccacattggg

SEQ ID NO: 440 - DNA, 73 bp, synthetic construct
  misc_feature 1..73: note = Description of Artificial Sequence: Synthetic oligonucleotide
  source 1..73: mol_type = other DNA
gtctagctagatcagttggtagagcataagactctaaatctcagggtcatgggtttgagccctacgttgggcg

SEQ ID NO: 441 - DNA, 73 bp, synthetic construct
  misc_feature 1..73: note = Description of Artificial Sequence: Synthetic oligonucleotide
  source 1..73: mol_type = other DNA
gcccagctagctcagccggtagagcacaagactctaaatctcagggtcgtgggtttgagccctgtgttgagca

SEQ ID NO: 442 - DNA, 69 bp, synthetic construct
  misc_feature 1..69: note = Description of Artificial Sequence: Synthetic oligonucleotide
  source 1..69: mol_type = other DNA
ccgaatagcttagttgatgaagcgtgagactctaaatctcagggtagtgggttcaagccccacattgga

SEQ ID NO: 443 - DNA, 74 bp, synthetic construct
  misc_feature 1..74: note = Description of Artificial Sequence: Synthetic oligonucleotide
  source 1..74: mol_type = other DNA
gcctggctacctcagttggtagagcatgggactctaaatcccagagtcagtgggttcaagcctcacattgagtg

SEQ ID NO: 444 - DNA, 73 bp, synthetic construct
  misc_feature 1..73: note = Description of Artificial Sequence: Synthetic oligonucleotide
  source 1..73: mol_type = other DNA
gcccggctagctcagtcggtagagcatgagaccctaaatctcagggtcgtgggttcgagccccacgttgggcg

SEQ ID NO: 445 - DNA, 73 bp, synthetic construct
  misc_feature 1..73: note = Description of Artificial Sequence: Synthetic oligonucleotide
  source 1..73: mol_type = other DNA
gcccggctagctcagtcggtagagcatgggactctaaatctcagggtcgtgggttcgagccccacgttgggcg

SEQ ID NO: 446 - DNA, 73 bp, synthetic construct
  misc_feature 1..73: note = Description of Artificial Sequence: Synthetic oligonucleotide
  source 1..73: mol_type = other DNA
gcccggctagctcagtcgatagagcatgagactctaaatctcagggtcgtgggttcgagccgcacgttgggcg

SEQ ID NO: 447 - DNA, 73 bp, synthetic construct
  misc_feature 1..73: note = Description of Artificial Sequence: Synthetic oligonucleotide
  source 1..73: mol_type = other DNA
gcccagctagctcagtcggtagagcatgagactctaaatctcagggtcatgggtttgagccccacgtttggtg

SEQ ID NO: 448 - DNA, 73 bp, synthetic construct
  misc_feature 1..73: note = Description of Artificial Sequence: Synthetic oligonucleotide
  source 1..73: mol_type = other DNA
gcctggctagctcagtcggcaaagcatgagactctaaatctcagggtcgtgggctcgagctccatgttgggcg

SEQ ID NO: 449 - DNA, 73 bp, synthetic construct
  misc_feature 1..73: note = Description of Artificial Sequence: Synthetic oligonucleotide
  source 1..73: mol_type = other DNA
gcccgactacctcagtcggtggagcatgggactctacatcccagggttgtgggttcgagccccacattgggca

SEQ ID NO: 450 - DNA, 73 bp, synthetic construct
  misc_feature 1..73: note = Description of Artificial Sequence: Synthetic oligonucleotide
  source 1..73: mol_type = other DNA
ccccggctggctcagtcagtagatcatgagactctaaatctcagggtcgtgggttcacgccccacactgggcg

SEQ ID NO: 451 - DNA, 65 bp, synthetic construct
  misc_feature 1..65: note = Description of Artificial Sequence: Synthetic oligonucleotide
  source 1..65: mol_type = other DNA
gcgctagtcagtagagcatgagactctaaatctcagggtcgtgggttcgagccccacatcgggcg

SEQ ID NO: 452 - DNA, 73 bp, synthetic construct
  misc_feature 1..73: note = Description of Artificial Sequence: Synthetic oligonucleotide
  source 1..73: mol_type = other DNA
gcctggatagctcagttggtagagcatcagactctaaatctgagggtccagggttcaagtccctgttcaggca

SEQ ID NO: 453 - DNA, 73 bp, synthetic construct
  misc_feature 1..73: note = Description of Artificial Sequence: Synthetic oligonucleotide
  source 1..73: mol_type = other DNA
gccaggatagttcaggtggtagagcatcagactctaaacctgagggttcagggttcaagtctctgtttgggcg

SEQ ID NO: 454 - DNA, 73 bp, synthetic construct
  misc_feature 1..73: note = Description of Artificial Sequence: Synthetic oligonucleotide
  source 1..73: mol_type = other DNA
acccagatagctcagtcagtagagcatcagactctaaatctgagggtccaaggttcatgtccctttttgggtg

SEQ ID NO: 455 - DNA, 73 bp, synthetic construct
  misc_feature 1..73: note = Description of Artificial Sequence: Synthetic oligonucleotide
  source 1..73: mol_type = other DNA
acctgggtagcttagttggtagagcattggactctaaatttgagggcccaggtttcaagtccctgtttgggtg

SEQ ID NO: 456 - DNA, 75 bp, synthetic construct
  misc_feature 1..75: note = Description of Artificial Sequence: Synthetic oligonucleotide
  source 1..75: mol_type = other DNA
gcctgggtagctcagtcggtagagctatcagactctaaagcctgaggattcagggttcaatcccttgctggggcg

SEQ ID NO: 457 - DNA, 62 bp, synthetic construct
  misc_feature 1..62: note = Description of Artificial Sequence: Synthetic oligonucleotide
  source 1..62: mol_type = other DNA
gatagctcagttgatagagcatcagactctaaatctgagggtccagggttcatgtccctgtt

SEQ ID NO: 458 - DNA, 73 bp, synthetic construct
  misc_feature 1..73: note = Description of Artificial Sequence: Synthetic oligonucleotide
  source 1..73: mol_type = other DNA
gttggggtaactcagttggtagagtagcagactctacatctgagggtccagggtttaagtccatgtccaggca

SEQ ID NO: 459 - DNA, 73 bp, synthetic construct
  misc_feature 1..73: note = Description of Artificial Sequence: Synthetic oligonucleotide
  source 1..73: mol_type = other DNA
gcctggatagctcagttggtagagcatcagactctaaatctgagggtccagggttcaagtccctgttcaggcg

SEQ ID NO: 460 - DNA, 73 bp, synthetic construct
  misc_feature 1..73: note = Description of Artificial Sequence: Synthetic oligonucleotide
  source 1..73: mol_type = other DNA
gcctggatagctcagtcggtagagcatcagactctaaatctgagggtccagggttcaagtccctgttcaggcg

SEQ ID NO: 461 - DNA, 73 bp, synthetic construct
  misc_feature 1..73: note = Description of Artificial Sequence: Synthetic oligonucleotide
  source 1..73: mol_type = other DNA
gcccggatagctcagtcggtagagcatcagactctaaatctgagggtccggggttcaagtccctgttcgggcg

SEQ ID NO: 462 - DNA, 73 bp, synthetic construct
  misc_feature 1..73: note = Description of Artificial Sequence: Synthetic oligonucleotide
  source 1..73: mol_type = other DNA
gcctgggtagctcagtcggtagagcatcagactctaaatctgagggtccagggttcaagtccctgtccaggcg

SEQ ID NO: 463 - DNA, 73 bp, synthetic construct
  misc_feature 1..73: note = Description of Artificial Sequence: Synthetic oligonucleotide
  source 1..73: mol_type = other DNA
gcctggatagctcagttggtagaacatcagactctaaatctgacggtgcagggttcaagtccctgttcaggcg

SEQ ID NO: 464 - DNA, 73 bp, synthetic construct
  misc_feature 1..73: note = Description of Artificial Sequence: Synthetic oligonucleotide
  source 1..73: mol_type = other DNA
gcccggagagctcagtgggtagagcatcagactctaaatctgagggtccagggttcaagtcctcgttcgggca

SEQ ID NO: 465 - DNA, 73 bp, synthetic construct
  misc_feature 1..73: note = Description of Artificial Sequence: Synthetic oligonucleotide
  source 1..73: mol_type = other DNA
acctgggtagctcagtaggtagaacatcagactctaaatctgagggtctagggttcaagtccctgtccaggcg

SEQ ID NO: 466 - DNA, 76 bp, synthetic construct
  misc_feature 1..76: note = Description of Artificial Sequence: Synthetic oligonucleotide
  source 1..76: mol_type = other DNA
gcctggatagctccttcggtagagcatcatcagactctaaatgtgagggtccagggttcaagttcctgtttgggcg

SEQ ID NO: 467 - DNA, 75 bp, synthetic construct
  misc_feature 1..75: note = Description of Artificial Sequence: Synthetic oligonucleotide
  source 1..75: mol_type = other DNA
ggcagaatggtgcagcggttcagcacccaggctcttcagccagctgttgcctgggctcaaatcccagctctgcca

SEQ ID NO: 468 - DNA, 176 bp, synthetic construct
  misc_feature 1..176: note = Description of Artificial Sequence: Synthetic oligonucleotide
  source 1..176: mol_type = other DNA
ggctgtatagctcagtggtagagcatttgacttcagaatcctatactcaggggaaggagaactgggggtttctcagtgggtcaaaggacttgtagtggtaaatcaaaagcaactctataagctatgtaacaaactttaaagtcatatgtagctgggttcaaatcctgtttctgcca

SEQ ID NO: 469 - DNA, 79 bp, synthetic construct
  misc_feature 1..79: note = Description of Artificial Sequence: Synthetic oligonucleotide
  source 1..79: mol_type = other DNA
ggctgtatagctcagtggtagagcatttgacttcagctttaaagtcatatgtagctgggttcaaatcctgtttctgcca

SEQ ID NO: 470 - DNA, 72 bp, synthetic construct
  misc_feature 1..72: note = Description of Artificial Sequence: Synthetic oligonucleotide
  source 1..72: mol_type = other DNA
gggggcatagctcagtggtagagcatttgacttcagatcaagaggtccctggttcaaatccaggtgccccct

SEQ ID NO: 471 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcagatcaagaggtccctggttcaaatccaggtgcccccc

SEQ ID NO: 472 - DNA, 72 bp, synthetic construct
  misc_feature 1..72: note = Description of Artificial Sequence: Synthetic oligonucleotide
  source 1..72: mol_type = other DNA
gggggtatagcttagcggtagagcatttgacttcagatcaagaggtccccggttcaaatccgggtgccccct

SEQ ID NO: 473 - DNA, 72 bp, synthetic construct
  misc_feature 1..72: note = Description of Artificial Sequence: Synthetic oligonucleotide
  source 1..72: mol_type = other DNA
gggggtatagcttaggggtagagcatttgacttcagatcaaaaggtccctggttcaaatccaggtgcccctt

SEQ ID NO: 474 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcagatcaagaggtccccagttcaaatctgggtgccccct

SEQ ID NO: 475 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcagatcaagaagtccccggttcaaatccgggtgccccct

SEQ ID NO: 476 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcagatcaagaggtctctggttcaaatccaggtgccccct

SEQ ID NO: 477 - DNA, 72 bp, synthetic construct
  misc_feature 1..72: note = Description of Artificial Sequence: Synthetic oligonucleotide
  source 1..72: mol_type = other DNA
gggggtatagctcaggggtagagcacttgacttcagatcaagaagtccttggttcaaatccaggtgccccct

SEQ ID NO: 478 - DNA, 72 bp, synthetic construct
  misc_feature 1..72: note = Description of Artificial Sequence: Synthetic oligonucleotide
  source 1..72: mol_type = other DNA
ggggatatagctcaggggtagagcatttgacttcagatcaagaggtccccggttcaaatccgggtgcccccc

SEQ ID NO: 479 - DNA, 72 bp, synthetic construct
  misc_feature 1..72: note = Description of Artificial Sequence: Synthetic oligonucleotide
  source 1..72: mol_type = other DNA
gggggtatagttcaggggtagagcatttgacttcagatcaagaggtccctggttcaaatccaggtgccccct

SEQ ID NO: 480 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcaaatcaagaggtccctgattcaaatccaggtgccccct

SEQ ID NO: 481 - DNA, 72 bp, synthetic construct
  misc_feature 1..72: note = Description of Artificial Sequence: Synthetic oligonucleotide
  source 1..72: mol_type = other DNA
gggcgtatagctcaggggtagagcatttgacttcagatcaagaggtccccagttcaaatctgggtgccccct

SEQ ID NO: 482 - DNA, 72 bp, synthetic construct
  misc_feature 1..72: note = Description of Artificial Sequence: Synthetic oligonucleotide
  source 1..72: mol_type = other DNA
gggggtatagctcacaggtagagcatttgacttcagatcaagaggtccccggttcaaatctgggtgccccct

SEQ ID NO: 483 - DNA, 70 bp, synthetic construct
  misc_feature 1..70: note = Description of Artificial Sequence: Synthetic oligonucleotide
  source 1..70: mol_type = other DNA
gggcgtatagctcaggggtagagcatttgacttcagatcaagaggtccccagttcaaatctgggtgccca

SEQ ID NO: 484 - DNA, 72 bp, synthetic construct
  misc_feature 1..72: note = Description of Artificial Sequence: Synthetic oligonucleotide
  source 1..72: mol_type = other DNA
gggggtatagctcacaggtagagcatttgacttcagatcaagaggtccccggttcaaatccggttactccct

SEQ ID NO: 485 - DNA, 72 bp, synthetic construct
  misc_feature 1..72: note = Description of Artificial Sequence: Synthetic oligonucleotide
  source 1..72: mol_type = other DNA
gggggtagggctcagggatagagcatttgacttcagatcaagaggtccccggttcgaatctaggtgccccct

SEQ ID NO: 486 - DNA, 66 bp, synthetic construct
  misc_feature 1..66: note = Description of Artificial Sequence: Synthetic oligonucleotide
  source 1..66: mol_type = other DNA
ggtatatctcagggggcagagcatttgacttcagatcaagaggtccccggttgaaatccgggtgct

SEQ ID NO: 487 - DNA, 72 bp, synthetic construct
  misc_feature 1..72: note = Description of Artificial Sequence: Synthetic oligonucleotide
  source 1..72: mol_type = other DNA
gggggtatagctcaggggtagagcacttgacttcagatcaagaggtccctggttcaaatccaggtgccccct

SEQ ID NO: 488 - DNA, 72 bp, synthetic construct
  misc_feature 1..72: note = Description of Artificial Sequence: Synthetic oligonucleotide
  source 1..72: mol_type = other DNA
gggggtatagctcagtggtagagcatttgacttcagatcaagaggtccctggttcaaatccgggtgccccct

SEQ ID NO: 489 - DNA, 73 bp, synthetic construct
  misc_feature 1..73: note = Description of Artificial Sequence: Synthetic oligonucleotide
  source 1..73: mol_type = other DNA
gggggtatagctcagtgggtagagcatttgacttcagatcaagaggtccccggttcaaatccgggtgccccct

SEQ ID NO: 490 - DNA, 72 bp, synthetic construct
  misc_feature 1..72: note = Description of Artificial Sequence: Synthetic oligonucleotide
  source 1..72: mol_type = other DNA
gggggtgtagctcagtggtagagcatttgacttcagatcaagaggtccctggttcaaatccaggtgccccct

SEQ ID NO: 491 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcagatcaagaggtccccggttcaaatccgggtgccccct

SEQ ID NO: 492 - DNA, 72 bp, synthetic construct
  misc_feature 1..72: note = Description of Artificial Sequence: Synthetic oligonucleotide
  source 1..72: mol_type = other DNA
gggggtatagctcaggggtagagcatttgacttcagatcaagaggtccctggttcaaatccaggtgccccct

SEQ ID NO: 493 - DNA, 72 bp, synthetic construct
  misc_feature 1..72: note = Description of Artificial Sequence: Synthetic oligonucleotide
  source 1..72: mol_type = other DNA
gacctcgtggcgcaatggtagcgcgtctgacttcagatcagaaggttgcgtgttcaaatcacgtcggggtca

SEQ ID NO: 494 - DNA, 72 bp, synthetic construct
  misc_feature 1..72: note = Description of Artificial Sequence: Synthetic oligonucleotide
  source 1..72: mol_type = other DNA
gacctcgtggcacaatggtagcacgtctgacttcagatcagaaggttgcgtgttcaaatcacgtcggggtca

SEQ ID NO: 495 - DNA, 106 bp, synthetic construct
  misc_feature 1..106: note = Description of Artificial Sequence: Synthetic oligonucleotide
  source 1..106: mol_type = other DNA
gaagcggtggctcaatggtagagctttcgacttcaattaaatcttggaaattccacggaataagattgcaatcgaagggttgcaggttcaattcctgtccgtttca

SEQ ID NO: 496 - DNA, 72 bp, synthetic construct
  misc_feature 1..72: note = Description of Artificial Sequence: Synthetic oligonucleotide
  source 1..72: mol_type = other DNA
gaagcggtggctcaatggtagagctttcgacttcaaatcgaagggttgcaggttcaattcctgtccgtttca

SEQ ID NO: 497 - DNA, 72 bp, synthetic construct
  misc_feature 1..72: note = Description of Artificial Sequence: Synthetic oligonucleotide
  source 1..72: mol_type = other DNA
ggcctcatggtgcaacagtagtgtgtctgacttcagatcagaaggttgtatgttcaaatcacataggggtca

SEQ ID NO: 498 - DNA, 72 bp, synthetic construct
  misc_feature 1..72: note = Description of Artificial Sequence: Synthetic oligonucleotide
  source 1..72: mol_type = other DNA
gacctcgtggtgaaatggtagcatgtttgacttcaaatcaggaggttgtgtgttcaagtcacatcagggtca

SEQ ID NO: 499 - DNA, 72 bp, synthetic construct
  misc_feature 1..72: note = Description of Artificial Sequence: Synthetic oligonucleotide
  source 1..72: mol_type = other DNA
gaccttgtggcgcaatggtagcatgtttgacttcaaatcaggaggttgtgtgttcaagtcacatcagggtca

SEQ ID NO: 500 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgacttcagatcagaaggctgcgtgttcgaatcacgccggggtca

SEQ ID NO: 501 - DNA, 72 bp, synthetic construct
  misc_feature 1..72: note = Description of Artificial Sequence: Synthetic oligonucleotide
  source 1..72: mol_type = other DNA
gaccttgtggctcaatggtagcgcatctgacttcagatcaggaggttgcacgttcaaatcatgccggggtca

SEQ ID NO: 502 - DNA, 72 bp, synthetic construct
  misc_feature 1..72: note = Description of Artificial Sequence: Synthetic oligonucleotide
  source 1..72: mol_type = other DNA
gaccttgtggcgcaacggtagcgcgtctgacttcagatcagaaggttgcgtgttcaaatcacgtcggggtca

SEQ ID NO: 503 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgacttcagatcagaaggttgcgtattcaaatcacgtcggggtca

SEQ ID NO: 504 - DNA, 72 bp, synthetic construct
  misc_feature 1..72: note = Description of Artificial Sequence: Synthetic oligonucleotide
  source 1..72: mol_type = other DNA
gacctcgtggcgcaacggcagcgcgtctgacttcacattagaaggttgcgtgttcaaatcacgtcggggtca

SEQ ID NO: 505 - DNA, 72 bp, synthetic construct
  misc_feature 1..72: note = Description of Artificial Sequence: Synthetic oligonucleotide
  source 1..72: mol_type = other DNA
gacctcatggcgcaacggtagcgcgtctgacttcagatcagaaggttgcgtgttcaaatcacatcggggtca

SEQ ID NO: 506 - DNA, 72 bp, synthetic construct
  misc_feature 1..72: note = Description of Artificial Sequence: Synthetic oligonucleotide
  source 1..72: mol_type = other DNA
gacctcgtggtgcaacggtagcgcgtatgatttcagatcagaaggttgcgtgttcaaatcacgtcggggtca

SEQ ID NO: 507 - DNA, 72 bp, synthetic construct
  misc_feature 1..72: note = Description of Artificial Sequence: Synthetic oligonucleotide
  source 1..72: mol_type = other DNA
gacctcgtagcgcaacggtagcgcgtctgacttcagatcagaaggttgcgtgttcaaatcacgtcggggtca

SEQ ID NO: 508 - DNA, 76 bp, synthetic construct
  misc_feature 1..76: note = Description of Artificial Sequence: Synthetic oligonucleotide
  source 1..76: mol_type = other DNA
aggggtatagctcaattggcagagcgtcggtcttcaaaaccgaaggttgtaggttcgattcctactgcccctgcca

SEQ ID NO: 509 - DNA, 72 bp, synthetic construct
  misc_feature 1..72: note = Description of Artificial Sequence: Synthetic oligonucleotide
  source 1..72: mol_type = other DNA
gacctcatggcgcaacggtagcgcgtctgacttcagatcagaaggttgcgtgttcaaatcacgtcggggtca

SEQ ID NO: 510 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctaacttcagatcagaaggttgcgtgttcaaatcacgtcggggtca

SEQ ID NO: 511 - DNA, 73 bp, synthetic construct
  misc_feature 1..73: note = Description of Artificial Sequence: Synthetic oligonucleotide
  source 1..73: mol_type = other DNA
acgggagtagctcagttggtagagcaccggtcttcaaaaccgggtgtcgggagttcgagcctctcctcccgtg

SEQ ID NO: 512 - DNA, 72 bp, synthetic construct
  misc_feature 1..72: note = Description of Artificial Sequence: Synthetic oligonucleotide
  source 1..72: mol_type = other DNA
gacctcgtggcgcaacggtagcgcgtctgacttcagatcagaaggttgcatgttcaaatcacgtcggggtca

SEQ ID NO: 513 - DNA, 72 bp, synthetic construct
  misc_feature 1..72: note = Description of Artificial Sequence: Synthetic oligonucleotide
  source 1..72: mol_type = other DNA
gactccgtggcgcaacggtagcgcgtccgacttcagatcggaaggttgcgtgttcaaatcacgtcggggtca

SEQ ID NO: 514 - DNA, 72 bp, synthetic construct
  misc_feature 1..72: note = Description of Artificial Sequence: Synthetic oligonucleotide
  source 1..72: mol_type = other DNA
gactccgtggcgcaacggtagcgcgtctgacttcagatcagaaggttgcgtgttcaaatcacgtcggggtca

SEQ ID NO: 515 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcgcgtctgactccagatcagaaggttgcgtgttcaaatcacgtcggggtca

SEQ ID NO: 516 - DNA, 72 bp, synthetic construct
  misc_feature 1..72: note = Description of Artificial Sequence: Synthetic oligonucleotide
  source 1..72: mol_type = other DNA
ggcctcgtggcgcaacggtagcacgtctgactccagatcagaaggttgcgtgttcaaatcacgtcggggtca

SEQ ID NO: 517 - DNA, 73 bp, synthetic construct
  misc_feature 1..73: note = Description of Artificial Sequence: Synthetic oligonucleotide
  source 1..73: mol_type = other DNA
cggcctcgtggcgcaacggtagcacgtctgacttcagatcagaaggttgcgtgttcaaatcacgtcggggtca

SEQ ID NO: 518 - DNA, 72 bp, synthetic construct
  misc_feature 1..72: note = Description of Artificial Sequence: Synthetic oligonucleotide
  source 1..72: mol_type = other DNA
ggcctcgtcgcgcaacggtagcgcgtctgactccagatcagaaggttgcgtgttcaaatcacgtcggggtca

SEQ ID NO: 519 - DNA, 72 bp, synthetic construct
  misc_feature 1..72: note = Description of Artificial Sequence: Synthetic oligonucleotide
  source 1..72: mol_type = other DNA
ggcctcgtcgcgcaacggtagcgcgtctgacttcagatcagaaggttgcgtgttcaaatcacgtcggggtca

SEQ ID NO: 520 - DNA, 72 bp, synthetic construct
  misc_feature 1..72: note = Description of Artificial Sequence: Synthetic oligonucleotide
  source 1..72: mol_type = other DNA
ggcctcgtcgcgcaacggtagcacgtctgactccagatcagaaggttgcgtgttcaaatcacgtcggggtca

SEQ ID NO: 521 - DNA, 72 bp, synthetic construct
  misc_feature 1..72: note = Description of Artificial Sequence: Synthetic oligonucleotide
  source 1..72: mol_type = other DNA
ggcctcgtcgcgcaacggtagcacgtctgacttcagatcagaaggttgcgtgttcaaatcacgtcggggtca

SEQ ID NO: 522 - DNA, 73 bp, synthetic construct
  misc_feature 1..73: note = Description of Artificial Sequence: Synthetic oligonucleotide
  source 1..73: mol_type = other DNA
taatacgactcactatagagcgctccggtttttctgtgctgaacctcaggggacgccgacacacgtacacgtc

SEQ ID NO: 523 - DNA, 75 bp, synthetic construct
  misc_feature 1..75: note = Description of Artificial Sequence: Synthetic oligonucleotide
  source 1..75: mol_type = other DNA
gttaagatggcagagcctggtaattgcatcaaacttaaaattttataatcagaggttcaactcctcttcttaaca

SEQ ID NO: 524 - DNA, 75 bp, synthetic construct
  misc_feature 1..75: note = Description of Artificial Sequence: Synthetic oligonucleotide
  source 1..75: mol_type = other DNA
gttaagatggcagagcccggcaattgcatcagacttaaaactttataatcagaggttcaactcctctcattaaca

SEQ ID NO: 525 - DNA, 82 bp, synthetic construct
  misc_feature 1..82: note = Description of Artificial Sequence: Synthetic oligonucleotide
  source 1..82: mol_type = other DNA
ggtagcgtggccgagcggtctaaggcgctggatttcagctccagtctcttcgggggcgtgggttcaaatcccaccgctgcca

SEQ ID NO: 526 - DNA, 82 bp, synthetic construct
  misc_feature 1..82: note = Description of Artificial Sequence: Synthetic oligonucleotide
  source 1..82: mol_type = other DNA
ggtagcgtggccgagtggtctaagacgctggatttcagctccagtctcttcgggggcgtgggtttgaatcccaccgctgcca

SEQ ID NO: 527 - DNA, 73 bp, synthetic construct
  misc_feature 1..73: note = Description of Artificial Sequence: Synthetic oligonucleotide
  source 1..73: mol_type = other DNA
gggccagtggctcaatggataatgcgtctgacttcaaatcagaagattccagccttgactcctggctggctca

SEQ ID NO: 528 - DNA, 82 bp, synthetic construct
  misc_feature 1..82: note = Description of Artificial Sequence: Synthetic oligonucleotide
  source 1..82: mol_type = other DNA
ggtagggtggccgagcggtctaaggcactgtatttcaactccagtctcttcagaggcatgggtttgaatcccactgctgcca

SEQ ID NO: 529 - DNA, 65 bp, synthetic construct
  misc_feature 1..65: note = Description of Artificial Sequence: Synthetic oligonucleotide
  source 1..65: mol_type = other DNA
gccgagcggtctaaggctccggatttcagcgccggtgtcttcggaggcatgggttcgaattccac

SEQ ID NO: 530 - DNA, 106 bp, synthetic construct
  misc_feature 1..106: note = Description of Artificial Sequence: Synthetic oligonucleotide
  source 1..106: mol_type = other DNA
gtcaggatggccgagtggtctaaggcgccagacttcagctaagcttcctccgcggtggggattctggtctccaatggaggcgtgggttcgaatcccacttctgaca

SEQ ID NO: 531 - DNA, 83 bp, synthetic construct
  misc_feature 1..83: note = Description of Artificial Sequence: Synthetic oligonucleotide
  source 1..83: mol_type = other DNA
gtcaggatggccgagtggtctaaggcgccagacttcagttctggtctccaatggaggcgtgggttcgaatcccacttctgaca

SEQ ID NO: 532 - DNA, 105 bp, synthetic construct
  misc_feature 1..105: note = Description of Artificial Sequence: Synthetic oligonucleotide
  source 1..105: mol_type = other DNA
gtcaggatggccgagtggtctaaggcgccagacttcagcttggcttcctcgtgttgaggattctggtctccaatggaggcgtgggttcgaatcccacttctgaca

SEQ ID NO: 533 - DNA, 83 bp, synthetic construct; identical to SEQ ID NO: 531
  misc_feature 1..83: note = Description of Artificial Sequence: Synthetic oligonucleotide
  source 1..83: mol_type = other DNA

SEQ ID NO: 534 - DNA, 108 bp, synthetic construct
  misc_feature 1..108: note = Description of Artificial Sequence: Synthetic oligonucleotide
  source 1..108: mol_type = other DNA
gtcaggatggccgagtggtctaaggcgccagacttcagcttactgcttcctgtgttcgggtcttctggtctccgtatggaggcgtgggttcgaatcccacttctgaca

SEQ ID NO: 535 - DNA, 84 bp, synthetic construct
  misc_feature 1..84: note = Description of Artificial Sequence: Synthetic oligonucleotide
  source 1..84: mol_type = other DNA
gtcaggatggccgagtggtctaaggcgccagacttcagttctggtctccgtatggaggcgtgggttcgaatcccacttctgaca

SEQ ID NO: 536 - DNA, 107 bp, synthetic construct
  misc_feature 1..107: note = Description of Artificial Sequence: Synthetic oligonucleotide
  source 1..107: mol_type = other DNA
gtcaggatggccgagtggtctaaggcgccagacttcagttgctacttcccaggtttggggcttctggtctccgcatggaggcgtgggttcgaatcccacttctgaca

SEQ ID NO: 537 - DNA, 84 bp, synthetic construct
  misc_feature 1..84: note = Description of Artificial Sequence: Synthetic oligonucleotide
  source 1..84: mol_type = other DNA
gtcaggatggccgagtggtctaaggcgccagacttcagttctggtctccgcatggaggcgtgggttcgaatcccacttctgaca

SEQ ID NO: 538 - DNA, 106 bp, synthetic construct
  misc_feature 1..106: note = Description of Artificial Sequence: Synthetic oligonucleotide
  source 1..106: mol_type = other DNA
gtcaggatggccgagtggtctaaggcgccagacttcaggtaagcaccttgcctgcgggctttctggtctccggatggaggcgtgggttcgaatcccacttctgaca

SEQ ID NO: 539 - DNA, 85 bp, synthetic construct
  misc_feature 1..85: note = Description of Artificial Sequence: Synthetic oligonucleotide
  source 1..85: mol_type = other DNA
gtcaggatggccgagtggtctaaggcgccagacttcagtttctggtctccggatggaggcgtgggttcgaatcccacttctgaca

SEQ ID NO: 540 - DNA, 74 bp, synthetic construct
  misc_feature 1..74: note = Description of Artificial Sequence: Synthetic oligonucleotide
  source 1..74: mol_type = other DNA
gcctccttagtgcagtaggtagcgcatcagtcttcaaatctgaatggtcctgagttcaagcctcagagggggca

SEQ ID NO: 541 - DNA, 84 bp, synthetic construct
  misc_feature 1..84: note = Description of Artificial Sequence: Synthetic oligonucleotide
  source 1..84: mol_type = other DNA
gtcaggatggccgagcagtcttaaggcgctgcgtttcaatcgcaccctccgctggaggcgtgggttcgaatcccacttttgaca

SEQ ID NO: 542 - DNA, 60 bp, synthetic construct
  misc_feature 1..60: note = Description of Artificial Sequence: Synthetic oligonucleotide
  source 1..60: mol_type = other DNA
ggttccatggtgtaatggtgagcactctggacttcaaatccagaagtagtgctggaacaa

SEQ ID NO: 543 - DNA, 83 bp, synthetic construct
  misc_feature 1..83: note = Description of Artificial Sequence: Synthetic oligonucleotide
  source 1..83: mol_type = other DNA
gtcagggtggctgagcagtctgaggggctgcgtttcagtcgcagtctgccctggaggcgtgggttcgaatcccactcctgaaa

SEQ ID NO: 544 - DNA, 83 bp, synthetic construct
  misc_feature 1..83: note = Description of Artificial Sequence: Synthetic oligonucleotide
  source 1..83: mol_type = other DNA
accaggatggccgagtggttaaggcgttggacttcagatccaatggacatatgtccgcgtgggttcgaaccccactcctggta

SEQ ID NO: 545 - DNA, 83 bp, synthetic construct
  misc_feature 1..83: note = Description of Artificial Sequence: Synthetic oligonucleotide
  source 1..83: mol_type = other DNA
accgggatggccgagtggttaaggcgttggacttcagatccaatgggctggtgcccgcgtgggttcgaaccccactctcggta

SEQ ID NO: 546 - DNA, 83 bp, synthetic construct
  misc_feature 1..83: note = Description of Artificial Sequence: Synthetic oligonucleotide
  source 1..83: mol_type = other DNA
accagaatggccgagtggttaaggcgttggacttcagatccaatggattcatatccgcgtgggttcgaaccccacttctggta

SEQ ID NO: 547 - DNA, 83 bp, synthetic construct
  misc_feature 1..83: note = Description of Artificial Sequence: Synthetic oligonucleotide
  source 1..83: mol_type = other DNA
accgggatggctgagtggttaaggcgttggacttcagatccaatggacaggtgtccgcgtgggttcgagccccactcccggta

SEQ ID NO: 548 - DNA, 83 bp, synthetic construct
  misc_feature 1..83: note = Description of Artificial Sequence: Synthetic oligonucleotide
  source 1..83: mol_type = other DNA
actcatttggctgagtggttaaggcattggacttcagatccaatggagtagtggctgtgtgggtttaaaccccactactggta

SEQ ID NO: 549 - DNA, 69 bp, synthetic construct
  misc_feature 1..69: note = Description of Artificial Sequence: Synthetic oligonucleotide
  source 1..69: mol_type = other DNA
gagaaagtcatcgtagttacgaagttggcttcaacccagttttgggaggttcaattccttcctttctct

SEQ ID NO: 550 - DNA, 84 bp, synthetic construct
  misc_feature 1..84: note = Description of Artificial Sequence: Synthetic oligonucleotide
  source 1..84: mol_type = other DNA
accaggatggccaagtagttaaaggcactggacttcagagccaatggacatatgtctgtgtgggtttgaaccccactcctggtg

SEQ ID NO: 551 - DNA, 82 bp, synthetic construct
  misc_feature 1..82: note = Description of Artificial Sequence: Synthetic oligonucleotide
  source 1..82: mol_type = other DNA
ggtagcgtggccgagcggtctaaggcgctggatttcagctccagtctcttcggaggcgtgggttcgaatcccaccgctgcca

SEQ ID NO: 552 - DNA, 82 bp, synthetic construct
  misc_feature 1..82: note = Description of Artificial Sequence: Synthetic oligonucleotide
  source 1..82: mol_type = other DNA
ggtagtgtggccgagcggtctaaggcgctggatttcagctccagtctcttcgggggcgtgggttcgaatcccaccactgcca

SEQ ID NO: 553 - DNA, 82 bp, synthetic construct
  misc_feature 1..82: note = Description of Artificial Sequence: Synthetic oligonucleotide
  source 1..82: mol_type = other DNA
ggtagcgtggccgagtggtctaaggcgctggatttcagctccagtcatttcgatggcgtgggttcgaatcccaccgctgcca

SEQ ID NO: 554 - DNA, 82 bp, synthetic construct
  misc_feature 1..82: note = Description of Artificial Sequence: Synthetic oligonucleotide
  source 1..82: mol_type = other DNA
ggtagtgtggttgaatggtctaaggcactgaatttcagctccagtctctttggggacgtgggtttaaatcccactgctgcaa

SEQ ID NO: 555 - DNA, 70 bp, synthetic construct
  misc_feature 1..70: note = Description of Artificial Sequence: Synthetic oligonucleotide
  source 1..70: mol_type = other DNA
gagaaggtcacagaggttatgggattggctttaaaccagtctgtggggggttcgattccctcctttttca

SEQ ID NO: 556 - DNA, 70 bp, synthetic construct
  misc_feature 1..70: note = Description of Artificial Sequence: Synthetic oligonucleotide
  source 1..70: mol_type = other DNA
gagaaggtcatagaggttatgggattggctttaaaccagtctctggggggttcgattccctcctttttca

SEQ ID NO: 557 - DNA, 69 bp, synthetic construct
  misc_feature 1..69: note = Description of Artificial Sequence: Synthetic oligonucleotide
  source 1..69: mol_type = other DNA
gaaaaagtcataggggttatgaggctggctttaaaccagccttaggaggttcaattccttccttttttg

SEQ ID NO: 558 - DNA, 74 bp, synthetic construct
  misc_feature 1..74: note = Description of Artificial Sequence: Synthetic oligonucleotide
  source 1..74: mol_type = other DNA
ggccggttagctcagttggttagagcgtgctgctttaaatgccagggtcgaggtttcgatccccgtacgggcct

SEQ ID NO: 559 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactttaaatccattggggtttccccgcgcaggttcgaatcctgccgactacg

SEQ ID NO: 560 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactttaaatccattggggtttccccacgcaggttcgaatcctgccgactacg

SEQ ID NO: 561 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggtgatggactttaaacccattggggtctccccgcgcaggttcgaatcctgccgactacg

SEQ ID NO: 562 - DNA, 72 bp, synthetic construct
  misc_feature 1..72: note = Description of Artificial Sequence: Synthetic oligonucleotide
  source 1..72: mol_type = other DNA
gggtgtatggctcaggggtagagaatttgactttagatcaagaggtccctggttcaaatccaggtgccccct

SEQ ID NO: 563 - DNA, 78 bp, synthetic construct
  misc_feature 1..78: note = Description of Artificial Sequence: Synthetic oligonucleotide
  source 1..78: mol_type = other DNA
agttgtagctgagtggttaaggcaacgagctttaaattcgttggtttctctctgtgcaggtttgaatcctgctaatta

SEQ ID NO: 564 - DNA, 70 bp, synthetic construct
  misc_feature 1..70: note = Description of Artificial Sequence: Synthetic oligonucleotide
  source 1..70: mol_type = other DNA
caagaaattcatagaggttatgggattggctttaaaccagtttcaggaggttcgattccttcctttttgg

SEQ ID NO: 565 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggcgttggactttaaatccaatggggtctccccgcgcaggttcgaatcctgctcacagcg

SEQ ID NO: 566 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggcgttggactttaaatccaatggggtctccccgcgcaggttcaaatcctgctcacagcg

SEQ ID NO: 567 - DNA, 82 bp, synthetic construct
  misc_feature 1..82: note = Description of Artificial Sequence: Synthetic oligonucleotide
  source 1..82: mol_type = other DNA
gctgtgatggccgagtggttaaggtgttggactttaaatccaatgggggttccccgcgcaggttcaaatcctgctcacagcg

SEQ ID NO: 568 - DNA, 82 bp, synthetic construct
  misc_feature 1..82: note = Description of Artificial Sequence: Synthetic oligonucleotide
  source 1..82: mol_type = other DNA
gtcacggtggccgagtggttaaggcgttggactttaaatccaatggggtttccccgcacaggttcgaatcctgttcgtgacg

SEQ ID NO: 569 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggactttaaatccattgtgctctgcacgcgtgggttcgaatcccaccctcgtcg

SEQ ID NO: 570 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggactttaaatccattgtgctctgcacgcgtgggttcgaatcccaccttcgtcg

SEQ ID NO: 571 - DNA, 74 bp, synthetic construct
  misc_feature 1..74: note = Description of Artificial Sequence: Synthetic oligonucleotide
  source 1..74: mol_type = other DNA
ggccggttagctcagttggttagagcgtgctttaactaatgccagggtcgaggtttcgatccccgtacgggcct

SEQ ID NO: 572 - DNA, 82 bp, synthetic construct
  misc_feature 1..82: note = Description of Artificial Sequence: Synthetic oligonucleotide
  source 1..82: mol_type = other DNA
gacgaggtggccgagtggttaaggcgatggactttaaatccattgtgctctgcacacgtgggttcgaatcccatcctcgtcg

SEQ ID NO: 573 - DNA, 78 bp, synthetic construct
  misc_feature 1..78: note = Description of Artificial Sequence: Synthetic oligonucleotide
  source 1..78: mol_type = other DNA
gaggcctggccgagtggttaaggcgatggactttaaatccattgtgctctgcacgcgtgggttcgaatcccatcctcg

SEQ ID NO: 574 - DNA, 82 bp, synthetic construct
  misc_feature 1..82: note = Description of Artificial Sequence: Synthetic oligonucleotide
  source 1..82: mol_type = other DNA
gcagcgatggccgagtggttaaggcgttggactttaaatccaatggggtctccccgcgcaggttcgaaccctgctcgctgcg

SEQ ID NO: 575 - DNA, 82 bp, synthetic construct; identical to SEQ ID NO: 559
  misc_feature 1..82: note = Description of Artificial Sequence: Synthetic oligonucleotide
  source 1..82: mol_type = other DNA

SEQ ID NO: 576 - DNA, 82 bp, synthetic construct
  misc_feature 1..82: note = Description of Artificial Sequence: Synthetic oligonucleotide
  source 1..82: mol_type = other DNA
gtagtcgtggccgagtggttaaggcgatggactttaaatccattggggtctccccgcgcaggttcgaatcctgccgactacg

SEQ ID NO: 577 - DNA, 82 bp, synthetic construct
  misc_feature 1..82: note = Description of Artificial Sequence: Synthetic oligonucleotide
  misc_feature 1..82: note = Description of Combined DNA/RNA Molecule: Synthetic oligonucleotide
  source 1..82: mol_type = other DNA
gtagtcgtggccgagtggttaaggcgatggactttaaatccattggggtttccccgcgcaggttcgaatcctgtcggctacg

SEQ ID NO: 578 - RNA, 82 nt, synthetic construct
  source 1..82: mol_type = other RNA
gtagtcgtggccgagtggttaaggcgatggactttaaatccattggggtctccccgcgcaggttcgaatcctgccgactacg

SEQ ID NO: 579 - RNA, 82 nt, synthetic construct
  source 1..82: mol_type = other RNA
gtagtcgtggccgagtggttaaggcgatggactttaaatccattggggtttccccgcgcaggttcgaatcctgtcggctacg

SEQ ID NO: 580 - protein, 10 aa, synthetic construct
  REGION 1..10: note = Description of Artificial Sequence: Synthetic oligonucleotide
NIETFHTAQK

SEQ ID NO: 581 - protein, 9 aa, synthetic construct
  REGION 1..9: note = Description of Artificial Sequence: Synthetic peptide
IETFHTAQK

SEQ ID NO: 582 - protein, 9 aa, synthetic construct
  REGION 1..9: note = Description of Artificial Sequence: Synthetic peptide
NIETFHTAQ

SEQ ID NO: 583 - protein, 8 aa, synthetic construct
  REGION 1..8: note = Description of Artificial Sequence: Synthetic peptide
ETFHTAQK

SEQ ID NO: 584 - protein, 8 aa, synthetic construct
  REGION 1..8: note = Description of Artificial Sequence: Synthetic peptide
IETFHTAQ

SEQ ID NO: 585 - DNA, 27 bp, synthetic construct
  misc_feature 1..27: note = Description of Artificial Sequence: Synthetic oligonucleotide
  misc_feature 1..6: note = a, c, t, g, unknown or other
  misc_feature 22..27: note = a, c, t, g, unknown or other
  source 1..27: mol_type = other DNA
nnnnnngtattcatcgaagacnnnnnn

SEQ ID NO: 586 - DNA, 27 bp, synthetic construct
  misc_feature 1..27: note = Description of Artificial Sequence: Synthetic oligonucleotide
  misc_feature 1..6: note = a, c, t, g, unknown or other
  misc_feature 22..27: note = a, c, t, g, unknown or other
  source 1..27: mol_type = other DNA
nnnnnngtcttcgatgaatacnnnnnn

SEQ ID NO: 587 - RNA, 72 nt, synthetic construct
  source 1..72: mol_type = other RNA
  misc_feature 34: map = 34 is "t"
acctcgtggcgcaacggttagcgcgtctgacttcagatcagaaggctccgggttcgaatcccggcggggtca

SEQ ID NO: 588 - protein, 10 aa, synthetic construct
  REGION 1..10: note = Description of Artificial Sequence: Synthetic peptide
  MOD_RES 1: note = Any amino acid
XIETFHTAQK

SEQ ID NO: 589 - RNA, 71 nt, Homo sapiens
  source 1..71: mol_type = tRNA
gcattggtggttcagtggtagaattctcgccttcaacgcgggagacccgggttcaattcccggccaatgca

SEQ ID NO: 590 - RNA, 71 nt, Homo sapiens
  source 1..71: mol_type = tRNA
gcgccgctggtgtagtggtatcatgcaagatttcaattcttgcgacccgggttcgattcccgggcggcgca

SEQ ID NO: 591 - RNA, 71 nt, Homo sapiens
  source 1..71: mol_type = tRNA
gcattggtggttcaatggtagaattctcgccttcaacgcaggagacccaggttcgattcctggccaatgca

SEQ ID NO: 592 - RNA, 71 nt, Homo sapiens
  source 1..71: mol_type = tRNA
gcgttggtggtttagtggtagaattctcgccttcaatgcgggagacccgggttcaattcccggccactgca

SEQ ID NO: 593 - RNA, 71 nt, Homo sapiens
  source 1..71: mol_type = tRNA
gccttggtggtgcagtggtagaattctcgccttcaacgtgggagacccgggttcaattcccggccaatgca

SEQ ID NO: 594 - RNA, 61 nt, Homo sapiens
  source 1..61: mol_type = tRNA
ggtggttcagtggtagaattctcgccttcaacgcgggagacccgggtttaattcccggtca

SEQ ID NO: 595 - RNA, 61 nt, Homo sapiens
  source 1..61: mol_type = tRNA
gtggtctagtggttaggattcagcgcttcaaccgccgcagcccgggttcgattcccggtca

SEQ ID NO: 596 - RNA, 71 nt, Homo sapiens
  source 1..71: mol_type = tRNA
gcgtcagtggtttagtggtggaattcctgccttcaatgcacgagatccgtgttcaactcctggttggtgca

SEQ ID NO: 597 - RNA, 72 nt, Homo sapiens
  source 1..72: mol_type = tRNA
gcgtcagtggttttagtggtggaattcctgccttcaatgcacgagatccgtgttcaactcctggttggtgca

SEQ ID NO: 598 - RNA, 71 nt, Homo sapiens
  source 1..71: mol_type = tRNA
gcgttggcagttcagtggtagaattctcgccttcaacccgggagacctggattccatttccggcaaatgca

SEQ ID NO: 599 - RNA, 71 nt, Homo sapiens
  source 1..71: mol_type = tRNA
gcatgggtggttcagtggtagaattctcgccttcaacgcgggaggcccgggttcgattcccggcccatgca

SEQ ID NO: 600 - RNA, 71 nt, Homo sapiens
  source 1..71: mol_type = tRNA
gcattggtggttcagtggtagaattctcgccttcaacgcgggaggcccgggttcgattcccggccaatgca

SEQ ID NO: 601 - RNA, 71 nt, Homo sapiens
  source 1..71: mol_type = tRNA
gcattggtggttcagtggtagaattctcgccttcaacgcgggaggcccgggtttgattcccggccagtgca

SEQ ID NO: 602 - RNA, 71 nt, Homo sapiens
  source 1..71: mol_type = tRNA
gcataggtggttcagtggtagaattcttgccttcaacgcaggaggcccaggtttgattcctggcccatgca

SEQ ID NO: 603 - RNA, 71 nt, Homo sapiens
  source 1..71: mol_type = tRNA
gcattggtggttcagtggtagaattctcgccttcaatgcgggcggccgggcttcgattcctggccaatgca

SEQ ID NO: 604 - RNA, 71 nt, Homo sapiens
  source 1..71: mol_type = tRNA
gcatgggtgattcagtggtagaattttcaccttcaatgcaggaggtccaggttcatttcctggcctatgca

SEQ ID NO: 605 - RNA, 72 nt, Homo sapiens
  source 1..72: mol_type = tRNA
gcgttggtggtatagtggttagcatagctgccttcaaagcagttgacccgggttcgattcccggccaacgca

SEQ ID NO: 606 - RNA, 72 nt, Homo sapiens
  source 1..72: mol_type = tRNA
gcgttggtggtatagtggtgagcatagctgccttcaaagcagttgacccgggttcgattcccggccaacgca

SEQ ID NO: 607 - RNA, 72 nt, Homo sapiens
  source 1..72: mol_type = tRNA
gcgttggtggtatagtggtaagcatagctgccttcaaagcagttgacccgggttcgattcccggccaacgca

SEQ ID NO: 608 - RNA, 72 nt, Homo sapiens
  source 1..72: mol_type = tRNA
gcgttggtggtatagtggtgagcatagttgccttcaaagcagttgacccgggctcgattcccgcccaacgca

SEQ ID NO: 609 - RNA, 72 nt, Homo sapiens
  source 1..72: mol_type = tRNA
gcgttggtggtatagtggtgagcatagttgccttcaaagcagttgacccgggctcgattcccggccaacgca

SEQ ID NO: 610 - RNA, 72 nt, synthetic construct
  misc_feature 1..72: note = Description of Artificial Sequence: Synthetic oligonucleotide
  source 1..72: mol_type = other RNA
gacctcgtggcgcaacggcagcgcgtctgacttcagatcagaaggttgcgtgttcaaatcacgtcggggtca

SEQ ID NO: 611 - RNA, 72 nt, synthetic construct
  misc_feature 1..72: note = Description of Artificial Sequence: Synthetic oligonucleotide
  source 1..72: mol_type = other RNA
gacctcgtggcgcaacggtagcgcgtctgacttcagatcagaaggttgcgtgttcaaatcacgtcggggtca

SEQ ID NO: 612 - RNA, 72 nt, synthetic construct
  misc_feature 1..72: note = Description of Artificial Sequence: Synthetic oligonucleotide
  source 1..72: mol_type = other RNA
ggcctcatggtgcaacagtagtgtgtctgacttcagatcagaaggttgtatgttcaaatcacgtaggggtca

SEQ ID NO: 613 - RNA, 72 nt, synthetic construct
  misc_feature 1..72: note = Description of Artificial Sequence: Synthetic oligonucleotide
  source 1..72: mol_type = other RNA
gacctcgtggcgcaatggtagcgcgtctgacttcagatcagaaggttgcgtgttcaagtcacgtcggggtca

SEQ ID NO: 614 - RNA, 72 nt, synthetic construct
  misc_feature 1..72: note = Description of Artificial Sequence: Synthetic oligonucleotide
  source 1..72: mol_type = other RNA
ggcctcgtggcgcaacggtagcgcgtctgacttcagatcagaaggttgcgtgttcaaatcacgtcggggtca

SEQ ID NO: 615 - RNA, 72 nt, synthetic construct
  misc_feature 1..72: note = Description of Artificial Sequence: Synthetic oligonucleotide
  source 1..72: mol_type = other RNA
gacctcgtggcgcaacggtagcgcgtctgacttcagatcagaaggctgcgtgttcgaatcacgtcggggtca

SEQ ID NO: 616 - RNA, 72 nt, synthetic construct
  misc_feature 1..72: note = Description of Artificial Sequence: Synthetic oligonucleotide
  source 1..72: mol_type = other RNA
gacctcgtggcgcaatggtagcgcgtctgacttcagatcagaaggttgcgtgttcaaatcacgtcggggtca

SEQ ID NO: 617 - RNA, 72 nt, synthetic construct
  misc_feature 1..72: note = Description of Artificial Sequence: Synthetic oligonucleotide
  source 1..72: mol_type = other RNA
gacctcgtggcacaatggtagcacgtctgacttcagatcagaaggttgcgtgttcaaatcacgtcggggtca

SEQ ID NO: 618 - RNA, 106 nt, synthetic construct
  misc_feature 1..106: note = Description of Artificial Sequence: Synthetic oligonucleotide
  source 1..106: mol_type = other RNA
gaagcggtggctcaatggtagagctttcgacttcaattaaatcttggaaattccacggaataagattgcaatcgaagggttgcaggttcaattcctgtccgtttca

SEQ ID NO: 619 - RNA, 72 nt, synthetic construct
  misc_feature 1..72: note = Description of Artificial Sequence: Synthetic oligonucleotide
  source 1..72: mol_type = other RNA
gaagcggtggctcaatggtagagctttcgacttcaaatcgaagggttgcaggttcaattcctgtcggtttca

SEQ ID NO: 620 - RNA, 72 nt, synthetic construct
  misc_feature 1..72: note = Description of Artificial Sequence: Synthetic oligonucleotide
  source 1..72: mol_type = other RNA
ggcctcatggtgcaacagtagtgtgtctgacttcagatcagaaggttgtatgttcaaatcacataggggtca

SEQ ID NO: 621 - RNA, 72 nt, synthetic construct
  misc_feature 1..72: note = Description of Artificial Sequence: Synthetic oligonucleotide
  source 1..72: mol_type = other RNA
gacctcgtggtgaaatggtagcatgtttgacttcaaatcaggaggttgtgtgttcaagtcacatcagggtca

SEQ ID NO: 622 - RNA, 72 nt, synthetic construct
  misc_feature 1..72: note = Description of Artificial Sequence: Synthetic oligonucleotide
  source 1..72: mol_type = other RNA
gaccttgtggcgcaatggtagcatgtttgacttcaaatcaggaggttgtgtgttcaagtcacatcagggtca

SEQ ID NO: 623 - RNA, 72 nt, synthetic construct
  misc_feature 1..72: note = Description of Artificial Sequence: Synthetic oligonucleotide
  source 1..72: mol_type = other RNA
gacctcgtggcgcaacggtagcgcgtctgacttcagatcagaaggctgcgtgttcgaatcacgccggggtca

SEQ ID NO: 624 - RNA, 72 nt, synthetic construct
  misc_feature 1..72: note = Description of Artificial Sequence: Synthetic oligonucleotide
  source 1..72: mol_type = other RNA
gaccttgtggctcaatggtagcgcatctgacttcagatcaggaggttgcacgttcaaatcatgccggggtca

SEQ ID NO: 625 - RNA, 72 nt, synthetic construct
  misc_feature 1..72: note = Description of Artificial Sequence: Synthetic oligonucleotide
  source 1..72: mol_type = other RNA
gaccttgtggcgcaacggtagcgcgtctgacttcagatcagaaggttgcgtgttcaaatcacgtcggggtca

SEQ ID NO: 626 - RNA, 72 nt, synthetic construct
  misc_feature 1..72: note = Description of Artificial Sequence: Synthetic oligonucleotide
  source 1..72: mol_type = other RNA
gacctcgtggcgcaacggtagcgcgtctgacttcagatcagaaggttgcgtattcaaatcacgtcggggtca

SEQ ID NO: 627 - RNA, 72 nt, synthetic construct
  misc_feature 1..72: note = Description of Artificial Sequence: Synthetic oligonucleotide
  source 1..72: mol_type = other RNA
gacctcgtggcgcaacggcagcgcgtctgacttcacattagaaggttgcgtgttcaaatcacgtcggggtca

SEQ ID NO: 628 - RNA, 72 nt, synthetic construct
  misc_feature 1..72: note = Description of Artificial Sequence: Synthetic oligonucleotide
  source 1..72: mol_type = other RNA
gacctcatggcgcaacggtagcgcgtctgacttcagatcagaaggttgcgtgttcaaatcacatcggggtca

SEQ ID NO: 629 - RNA, 72 nt, synthetic construct
  misc_feature 1..72: note = Description of Artificial Sequence: Synthetic oligonucleotide
  source 1..72: mol_type = other RNA
gacctcgtggtgcaacggtagcgcgtatgatttcagatcagaaggttgcgtgttcaaatcacgtcggggtca

SEQ ID NO: 630 - RNA, 72 nt, synthetic construct
  misc_feature 1..72: note = Description of Artificial Sequence: Synthetic oligonucleotide
  source 1..72: mol_type = other RNA
gacctcgtagcgcaacggtagcgcgtctgacttcagatcagaaggttgcgtgttcaaatcacgtcggggtca

SEQ ID NO: 631 - RNA, 76 nt, synthetic construct
  misc_feature 1..76: note = Description of Artificial Sequence: Synthetic oligonucleotide
  source 1..76: mol_type = other RNA
aggggtatagctcaattggcagagcgtcggtcttcaaaaccgaaggttgtaggttcgattcctactgcccctgcca

SEQ ID NO: 632 - RNA, 72 nt, synthetic construct
  misc_feature 1..72: note = Description of Artificial Sequence: Synthetic oligonucleotide
  source 1..72: mol_type = other RNA
gacctcatggcgcaacggtagcgcgtctgacttcagatcagaaggttgcgtgttcaaatcacgtcggggtca

SEQ ID NO: 633 - RNA, 72 nt, synthetic construct
  misc_feature 1..72: note = Description of Artificial Sequence: Synthetic oligonucleotide
  source 1..72: mol_type = other RNA
gacctcgtggcgcaacggtagcgcgtctaacttcagatcagaaggttgcgtgttcaaatcacgtcggggtca

SEQ ID NO: 634 - RNA, 73 nt, synthetic construct
  misc_feature 1..73: note = Description of Artificial Sequence: Synthetic oligonucleotide
  source 1..73: mol_type = other RNA
acgggagtagctcagttggtagagcaccggtcttcaaaaccgggtgtcgggagttcgagcctctcctcccgtg

SEQ ID NO: 635 - RNA, 72 nt, synthetic construct
  misc_feature 1..72: note = Description of Artificial Sequence: Synthetic oligonucleotide
  source 1..72: mol_type = other RNA
gacctcgtggcgcaacggtagcgcgtctgacttcagatcagaaggttgcatgttcaaatcacgtcggggtca

SEQ ID NO: 636 - RNA, 72 nt, synthetic construct
  misc_feature 1..72: note = Description of Artificial Sequence: Synthetic oligonucleotide
  source 1..72: mol_type = other RNA
gactccgtggcgcaacggtagcgcgtccgacttcagatcggaaggttgcgtgttcaaatcacgtcggggtca

SEQ ID NO: 637 - RNA, 72 nt, synthetic construct
  misc_feature 1..72: note = Description of Artificial Sequence: Synthetic oligonucleotide
  source 1..72: mol_type = other RNA
gactccgtggcgcaacggtagcgcgtctgacttcagatcagaaggttgcgtgttcaaatcacgtcggggtca

SEQ ID NO: 638 - RNA, 72 nt, unidentified
  misc_feature 1..72: note = Description of Unknown: TrpChr7.tRNA3-WT sequence
  source 1..72: mol_type = tRNA
ggcctcgtggcgcaacggtagcgcgtctgactccagatcagaaggttgcgtgttcaaatcacgtcggggtca

SEQ ID NO: 639 - RNA, 72 nt, synthetic construct
  misc_feature 1..72: note = Description of Artificial Sequence: Synthetic oligonucleotide
  source 1..72: mol_type = other RNA
ggcctcgtggcgcaacggtagcacgtctgactccagatcagaaggttgcgtgttcaaatcacgtcggggtca

SEQ ID NO: 640 - RNA, 72 nt, synthetic construct; identical to SEQ ID NO: 639
  misc_feature 1..72: note = Description of Artificial Sequence: Synthetic oligonucleotide
  source 1..72: mol_type = other RNA

SEQ ID NO: 641 - RNA, 72 nt, synthetic construct
  misc_feature 1..72: note = Description of Artificial Sequence: Synthetic oligonucleotide
  source 1..72: mol_type = other RNA
ggcctcgtggcgcaacggtagcacgtctgacttcagatcagaaggttgcgtgttcaaatcacgtcggggtca

SEQ ID NO: 642 - RNA, 72 nt, synthetic construct
  misc_feature 1..72: note = Description of Artificial Sequence: Synthetic oligonucleotide
  source 1..72: mol_type = other RNA
ggcctcgtcgcgcaacggtagcgcgtctgacttcagatcagaaggttgcgtgttcaaatcacgtcggggtca

SEQ ID NO: 643 - RNA, 72 nt, synthetic construct
  misc_feature 1..72: note = Description of Artificial Sequence: Synthetic oligonucleotide
  source 1..72: mol_type = other RNA
ggcctcgtcgcgcaacggtagcacgtctgacttcagatcagaaggttgcgtgttcaaatcacgtcggggtca

SEQ ID NO: 644 - protein, 455 aa, synthetic construct
  REGION 1..455: note = Description of Artificial Sequence: Synthetic polypeptide
MSFNTIIDWNSCTAEQQRQLLMRPAISASESITRTVNDILDNVKARGDEALREYSAKFDKTTVTALKVSAEEIAAASERLSDELKQAMAVAVKNIETFHTAQKLPPVDVETQPGVRCQQVTRPVASVGLYIPGGSAPLFSTVLMLATPASIAGCKKVVLCSPPPIADEILYAAQLCGVQDVFNVGGAQAIAALAFGTESVPKVDKIFGPGNAFVTEAKRQVSQRLDGAAIDMPAGPSEVLVIADSGATPDFVASDLLSQAEHGPDSQVILLTPAADMARRVAEAVERQLAELPRAETARQALNASRLIVTKDLAQCVEISNQYGPEHLIIQTRNARELVDSITSAGSVFLGDWSPESAGDYASGTNHVLPTYGYTATCSSLGLADFQKRMTVQELSKEGFSALASTIETLAAAERLTAHKNAVTLRVNALKEQAHHHHHHHHSGGSAWSHPQFEK

SEQ ID NO: 645 - protein, 8 aa, synthetic construct
  REGION 1..8: note = Description of Artificial Sequence: Synthetic 8xHis tag
HHHHHHHH

SEQ ID NO: 646 - protein, 13 aa, synthetic construct
  REGION 1..13: note = Description of Artificial Sequence: Synthetic peptide
KPINQWPANTHER

SEQ ID NO: 647 - DNA, 73 bp, synthetic construct; identical to SEQ ID NO: 522
  misc_feature 1..73: note = Description of Artificial Sequence: Synthetic oligonucleotide
  source 1..73: mol_type = other DNA

SEQ ID NO: 648 - DNA, 10 bp, synthetic construct
  misc_feature 1..10: note = Description of Artificial Sequence: Synthetic oligonucleotide
  source 1..10: mol_type = other DNA
tagtcttcgg

SEQ ID NO: 649 - DNA, 10 bp, synthetic construct
  misc_feature 1..10: note = Description of Artificial Sequence: Synthetic oligonucleotide
  source 1..10: mol_type = other DNA
aagaagaccg

SEQ ID NO: 650 - DNA, 30 bp, synthetic construct
  misc_feature 1..30: note = Description of Artificial Sequence: Synthetic oligonucleotide
  source 1..30: mol_type = other DNA
gtcctttttttgctttagtgagggttaatt

SEQ ID NO: 651 - DNA, 59 bp, synthetic construct
  misc_feature 1..59: note = Description of Artificial Sequence: Synthetic oligonucleotide
  source 1..59: mol_type = other DNA
gaattcttcccgagacgttccaagtcttcatgaagactacaggcgtctcccaggaagct

SEQ ID NO: 652 - DNA, 92 bp, synthetic construct
  misc_feature 1..92: note = Description of Artificial Sequence: Synthetic oligonucleotide
  source 1..92: mol_type = other DNA
attatgctgagtgatatccggagcaccgcgttgccatcgcgcagactgaagtctagtcttccaacgcacaagtttagtgcagccccagtggt

SEQ ID NO: 653 - DNA, 89 bp, synthetic construct
  misc_feature 1..89: note = Description of Artificial Sequence: Synthetic oligonucleotide
  source 1..89: mol_type = other DNA
attatgctgagtgatatcgcaaccaccatataccaatcgtatcgaggaagtttcgtaactgggcccaagctaagggccggttgcgtggt

SEQ ID NO: 654 - RNA, 76 nt, synthetic construct
  source 1..76: mol_type = other RNA
  misc_feature 64: map = nt 64 is "t"
gcggatttagctcagddgggagagcgccagactgaayatctggaggtcctgtgttcgatccacagaattcgcacca

SEQ ID NO: 655 - RNA, 73 nt, synthetic construct
  source 1..73: mol_type = other RNA
gacctcgtggcgcaacggttagcgcgtctgacttcagatcagaaggctgcgtgttcgaatcacgtcggggtca